(19) United States
(12) Patent Application Publication
(10) Pub. No.: US 2021/0002640 A1
(43) Pub. Date: Jan. 7, 2021

(54) LIPOSOMAL SPHERICAL NUCLEIC ACID (SNA) CONSTRUCTS FOR SURVIVAL OF MOTOR NEURON (SMA) INHIBITORS

(71) Applicant: Exicure, Inc., Skokie, IL (US)

(72) Inventors: Richard Kang, Wilmette, IL (US); Subbarao Nallagatla, Skokie, IL (US); Bart Anderson, Morton Grove, IL (US); Ekambar Kandimalla, Hopkinton, MA (US); Michael Mutolo, Skokie, IL (US)

(73) Assignee: Exicure, Inc., Skokie, IL (US)

(21) Appl. No.: 16/976,168

(22) PCT Filed: Feb. 28, 2019
(86) PCT No.: PCT/US2019/020164

§ 371 Date: Aug. 27, 2020

Related U.S. Application Data
(60) Provisional application No. 62/636,764, filed on Feb. 28, 2018.
(60) Provisional application No. 62/664,055, filed on Apr. 27, 2018.
(60) Provisional application No. 62/684,476, filed on Jun. 13, 2018.
(60) Provisional application No. 62/691,585, filed on Jun. 28, 2018.
(60) Provisional application No. 62/740,398, filed on Oct. 2, 2018.

(51) Int. Cl. C12N 15/113; A61K 47/69; A61K 9/00; A61P 21/00

(52) U.S. Cl. CPC C12N 15/113; A61K 47/6911; A61K 47/6923; A61K 9/0019; A61P 21/00; C12N 2320/33; C12N 2310/3515; C12N 2310/351; C12N 2310/315; C12N 2310/321; C12N 2310/3233; C12N 2310/11

(57) ABSTRACT

Compositions related to spherical nucleic acids (SNAs) and structures with antisense oligonucleotides and methods of treatment of diseases and disorders are disclosed herein.

### RELATED APPLICATION

This application claims the benefit under 35 U.S.C § 119(e) of the filing date of U.S. Provisional Application Ser. No. 62/636,764, filed Feb. 28, 2018, entitled “LIPOSOMAL SPHERICAL NUCLEIC ACID (SNA) CONSTRUCTS FOR SURVIVAL OF MOTOR NEURON (SMA) INHIBITORS”, of U.S. Provisional Application Ser. No. 62/664,055, filed Apr. 27, 2018, entitled “LIPOSOMAL SPHERICAL NUCLEIC ACID (SNA) CONSTRUCTS FOR SURVIVAL OF MOTOR NEURON (SMA) INHIBITORS”, of U.S. Provisional Application Ser. No. 62/684,476, filed Jun. 13, 2018, entitled “LIPOSOMAL SPHERICAL NUCLEIC ACID (SNA) CONSTRUCTS FOR SURVIVAL OF MOTOR NEURON (SMA) INHIBITORS”, of U.S. Provisional Application Ser. No. 62/691,585, filed Jun. 28, 2018, entitled “LIPOSOMAL SPHERICAL NUCLEIC ACID (SNA) CONSTRUCTS FOR SURVIVAL OF MOTOR NEURON (SMA) INHIBITORS”, and of U.S. Provisional Application Ser. No. 62/740,398, filed Oct. 2, 2018, entitled “LIPOSOMAL SPHERICAL NUCLEIC ACID (SNA) CONSTRUCTS FOR SURVIVAL OF MOTOR NEURON (SMA) INHIBITORS”, the entire contents of each of which are incorporated herein by reference.

### BACKGROUND

Spinal muscular atrophy (SMA) is an autosomal recessive neurodegenerative disorder characterized by progressive muscle wasting and loss of muscle function due to severe motor neuron dysfunction. SMA is the leading genetic cause of infant mortality. SMA is caused by low levels of Survival of Motor Neuron (SMN) due to deletion or loss of function of SMN1 gene.

### SUMMARY

Compositions and methods for SMN protein levels and treating diseases are provided herein. According to some aspects, spherical nucleic acids (SNA) are contemplated. In some embodiments, a SNA comprises a core and an antisense oligonucleotide comprised of 8 to 50 linked nucleosides in length targeted to ISS-N1 site of Survival of Motor Neuron 2 (SMN2) pre-mRNA, and wherein the antisense oligonucleotide is attached to the core and forms an oligonucleotide shell.

In some embodiments, the core has a minimal number mean diameter of about 8 nm. In some embodiments, wherein the core has a minimal number mean diameter of about 10 nm. In some embodiments, the core has a minimal number mean diameter of about 15 nm. In some embodiments, the core has a number mean diameter of about 10 nm to about 50 nm. In some embodiments, the core has a number mean diameter of about 20 nm to about 25 nm. in some embodiments, the core has a number mean diameter of about 20 nm. In some embodiments, the core has a number mean diameter of about 25 nm. In some embodiments, the core has a number mean diameter of about 10 nm to about 15 nm. In some embodiments, the core has a number mean diameter of about 13 nm.

In some embodiments, the regulatory site is a ISS-N1 site. In some embodiments, the regulatory site is a E1 site, a 3′ splice site of exon 8 site or a ISS+100 site.

In some embodiments, the core is a lipid bilayer containing core or liposomal core and the antisense oligonucleotide is attached to the lipid bilayer containing core or liposomal core. In some embodiments, the core is a metal core. In some embodiments, the core is an inorganic metal core. In some embodiments, the core is a gold core. In some embodiments, the antisense oligonucleotide is attached to the gold core through a covalent interaction.

In some embodiments, the antisense oligonucleotide is 18 nucleotides in length.

In some embodiments, the ISS-N1 site of the SMN2 pre-mRNA comprises a nucleic acid sequence of SEQ ID NO: 15.

In some embodiments, less than all of the internucleoside linkages are phosphodiester. In some embodiments, the antisense oligonucleotide has phosphorothioate internucleoside linkages. In some embodiments, less than all of the internucleoside linkages are phosphorothioate.

In some embodiments, the antisense oligonucleotide has 2′O (2-methoxyethyl) modifications. In some embodiments, less than all of the nucleotides include a 2′O (2-methoxyethyl) modification. In some embodiments, the antisense oligonucleotide has LNA modifications. In some embodiments, less than all of the nucleotides include a LNA modification. In some embodiments, the antisense oligonucleotide has morpholino modifications. In some embodiments, less than all of the nucleotides include a morpholino modification.

In some embodiments, the antisense oligonucleotide has 2′O methyl modifications. In some embodiments, less than all of the nucleotides include a 2′O methyl modification.

In some embodiments, the antisense oligonucleotide has 2′O (2-methoxyethyl) modifications. In some embodiments, less than all of the nucleotides include a 2′O (2-methoxyethyl) modification.

In some embodiments, the antisense oligonucleotide is comprised of 18 to 21 linked nucleosides. In other embodiments the antisense oligonucleotide is comprised of 1 to 10, 8-20, 8-30, 10-20, 10-30, 10-40, 15-20, 15-30, 15-40, 18-20, 18-25, 18-30, 18-35, 18-40, 18-45 or 18-50 linked nucleosides.

In some embodiments, the antisense oligonucleotides of the oligonucleotide shell are directly attached to the lipid bilayer containing core. In some embodiments, the antisense oligonucleotides of the oligonucleotide shell are indirectly attached to the lipid bilayer containing core through a linker.

In some embodiments, the linker comprises a molecular species at the 3′ or 5′ termini of the antisense oligonucleotide, wherein the molecular species is positioned in a core and the antisense oligonucleotide extends radially from the core.

In some embodiments, the molecular species is linked to the antisense oligonucleotide at the 5′ end of the antisense oligonucleotide. In some embodiments, the molecular species is a hydrophobic group.

In some embodiments, the hydrophobic group is selected from the group consisting of cholesterol, a cholesteryl or modified cholesteryl residue, a stearyl, a distearyl, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. In some embodiments, the hydrophobic group is cholesterol. In some embodiments, the hydrophobic group is distearyl.

In some embodiments, the linker moiety comprises a non-nucleotidic linker moiety linked to the molecular species. In some embodiments, the non-nucleotidic linker moiety is selected from the group consisting of an abasic residue (dSpacer), oligoethyleneglycol, triethyleneglycol, hexaethyleneglycol, polyethylene glycol, alkane-diol, or butanediol. In some embodiments, the non-nucleotidic linker moiety is a double linker. In some embodiments, the double linker is two oligoethyleneglycols. In some embodiments, the two oligoethyleneglycols are triethyleneglycol. In some embodiments, the two oligoethyleneglycols are hexaethyleneglycol. In some embodiments, the double linker is two alkane-diols. In some embodiments, the two alkane-diols are butanediol. In some embodiments, the double linker is linked in the center by a phosphodiester, phosphorothioate, methylphosphonate, or amide linkage.

In some embodiments, the non-nucleotidic linker moiety is a triple linker. In some embodiments, the triple linker is three oligoethyleneglycols. In some embodiments, the three oligoethyleneglycols are triethyleneglycol. In some embodiments, the three oligoethyleneglycols are hexaethyleneglycol. In some embodiments, the triple linker is three alkane-diols. In some embodiments, the three alkane-diols are butanediol. In some embodiments, the triple linker is linked in between each single linker by a phosphodiester, phosphorothioate, methylphosphonate, or amide linkage.

In some embodiments, the antisense oligonucleotides comprise the entire SNA such that no other structural components are part of the nanostructure and wherein the antisense oligonucleotide includes a molecular species and non-nucleotidic linker moiety that form the core, with the antisense oligonucleotides extending radially from the core.

In some embodiments, the SNA is free of lipids, cell penetrating peptides, cationic peptides, polymers or solid cores.

In some embodiments, oligonucleotide shell has a density of 5-1,000 oligonucleotides per SNA. In some embodiments, the oligonucleotide shell has a density of 100-1,000 oligonucleotides per SNA. In some embodiments, the oligonucleotide shell has a density of 500-1,000 oligonucleotides per SNA.

In some embodiments, the lipid bilayer containing core is comprised of one or more lipids selected from: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof.

In some embodiments, the lipid bilayer containing core or liposomal core is comprised of DOPC.

In some embodiments, the ratio of number of oligonucleotide molecules to the diameter of the lipid bilayer containing core or liposomal core of DOPC in nm is 30:20.

In some embodiments, a SNA described herein comprises an antisense oligonucleotide that comprises or consists of

(SEQ ID NO: 1)
5′ - TCA CTT TCA TAA TGC TGG - (Spacer 18)₂ -
3CholTEG.

According to another aspect, methods for treating a subject having spinal muscular atrophy (SMA) are provided. In some embodiments, the method comprises administering to a subject having SMA a spherical nucleic acid (SNA), in an effective amount to increase expression levels of SMN2 protein over a baseline level in the subject in order to treat the disorder.

In some embodiments, the baseline level is the level of SMN2 protein in the subject prior to treatment with the SNA. In some embodiments, the baseline level is the level of SMN2 protein in a subject having SMA and treated with a linear antisense oligonucleotide targeted to ISS-N1 site of SMN2 pre-mRNA.

In some embodiments, the SNA is delivered by a route selected from the group consisting of intrathecal, oral, nasal, sublingual, intravenous, subcutaneous, mucosal, respiratory, direct injection, and dermally.

According to another aspect, methods for treating a subject having spinal muscular atrophy (SMA) are provided.

In some embodiments, the method comprises administering to a subject having SMA at least two doses of a spherical nucleic acid (SNA), in an effective amount to increase expression levels of Survival of Motor Neuron 2 (SMN2) protein over a baseline level in the subject in order to treat the disorder, wherein the second dose is administered about 3 months to 2 years after the first dose, and wherein the SNA comprises a core and an antisense oligonucleotide comprised of 10 to 40 linked nucleosides in length targeted to a region of SMN2 pre-mRNA, such that a level of exon 7-containing SMN2 mRNA relative to exon 7-deleted SMN2 mRNA in the subject is enhanced, and wherein the oligonucleotides are attached to the core and thus form an oligonucleotide shell.

According to another aspect, methods of enhancing a level of exon 7-containing Survival of Motor Neuron 2 (SMN2) mRNA relative to exon-deleted SMN2 mRNA in a cell are provided.

In some embodiments, the method comprises contacting the cell with a spherical nucleic acid (SNA) comprising a core and an antisense oligonucleotide comprised of 10 to 40 linked nucleosides in length targeted to a region of SMN2 pre-mRNA, such that the level of exon 7-containing SMN2 mRNA relative to exon 7-deleted SMN2 mRNA in the cell is enhanced.

In some embodiments, the core is a lipid bilayer containing core or liposomal core and the antisense oligonucleotide is attached to the lipid bilayer containing core or liposomal core. In some embodiments, the core is a metal core. In some embodiments, the core is an inorganic metal core. In some embodiments, the core is a gold core. In some embodiments, the antisense oligonucleotide is attached to the gold core through a covalent interaction. In some embodiments, the cell is a cell in connective tissue. In some embodiments, the cell is a motor neuron. In some embodiments, the cell is a spinal motor neuron.

In some embodiments, the antisense oligonucleotide comprises a sequence which is complementary to a portion of intron 7 of the SMN2 gene or the SMN2 pre-mRNA.

According to another aspect, a spherical nucleic acid (SNA) comprising a core and an antisense oligonucleotide comprised of 8 to 50 linked nucleosides in length targeted to a regulator of splicing of Survival of Motor Neuron 2 (SMN2) pre-mRNA, and wherein the antisense oligonucleotide is attached to the core and forms an oligonucleotide shell is contemplated herein.

In some embodiments, the regulator of splicing of SMN2 pre-mRNA regulates inclusion of exon 7 in the SMN2 mRNA. In some embodiments, the regulator of splicing of SMN2 pre-mRNA is an RNA binding protein. In some embodiments, the RNA binding protein is RBM10.

In some embodiments, the regulator of splicing of SMN2 pre-mRNA is a serine/arginine (SR) splicing factor or a heterogeneous ribonucleoprotein (hnRNP) protein. In some embodiments, the SR splicing factor is SRSF1, SRSF2, SRSF3, SRSF4, SRSF5, SRSF6, SRSF7 or SRSF11. In some embodiments, the hnRNP protein is hnRNPA1, hnRNP A2B1, hnRNP C or hnRNP U.

In some embodiments, the regulator of splicing of SMN2 pre-mRNA is HuR/ELAVL1, Puf60, Sam68, SF1, SON, U2AF35 or ZIS2/ZNF265.

In some embodiments the SNA has an average or number mean diameter (average or number mean diameter are used interchangeably herein) on the order of 10-100 nanometers. In some embodiments, the number mean diameter of the nanoparticle is from about 15 nm to about 100 nm, about 20 nm to about 100 nm, about 25 nm to about 100 nm, about 150 nm to about 50 nm, about 15 nm to about 50 nm, about 20 nm to about 50 nm, about 10 nm to about 70 nm, about 15 nm to about 70 nm about 20 nm to about 70 nm, about 10 nm to about 30 nm, about 15 nm to about 30 nm, about 20 nm to about 30 nm, about 10 nm to about 40 nm, about 15 nm to about 40 nm, about 20 nm to about 40 nm, about 10 nm to about 80 nm, about 15 nm to about 80 nm, or about 20 nm to about 80 nm in number mean diameter.

In some aspects, the invention is a spherical nucleic acid (SNA), comprising a core and a first antisense oligonucleotide comprised of 8 to 50 linked nucleosides in length targeted to a regulatory site of Survival of Motor Neuron 2 (SMN2) pre-mRNA, and a second antisense oligonucleotide comprised of 8 to 50 linked nucleosides in length targeted to a region of a lncRNA, and wherein the antisense oligonucleotides are attached to the core and form an oligonucleotide shell.

The core in some embodiments has a minimal number mean diameter of about 8 nm, about 10 nm, or about 15 nm, or about 25 nm, or has a number mean diameter of about 10 nm to about 50 nm, about 20 nm to about 25 nm, or about 20 nm.

In some embodiments the core is a lipid bilayer containing core and the antisense oligonucleotide is attached to the lipid bilayer containing core.

In some embodiments, the lncRNA is SMN-AS1, GenBank accession #BC045789.1. In embodiments the second antisense oligonucleotide is selected from SEQ ID NO: 81 to SEQ ID NO:160. In other embodiments the second antisense oligonucleotide is selected from oligonucleotides having 85%, 90%, 95%, 96%, 97%, 98%, or 99% sequence identity with oligonucleotides of SEQ ID NO: 81 to SEQ ID NO: 160. In some embodiments, the second antisense oligonucleotide has a 5-10-5 MOE gapmer design, wherein the central gap segment comprises of ten 2′-deoxynucleosides and is flanked by wing segments on the 5′ direction and the 3′ direction comprising five nucleosides each. Each nucleoside in the 5′ wing segment and/or each nucleoside in the 3′ wing segment may in some embodiments have a 2′-MOE modification. The internucleoside linkages throughout each gapmer are phosphorothioate (P═S) linkages in some embodiments. In some embodiments, the gapmers have mixed backbone, including phosphorothioate and phosphodiester internucleotide linkages. In some embodiments, one or more or all cytosine residues throughout each gapmer are 5-methylcytosines.

In other aspects the invention is a method of increasing expression of full length SMN2 mRNA in a cell comprising contacting the cell with an SNA disclosed herein.

A method of increasing expression of full length SMN2 mRNA in a cell is provided in other aspects of the invention. The method involves contacting the cell with a spherical nucleic acid (SNA) comprising a core and an antisense oligonucleotide comprised of 10 to 40 linked nucleosides in length targeted to a region of SMN2 pre-mRNA and another SNA comprising a core and an antisense oligonucleotide comprised of 10 to 40 linked nucleosides in length targeted to a region of SMN-AS1.

In some embodiments, the core is a lipid bilayer containing core or liposomal core and the antisense oligonucleotide is attached to the lipid bilayer containing core or liposomal core. In some embodiments, the core is a metal core. In some embodiments, the core is an inorganic metal core. In some embodiments, the core is a gold core. In some embodiments, the antisense oligonucleotide is attached to the gold core through a covalent interaction. In some embodiments, the cell is a cell in connective tissue. In some embodiments, the cell is a motor neuron. In some embodiments, the cell is a spinal motor neuron. In some embodiments, the molecular species is linked to the antisense oligonucleotide at the 3′ end of the antisense oligonucleotide.

According to another aspect, methods for delivering a stable level of therapeutic oligonucleotides are provided herein.

In some embodiments, a stable level of therapeutic oligonucleotides is delivered to a CNS of a subject, wherein the method comprises administering to the subject having SMA a spherical nucleic acid (SNA), wherein the SNA comprises a core and therapeutic oligonucleotides comprised of 10 to 60 linked nucleosides in length, wherein the therapeutic oligonucleotides are attached to the core and thus form an oligonucleotide shell, wherein the SNA is administered in an effective amount to deliver a stable level of the therapeutic oligonucleotide to the CNS of the subject, wherein the stable level of the therapeutic oligonucleotides is achieved when at least 50% of the therapeutic oligonucleotides are present in a tissue of the CNS within seven days of administration of the SNA to the subject, relative to the amount of therapeutic oligonucleotides present in the tissue of the CNS within one hour of administration of the SNA to the subject.

In some embodiments, the SNA is administered intrathecally (IT).

In some embodiments, the SNA is administered in the lower lumbar region. In some embodiments, the SNA is IT-administered through a lumbar puncture.

In some embodiments, the subject is a mammal. In some embodiments, the subject is a rat. In some embodiments, the subject is a human.

In some embodiments, a stable level comprises having at least 50% of the therapeutic oligonucleotides present in a tissue of the CNS within three days of administration of the SNA to the subject, relative to the amount of therapeutic oligonucleotides present in the tissue of the CNS within one hour of administration of the SNA to the subject. In some embodiments, a stable level is achieved when at least 50% of the therapeutic oligonucleotides are present in a tissue of the CNS within 48 hours of administration of the SNA to the subject, relative to the amount of therapeutic oligonucleotides present in the tissue of the CNS within one hour of administration of the SNA to the subject. In some embodiments, a stable level is achieved when at least 50% of the therapeutic oligonucleotides are present in a tissue of the CNS within 24 hours of administration of the SNA to the subject, relative to the amount of therapeutic oligonucleotides present in the tissue of the CNS within one hour of administration of the SNA to the subject.

In some embodiments, the therapeutic olligonucleotide is an antisense oligonucleotide comprised of 10 to 40 linked nucleosides in length targeted to a regulatory site of Survival of Motor Neuron 2 (SMN2) pre-mRNA. In some embodiments, the level of exon 7-containing SMN2 mRNA relative to exon 7-deleted SMN2 mRNA in the subject is enhanced.

In some embodiments, less than 50% of the therapeutic oligonucleotides are detectable within six hours of administration to the subject in one or both kidneys of the subject. In some embodiments, less than 40% of the therapeutic oligonucleotides are detectable within six hours of administration to the subject in one or both kidneys of the subject. In some embodiments, less than 30% of the therapeutic oligonucleotides are detectable within six hours of administration to the subject in one or both kidneys of the subject. In some embodiments, less than 20% of the therapeutic oligonucleotides are detectable within six hours of administration to the subject in one or both kidneys of the subject. In some embodiments, less than 10% of the therapeutic oligonucleotides are detectable within six hours of administration to the subject in one or both kidneys of the subject. In some embodiments, less than 5% of the therapeutic oligonucleotides are detectable within six hours of administration to the subject in one or both kidneys of the subject.

In some embodiments, any of the SNAs described herein are used.

In some embodiments, the SNA is in a formulation and wherein the formulation comprises artificial cerebral spinal fluid (aCSF).

According to another aspect, methods for delivering a stable level of therapeutic oligonucleotides are provided herein

in some embodiments, a method for delivering a stable level of therapeutic oligonucleotides to a central nervous system (CNS) of a subject having spinal muscular atrophy (SMA) comprises administering to a subject having SMA a spherical nucleic acid (SNA) in an effective amount to deliver therapeutic oligonucleotides to one or more tissues or regions of the CNS of the subject, wherein the administration of SNA delivers about 2% to about 150% more therapeutic oligonucleotides to one or more tissues or regions of the CNS of the subject than administration of linear therapeutic oligonucleotides that are not in a SNA, wherein the SNA comprises a core and therapeutic oligonucleotides comprised of 10 to 60 linked nucleosides in length, wherein the therapeutic oligonucleotides are attached to the core and thus form an oligonucleotide shell.

In some embodiments, the one or more tissues or regions of the CNS is one or more regions of the brain. In some embodiments, the one or more regions of the brain is selected from the group consisting of the amygdala, basal ganglia, cerebellum, corpus callosum, cortex, hippocampus, hypothalamus, midbrain, olfactory region, one or more ventricles, septal area, white matter and thalamus. In some embodiments, the one or more tissues or regions of the CNS are the cervical cerebral spinal fluid (CSF) or thoracic CSF.

In some embodiments, the therapeutic oligonucleotides in the SNA and the linear therapeutic oligonucleotides that are not in a SNA have different routes of distribution and clearance.

According to another aspect, methods for treating a subject having spinal muscular atrophy (SMA) are provided herein.

In some embodiments, a method for treating a subject having spinal muscular atrophy (SMA) comprises administering to the subject having SMA a spherical nucleic acid (SNA) in an effective amount to increase the expression level of survival of motor neuron 2 (SMN2) protein over a baseline level of SMN2 protein in the central nervous system (CNS) of the subject to treat SMA, wherein the effective amount of SNA is greater than 12 mg/dose, and wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and wherein the antisense oligonucleotides are attached to the core and thus form an oligonucleotide shell.

According to another aspect, methods for treating a subject having spinal muscular atrophy (SMA) are provided herein.

In some embodiments, the method for treating a subject having spinal muscular atrophy (SMA) comprises administering to the subject having SMA a spherical nucleic acid (SNA) in an effective amount to increase the expression level of survival of motor neuron 2 (SMN2) protein over a baseline level of SMN2 protein in the central nervous system (CNS) of the subject to treat SMA, wherein the effective amount of SNA is less than 12 mg/dose, and wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and wherein the antisense oligonucleotides are attached to the core and thus form an oligonucleotide shell.

According to another aspect, methods for treating a subject having spinal muscular atrophy (SMA) are provided herein.

In some embodiments, a method for treating a subject having spinal muscular atrophy (SMA) comprises administering to a subject having SMA at least two doses of a spherical nucleic acid (SNA) in an effective amount to increase expression levels of survival of motor neuron 2 (SMN2) protein over a baseline level in the subject in order to treat SMA, wherein the second dose is administered about 15 days to about three months after the first dose, and wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and wherein the oligonucleotides are attached to the core and thus form an oligonucleotide shell.

In some embodiments, the second dose is administered about two years after the first dose. In some embodiments, the second dose is administered about 1.5 years after the first dose. In some embodiments, the second dose is administered about one year after the first dose. In some embodiments, the second dose is administered about six months after the first dose. In some embodiments, the second dose is administered about four months after the first dose. In some embodiments, the second dose is administered about three months after the first dose. In some embodiments, the second dose is administered about two months after the first dose. In some embodiments, the second dose is administered about one month after the first dose.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

According to some aspects, a structure is also contemplated herein.

In some embodiments, the structure comprises an antisense oligonucleotide comprised of 8 to 50 linked nucleosides in length targeted to a regulatory site of Survival of Motor Neuron 2 (SMN2) pre-mRNA and a linker comprising a molecular species at the 3′-end or the 5′-end of the antisense oligonucleotide, wherein the linker comprises two oligoethylene glycols. In some embodiments, the oligoethylene glycol is a hexaethylene glycol.

In some embodiments, the structure comprises an antisense oligonucleotide comprising the nucleotide sequence 5′-TCACTTTCATAATGCTGG-3′ (SEQ ID NO: 172) or the nucleotide sequence 5′-Tes mCes Aes mCes Tes Tes Tes mCes Aes Tes Aes Aes Tes Ges mCes Tes Ges Ge-3′ (SEQ ID NO: 16) and a linker at the 3′-end or the 5′-end of the antisense oligonucleotide comprising two oligoethylene glycols and a cholesterol. In some embodiments, the oligoethylene glycol is a hexaethylene glycol. In some embodiments, the structure comprises an antisense oligonucleotide comprising the nucleotide sequence 5′-UCACUUUCAUAAUGCUGG-3′ (SEQ ID NO: 173)

In some embodiments, the structure comprises an antisense oligonucleotide comprising or consisting of the nucleotide sequence 5′-TCA CTT TCA TAA TGC TGG-(Spacer 18)2-3CholTEG (SEQ ID NO: 1) or the nucleotide sequence moeT*/5-Me-moeC/*moeA*/5-Me-moeC/*moeT*moeT*moeT*/5-Me-moeC/*moeA*moeT*moeA*moeA*moeT*moeG*/5-Me-moeC/*moeT*moeG*moeG/isp18//isp18//3CholTEG/(SEQ ID NO: 164).

In some embodiments, the structure comprises an antisense oligonucleotide comprised of 8 to 50 linked nucleosides in length targeted to a regulatory site of Survival of Motor Neuron 2 (SMN2) pre-mRNA and a linker comprising a molecular species at the 3′-end or the 5′-end of the antisense oligonucleotide, wherein the linker comprises two oligoethylene glycols and wherein the molecular species comprises a cholesterol. In some embodiments, the oligoethylene glycol is a hexaethylene glycol.

In some embodiments, the structure comprises an antisense oligonucleotide comprised of 8 to 50 linked nucleosides in length targeted to a regulatory site of Survival of Motor Neuron 2 (SMN2) pre-mRNA and a linker comprising a molecular species at the 3′-end or the 5′-end of the antisense oligonucleotide, wherein the molecular species is a hydrophobic group comprising a stearyl. In some embodiments, the stearyl is a distearyl.

According to some aspects, methods for treating a subject having spinal muscular atrophy (SMA) are also contemplated herein.

In some embodiments, the method for treating a subject having SMA comprises administering to a subject having SMA a structure disclosed herein in an effective amount to increase expression levels of SMN2 protein over a baseline level in the subject in order to treat the disorder.

In some embodiments, the method for treating a subject having SMA comprises administering to a subject having SMA at least two doses of a structure in an effective amount to increase expression levels of Survival of Motor Neuron 2 (SMN2) protein over a baseline level in the subject in order to treat the disorder, wherein the second dose is administered about 3 months to 2 years after the first dose, and wherein the structure comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced.

According to some aspects, methods of enhancing a level of exon 7-containing SMN2 mRNA are also contemplated herein.

In some embodiments, the method of enhancing a level of exon 7-containing SMN2 mRNA relative to exon-deleted Survival of Motor Neuron 2 (SMN2) mRNA in a cell comprises contacting the cell with a structure disclosed herein, such that the level of exon 7-containing SMN2 mRNA relative to exon 7-deleted SMN2 mRNA in the cell is enhanced.

According to some aspects, a structure comprising an antisense oligonucleotide comprised of 8 to 50 linked nucleosides in length targeted to a region of a lncRNA, wherein the structure comprises a linker, is contemplated herein.

According to some aspects, methods for increasing expression of full length SMN2 mRNA in a cell are contemplated herein. In some embodiments, the method comprises contacting the cell with structure comprising an antisense oligonucleotide comprised of 10 to 40 linked nucleosides in length targeted to a region of SMN2 pre-mRNA and contacting the cell with another structure comprising an antisense oligonucleotide comprised of 10 to 40 linked nucleosides in length targeted to a region of SMN-AS1.

According to some aspects, methods for delivering a stable level of therapeutic oligonucleotides to a central nervous system (CNS) of a subject are contemplated herein.

In some embodiments, the method for delivering a stable level of therapeutic oligonucleotides to a CNS of a subject comprises administering to the subject a structure disclosed herein in an effective amount to deliver a stable level of the therapeutic oligonucleotide to the CNS of the subject, wherein the stable level of the therapeutic oligonucleotides is achieved when at least 50% of the therapeutic oligonucleotides are present in a tissue of the CNS within seven days of administration of the structure to the subject, relative to the amount of therapeutic oligonucleotides present in the tissue of the CNS within one hour of administration of the structure to the subject.

According to some aspects, methods for treating a subject having SMA are contemplated herein.

In some embodiments, the method for treating a subject having SMA comprises administering to the subject having SMA a structure disclosed herein in an effective amount to increase the expression level of survival of motor neuron 2 (SMN2) protein over a baseline level of SMN2 protein in the central nervous system (CNS) of the subject to treat SMA, wherein the effective amount of structure is greater than 12 mg/dose, such that a level of exon 7-containing SMN2 mRNA relative to exon 7-deleted SMN2 mRNA in the subject is enhanced.

In some embodiments, the method for treating a subject having spinal muscular atrophy (SMA) comprises administering to the subject having SMA a structure disclosed herein in an effective amount to increase the expression level of survival of motor neuron 2 (SMN2) protein over a baseline level of SMN2 protein in the CNS of the subject to treat SMA, wherein the effective amount of structure is less than 12 mg/dose, such that a level of exon 7-containing SMN2 mRNA relative to exon 7-deleted SMN2 mRNA in the subject is enhanced.

In some embodiments, the method for treating a subject having SMA comprises administering to a subject having SMA at least two doses of a structure disclosed herein in an effective amount to increase expression levels of survival of motor neuron 2 (SMN2) protein over a baseline level in the subject in order to treat SMA, wherein the second dose is administered about 15 days to about three months after the first dose, such that a level of exon 7-containing SMN2 mRNA relative to exon 7-deleted SMN2 mRNA in the subject is enhanced.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show the fold increase of SMN2 mRNA over SMN Δ7 mRNA following 48 hour treatment of SMA patient-derived fibroblasts with the compounds. FIG. 1A (Full-length SMN mRNA) and FIG. 1B (Δ7 SMN mRNA)

FIGS. 2A-2B show SMN2 protein, mRNA detection and quantification (72 hours). FIG. 2A Western blot showing total SMN protein and loading control GRP94. FIG. 2B is a densitometric quantification of SMN western blot (solid bars) and qRT-PCR of full-length SMN mRNA (hashed bars) from identically treated wells.

FIGS. 3A-3B show Kaplan-Meier survival plots of SMA mice treated with a single intracerebro-ventricular (ICV) injection of SNA-ASO or linear ASO at 10, 20 or 30 μg doses at age P0 (post natal day 0). Linear represents linear ASO and SNA represents SNA-ASO. FIG. 3A shows Δ7SMA mice treated with the 30 μg dose Nusinersen-SNA had increased survival to a maximum of 82 days while scramble SNA has no effect on survival. FIG. 3B shows that linear Nusinersen improved survival of Δ7 SMA mice to a maximum of 28 days.

FIGS. 4A-4B show increase in body weight of SMA mice treated with a single ICV injection of SNA-ASO at 10, 20 or 30 μg or linear ASO at 10 or 20 μg doses. Mice in 30 μg SNA-ASO group have not reached end point. Linear represents linear ASO and SNA represents SNA-ASO. FIG. 4A shows that weights are similar in Δ7SMA mice treated with linear or Nusinersen-SNA treated mice. FIG. 4B shows that weights are similar in Δ7SMA mice treated with morpholino to ISS-N1 or Nusinersen-SNA.

FIG. 5 shows a bar graph depicting increased exon 7 incorporation in SMN2 mRNA transcript in SMA mice treated with SNA-ASO (30 μg single dose on P0) compared with untreated mice on P10. SNA ISS-N1 represents SNA-ASO.

FIG. 6 shows distribution of linear ASO and SNA ASO in the whole brain over 7 day period following single intrathecal administration in lower lumbar region of SD rat. ID=injected dose.

FIG. 7 shows ¹²⁵I-ASO distribution in Sprague Dawley rats.

FIG. 8 shows ¹²⁵I-ASO concentration in kidneys (% ID/g).

FIG. 9 shows ¹²⁵I-ASO group mean for all brain regions in % ID/g.

FIG. 10 shows ¹²⁵I-ASO concentration in the olefactory region (% ID/g).

FIG. 11 shows ¹²⁵I-ASO concentration in the whole brain (% ID/g).

FIG. 12 shows ¹²⁵I-ASO concentration in the ventricles (% ID/g).

FIG. 13 shows ¹²⁵I-ASO concentration in whole blood and plasma at 168 h.

FIG. 14 shows ¹²⁵I-ASO concentration in the spleen (% ID/g).

FIG. 15 shows ¹²⁵I-ASO concentration in the liver (% ID/g).

FIG. 16 shows ¹²⁵I-ASO concentration in the thyroid (% ID/g).

FIG. 17 shows ¹²⁵I-ASO in superficial cervical lymph nodes (% ID/g).

FIG. 18 shows ¹²⁵I-ASO in deep cervical lymph nodes (% ID/g).

FIG. 19 shows ¹²⁵I-ASO concentration in the CSF and thoracic region (% ID/g).

FIG. 20 shows ¹²⁵I-ASO in lumbar CSF (% ID/g).

FIG. 21 shows ¹²⁵I-ASO in cervical CSF (% ID/g).

FIG. 22 shows ¹²⁵I-ASO concentration in the septal area (% ID/g).

FIGS. 23A-23B are a table showing the average percent injected dose per gram of tissue over 7 days for various organs and regions of brain and spinal cord in rats. The top third of the table shows the values for linear ASO, middle third for SNA ASO and bottom third shows the ratio of SNA ASO to linear ASO.

FIGS. 24A and 24B show quantification of full-length and Δ7 variants of SMN2 mRNA transcripts in SMA patient fibroblasts after treatment with liposomal or gold SNAs. Fold changes in SMN2 mRNA levels were calculated relative to the untreated fibroblasts. Lipidated oligonucleotides were also tested alone without being functionalized on an SNA core. FIG. 24A shows Liposomal vs Gold SNA: Full-length SMN2 mRNA and FIG. 24B shows Liposomal vs Gold SNA: Δ7 SMN2 mRNA.

### DETAILED DESCRIPTION

Spinal Muscular Atrophy (SMA) is an autosomal recessive neurodegenerative disorder characterized by progressive muscle wasting and loss of muscle function due to severe motor neuron dysfunction. SMA is caused by low levels of Survival of Motor Neuron (SMN) due to deletion or loss of function of SMN1 gene. Humans carry a second copy of SMN gene, SMN2. However, due to a mutation in exon 7, SMN2 exon 7 is inefficiently spliced producing a truncated protein SMNΔ7, which is unstable and only partially functional. While several additional splice isoforms are generated by alternative splicing of both SMN1 and SMN2, SMNΔ7 transcript appears to be the major isoform produced by SMN2.

Due to the potential for SMN2 to produce full-length SMN protein, it has been the principal target for therapies designed to increase the production of functional SMN protein in SMA. Antisense oligonucleotides targeting regulatory sites within SMN2 pre-mRNA, such as the ISS-N1 or E1 sites, have been shown to increase full-length SMN2 mRNA and protein expression in mouse models of SMA as well as in SMA patients in clinical trials. An antisense oligonucleotide (linear ASO), Spinraza, was approved by the Food and Drug Administration (FDA) for SMA treatment.

It has been discovered, quite unexpectedly, that splice modulating antisense oligonucleotides described herein are more potent when arranged in a SNA format. It was discovered that these splice modulating antisense oligonucleotides are more active in a SNA format relative to same linear splice modulating antisense oligonucleotides. This unexpected finding, demonstrated herein shows that splice modulating antisense oligonucleotides comprised of a variety of lipid containing or other cores, oligonucleotide sequences, oligonucleotide lengths, and oligonucleotide densities are capable of enhancing the expression of a protein, whose low levels are associated with SMA.

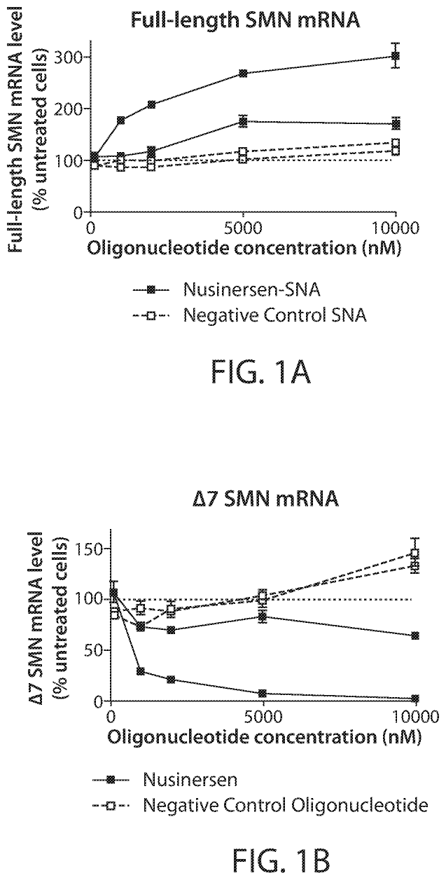

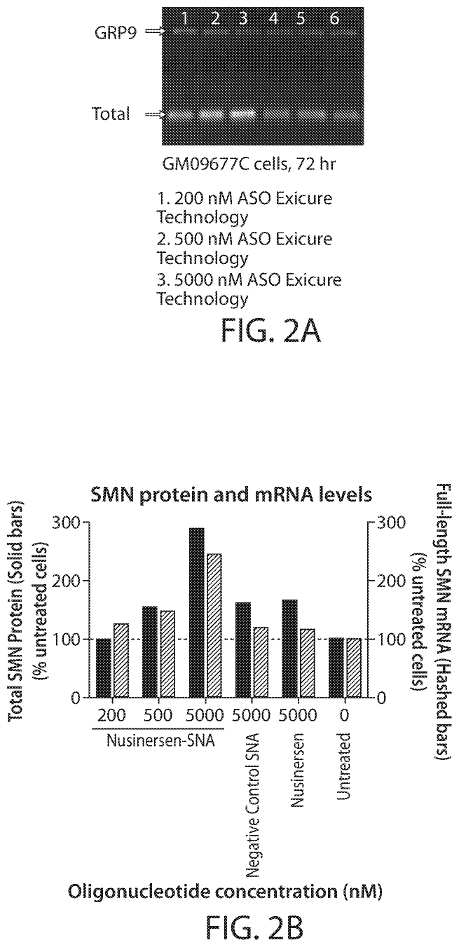

Also, unexpectedly, the antisense oligonucleotides disclosed herein have a different distribution and persistence compared to the corresponding linear or free antisense oligonucleotides in vivo. Exemplary antisense oligonucleotides in the SNA format disclosed herein are distributed away from the site of administration relatively slowly and are maintained in the target region/organ for a time longer than the corresponding linear or free antisense oligonucleotide. Furthermore, less antisense oligonucleotide in the SNA format is observed in the kidneys which, without wishing to be bound by theory, likely indicates a relatively slow clearance rate from the CNS. The slower clearance and accumulation in the kidneys of antisense oligonucleotide in the SNA format relative to linear or free antisense oligonucleotide could also result in lower renal toxicity. Overall, disclosed herein is that antisense oligonucleotides in the SNA format persist in the CNS longer and at higher levels compared to the corresponding free or linear antisense oligonucleotide. Importantly, the data presented herein show that having the splice modulating antisense oligonucleotide in a SNA structure enhanced the inclusion of an exon normally excluded from the SMN2 gene in SMA and that the antisense oligonucleotide in a SNA also persists in the CNS longer and at higher levels compared to the corresponding free or linear antisense oligonucleotide. Linear splice modulating antisense oligonucleotides which lack the oligonucleotide shell do not show similar activity (FIGS. 1-2). The results suggest that antisense oligonucleotide SNAs are uniquely able to achieve the desired inclusion of exon 7 in the SMN2 gene and ultimately lead to increased expression of SMN protein for the treatment of SMA.

Further, in vivo data (described in the Examples) has demonstrated that the splice modulating oligonucleotide SNA (also referred to as Nusinersen-SNA or Spinraza-SNA) exhibits significantly improved therapeutic properties as compared with the linear oligonucleotide (nusinersen) in a mouse model of SMA. Because nusinersen is clinically administered to the CSF, the constructs were delivered to the CSF via intracerebral ventricular (ICV) injection in post-natal day 0 (P0) mice. Mice treated with 20 μg of nusinersen had a median survival of 17 days, compared to 14 days in untreated mice. In contrast, 10 μg of nusinersen-SNA increased median survival to 26 days whereas 20 μg increased survival to 69 days. Increasing the nusinersen dose to 30 μg resulted in toxicity and a median survival of 2 days. Thus, nusinersen-SNA treatment resulted in substantially increased median survival over nusinersen at the same dose. Unlike Nusinersen and quite unexpectedly, administration of nusinersen-SNA by ICV injection to the CSF at 30 μg dose did not lead to acute toxicity. In vitro and in vivo, nusinersen-SNA treatment elicited more full length SMN mRNA compared to nusinersen. Given that SNAs improve the efficacy and safety of nusinersen in the central nervous system (CNS), the SNAs of the invention may improve the therapeutic window of existing splice modulating oligonucleotides and thus, may be used as novel therapies for CNS disorders.

Thus, the data show that the SNA of the invention demonstrated increased survival and decreased toxicity in a translationally-relevant SNA mouse model. In brief, the data demonstrated prolonged survival by four-fold (maximal survival of 115 days compared to 28 days for nusinersen-treated mice), doubled the levels of healthy full-length SMN2 mRNA and protein in SMA patient fibroblasts when compared to nusinersen, doubled the quantity of healthy full-length SMN mRNA levels in spinal cord tissue compared to untreated mice and mitigated toxicity of nusinersen at the highest dose tested in mice.

Spherical nucleic acids (SNA) are three-dimensional arrangements of nucleic acids, with densely packed and radially arranged oligonucleotides on a central nanoparticle core. In its simplest form the SNA is composed of oligonucleotides and a core. The core may be a hollow core which is produced by a 3-dimensional arrangement of molecules which form the outer boundary of the core. For instance, the molecules may be in the form of a lipid layer or bilayer which has a hollow center. Alternatively, the molecules may be in the form of lipids, such as amphipathic lipids, i.e., sterols which are linked to an end the oligonucleotide. Sterols such as cholesterol linked to an end of an oligonucleotide may associate with one another and form the outer edge of a hollow core with the oligonucleotides radiating outward from the core. The core may also be a solid or semi-solid core.

The oligonucleotides are associated with the core. An oligonucleotide that is associated with the core may be covalently linked to the core or non-covalently linked to the core, i.e., potentially through hydrophobic interactions. For instance, when a sterol forms the outer edge of the core an oligonucleotide may be covalently linked to the sterol directly or indirectly. When a lipid layer forms the core, the oligonucleotide may be covalently linked to the lipid or may be non-covalently linked to the lipids e.g., by interactions with the oligonucleotide or a molecule such as a cholesterol attached to the oligonucleotide directly or indirectly through a linker.

SNAs are taken up by cells to a greater extent than the same oligonucleotides that are not in the SNA format. Nontoxic, biocompatible, and biodegradable lipid-containing SNAs that are useful for treating neurodegenerative diseases and disorders, such as spinal muscular atrophy (SMA) are disclosed herein. Antisense technology is an effective means for modulating the expression of one or more specific gene products, including alternative splice products, and is uniquely useful in a number of therapeutic, diagnostic, and research applications. The principle behind antisense technology is that an antisense compound, which hybridizes to a target nucleic acid, modulates gene expression activities such as transcription, splicing or translation through one of a number of antisense mechanisms. The sequence specificity of antisense compounds makes them extremely attractive as tools for target validation and gene functionalization, as well as therapeutics to selectively modulate the expression of genes involved in disease.

As used herein, “antisense activity” refers to any detectable and/or measurable change attributable to the hybridization of an antisense compound to its target nucleic acid related to splice modulating. In some embodiments, antisense activity is an increase in the amount or expression of a target nucleic acid or protein encoded by such target nucleic acid compared to target nucleic acid levels or target protein levels in the absence of the antisense compound.

As used herein, “antisense compound” refers to a compound comprising a splice modulating antisense oligonucleotide in a spherical nucleic acid (SNA). The terms “antisense compound” or “oligonucleotide” and “splice modulating compound” or “oligonucleotide” are used interchangeably to refer to a splice modulating oligonucleotide. As used herein, “antisense oligonucleotide” refers to an oligonucleotide having a nucleobase sequence that is at least partially complementary to a target nucleic acid. In some embodiments, the antisense oligonucleotide contains one or more additional features, or one or more additional modifications.

Splice-switching or splice modulating oligonucleotides direct pre-mRNA splicing by binding sequence elements and blocking access to the transcript by the spliceosome and other splicing factors. They can be applied to (1) restore correct splicing of an aberrantly spliced transcript, (2) produce a novel splice variant that is not normally expressed, or (3) manipulate alternative splicing from one splice variant to another. Through the latter mechanism, splice-switching oligonucleotides may therefore downregulate a deleterious transcript while simultaneously upregulating expression of a preferred transcript. Notably, their activity is enhanced with increased target gene expression because this enables increased production of the preferred splice variant. This is in contrast to traditional anti-sense approaches and small-interfering RNA, which exhibit decreased potency with increased target gene expression.

In some embodiments, an antisense oligonucleotide refers to an antisense oligonucleotide that comprises or consists of the nucleic acid sequence of SEQ ID NO: 1 below.

(SEQ ID NO: 1)
5′ - TCA CTT TCA TAA TGC TGG - (Spacer 18)₂ -
3CholTEG.

In some embodiments, a SNA described herein comprises an oligonucleotide that is 2% to 100% identical to the nucleic acid sequence of SEQ ID NO: 1. In some embodiments, a SNA described herein comprises an oligonucleotide that is 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 1. In some embodiments, the oligonucleotide of SEQ ID NO: 1 or the oligonucleotide that is 2% to 100% identical 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 1 disclosed herein is not in a SNA configuration or is not part of a SNA. In some embodiments, such oligonucleotide is a free or linear oligonucleotide.

In some embodiments, the antisense oligonucleotide refers to the nucleic acid sequence of ISIS 396443. As used herein, “ISIS 396443” refers to an oligonucleotide having the following structure:

(SEQ ID NO: 16)
Tes ^{m}Ces Aes ^{m}Ces Tes Tes Tes ^{m}Ces Aes Tes Aes Aes
Tes Ges ^{m}Ces Tes Ges Ge,

wherein “^{m}C” indicates 5-methyl cytosine; “e” indicates a 2′-MOE modification; “C” indicates cytidine, “T” indicates thymidine, “A” indicates adenosine, “G” indicates guanosine, and “s” indicates phosphorothioate linkage. Isis 396443 is also referred to in the art as Nusinersen, which is the International Nonproprietary Name (INN), as Ionis-SMNRx, and as BIIB058. As used herein, “MOE” refers to methoxyethyl. “2′-MOE” means a —OCH₂CH₂OCH₃ group at the 2′ position of a furanosyl ring.

In some embodiments, a SNA described herein comprises an oligonucleotide that is 2% to 100% identical to the nucleic acid sequence of SEQ ID NO: 16. In some embodiments, a SNA described herein comprises an oligonucleotide that is 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 16. In some embodiments, the oligonucleotide of SEQ ID NO: 16 or the oligonucleotide that is 2% to 100% identical 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 16 disclosed herein is not in a SNA configuration or is not part of a SNA. In some embodiments, such oligonucleotide is a free or linear oligonucleotide.

In some embodiments, the antisense oligonucleotide refers to an antisense oligonucleotide that comprises or consists of the nucleic acid sequence of SEQ ID NO: 17 below.

(SEQ ID NO: 17)
5′-CUA UAU AUA GAU AGU UAU UCA ACA AA-3′

The following oligos were modified at every base with Morpholino chemistry groups:

E1MO-ASO (26-mer)
(SEQ ID NO: 17)
5′ CUA UAU AUA GAU AGU UAU UCA ACA AA 3′.

In some embodiments, a SNA described herein comprises an oligonucleotide that is 2% to 100% identical to the nucleic acid sequence of SEQ ID NO: 17. In some embodiments, a SNA described herein comprises an oligonucleotide that is 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 17. In some embodiments, the oligonucleotide of SEQ ID NO: 17 or the oligonucleotide that is 2% to 100% identical 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 17 disclosed herein is not in a SNA configuration or is not part of a SNA. In some embodiments, such oligonucleotide is a free or linear oligonucleotide.

In some embodiments, a SNA described herein comprises an oligonucleotide that is 2% to 100% identical to the nucleic acid sequence 5′-TCACTTTCATAATGCTGG-3′ (SEQ ID NO: 172). In some embodiments, a SNA described herein comprises an oligonucleotide that is 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 172. In some embodiments, the oligonucleotide of SEQ ID NO: 172 or the oligonucleotide that is 2% to 100% identical 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 172 disclosed herein is not in a SNA configuration or is not part of a SNA. In some embodiments, such oligonucleotide is a free or linear oligonucleotide.

In some embodiments, a SNA described herein comprises an oligonucleotide that is 2% to 100% identical to the nucleic acid sequence that is or is 2% to 100% identical to the sequence 5′-moeT*/5-Me-moeC/*moeA*/5-Me-moeC/*moeT*moeT*moeT*/5-Me-moeC/*moeA*moeT*moeA*moeA*moeT*moeG*/5-Me-moeC/*moeT*moeG*moeG/isp18//isp18//3CholTEG/(SEQ ID NO: 164). In some embodiments, a SNA described herein comprises an oligonucleotide that is 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 164. In some embodiments, the oligonucleotide of SEQ ID NO: 164 or the oligonucleotide that is 2% to 100% identical 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of SEQ ID NO: 164 disclosed herein is not in a SNA configuration or is not part of a SNA. In some embodiments, such oligonucleotide is a free or linear oligonucleotide.

In some embodiments, a SNA described herein comprises an oligonucleotide that is 2% to 100% identical to the nucleic acid sequence of any of the oligonucleotides disclosed herein. In some embodiments, a SNA described herein comprises an oligonucleotide that is 2% identical, 5% identical, 10% identical, 15% identical, 20% identical, 25% identical, 30% identical, 35% identical, 40% identical, 45% identical, 50% identical, 55% identical, 60% identical, 65% identical, 70% identical, 75% identical, 80% identical, 85% identical, 86% identical, 87% identical, 88% identical, 89% identical, 90% identical, 91% identical, 92% identical, 93% identical, 94% identical, 95% identical, 96% identical, 97% identical, 98% identical, 99% identical, 99.5% identical, or 100% identical to the nucleic acid sequence of any of the oligonucleotide sequences disclosed herein. In some embodiments, any of the oligonucleotides disclosed herein, such as antisense oligonucleotides, are not in a SNA configuration or part of a SNA. In some embodiments, any of the oligonucleotides disclosed herein are free oligonucleotides or linear oligonucleotides.

In some embodiments, each base of the antisense oligonucleotide of SEQ ID NO: 17 is modified with morpholino chemistry groups. A “morpholino oligomer” or “PMO” refers to an oligonucleotide having a backbone which supports a nucleobase capable of hydrogen bonding to typical oligonucleotides, wherein the polymer lacks a pentose Sugar backbone moiety, but instead contains a morpholino ring. An exemplary “morpholino oligomer comprises morpholino subunit structures linked together by phosphoramidate or phosphorodiamidate linkages, joining the morpholino nitrogen of one subunit to the 4′ exocyclic carbon of an adjacent subunit, each subunit comprising a purine or pyrimidine nucleobase effective to bind, by base-specific hydrogen bonding, to a base in a polynucleotide. Morpholino oligomers (including antisense oligomers) are detailed, for example, in U.S. Pat. Nos. 5,698,685: 5,217,866; 5,142,047; 5,034,506; 5,166,315; 5,185,444; 5,521,063: 5,506,337 and pending U.S. patent application Ser. Nos. 12/271,036: 12/271,040; and PCT publication number WO/2009/064471 all of which are incorporated herein by reference in their entirety.

In some embodiments, each base of the antisense oligonucleotide of SEQ ID NO: 17 is modified with locked nucleic acid (LNA), in which the 2′-hydroxyl group of the RNA is linked to the 4′ carbon atom of the sugar ring, thereby forming a bicyclic sugar moiety. The linkage is in certain aspects is a methylene (—CH2-)n group bridging the 2′ oxygen atom and the 4′ carbon atom wherein n is 1, 2 or 3. LNAs and preparation thereof are described in WO 98/39352 and WO 99/14226.

In other embodiments, each base of the antisense oligonucleotide of SEQ ID NO:17 is a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In some embodiments, the present invention provides antisense compounds, which comprise or consist of an oligomeric compound comprising an antisense oligonucleotide, having a nucleobase sequences complementary to that of a target nucleic acid. In some embodiments, antisense compounds are single-stranded. Such single-stranded antisense compounds typically comprise or consist of an oligomeric compound that comprises or consists of a modified oligonucleotide and optionally a conjugate group. In some embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In some embodiments, oligomeric compounds of antisense compounds are capable of hybridizing to a target nucleic acid, resulting in at least one antisense activity. In some embodiments, antisense compounds selectively affect one or more target nucleic acid. Such selective antisense compounds comprises a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In some embodiments, hybridization of an antisense compound to a target nucleic acid results in alteration of processing, e.g., splicing, of the target precursor transcript. In some embodiments, hybridization of an antisense compound to a target precursor transcript results in inhibition of a binding interaction between the target nucleic acid and a protein or other nucleic acid. In certain such embodiments, hybridization of an antisense compound to a target precursor transcript results in alteration of translation of the target nucleic acid.

Antisense activities may be observed directly or indirectly. In some embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

In some embodiments, antisense compounds and/or oligomeric compounds comprise antisense oligonucleotides that are complementary to the target nucleic acid over the entire length of the oligonucleotide. In some embodiments, such oligonucleotides are 99% complementary to the target nucleic acid. In some embodiments, such oligonucleotides are 95% complementary to the target nucleic acid. In some embodiments, such oligonucleotides are 90% complementary to the target nucleic acid. In some embodiments, such oligonucleotides are 85% complementary to the target nucleic acid. In some embodiments, such oligonucleotides are 80% complementary to the target nucleic acid. In some embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In some embodiments, oligomeric compounds and/or antisense compounds comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In some embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

In some embodiments, the antisense oligonucleotide is two to 100 nucleotides in length. In some embodiments, the antisense oligonucleotide is three nucleotides in length, four nucleotides in length, five nucleotides in length, six nucleotides in length, seven nucleotides in length, eight nucleotides in length, nine nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, 41 nucleotides in length, 42 nucleotides in length, 43 nucleotides in length, 44 nucleotides in length, 45 nucleotides in length, 46 nucleotides in length, 47 nucleotides in length, 49 nucleotides in length, 50 nucleotides in length, 52 nucleotides in length, 54 nucleotides in length, 56 nucleotides in length, 58 nucleotides in length, 60 nucleotides in length, 62 nucleotides in length, 64 nucleotides in length, 66 nucleotides in length, 68 nucleotides in length, 70 nucleotides in length, 72 nucleotides in length, 74 nucleotides in length, 76 nucleotides in length, 78 nucleotides in length, 80 nucleotides in length, 82 nucleotides in length, 84 nucleotides in length, 86 nucleotides in length, 88 nucleotides in length, 90 nucleotides in length, 92 nucleotides in length, 94 nucleotides in length, 96 nucleotides in length, 100 nucleotides or more than 100 nucleotides in length, or any range or combination thereof.

In some embodiments, oligomeric compounds comprise or consist of a modified oligonucleotide that is complementary to a target precursor transcript. In certain such embodiments, the target precursor transcript is a target pre-mRNA. In some embodiments, contacting a cell with a compound complementary to a target precursor transcript modulates processing of the target precursor transcript. In certain such embodiments, the resulting target processed transcript has a different nucleobase sequence than the target processed transcript that is produced in the absence of the compound. In some embodiments, the target precursor transcript is a target pre-mRNA and contacting a cell with a compound complementary to the target pre-mRNA modulates splicing of the target pre-mRNA. In certain such embodiments, the resulting target mRNA has a different nucleobase sequence than the target mRNA that is produced in the absence of the compound. In certain such embodiments, an exon is excluded from the target mRNA. In some embodiments, an exon is included in the target mRNA. In some embodiments, the exclusion or inclusion of an exon induces or prevents nonsense mediated decay of the target mRNA, removes or adds a premature termination codon from the target mRNA, and/or changes the reading frame of the target mRNA.

As used herein, “double-stranded antisense compound” refers to an antisense compound comprising two oligomeric compounds that are complementary to each other and form a duplex, and wherein one of the two said oligomeric compounds comprises an antisense oligonucleotide.

As used herein, “hybridization” refers to the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression of activity in an untreated or control sample and does not necessarily indicate a total elimination of expression or activity.

As used herein, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. When “decrease” or “inhibition” is used in the context of the level of expression or activity of a gene or a protein, it refers to a reduction in protein or nucleic acid level or activity in a cell, a cell extract, or a cell supernatant. For example, such a decrease may be due to reduced RNA stability, transcription, or translation, increased protein degradation, or RNA interference.

As used herein, “up-regulate”, “increase” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or a 100% increase or more, or any increase between 10-100% as compared to a reference level, or an increase greater than 100%, for example, an increase at least about a 2-fold, or at least about a 3-fold, or at least about a 4-fold, or at least about a 5-fold or at least about a 10-fold increase, or any increase between 2-fold and 10-fold or greater as compared to a reference level. When “increase” is used in the context of the expression or activity of a gene or protein, it refers to a positive change in protein or nucleic acid level or activity in a cell, a cell extract, or a cell supernatant. For example, such an increase may be due to increased RNA stability, transcription, or translation, or decreased protein degradation. Preferably, this increase is at least 5%, at least about 10%, at least about 25%, at least about 50%, at least about 75%, at least about 80%, at least about 100%, at least about 200%, or even about 500% or more over the level of expression or activity under control conditions.

As used herein, “oligonucleotide” refers to a strand of linked nucleosides connected via internucleoside linkages, wherein each nucleoside and internucleoside linkage may be modified or unmodified. In some embodiments, the length of an oligonucleotide described herein, such as an antisense oligonucleotide, is of 2-500 linked nucleosides. In some embodiments, the length of an oligonucleotide described herein, is of 2-200, 2-195, 2-190, 2-185, 2-180, 2-175, 2-170, 2-165, 2-160, 2-155, 2-150, 2-145, 2-140, 2-135, 2-130, 2-125, 2-120, 2-115, 2-110, 2-105, 2-100, 2-95, 2-90, 2-85, 2-80, 2-75, 2-70, 2-65, 2-60, 2-55, 2-50, 2-45, 2-40, 2-39, 2-38, 2-37, 2-36, 2-35, 2-34, 2-33, 2-32, 2-31, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4, 4-200, 4-195, 4-190, 4-185, 4-180, 4-175, 4-170, 4-165, 4-160, 4-155, 4-150, 4-145, 4-140, 4-135, 4-130, 4-125, 4-120, 4-115, 4-110, 4-105, 4-100, 4-95, 4-90, 4-85, 4-80, 4-75, 4-70, 4-65, 4-60, 4-55, 4-50, 4-45, 4-40, 4-39, 4-38, 4-37, 4-36, 4-35, 4-34, 4-33, 4-32, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 4-7, 4-6, 4-5, 6-200, 6-195, 2-190, 6-185, 6-180, 6-175, 6-170, 6-165, 6-160, 6-155, 6-150, 6-145, 6-140, 6-135, 6-130, 6-125, 6-120, 6-115, 6-110, 6-105, 6-100, 6-95, 6-90, 6-85, 6-80, 6-75, 6-70, 6-65, 6-60, 6-55, 6-50, 6-45, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 2-200, 8-195, 8-190, 8-185, 8-180, 8-175, 8-170, 8-165, 8-160, 8-155, 8-150, 8-145, 8-140, 8-135, 8-130, 8-125, 8-120, 8-115, 8-110, 8-105, 8-100, 8-95, 8-90, 8-85, 8-80, 8-75, 8-70, 8-65, 8-60, 8-55, 8-50, 8-45, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 2-200, 10-195, 10-190, 10-185, 10-180, 10-175, 10-170, 10-165, 10-160, 10-155, 10-150, 10-145, 10-140, 10-135, 10-130, 10-125, 10-120, 10-115, 10-110, 10-105, 10-100, 10-95, 10-90, 10-85, 10-80, 10-75, 10-70, 10-65, 10-60, 10-55, 10-50, 10-45, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, or 10-12 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. In some embodiments, modified oligonucleotides having one or more modified sugar moieties at the 2′ position have enhanced pharmacologic activity for modulation of SMN2 pre-mRNA, including increasing the percentage of SMN2 transcripts containing exon 7.

As used herein, “mismatch” or “non-complementary” means a nucleobase of a first oligonucleotide that is not complementary with the corresponding nucleobase of a second oligonucleotide or target nucleic acid when the first and second oligomeric compound are aligned. As used herein, “naturally occurring” means found in nature.

As used herein, “ameliorate” in reference to a treatment improvement in at least one symptom relative to the same symptom in the absence of the treatment. In some embodiments, the treatment is of a neurodegenerative disorder described herein, such as treatment of spinal muscular atrophy (SMA). In some embodiments, amelioration is the reduction in the severity or frequency of a symptom or the delayed onset or slowing of progression in the severity or frequency of a symptom associated with a neurodegenerative disorder, such as SMA.

As used herein, a “cell-targeting moiety” refers to a conjugate group or portion of a conjugate group that results in improved uptake to a particular cell type and/or distribution to a particular tissue relative to an oligomeric compound lacking the cell-targeting moiety.

As used herein, “complementary” to an oligonucleotide described means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to an oligonucleotide described herein means that such oligonucleotides are complementary to another oligonucleotide or nucleic acid at each nucleoside of the oligonucleotide.

As used herein, the terms “internucleoside linkage” refers to a group or bond that forms a covalent linkage between adjacent nucleosides in an oligonucleotide. As used herein, a “modified intemucleoside linkage” refers to any intemucleoside linkage other than a naturally occurring, phosphate intemucleoside linkage or phosphodiester linkage. Non-phosphate linkages are referred to herein as modified intemucleoside linkages.

In some embodiments, the internucleoside linkage is a phosphorothioate linkage. As used herein, “phosphorothioate linkage” refers to a modified phosphate linkage in which one of the non-bridging oxygen atoms is replaced with a sulfur atom. A phosphorothioate intemucleoside linkage is a modified intemucleoside linkage. In some embodiments, all or 100% of the internucleoside linkages of an antisense oligonucleotide described herein are phosphodiesters. In some embodiments, less than all or less than 100% of the internucleoside linkages of an antisense oligonucleotide described herein are phosphodiester linkages. In some embodiments, 5-20%, 5-50%, 5-75%, 5-100%, 10-20%, 10-50%, 10-75% or 10-100% of the internucleoside linkages are phosphodiester linkages.

In some embodiments, one of the internucleoside linkages, two of the internucleoside linkages, three of the internucleoside linkages, four of the internucleoside linkages, five of the internucleoside linkages, six of the internucleoside linkages, seven of the internucleoside linkages, eight of the internucleoside linkages, nine of the internucleoside linkages, 10 of the internucleoside linkages, 11 of the internucleoside linkages, 12 of the internucleoside linkages, 13 of the internucleoside linkages, 14 of the internucleoside linkages, 15 of the internucleoside linkages, 16 of the internucleoside linkages, 17 of the internucleoside linkages, 18 of the internucleoside linkages, 19 of the internucleoside linkages, 20 of the internucleoside linkages, 21 of the internucleoside linkages, 22 of the internucleoside linkages, 23 of the internucleoside linkages, 24 of the internucleoside linkages, 25 of the internucleoside linkages, 26 of the internucleoside linkages, 27 of the internucleoside linkages, 28 of the internucleoside linkages, 29 of the internucleoside linkages, 30 of the internucleoside linkages, 31 of the internucleoside linkages, 32 of the internucleoside linkages, 33 of the internucleoside linkages, 34 of the internucleoside linkages, 35 of the internucleoside linkages, 36 of the internucleoside linkages, 37 of the internucleoside linkages, 38 of the internucleoside linkages, 39 of the internucleoside linkages, 40 of the internucleoside linkages, 41 of the internucleoside linkages, 42 of the internucleoside linkages, 43 of the internucleoside linkages, 44 of the internucleoside linkages, 45 of the internucleoside linkages, 46 of the internucleoside linkages, 47 of the internucleoside linkages, 49 of the internucleoside linkages, 50 of the internucleoside linkages, 52 of the internucleoside linkages, 54 of the internucleoside linkages, 56 of the internucleoside linkages, 58 of the internucleoside linkages, 60 of the internucleoside linkages, 62 of the internucleoside linkages, 64 of the internucleoside linkages, 66 of the internucleoside linkages, 68 of the internucleoside linkages, 70 of the internucleoside linkages, 72 of the internucleoside linkages, 74 of the internucleoside linkages, 76 of the internucleoside linkages, 78 of the internucleoside linkages, 80 of the internucleoside linkages, 82 of the internucleoside linkages, 84 of the internucleoside linkages, 86 of the internucleoside linkages, 88 of the internucleoside linkages, 90 of the internucleoside linkages, 92 of the internucleoside linkages, 94 of the internucleoside linkages, 96 of the internucleoside linkages, 100 nucleotides or more than 100 of the internucleoside linkages, or any range or combination thereof of an antisense oligonucleotide described herein are phosphodiester linkages.

In some embodiments, 5-20%, 5-50%, 5-75%, 5-100%, 10-20%, 10-50%, 10-75% or 10-100% of the internucleoside linkages of an antisense oligonucleotide described herein are phosphorothioate linkages.

In some embodiments, one of the internucleoside linkages, two of the internucleoside linkages, three of the internucleoside linkages, four of the internucleoside linkages, five of the internucleoside linkages, six of the internucleoside linkages, seven of the internucleoside linkages, eight of the internucleoside linkages, nine of the internucleoside linkages, 10 of the internucleoside linkages, 11 of the internucleoside linkages, 12 of the internucleoside linkages, 13 of the internucleoside linkages, 14 of the internucleoside linkages, 15 of the internucleoside linkages, 16 of the internucleoside linkages, 17 of the internucleoside linkages, 18 of the internucleoside linkages, 19 of the internucleoside linkages, 20 of the internucleoside linkages, 21 of the internucleoside linkages, 22 of the internucleoside linkages, 23 of the internucleoside linkages, 24 of the internucleoside linkages, 25 of the internucleoside linkages, 26 of the internucleoside linkages, 27 of the internucleoside linkages, 28 of the internucleoside linkages, 29 of the internucleoside linkages, 30 of the internucleoside linkages, 31 of the internucleoside linkages, 32 of the internucleoside linkages, 33 of the internucleoside linkages, 34 of the internucleoside linkages, 35 of the internucleoside linkages, 36 of the internucleoside linkages, 37 of the internucleoside linkages, 38 of the internucleoside linkages, 39 of the internucleoside linkages, 40 of the internucleoside linkages, 41 of the internucleoside linkages, 42 of the internucleoside linkages, 43 of the internucleoside linkages, 44 of the internucleoside linkages, 45 of the internucleoside linkages, 46 of the internucleoside linkages, 47 of the internucleoside linkages, 49 of the internucleoside linkages, 50 of the internucleoside linkages, 52 of the internucleoside linkages, 54 of the internucleoside linkages, 56 of the internucleoside linkages, 58 of the internucleoside linkages, 60 of the internucleoside linkages, 62 of the internucleoside linkages, 64 of the internucleoside linkages, 66 of the internucleoside linkages, 68 of the internucleoside linkages, 70 of the internucleoside linkages, 72 of the internucleoside linkages, 74 of the internucleoside linkages, 76 of the internucleoside linkages, 78 of the internucleoside linkages, 80 of the internucleoside linkages, 82 of the internucleoside linkages, 84 of the internucleoside linkages, 86 of the internucleoside linkages, 88 of the internucleoside linkages, 90 of the internucleoside linkages, 92 of the internucleoside linkages, 94 of the internucleoside linkages, 96 of the internucleoside linkages, 100 nucleotides or more than 100 of the internucleoside linkages, or any range or combination thereof of an antisense oligonucleotide described herein are phosphorothioate linkages.

As used herein, “phosphodiester internucleoside linkage” means a phosphate group that is covalently bonded to two adjacent nucleosides of a modified oligonucleotide.

In some embodiments, an antisense oligonucleotide described herein is attached or inserted in to the surface of the lipid-containing core through conjugation to one or more linkers. Non-limiting examples of linkers contemplated herein include: tocopherols, sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

A spherical nucleic acid (SNA) can be functionalized in order to attach a polynucleotide. Alternatively or additionally, the polynucleotide can be functionalized. One mechanism for functionalization is the alkanethiol method, whereby oligonucleotides are functionalized with alkanethiols at their 3′ or 5′ termini prior to attachment to gold nanoparticles or nanoparticles comprising other metals, semiconductors or magnetic materials. Such methods are described, for example Whitesides, Proceedings of the Robert A. Welch Foundation 39th Conference On Chemical Research Nanophase Chemistry, Houston, Tex., pages 109-121 (1995), and Mucic et al. Chem. Commun. 555-557 (1996). Oligonucleotides can also be attached to nanoparticles using other functional groups such as phosophorothioate groups, as described in and incorporated by reference from U.S. Pat. No. 5,472,881, or substituted alkylsiloxanes, as described in and incorporated by reference from Burwell, Chemical Technology, 4, 370-377 (1974) and Matteucci and Caruthers, J. Am. Chem. Soc., 103, 3185-3191 (1981). In some instances, oligonucleotides are attached to nanoparticles by terminating the polynucleotide with a 5′ or 3′ thionucleoside. In other instances, an aging process is used to attach oligonucleotides to nanoparticles as described in and incorporated by reference from U.S. Pat. Nos. 6,361,944, 6,506,569, 6,767,702 and 6,750,016 and PCT Publication Nos. WO 1998/004740, WO 2001/000876, WO 2001/051665 and WO 2001/073123. In some embodiments, the core is a metal core. In some embodiments, the core is an inorganic metal core. In some embodiments, the core is a gold core.

In some instances, the oligonucleotide is attached or inserted in the SNA. A spacer sequence can be included between the attachment site and the oligonucleotide. In some embodiments, a spacer sequence comprises or consists of an oligonucleotide, a peptide, a polymer or an oligoethylene glycol. In a preferred embodiment, the spacer is oligoethylene glycol and more preferably, hexaethyleneglycol.

As used herein, “precursor transcript” means a coding or non-coding RNA that undergoes processing to form a processed or mature form of the transcript. Precursor transcripts include but are not limited to pre-mRNAs, long non-coding RNAs, pri-miRNAs, and intronic RNAs.

As used herein, “processing” in reference to a precursor transcript means the conversion of a precursor transcript to form the corresponding processed transcript. Processing of a precursor transcript includes but is not limited to nuclease cleavage events at processing sites of the precursor transcript.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis).

A polynucleotide of the nanoscale construct and optionally attached to a nanoparticle core can be single stranded or double stranded. A double stranded polynucleotide is also referred to herein as a duplex. Double-stranded oligonucleotides of the invention can comprise two separate complementary nucleic acid strands.

As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded polynucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded polynucleotide of the invention contains at least or up to 1, 2, 3, 4, 5, 6, 7, 8, 9, 10, 11, 12, 13, 14, or 15 mismatches.

Oligonucleotides associated with the invention can be modified such as at the sugar moiety, the phosphodiester linkage, and/or the base. As used herein, “sugar moieties” includes natural, unmodified sugars, including pentose, hexose, conformationally flexible sugars, conformationally locked sugars, arabinose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, oligonucleotides associated with the invention may only contain modified or unmodified sugar moieties, while in other instances, oligonucleotides contain some sugar moieties that are modified and some that are not.

In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH2, NHR, NR2,), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like. The sugar moiety can also be a hexose or arabinose.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In some embodiments, a straight chain or branched chain alkyl has 6 or fewer carbon atoms in its backbone (e.g., C1-C6 for straight chain, C3-C6 for branched chain), and more preferably 4 or fewer. Likewise, preferred cycloalkyls have from 3-8 carbon atoms in their ring structure, and more preferably have 5 or 6 carbons in the ring structure. The term C1-C6 includes alkyl groups containing 1 to 6 carbon atoms.

Unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In some embodiments, a straight chain or branched chain alkenyl group has 6 or fewer carbon atoms in its backbone (e.g., C2-C6 for straight chain, C3-C6 for branched chain) Likewise, cycloalkenyl groups may have from 3-8 carbon atoms in their ring structure, and more preferably have 5 or 6 carbons in the ring structure. The term C2-C6 includes alkenyl groups containing 2 to 6 carbon atoms.

Unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “hydrophobic modifications” refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C6H5OH); tryptophanyl (C8H6N)CH2CH(NH2)CO), Isobutyl, butyl, aminobenzyl; phenyl; naphthyl,

The term “heteroatom” includes atoms of any element other than carbon or hydrogen. In some embodiments, preferred heteroatoms are nitrogen, oxygen, sulfur and phosphorus. The term “hydroxy” or “hydroxyl” includes groups with an —OH or —O— (with an appropriate counterion). The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)0-3NR′R″, (CR′R″)0-3CN, NO2, halogen, (CR′R″)0-3C(halogen)3, (CR′R″)0-3CH(halogen)2, (CR′R″)0-3CH2(halogen), (CR′R″)0-3CONR′R″, (CR′R″)0-3S(O)1-2NR′R″, (CR′R″)0-3CHO, (CR′R″)0-3O(CR′R″)0-3H, (CR′R″)0-3S(O)0-2R′, (CR′R″)0-3O(CR′R″)0-3H, (CR′R″)0-3COR′, (CR′R″)0-3CO2R′, or (CR′R″)0-3OR′ groups; wherein each R′ and R″ are each independently hydrogen, a C1-C5 alkyl, C2-C5 alkenyl, C2-C5 alkynyl, or aryl group, or R′ and R″ taken together are a benzylidene group or a —(CH2)2O(CH2)2- group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In some aspects, the nucleomonomers of a polynucleotide of the invention are RNA nucleotides, including modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2nd Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO2-)—O—) that covalently couples adjacent nucleoside monomers. As used herein, the term “substitute linkage” includes any analog or derivative of the native phosphodiester group that covalently couples adjacent nucleomonomers. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In some embodiments, non-hydrolysable linkages are preferred, such as phosphorothioate linkages.

In some aspects, oligonucleotides of the invention comprise 3′ and 5′ termini (except for circular oligonucleotides). The 3′ and 5′ termini of a polynucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH2-CH2-CH3), glycol (—O—CH2-CH2-O—) phosphate (P032-), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleomonomer can comprise a modified sugar moiety. The 3′ terminal nucleomonomer comprises a 3′-0 that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some embodiments, modified oligonucleotides comprise one or more nucleoside comprising an unmodified nucleobase. In some embodiments, modified oligonucleotides comprise one or more nucleoside comprising a modified nucleobase.

In some embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In some embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In some embodiments, oligonucleotides comprise modified and/or unmodified nucleobases arranged along the oligonucleotide or region thereof in a defined pattern or motif. In some embodiments, each nucleobase is modified. In some embodiments, none of the nucleobases are modified. In some embodiments, each purine or each pyrimidine is modified. In some embodiments, each adenine is modified. In some embodiments, each guanine is modified. In some embodiments, each thymine is modified. In some embodiments, each uracil is modified. In some embodiments, each cytosine is modified. In some embodiments, some or all of the cytosine nucleobases in a modified oligonucleotide are 5-methylcytosines.

In some embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In some embodiments, the block is at the 5′-end of the oligonucleotide. In certain embodiments the block is within 3 nucleosides of the 5′-end of the oligonucleotide.

In some embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In some embodiments, the modified nucleobase is selected from: a 2-thiopyrimidine and a 5-propynepyrimidine.

In some aspects, oligonucleotides can comprise both DNA and RNA.

In some aspects, at least a portion of the contiguous oligonucleotides are linked by a substitute linkage, e.g., a phosphorothioate linkage. The presence of substitute linkages can improve pharmacokinetics due to their higher affinity for serum proteins.

In some embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P=0”) (also referred to as unmodified or naturally occurring linkages), phosphotriesters, methylphosphonates, phosphoramidates, and phosphorothioates (“P═S”), and phosphorodithioates (“HS-P═S”). Representative nonphosphorus containing internucleoside linking groups include but are not limited to methylenemethylimino (—CH2-N(CH3)-0-CH2-), thiodiester, thionocarbamate (-0-C(=0)(NH)—S—); siloxane (—O—S1H2-O—); and N,N′-dimethylhydrazine (—CH2-N(CH3)—N(CH3)-). Modified internucleoside linkages, compared to naturally occurring phosphate linkages, can be used to alter, typically increase, nuclease resistance of the oligonucleotide. In some embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral intemucleoside linkages include, without limitation, phosphotriesters, methylphosphonates, MMI (3′-CH2-N(CH3)-0-5′), amide-3 (3′-CH2-C(=0)—N(H)-5′), amide-4 (3′-CH2-N(H)—C(=0)-5′), formacetal (3′-0-CH2-0-5′), methoxypropyl, and thioformacetal (3′-S-CH2-0-5′). Further neutral intemucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral intemucleoside linkages include nonionic linkages comprising mixed N, O, S and CH2 component parts.

As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In some embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

In some embodiments, modified oligonucleotides comprise one or more modified nucleoside comprising a modified sugar. In some embodiments, modified oligonucleotides comprise one or more modified nucleosides comprising a modified nucleobase. In some embodiments, modified oligonucleotides comprise one or more modified intemucleoside linkage. In such embodiments, the modified, unmodified, and differently modified sugar moieties, nucleobases, and/or intemucleoside linkages of a modified oligonucleotide define a pattern or motif. In some embodiments, the patterns of sugar moieties, nucleobases, and intemucleoside linkages are each independent of one another. Thus, a modified oligonucleotide may be described by its sugar motif, nucleobase motif and/or intemucleoside linkage motif (as used herein, nucleobase motif describes the modifications to the nucleobases independent of the sequence of nucleobases).

In some embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In some embodiments, modified oligonucleotides comprise or consist of a region having a gapmer motif, which comprises two external regions or “wings” and a central or internal region or “gap.” The three regions of a gapmer motif (the 5 ‘-wing, the gap, and the 3’-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3 ‘-most nucleoside of the 5’-wing and the 5 ‘-most nucleoside of the 3’-wing) are modified sugar moieties and differ from the sugar moieties of the neighboring gap nucleosides, which are unmodified sugar moieties, thus defining the boundary between the wings and the gap (i.e., the wing/gap junction). In some embodiments, the sugar moieties within the gap are the same as one another. In some embodiments, the gap includes one or more nucleoside having a sugar moiety that differs from the sugar moiety of one or more other nucleosides of the gap. In some embodiments, the sugar motifs of the two wings are the same as one another (symmetric gapmer). In some embodiments, the sugar motif of the 5 ‘-wing differs from the sugar motif of the 3’-wing (asymmetric gapmer).

In some embodiments, the wings of a gapmer comprise 1-5 nucleosides. In some embodiments, the wings of a gapmer comprise 2-5 nucleosides. In some embodiments, the wings of a gapmer comprise 3-5 nucleosides. In some embodiments, the nucleosides of a gapmer are all modified nucleosides.

In some embodiments, the gap of a gapmer comprises 7-12 nucleosides. In some embodiments, the gap of a gapmer comprises 7-10 nucleosides. In some embodiments, the gap of a gapmer comprises 8-10 nucleosides. In some embodiments, the gap of a gapmer comprises 10 nucleosides. In certain embodiment, each nucleoside of the gap of a gapmer is an unmodified 2′-deoxy nucleoside.

In some embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2 ‘-deoxy nucleoside. In certain such embodiments, each nucleoside of each wing is a modified nucleoside.

In some embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside in the entire modified oligonucleotide comprises a modified sugar moiety. In some embodiments, modified oligonucleotides comprise or consist of a region having a fully modified sugar motif, wherein each nucleoside within the fully modified region comprises the same modified sugar moiety, referred to herein as a uniformly modified sugar motif. In some embodiments, a fully modified oligonucleotide is a uniformly modified oligonucleotide. In some embodiments, each nucleoside of a uniformly modified oligonucleotide comprises the same 2′-modification. In some embodiments, each nucleoside of a uniformly modified oligonucleotide comprises a 2′-0-(N-alkyl acetamide) group. In some embodiments, each nucleoside of a uniformly modified oligonucleotide comprises a 2′-0-(N-methyl acetamide) group.

In some embodiments, the invention provides oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In some embodiments, conjugate groups are attached to the 2′-position of a nucleoside of a modified oligonucleotide. In some embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In some embodiments, conjugate groups are attached near the 3′-end of oligonucleotides. In some embodiments, conjugate groups (or terminal groups) are attached at the 5′-end of oligonucleotides. In some embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

In some embodiments, oligonucleotides are covalently attached to one or more conjugate groups. In some embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In some embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBSLett., 1990, 259, 327-330; Svinarchuk et al., Biochimie, 1993, 75, 49-54), a phospholipid, e.g., di-hexadecyl-rac-glycerol or triethyl-ammonium 1,2-di-0-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In some embodiments, conjugate groups may be selected from any of a C22 alkyl, C20 alkyl, C16 alkyl, CIO alkyl, C21 alkyl, C19 alkyl, CI 8 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, CIO alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In some embodiments, conjugate groups may be selected from any of C22 alkyl, C20 alkyl, C16 alkyl, CIO alkyl, C21 alkyl, C19 alkyl, CI 8 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In some embodiments, conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In some embodiments, a conjugate moiety comprises an active drug substance, for example, aspirin, warfarin, phenylbutazone, ibuprofen, suprofen, fen-bufen, ketoprofen, (<S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

Antisense oligonucleotide SNAs are nanoscale constructs composed of: (1) a lipid-containing core, which is formed by arranging non-toxic carrier lipids into a small hollow structure, (2) a shell of oligonucleotides, which is formed by arranging oligonucleotides such that they point radially outwards from the core, and (3) optionally a hydrophobic (e.g. lipid) anchor group attached to either the 5′- or 3′-end of the oligonucleotide, depending on whether the oligonucleotides are arranged with the 5′- or 3′-end facing outward from the core. The anchor drives the insertion into the liposome and to anchor the oligonucleotides to the lipid-containing core.

A liposomal core as used herein refers to a centrally located core compartment formed by a component of the lipids or phospholipids that form a lipid bilayer. “Liposomes” are artificial, self closed vesicular structure of various sizes and structures, where one or several membranes encapsulate an aqueous core. Most typically liposome membranes are formed from lipid bilayers membranes, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo- or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agent.

The lipid-containing core can be constructed from a wide variety of lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, 1,2-dioleoyl-sn-glycero-3-phosphocholine (DOPC) and polyunsaturated sterols of different lengths, saturation states, and their derivatives.

In some embodiments, the oligonucleotides may be positioned on the exterior of the core, within the walls of the core and/or in the center of the core. An oligonucleotide that is positioned on the core is typically referred to as attached to the core. Attached may be direct or indirect. In some embodiments at least 5, 10, 15, 25, 50, 75, 100, 200, 300, 400, 500, 600, 700, 800, 900, 1000, 5000 or 10,000 oligonucleotides or any range combination thereof are on the exterior of the core. In some embodiments, 1-1000, 10-500, 50-250, or 50-300 oligonucleotides are present on the surface.

The oligonucleotides of the oligonucleotide shell may be oriented in a variety of directions. In some embodiments the oligonucleotides are oriented radially outwards. The orientation of these oligonucleotides can be either 5′ distal/3′ terminal in relation to the core, or 3′ distal/5′terminal in relation to the core, or laterally oriented around the core. In one embodiment one or a multiplicity of different oligonucleotides are present on the same surface of a single SNA. In all cases, at least 1 oligonucleotide is present on the surface but up to 10,000 can be present.

The oligonucleotides may be linked to the core or to one another and/or to other molecules such an active agents either directly or indirectly through a linker. The oligonucleotides may be conjugated to a linker via the 5′ end or the 3′ end. Some or all of the oligonucleotides of the nanostructure may be linked to one another or the core either directly or indirectly through a covalent or non-covalent linkage or covalent or non-covalent interaction. In some embodiments, an oligonucleotide disclosed herein is attached to the core through a covalent interaction. In some embodiments, an oligonucleotide disclosed herein is attached to the core through a non-covalent interaction, such as a van der Waals interaction ionic interaction or electrostatic interaction. In some embodiments, the non-covalent interaction is reversible. In some embodiments, an oligonucleotide disclosed herein is uniformly dispersed or suspended around a core, such as a liposomal core or a gold core. In some embodiments, the oligonucleotide is not uniformly dispersed or suspended around a core, such as a liposomal core or gold core. The linkage of one oligonucleotide to another oligonucleotide may be in addition to or alternatively to the linkage of that oligonucleotide to the core or liposomal core.

In some embodiments, the linker comprises a molecular species at the 3′ or 5′ termini of an oligonucleotide disclosed herein. In some embodiments, the molecular species is positioned in a core and the oligonucleotide extends radially from the core. Oligonucleotides of the nanostructure may be linked to one another or the core either directly or indirectly through a covalent or non-covalent linkage or covalent or non-covalent interaction. In some embodiments, an oligonucleotide disclosed herein is attached to the core through a covalent interaction (e.g., thiol-gold interaction). In some embodiments, an oligonucleotide disclosed herein is attached to the core through a non-covalent interaction (e.g., van der Waals interaction, ionic interaction or electrostatic interaction).

In some embodiments, the molecular species is at the 5′ end of the oligonucleotide. In some embodiments, the molecular species is a hydrophobic group. In some embodiments, the hydrophobic group is selected from the group consisting of cholesterol, a cholesteryl or modified cholesteryl residue, stearyl, distearyl, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

In some embodiments, an oligonucleotide disclosed herein, such as an antisense oligonucleotide, is a free antisense oligonucleotide or a linear antisense oligonucleotide, which is not associated with a SNA disclosed herein, is not part of a SNA disclosed herein, or is not in a SNA configuration. In some embodiments, a free antisense oligonucleotide or a linear antisense oligonucleotide, which is not associated with a SNA disclosed herein, is not part of a SNA disclosed herein, or is not in a SNA configuration is referred to herein as a “structure”. Thus, a structure is contemplated herein according to some aspects. The use of a structure disclosed herein in any of the methods disclosed herein is also contemplated according to some aspects.

In some embodiments, an oligonucleotide disclosed herein is in an oligonucleotide shell. The oligonucleotide shell may be anchored to the surface of the core through one or multiple of linker molecules, including but not limited to: any chemical structure containing one or multiple thiols, such as the various chain length alkane thiols, cyclic dithiol, lipoic acid, or other thiol linkers known to those skilled in the art.

The exterior of the lipid-containing core has an oligonucleotide shell. The oligonucleotide shell can be constructed from a wide variety of nucleic acids including, but not limited to: single-stranded deoxyribonucleotides, ribonucleotides, and other single-stranded oligonucleotides incorporating one or a multiplicity of modifications known to those in the art, double-stranded deoxyribonucleotides, ribonucleotides, and other double-stranded oligonucleotides incorporating one or a multiplicity of modifications known to those in the art, oligonucleotide triplexes incorporating deoxyribonucleotides, ribonucleotides, or oligonucleotides that incorporate one or a multiplicity of modifications known to those in the art. In this particular invention, the SNAs described herein are constructed from oligonucleotides that are not as potent on their own.

The surface density of the oligonucleotides may depend on the size and type of the core and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 100 oligonucleotides per particle will be adequate to provide stable core-oligonucleotide conjugates. In some embodiments, the surface density is at least, or about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 1 to 17, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 155, 160, 165, 170, 175, 180, 185, 190, 195, 196, 197, 198, 199, 200, 201, 202, 203, 204, 205, 210, 215, 220, 225, 230, 235, 240, 245, 250, 300, 350, 400, 450, 500, 550, 600, 650, 700, 750, 800, 850, 900, 950, 1,000, 1,200, 1,400, 1,600, 1,800, 2,000, 3,000, 4,000, 5,000, 6,000, 7,000, 8,000, 9,000 or 10,000 oligonucleotides (e.g., antisense oligonucleotides) or any range combination thereof per SNA. In some embodiments, the surface density is 1-10,000, 1-9,000, 1-8,000, 1-7,000, 1-6,000, 1-5,000, 1-4,000, 1-3,000, 1-2,000, 1-1,000, 5-10,000, 5-9,000, 5-8,000, 5-7,000, 5-6,000, 5-5,000, 5-4,000, 5-3,000, 5-2,000, 5-1,000, 100-10,000, 100-9,000, 100-8,000, 100-7,000, 100-6,000, 100-5,000, 100-4,000, 100-3,000, 100-2,000,100-1,000, 500-10,000, 500-9,000, 500-8,000, 500-7,000, 500-6,000, 500-5,000, 500-4,000, 500-3,000, 500-2,000, 500-1,000, 10-10,000, 10-500, 50-10,000, 50-300, or 50-250 oligonucleotides per SNA.

In some embodiments, the surface density is at least 5, 10, 15, 20, 25, 30, 35, 40, 45, 50, 55, 60, 65, 70, 75, 80, 85, 100, 200, 300, 400, 500, 600, 700, 800, 900, 1,000, 1,200, 1,400, 1,600, 1,800, 2,000, 3,000, 4,000, 5,000, 6,000, 7,000, 8,000, 9,000 or 10,000 oligonucleotides or any range combination thereof per 20 nm liposome. In some embodiments, the surface density is 1-10,000, 1-9,000, 1-8,000, 1-7,000, 1-6,000, 1-5,000, 1-4,000, 1-3,000, 1-2,000, 1-1,000, 5-10,000, 5-9,000, 5-8,000, 5-7,000, 5-6,000, 5-5,000, 5-4,000, 5-3,000, 5-2,000, 5-1,000, 100-10,000, 100-9,000, 100-8,000, 100-7,000, 100-6,000, 100-5,000, 100-4,000, 100-3,000, 100-2,000,100-1,000, 500-10,000, 500-9,000, 500-8,000, 500-7,000, 500-6,000, 500-5,000, 500-4,000, 500-3,000, 500-2,000, 500-1,000, 10-10,000, 10-500, 50-10,000, 50-300, or 50-250 oligonucleotides per 20 nm liposome.

In some embodiments, a SNA described herein has an average or number mean diameter on the order of nanometers (i.e., between about 1 nm and about 1 micrometer). For example, in some instances, the number mean diameter of the nanoparticle is from about 1 nm to about 250 nm in number mean diameter, about 1 nm to about 240 nm in number mean diameter, about 1 nm to about 230 nm in number mean diameter, about 1 nm to about 220 nm in number mean diameter, about 1 nm to about 210 nm in number mean diameter, about 1 nm to about 200 nm in number mean diameter, about 1 nm to about 190 nm in number mean diameter, about 1 nm to about 180 nm in number mean diameter, about 1 nm to about 170 ran in number mean diameter, about 1 nm to about 160 nm in number mean diameter, about 1 nm to about 150 nm in number mean diameter, about 1 nm to about 140 nm in number mean diameter, about 1 nm to about 130 nm in number mean diameter, about 1 nm to about 120 nm in number mean diameter, about 1 nm to about 110 nm in number mean diameter, about 1 nm to about 100 nm in number mean diameter, about 1 nm to about 90 nm in number mean diameter, about 1 nm to about 80 nm in number mean diameter, about 1 nm to about 70 nm in number mean diameter, about 1 nm to about 60 nm in number mean diameter, about 1 nm to about 50 nm in number mean diameter, about 1 nm to about 40 nm in number mean diameter, about 1 nm to about 30 nm in number mean diameter, about 1 nm to about 25 nm in number mean diameter, about 1 nm to about 20 nm in number mean diameter, about 1 nm to about 15, about 1 nm to about 10 nm in number mean diameter, about 5 nm to about 150 nm in number mean diameter, about 5 to about 50 nm in number mean diameter, about 10 to about 30 nm in number mean diameter, about 10 to 150 nm in number mean diameter, about 10 to about 100 nm in number mean diameter, about 10 to about 50 nm in number mean diameter, about 30 to about 100 nm in number mean diameter, or about 40 to about 80 nm in number mean diameter.

In some embodiments, a SNA described herein has an average or number mean diameter of or about 5 nm in number mean diameter, 6 nm in number mean diameter, 7 nm in number mean diameter, 8 nm in number mean diameter, 9 nm in number mean diameter, 10 nm in number mean diameter, 11 nm in number mean diameter, 12 nm in number mean diameter, 13 nm in number mean diameter, 14 nm in number mean diameter, 15 nm in number mean diameter, 16 nm in number mean diameter, 17 nm in number mean diameter, 18 nm in number mean diameter, 19 nm in number mean diameter, 20 nm in number mean diameter, 21 nm in number mean diameter, 22 nm in number mean diameter, 23 nm in number mean diameter, 24 nm in number mean diameter, 25 nm in number mean diameter, 26 nm in number mean diameter, 27 nm in number mean diameter, 28 nm in number mean diameter, 29 nm in number mean diameter, 30 nm in number mean diameter, 31 nm in number mean diameter, 32 nm in number mean diameter, 33 nm in number mean diameter, 34 nm in number mean diameter, 35 nm in number mean diameter, 36 nm in number mean diameter, 37 nm in number mean diameter, 38 nm in number mean diameter, 39 nm in number mean diameter, 40 nm in number mean diameter, 41 nm in number mean diameter, 42 nm in number mean diameter, 43 nm in number mean diameter, 44 nm in number mean diameter, 45 nm in number mean diameter, 46 nm in number mean diameter, 47 nm in number mean diameter, 48 nm in number mean diameter, 49 nm in number mean diameter, 50 nm in number mean diameter, 55 nm in number mean diameter, 60 nm in number mean diameter, 65 nm in number mean diameter, 70 nm in number mean diameter, 75 nm in number mean diameter, 80 nm in number mean diameter, 85 nm in number mean diameter, 90 nm in number mean diameter, 95 nm in number mean diameter, 100 nm in number mean diameter, 110 nm in number mean diameter, 120 nm in number mean diameter, 130 nm in number mean diameter, 140 nm in number mean diameter, 150 nm in number mean diameter, 160 nm in number mean diameter, 170 nm in number mean diameter, 180 nm in number mean diameter, 190 nm in number mean diameter, 200 nm in number mean diameter or more than 200 nm in number mean diameter.

In some embodiments, the core comprises or consists of a metal core. Non-limiting examples of a metal include gold, silver, platinum, aluminum, palladium, copper, cobalt, indium, nickel and mixtures thereof. In some embodiments, the core comprises or consists of gold. In some embodiments, a nanostructure disclosed herein is degradable. In some embodiments, the core is a solid core. In some embodiments, the core is a hollow core. In some embodiments, a nanostructure or core disclosed herein comprises a semiconductor or magnetic material. In some embodiments, the core is a liposomal core.

In some embodiments, a core (e.g., a liposomal core or gold core) has an average or number mean diameter in the order of nanometers (i.e., between about 1 nm and about 1 micrometer). For example, in some instances, the number mean diameter of the core is from about 1 nm to about 250 nm in number mean diameter, about 1 nm to about 240 nm in number mean diameter, about 1 nm to about 230 nm in number mean diameter, about 1 nm to about 220 nm in number mean diameter, about 1 nm to about 210 nm in number mean diameter, about 1 nm to about 200 nm in number mean diameter, about 1 nm to about 190 nm in number mean diameter, about 1 nm to about 180 nm in number mean diameter, about 1 nm to about 170 ran in number mean diameter, about 1 nm to about 160 nm in number mean diameter, about 1 nm to about 150 nm in number mean diameter, about 1 nm to about 140 nm in number mean diameter, about 1 nm to about 130 nm in number mean diameter, about 1 nm to about 120 nm in number mean diameter, about 1 nm to about 110 nm in number mean diameter, about 1 nm to about 100 nm in number mean diameter, about 1 nm to about 90 nm in number mean diameter, about 1 nm to about 80 nm in number mean diameter, about 1 nm to about 70 nm in number mean diameter, about 1 nm to about 60 nm in number mean diameter, about 1 nm to about 50 nm in number mean diameter, about 1 nm to about 40 nm in number mean diameter, about 1 nm to about 30 nm in number mean diameter, about 1 nm to about 25 nm in number mean diameter, about 1 nm to about 20 nm in number mean diameter, about 1 nm to about 15, about 1 nm to about 10 nm in number mean diameter, about 5 nm to about 150 nm in number mean diameter, about 5 to about 50 nm in number mean diameter, about 10 to about 30 nm in number mean diameter, about 10 to 150 nm in number mean diameter, about 10 to about 100 nm in number mean diameter, about 10 to about 50 nm in number mean diameter, about 30 to about 100 nm in number mean diameter, or about 40 to about 80 nm in number mean diameter.

In some embodiments, a core (e.g., a liposomal core or gold core) disclosed herein, has an average or number mean diameter of or about 5 nm in number mean diameter, 6 nm in number mean diameter, 7 nm in number mean diameter, 8 nm in number mean diameter, 9 nm in number mean diameter, 10 nm in number mean diameter, 11 nm in number mean diameter, 12 nm in number mean diameter, 13 nm in number mean diameter, 14 nm in number mean diameter, 15 nm in number mean diameter, 16 nm in number mean diameter, 17 nm in number mean diameter, 18 nm in number mean diameter, 19 nm in number mean diameter, 20 nm in number mean diameter, 21 nm in number mean diameter, 22 nm in number mean diameter, 23 nm in number mean diameter, 24 nm in number mean diameter, 25 nm in number mean diameter, 26 nm in number mean diameter, 27 nm in number mean diameter, 28 nm in number mean diameter, 29 nm in number mean diameter, 30 nm in number mean diameter, 31 nm in number mean diameter, 32 nm in number mean diameter, 33 nm in number mean diameter, 34 nm in number mean diameter, 35 nm in number mean diameter, 36 nm in number mean diameter, 37 nm in number mean diameter, 38 nm in number mean diameter, 39 nm in number mean diameter, 40 nm in number mean diameter, 41 nm in number mean diameter, 42 nm in number mean diameter, 43 nm in number mean diameter, 44 nm in number mean diameter, 45 nm in number mean diameter, 46 nm in number mean diameter, 47 nm in number mean diameter, 48 nm in number mean diameter, 49 nm in number mean diameter, 50 nm in number mean diameter, 55 nm in number mean diameter, 60 nm in number mean diameter, 65 nm in number mean diameter, 70 nm in number mean diameter, 75 nm in number mean diameter, 80 nm in number mean diameter, 85 nm in number mean diameter, 90 nm in number mean diameter, 95 nm in number mean diameter, 100 nm in number mean diameter, 110 nm in number mean diameter, 120 nm in number mean diameter, 130 nm in number mean diameter, 140 nm in number mean diameter, 150 nm in number mean diameter, 160 nm in number mean diameter, 170 nm in number mean diameter, 180 nm in number mean diameter, 190 nm in number mean diameter, 200 nm in number mean diameter or more than 200 nm in number mean diameter.

In some embodiments, the ratio of oligonucleotide molecules to the diameter in nm of a core (e.g., liposomal core of a SNA, gold core of a SNA, etc.) disclosed herein is 30:20 (i.e., 30 oligonucleotide molecules per 20 nm diameter of SNA core), 6:1, 30:5, 3:1, 30:10, 15:2, 30:15, 3:2, 30:20, 6:5, 30:25, 1:1, 6:7, 30:35, 3:4, 30:40, 2:3, 30:45, 3:5, 30:50, 6:11, 30:55, 1:2, 6:13, 30:65, 3:7, 30:70, 2:5, 30:75, 3:8, 30:80, 6:17, 30:85, 1:3, 6:19, 30:95, 3:10, 30:100, 1:5, 3:20, 30:200, 1:10, 30:300, 1:4, 1:2, 3:4, 5:4, 1:1, 7:4, 2:1, 9:4, 5:2, 11:4, 3:1, 13:4, 7:2, 15:4, 4:1, 17:4, 19:4, 5:1, 10:1, or 15:1 or any range or combination thereof.

In some embodiments, an oligonucleotide or antisense oligonucleotide disclosed herein comprises a linker. In some embodiments, the linker is between a group that associates with a core described herein and an oligonucleotide or antisense oligonucleotide disclosed herein. In some embodiments, the linker comprises or consists of an oligonucleotide, a peptide, a polymer or an oligoethylene (e.g., hexaethylene glycol or iSp18). In some embodiments, the linker does not comprise or does not consist of an oligonucleotide (e.g., non-nucleotidic linker), a peptide, a polymer or an oligoethylene. In some embodiments, an oligonucleotide or antisense oligonucleotide disclosed herein comprises a linker. In some embodiments, the linker forms a covalent bond with a core, such as a gold-thiol bond that forms in a gold core.

Non-limiting examples of constructs compatible with aspects of the invention are described in and incorporated by reference from: U.S. Pat. No. 7,238,472, US Patent Publication No. 2003/0147966, US Patent Publication No. 2008/0306016, US Patent Publication No. 2009/0209629, US Patent Publication No. 2010/0136682, US Patent Publication No. 2010/0184844, US Patent Publication No. 2010/0294952, US Patent Publication No. 2010/0129808, US Patent Publication No. 2010/0233270, US Patent Publication No. 2011/0111974, PCT Publication No. WO 2002/096262, PCT Publication No. WO 2003/08539, PCT Publication No. WO 2006/138145, PCT Publication No. WO 2008/127789, PCT Publication No. WO 2008/098248, PCT Publication No. WO 2011/079290, PCT Publication No. WO 2011/053940, PCT Publication No. WO 2011/017690 and PCT Publication No. WO 2011/017456. Constructs, such as SNAs, associated with the invention can be synthesized according to any means known in the art or can be obtained commercially. For example, several non-limiting examples of commercial suppliers of nanoparticles include: Ted Pella, Inc., Redding, Calif., Nanoprobes, Inc., Yaphank, N.Y., Vacuum Metallurgical Co. Ltd., Chiba, Japan and Vector Laboratories, Inc., Burlington, Calif.

In some embodiments, a SNA containing a first oligonucleotide, such as a first antisense oligonucleotide, described herein is co-administered with one or more oligonucleotides, such as antisense oligonucleotides. In some embodiments, the second oligonucleotide is designed to treat the same disease, disorder, or condition as the first oligonucleotide described herein. In some embodiments, the first oligonucleotide (e.g., first antisense oligonucleotide) and the second oligonucleotide (e.g., second antisense oligonucleotide) are in the same SNA. In some embodiments, the first oligonucleotide is more abundant in the SNA than the second oligonucleotide. In some embodiments, the second oligonucleotide is more abundant in the SNA than the first oligonucleotide. In some embodiments, the SNA contains about the same amounts of the first oligonucleotide and the second oligonucleotide. In some embodiments, the first oligonucleotide affects a first region of the SMN2 pre-mRNA and the second oligonucleotide affects a second region of the SMN2 pre-mRNA. In some embodiments, the first region of the SMN2 pre-mRNA is ISS-N1. In some embodiments, the second region of the SMN2 pre-mRNA comprises the genetic region upstream of SMN2 exon 7 called Element 1 (E1). (See e.g., Osman et al., Human Molecular Genetics (2014) 23(18):4832-45). In some embodiments, the nucleotide sequence for E1 corresponds to the nucleic acid sequence of SEQ ID NO: 10:

(SEQ ID NO: 10)
5′ - CTA TAT ATA GAT AGC TTT ATA TGG ATG TTA AAA
AGC ATT TTG TTT CAC AAG ACA TTT TAC TTA TTT TAT
TCA ACA AA - 3′

In some embodiments, the first region or second region of the SMN2 gene is a 3′ splice site of exon 8, also known as ex8 3′ ss. In some embodiments, the first region or second region of the SMN2 gene is ISS+100. (See e.g., Pao et al., Molecular Therapy (2014) 22(4):855-61). In some embodiments, the first oligonucleotide is in a first SNA and the second oligonucleotide is in a second SNA. In some embodiments, a plurality of different oligonucleotides are in one SNA. In some embodiments, a plurality of different oligonucleotides are in more than one SNA.

In some embodiments, a SNA containing a first oligonucleotide, such as a first antisense oligonucleotide, described herein is co-administered with one or more secondary agents, such as a drug or compound.

In some embodiments, one or more of secondary oligonucleotides or agents are co-administered with the first oligonucleotide to produce a combinational effect. In some embodiments, second oligonucleotides are co-administered with the first oligonucleotide to produce a synergistic effect. In some embodiments, the co-administration of the first and second oligonucleotides permits use of lower dosages than would be required to achieve a therapeutic or prophylactic effect if the oligonucleotides were administered as independent therapy.

In some embodiments, inclusion of exon 7 in the SMN2 pre-mRNA is achieved through targeting a regulator of SMN2 pre-mRNA splicing. In some embodiments, an oligonucleotide targeting a regulator of mRNA splicing, such as an oligonucleotide that regulates exon 7 inclusion, is in a SNA described herein. In some embodiments, the oligonucleotide improves exon 7 inclusion in the SMN2 pre-mRNA through downregulation of an RNA binding protein. In some embodiments, the RNA binding protein is RBM10. (See e.g., Sutherland et al. BMC Molecular Biol (2017) 18:19).

In some embodiments, RBM10 is downregulated using an siRNA of SEQ ID NO: 18, targeting exon 7 or SEQ ID NO: 19, targeting exon 23:

(SEQ ID NO: 18)
5′-AAG GUG UCG AUG CAC UAC A-3′
(SEQ ID NO: 19)
5′-GCA UUG UAA CGC CUA UCG A-3′

In some embodiments, the regulator of mRNA splicing is a serine/arginine (SR) splicing factor or a heterogeneous ribonucleoprotein (hnRNP) protein. (See e.g., Wee et al., PLoS ONE (2014) 9(12):e115205). In some embodiments, an oligonucleotide in an SNA described herein improves exon 7 inclusion in the SMN2 pre-mRNA through downregulation of an SR splicing factor or a hnRNP protein. In some embodiments, the SR splicing factor is SRSF1, SRSF2, SRSF3, SRSF4, SRSF5, SRSF6, SRSF7 or SRSF11. (See e.g., Cartegni et al. American journal of human genetics (2006) 78:63-77; Kashima et al. Nature genetics (2003) 34:460-3; Young et al. (2002) Hum Mol Genet 11: 577-87; and Cartegni et al. Nat Genet (2002) 30: 377-84). In some embodiments, the hnRNP protein is hnRNPA1, hnRNP A2B1, hnRNP C or hnRNP U. (See e.g., Kashima et al. Hum Mol Genet (2007) 16:3149-59; Hua et al. American journal of human genetics 82: 834-48; Irimura et al. The Kobe journal of medical sciences (2009) 54: E227-236; and Xiao et al. Mol Cell (2012) 45:656-68).

In some embodiments, the regulator of mRNA splicing is HuR/ELAVL1, Puf60, Sam68, SF1, SON, U2AF35 or ZIS2/ZNF265. (See e.g., Wee et al., PLoS ONE (2014) 9(12):e115205). In some embodiments, an oligonucleotide in an SNA described herein improves exon 7 inclusion in the SMN2 pre-mRNA through downregulation of HuR/ELAVL1, Puf60, Sam68, SF1, SON, U2AF35 or ZIS2/ZNF265.

In some embodiments, the regulator of mRNA splicing is targeted with one or more oligonucleotides, such as one or more of the siRNAs disclosed in Table 1 below. (See e.g., Wee et al., PLoS ONE (2014) 9(12):e115205). In some embodiments the one or more oligonucleotides are in one or more SNAs described herein.

TABLE 1

| | SEQ ID | | SEQ ID | |
|---|---|---|---|---|
| Gene | NO | SiRNA Sequence | NO | Primer Sequence |
| SRSF1 | 20 | 5′-GCAGAUGAACUCGGGAUG-3′ | 42 | F 5′-CAGAGTGGTTGTCTCTG-3′ |
| | 21 | 3′-CGUCUACUUGAGCCACUAC-5′ | 43 | R 5′-CTCCACGACACCAGTGCC-3′ |
| SRSF2 | 22 | 5′-CCGCACUCGUUCUCGAUCUTT-3′ | 44 | F 5′-GGACGCCGGAGCCGCAG-3′ |
| | 23 | 3′-AGGGCGUGAGCAAGAGCUAGA-5′ | 45 | R 5′-GAGATCGAGAACGAGTGC-3′ |
| SRSF3 | 24 | 5′-GCUAGAUGGAAGAACACUAT-3′ | 46 | F 5′-ATGCATCGTGATTCCTG-3′ |
| | 25 | 3′-CTCGAUCUACCUUCUUGUGAU-5′ | 47 | R 5′-CTGCGACGAGGTGGAGG-3′ |
| SRSF4 | 26 | 5′-GGACUGCCUCCAAGUGGAATT-3′ | 48 | F 5′-GTTACGGTTCTGGACGC-3′ |
| | 27 | 3′-GACCUGACGGAGGUUCACCUU-5′ | 49 | R 5′-GCTCCGGGAGCGGGAG-3′ |
| SRSF5 | 28 | 5′-CCUCGAAAUGAUAGACGAATT-3′ | 50 | F 5′-GATCCAAGGGATGCAGATG-3′ |
| | 29 | 3′-TTGGAGCUUUACUAUCUGCUU-5′ | 51 | R 5′-CTATCATTTCGAGGTCTGCG-3′ |
| SRSF6 | 30 | 5′-GCAUAGGGUUGACUGAUAATT-3′ | 52 | F 5′-GTGGATACAGCAGTCGG-3′ |
| | 31 | 3′-CTCGUAUCCCAACUGACUAUU-5′ | 53 | R 5′-CTGGATCTGCTTCCAGAG-3′ |
| SRSF7 | 32 | 5′-CGACGUCCCUUUGAUCCAATT-3′ | 54 | F 5′-GGTCTAGATCACATTCTCG-3′ |
| | 33 | 3′-GGGCUGCAGGGAAACUAGGUU-5′ | 55 | R 5′-CCAGACCTAGATCTTCTG-3′ |
| SRSF11 | 34 | 5′-GGAUACCUCUAGUAAAGAATT-3′ | 56 | F 5′-CAGGAGCGAGAACCCGAG-3′ |
| | 35 | 3′-AGCCUAUGGAGAUCAUUUCUU-5′ | 57 | R 5′-CTTCTGCATATGGTACGAC-3′ |
| hnRNP | 36 | 5′-GGAUUAUUUAAUAACAUUATT-3′ | 58 | F n/a |
| A2B1 | 37 | 3′-AACCUAAUAAAUUAUUGUAAU-5′ | 59 | R |
| hnRNP C | 38 | 5′-CGUCAGCGUGUAUCAGGAATT-3′ | 60 | F 5′-GTTACCCAGCACGTGTACC-3′ |
| | 39 | 3′-GCAGUCGCACAUAGUCCUU-5′ | 61 | R 5′-GGCCTGAAGGTCATCTCC-3′ |
| hnRNP U | 40 | 5′-GGCCGUGGUAGUUACUCAATT-3′ | 62 | F 5′-GAGTACATTGAAGAGAACAAG-3′ |
| | 41 | 3′-GACCGGCACCAUCAAUGAGUU-5′ | 63 | R 5′-CACTGTGTCATCGAAGTGTTC-3′ |

In some embodiments, an oligonucleotide targeting a regulator of mRNA splicing, such as an oligonucleotide that regulates exon 7 inclusion, is in a SNA described herein. In some embodiments, an oligonucleotide targeting a regulator of mRNA splicing and one or more oligonucleotides targeting a region of the SMN2 pre-mRNA are in different SNAs. In some embodiments, an oligonucleotide targeting a regulator of mRNA splicing and one or more oligonucleotides targeting a region of the SMN2 pre-mRNA are in the same SNA.

In some embodiments, the second oligonucleotide targets a long non-coding RNA (lncRNA), which results in an increase in SMN expression in vitro and in vivo. In some embodiments, the second oligonucleotide is an antisense oligonucleotide (traditional antisense) that targets a lncRNA by binding to the lncRNA, forming a duplex that is susceptible to RNAse H cleavage or siRNA that leads to RISC-catalyzed mRNA degradation. In other embodiments, the second oligonucleotide is siRNA that targets a lncRNA. In some embodiments, the lncRNA is SMN-AS1, GenBank accession #BC045789.1 (d'Ydewalle et al., 2017, Neuron 93, 66-79). In embodiments the second oligonucleotide is chosen from SEQ ID NO: 81 to SEQ ID NO: 160 or oligonucleotides having 85%, 90%, 95%, 96%, 97%, 98%, or 99% sequence identity with oligonucleotides of SEQ ID NO: 81 to SEQ ID NO: 160. In some embodiments, the second oligonucleotide has a 5-10-5 MOE gapmer design, wherein the central gap segment comprises of ten 2′-deoxynucleosides and is flanked by wing segments on the 5′ direction and the 3′ direction comprising five nucleosides each. Each nucleoside in the 5′ wing segment and/or each nucleoside in the 3′ wing segment may have a 2′-MOE modification. The internucleoside linkages throughout each gapmer are phosphorothioate (P═S) linkages. In some embodiments, the gapmers have mixed backbone, including phosphorothioate and phosphodiester internucleotide linkages. In some embodiments, one or more or all cytosine residues throughout each gapmer are 5-methylcytosines.

| Seq ID | |
|---|---|
| No | Sequence (5′→ 3′) |
| 81 | G*T*A*C*T*A*C*A*C*T*T*T*T*A*A*T*T*A*C*T- (Spacer 18)2 - 3CholTEG |
| 82 | T*G*T*A*T*A*T*T*G*A*T*G*T*C*A*G*T*A*C*T- (Spacer 18)2 - 3CholTEG |
| 83 | T*A*C*A*T*T*G*T*C*T*A*T*T*A*G*T*G*T*A*T- (Spacer 18)2 - 3CholTEG |
| 84 | T*G*A*C*T*C*T*C*A*A*T*T*C*T*G*T*T*A*C*A- (Spacer 18)2 - 3CholTEG |
| 85 | T*C*A*C*A*G*G*G*C*T*A*T*T*T*C*T*G*A*C*T- (Spacer 18)2 - 3CholTEG |
| 86 | A*A*T*C*A*G*T*C*A*C*A*T*A*T*A*T*C*A*C*A- (Spacer 18)2 - 3CholTEG |
| 87 | T*G*T*A*A*C*T*T*T*A*G*T*T*A*A*A*A*T*C*A- (Spacer 18)2 - 3CholTEG |
| 88 | C*T*A*T*T*A*A*A*C*C*A*C*A*T*T*T*G*T*A*A- (Spacer 18)2 - 3CholTEG |
| 89 | A*C*T*A*C*T*A*T*G*C*T*T*T*C*T*C*T*A*T*T- (Spacer 18)2 - 3CholTEG |
| 90 | C*C*A*C*C*A*T*T*T*C*T*T*G*A*A*A*C*T*A*C- (Spacer 18)2 - 3CholTEG |
| 91 | T*T*T*C*C*A*A*T*A*G*T*T*T*T*A*C*C*A*C*C- (Spacer 18)2 - 3CholTEG |
| 92 | G*T*T*T*T*T*G*C*A*T*A*A*G*G*A*T*T*T*C*C- (Spacer 18)2 - 3CholTEG |
| 93 | G*T*G*G*A*A*A*T*T*T*G*G*T*T*T*G*T*T*T*T- (Spacer 18)2 - 3CholTEG |
| 94 | T*G*T*G*G*C*T*C*A*G*T*G*T*A*G*G*T*G*G*A- (Spacer 18)2 - 3CholTEG |
| 95 | G*T*A*T*T*A*A*T*T*C*T*T*A*T*A*T*G*T*G*G- (Spacer 18)2 - 3CholTEG |
| 96 | T*T*A*G*T*T*T*T*A*C*A*C*T*T*A*G*G*T*C*T- (Spacer 18)2 - 3CholTEG |
| 97 | A*C*A*C*A*G*T*T*T*A*G*A*G*T*T*T*T*A*G*T- (Spacer 18)2 - 3CholTEG |
| 98 | G*A*T*C*A*C*A*G*A*T*T*T*T*T*C*T*C*T*C*T- (Spacer 18)2 - 3CholTEG |
| 99 | T*T*A*T*A*G*G*C*A*A*T*C*C*A*T*G*A*T*C*A- (Spacer 18)2 - 3CholTEG |
| 100 | C*A*T*T*T*C*A*G*T*T*T*G*T*T*C*T*T*T*T*G- (Spacer 18)2 - 3CholTEG |
| 101 | C*C*C*A*G*G*C*A*A*C*A*A*G*G*C*C*A*T*T*T- (Spacer 18)2 - 3CholTEG |
| 102 | G*A*A*C*C*T*C*G*G*G*T*G*C*C*A*C*C*C*C*A- (Spacer 18)2 - 3CholTEG |
| 103 | C*G*T*C*C*T*T*G*A*T*T*T*C*C*T*C*A*G*C*G- (Spacer 18)2 - 3CholTEG |
| 104 | A*C*A*C*C*C*T*T*G*G*T*G*T*G*T*C*A*G*C*G- (Spacer 18)2 - 3CholTEG |
| 105 | T*T*C*T*G*C*T*C*T*A*G*C*C*T*C*A*C*A*C*C- (Spacer 18)2 - 3CholTEG |
| 106 | G*G*A*G*A*G*A*G*C*T*A*G*T*C*T*C*T*T*T*C- (Spacer 18)2 - 3CholTEG |
| 107 | A*G*G*A*C*C*T*C*T*C*T*C*T*G*C*A*G*G*A*G- (Spacer 18)2 - 3CholTEG |
| 108 | A*T*G*G*G*A*A*C*T*C*T*T*T*T*C*A*G*G*A*C- (Spacer 18)2 - 3CholTEG |
| 109 | G*C*A*T*T*T*C*A*C*T*G*T*G*G*A*A*T*G*G*G- (Spacer 18)2 - 3CholTEG |
| 110 | T*T*T*A*T*A*A*A*A*A*T*G*C*T*T*G*C*A*T*T- (Spacer 18)2 - 3CholTEG |
| 111 | C*T*T*C*C*C*A*T*T*A*G*C*T*C*A*T*T*T*A*T- (Spacer 18)2 - 3CholTEG |
| 112 | T*A*G*A*T*A*A*G*C*T*A*C*C*C*C*C*T*T*C*C- (Spacer 18)2 - 3CholTEG |
| 113 | T*T*T*G*C*T*C*C*C*T*A*T*G*T*G*T*A*G*A*T- (Spacer 18)2 - 3CholTEG |
| 114 | G*G*T*C*C*T*A*A*C*T*G*G*T*T*T*T*T*T*G*C- (Spacer 18)2 - 3CholTEG |
| 115 | C*A*G*A*T*G*G*C*A*A*C*A*C*C*T*G*G*T*C*C- (Spacer 18)2 - 3CholTEG |
| 116 | G*A*T*T*C*A*C*G*C*T*C*T*G*T*G*C*A*G*A*T- (Spacer 18)2 - 3CholTEG |
| 117 | G*C*C*T*G*C*A*T*A*A*T*A*A*A*A*G*G*T*T*G- (Spacer 18)2 - 3CholTEG |
| 118 | T*C*A*G*G*C*C*A*A*G*G*A*C*C*T*G*C*C*T*G- (Spacer 18)2 - 3CholTEG |
| 119 | T*A*A*G*C*A*A*T*G*T*G*G*A*G*T*A*G*C*T*C- (Spacer 18)2 - 3CholTEG |
| 120 | A*C*A*A*T*A*G*G*A*A*A*G*A*G*A*T*A*A*G*C- (Spacer 18)2 - 3CholTEG |
| 121 | T*T*A*T*T*T*A*G*C*A*C*A*T*G*C*A*C*A*A*T- (Spacer 18)2 - 3CholTEG |
| 122 | T*G*G*C*T*C*C*A*C*C*T*C*C*C*C*T*T*A*T*T- (Spacer 18)2 - 3CholTEG |
| 123 | G*C*A*T*G*T*C*C*A*C*C*A*T*G*G*T*G*G*C*T- (Spacer 18)2 - 3CholTEG |
| 124 | A*G*C*T*G*C*A*C*G*G*A*G*A*G*A*A*A*G*G*G- (Spacer 18)2 - 3CholTEG |
| 125 | G*C*A*T*G*T*T*G*T*G*A*G*T*T*G*T*T*G*G*G- (Spacer 18)2 - 3CholTEG |
| 126 | T*C*A*G*A*T*A*A*G*G*A*A*G*C*T*G*G*A*A*G- (Spacer 18)2 - 3CholTEG |
| 127 | G*A*C*C*T*T*A*G*T*A*C*A*T*A*C*T*C*A*G*A- (Spacer 18)2 - 3CholTEG |
| 128 | G*A*A*G*T*A*A*A*C*A*C*A*G*T*G*G*A*C*C*T- (Spacer 18)2 - 3CholTEG |
| 129 | G*T*A*T*G*T*G*A*A*G*T*A*A*A*C*A*C*A*G*T- (Spacer 18)2 - 3CholTEG |
| 130 | G*T*A*A*A*C*A*C*A*G*T*A*T*G*T*G*A*A*G*T- (Spacer 18)2 - 3CholTEG |
| 131 | A*G*G*T*G*G*G*T*A*T*G*T*G*A*A*G*T*A*A*A- (Spacer 18)2 - 3CholTEG |
| 132 | A*T*C*A*G*C*A*A*G*C*T*T*C*A*C*A*T*A*C*G- (Spacer 18)2 - 3CholTEG |
| 133 | G*G*A*G*C*T*T*C*C*T*G*G*G*T*A*A*T*C*A*G- (Spacer 18)2 - 3CholTEG |
| 134 | A*G*C*A*G*C*T*C*T*G*G*C*A*C*A*G*A*G*G*G- (Spacer 18)2 - 3CholTEG |
| 135 | A*A*A*C*A*T*G*T*A*T*A*A*G*G*A*A*G*C*A*G- (Spacer 18)2 - 3CholTEG |
| 136 | G*G*A*A*G*A*T*C*G*G*G*C*T*G*T*A*A*A*C*A- (Spacer 18)2 - 3CholTEG |
| 137 | A*C*T*T*C*T*C*T*T*C*T*A*A*C*A*A*G*G*A*G- (Spacer 18)2 - 3CholTEG |
| 138 | C*A*G*A*G*T*C*C*T*C*G*G*T*A*G*A*A*C*T*T- (Spacer 18)2 - 3CholTEG |
| 139 | A*A*G*C*C*G*A*T*A*G*T*T*A*G*A*C*A*G*A*G- (Spacer 18)2 - 3CholTEG |
| 140 | A*A*A*A*A*A*A*G*A*C*T*A*G*G*T*A*A*G*C*C- (Spacer 18)2 - 3CholTEG |
| 141 | G*T*T*T*T*G*A*G*A*G*A*G*G*A*G*G*T*A*A*A- (Spacer 18)2 - 3CholTEG |
| 142 | G*T*T*T*T*T*T*C*T*T*T*G*A*T*G*G*T*T*T*T- (Spacer 18)2 - 3CholTEG |
| 143 | G*A*A*A*T*C*T*A*A*T*T*T*T*T*C*A*G*T*T*T- (Spacer 18)2 - 3CholTEG |
| 144 | A*A*T*C*T*T*A*A*T*T*T*T*G*C*T*G*A*A*A*T- (Spacer 18)2 - 3CholTEG |
| 145 | T*T*T*T*T*A*A*G*A*A*C*A*G*A*A*A*A*T*C*T- (Spacer 18)2 - 3CholTEG |
| 146 | A*C*A*C*T*T*T*G*G*T*T*T*T*T*C*A*T*T*T*T- (Spacer 18)2 - 3CholTEG |
| 147 | A*T*T*T*T*C*T*C*C*C*G*G*T*T*T*A*C*A*C*T- (Spacer 18)2 - 3CholTEG |
| 148 | A*G*G*T*A*A*C*T*T*G*C*A*T*G*T*A*T*T*T*T- (Spacer 18)2 - 3CholTEG |
| 149 | A*A*T*A*T*C*T*T*T*A*T*C*A*G*A*T*A*G*G*T- (Spacer 18)2 - 3CholTEG |
| 150 | A*T*G*T*T*T*G*C*T*G*G*G*T*A*C*A*A*T*A*T- (Spacer 18)2 - 3CholTEG |
| 151 | G*T*T*T*G*A*G*A*G*T*T*C*T*T*C*A*T*G*T*T- (Spacer 18)2 - 3CholTEG |
| 152 | C*A*T*C*T*T*T*T*A*A*T*T*G*A*A*T*T*T*T*T- (Spacer 18)2 - 3CholTEG |
| 153 | C*C*C*G*G*C*C*A*A*C*T*T*A*C*C*C*A*T*C*T- (Spacer 18)2 - 3CholTEG |
| 154 | G*A*T*T*G*G*G*A*T*T*G*C*A*A*G*T*A*T*G*A- (Spacer 18)2 - 3CholTEG |
| 155 | G*A*G*C*A*C*A*C*G*C*C*A*C*A*A*T*G*C*C*T- (Spacer 18)2 - 3CholTEG |
| 156 | A*G*T*C*T*T*C*T*T*G*T*C*T*C*A*G*C*C*T*T- (Spacer 18)2 - 3CholTEG |
| 157 | C*C*A*C*C*T*C*C*T*G*C*G*C*T*C*A*G*T*C*T- (Spacer 18)2 - 3CholTEG |
| 158 | T*C*A*C*A*C*A*G*C*C*T*A*C*T*G*C*A*G*C*C- (Spacer 18)2 - 3CholTEG |
| 159 | C*A#G#A#G#T*C*C*T*C*G*G*T*A*G*A#A#C*T*T- (Spacer 18)2 - 3CholTEG |
| 160 | G*A#A#A#T#C*T*A*A*T*T*T*T*T*C*A#G#T*T*T- (Spacer 18)2 - 3CholTEG |

*= phosphorothioate internucleotide linkage;
#= phosphodiester internucleotide linkage;
Spacer 18 = internal hexaethyleneglycol spacer;
3CholTEG = 3′-Cholesteryl-TEG;
TEG = tetraethylene glycol

In some embodiments, the first oligonucleotide and the second oligonucleotides are in the same SNA. In some embodiments, the first oligonucleotide and the second oligonucleotide are in separate SNAs, where such SNAs can be administered as a mixture, or one SNA after the other. In some embodiments, the SNA contains more than two distinct oligonucleotides. In some embodiments, the SNA contains oligonucleotides that target more than two distinct targets.

In some embodiments, a modification to one or more of the nucleotides of an oligonucleotide or antisense oligonucleotide described herein decreases or prevents RNAse H-catalyzed mRNA degradation. In some embodiments, the modification is a 2′-methoxyethyl (2′-MOE) modification, such as the 2′-MOE modification used in Spinraza (nusinersen). In some embodiments, the modification is a 2′-O-methyl modification. In some embodiments, other modifications, such as modifications known to one of ordinary skill in the art, decrease or prevent RNAse H catalyzed mRNA degradation. Without wishing to be bound by theory, oligonucleotides or antisense oligonucleotides, such as the oligonucleotides or antisense oligonucleotides described herein, that are less prone or completely protected from RNAse H-catalyzed mRNA degradation are useful in therapy that modifies mRNA splicing. In some embodiments, the modification is used in combination with traditional antisense/siRNA therapy. As described herein, traditional antisense/siRNA therapy relates to RNAse H dependent cleavage of mRNA; traditional antisense/siRNA therapy is the RISC-catalyzed mRNA degradation. In exon modulation or splice modulation, the aim is not to degrade the target mRNA. In some embodiments, only the splicing patterns are altered.

In some embodiments, the present disclosure provides administration of a first SNA into the cerebrospinal fluid (CSF), in combination with systemic delivery of a second SNA. Systemic administration and CSF administration can occur simultaneously, separately or sequentially. In some embodiments, a subject receives a first dose of a SNA in the CSF and subsequently receives a second dose of a SNA through a different route of administration. In some embodiments, a subject receives a first dose of a SNA in the CSF and subsequently receives a second dose of an antisense compound systemically. In some embodiments, the SNA administered into the CSF comprises the oligonucleotide of SEQ ID NO:1 or SEQ ID NO: 16.

In some embodiments, a target precursor transcript is associated with a disease or condition. In certain such embodiments, an oligomeric compound comprising or consisting of a modified oligonucleotide that is complementary to the target precursor transcript is used to treat the disease or condition. In certain such embodiments, the compound modulates processing of the target precursor transcript to produce a beneficial target processed transcript. In certain such embodiments, the disease or condition is associated with aberrant processing of a precursor transcript. In certain such embodiments, the disease or condition is associated with aberrant splicing of a pre-mRNA.

In some embodiments, a SNA described herein is used for the treatment of a disease or disorder associated with a decrease in survival motor neuron (SMN) protein or a disease or disorder associated with a deletion of the SMN1 gene that results in reduced or eliminated SMN protein expression. A non-limiting example includes spinal muscular atrophy (SMA). SMA is a genetic disorder characterized by degeneration of spinal motor neurons. SMA is caused by the loss of both functional copies of the survival motor neuron 1 (SMN1) gene, which may also be known as SMN Telomeric, a protein that is part of a multi-protein complex thought to be involved in snRNP biogenesis and recycling. A nearly identical gene, SMN2, which may also be known as SMN Centromeric, exists in a duplicated region on chromosome 5ql3 and modulates disease severity. Expression of the normal SMN1 gene results solely in expression of survival motor neuron (SMN) protein. Although SMN1 and SMN2 have the potential to code for the same protein, SMN2 contains a translationally silent mutation at position +6 of exon 7, which results in inefficient inclusion of exon 7 in SMN2 transcripts. Thus, the predominant form of SMN2 is a truncated version, lacking exon 7 (SMNΔ7), which is unstable and inactive (Cartegni et al. Nat Genet (2002) 30:377-84). Expression of the SMN2 gene results in approximately 10-20% of the SMN protein and 80-90% of the unstable/non-functional SMNΔ7 protein. SMN protein plays a well-established role in assembly of the spliceosome and may also mediate mRNA trafficking in the axon and nerve terminus of neurons. Thus, therapeutic compounds capable of modulating SMN2 splicing such that the percentage of SMN2 transcripts containing exon 7 is increased would be useful for the treatment of SMA.

In one embodiment, SMA is caused by a reduction of the SMN protein. In another embodiment, SMA is caused by a mutation in the SMN1 gene. In one embodiment, the type of SMA can be SMA1, SMA2, SMA3, SMA4, SMARD, SBMA, or DSMA.

SMA1 (also known as Werdnig-Hoffmann disease) is believed to be the most common form. It causes severe muscle weakness, which can result in problems moving, eating, breathing and swallowing. These symptoms are usually apparent at birth or during the first few months of life. The muscles of babies with SMA1 are thin and weak. They're usually unable to raise their head or sit without support. Breathing problems can be caused by weakness in the baby's chest muscles, and difficulty swallowing can be made worse by weakness of the muscles in the tongue and throat. Because of the high risk of serious respiratory problems, most children with SMA1 die in the first few years of life.

Symptoms of SMA2 usually appear when an infant is 7-18 months old. The symptoms are less severe than SMA1, but become more noticeable in older children. Infants with SMA2 are usually able to sit, but cannot stand or walk unaided. They may also have the following symptoms: breathing problems, weakness in their arms and, particularly, their legs, swallowing or feeding problems, and/or a slight tremor (shaking) of their fingers. In some cases, deformities of the hands, feet, chest and joints develop as the muscles shrink. As they grow, many children with SMA2 develop scoliosis. This is an abnormal curvature of the spine caused by the muscles supporting the bones of the spine becoming weaker. A child with SMA2 has weak respiratory muscles, which can make it difficult for them to cough effectively. This can make them more vulnerable to respiratory infections. Although SMA2 may shorten life expectancy, improvements in care standards mean most people can live long, fulfilling and productive lives. The majority of children with SMA2 are now expected to survive into adulthood.

SMA3 (also known as Kugelberg-Welander disease) is the mildest form of childhood SMA. Symptoms of muscle weakness usually appear after 18 months of age, but this is very variable and sometimes the symptoms may not appear until late childhood or early adulthood. Most children with SMA3 are able to stand unaided and walk, although many find walking or getting up from a sitting position difficult. They may also have: balance problems, difficulty walking, difficulty running or climbing steps, and/or a slight tremor (shaking) of their fingers. Over time, the muscles of children with SMA3 become weaker, resulting in some children losing the ability to walk when they get older. Breathing and swallowing difficulties are very rare and the condition doesn't usually affect life expectancy.

SMA4 is a less common form that begins in adulthood. The symptoms are usually mild to moderate, and may include: muscle weakness in the hands and feet, difficulty walking, and/or muscle tremor (shaking) and twitching. SMA4 doesn't affect life expectancy.

Spinal muscular atrophy with respiratory distress (SMARD) is a very rare form of SMA that severely affects the muscles used in breathing. It's usually diagnosed within the first year of life.

Kennedy's syndrome, or spinobulbar muscular atrophy (SBMA), is a rare type of adult SMA. SBMA only affects men. It usually develops very gradually between the ages of 20 and 40. Rarely, it can affect teenage boys or sometimes only become obvious after 40. The initial symptoms of Kennedy's syndrome may include tremor (shaking) of the hands, muscle cramps on exertion, and/or muscle twitches and weakness of the limb muscles. As the condition progresses, it may cause other symptoms, including: weakness of the facial and tongue muscles, which may cause difficulty swallowing (dysphagia) and slurred speech, and/or recurring pneumonia (infection of lung tissue). Some people with Kennedy's syndrome also develop enlarged male breasts (gynaecomastia), diabetes, and a low sperm count or infertility. Kennedy's syndrome doesn't usually affect life expectancy.

Distal spinal muscular atrophy (DSMA) is a rare form of SMA that affects the distal muscles, such as the hands, feet, lower arms and lower legs. This leads to reduced mobility and range of movement. Some types of DSMA can affect the muscles used for speaking or swallowing.

In some embodiments, a SNA described herein is used for the treatment of a genetic disorder. Non-liminting examples include achondroplasia, alpha-1 antitrypsin deficiency, antiphospholipid syndrome, autism, autosomal dominant polycystic kidney disease, breast cancer, charcot-marie-tooth, colon cancer, cri du chat, crohn's disease, cystic fibrosis, dercum disease, down syndrome, duane syndrome, duchenne muscular dystrophy, factor v leiden, thrombophilia, familial hypercholesterolemia, familial mediterranean fever, fragile x syndrome, gaucher disease, hemochromatosis, hemophilia, holoprosencephaly, huntington's disease, klinefelter syndrome, marfan syndrome, myotonic dystrophy, neurofibromatosis, noonan syndrome, osteogenesis imperfecta, parkinson's disease, phenylketonuria, poland anomaly, porphyria, progeria, prostate cancer, retinitis pigmentosa, severe combined immunodeficiency (scid), sickle cell disease, skin cancer, SMA, tay-sachs, thalassemia, trimethylaminuria, turner syndrome, velocardiofacial syndrome, wagr syndrome, and wilson disease.

Aspects of the invention relate to delivery of SNAs to a subject for therapeutic and/or diagnostic use. The SNAs may be administered alone or in any appropriate pharmaceutical carrier, such as a liquid, for example saline, or a powder, for administration in vivo. The SNAs can also be co-delivered with larger carrier particles or within administration devices. The SNAs may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. It should be appreciated that any method of delivery of SNAs known in the art may be compatible with aspects of the invention.

As used herein, a “patient,” “individual,” “subject” or “host” refers to either a human, a nonhuman animal, a primate or a mammal. In some embodiments, the mammal is a vertebrate animal including but not limited to a mouse, rat, dog, cat, horse, cow, pig, sheep, goat, turkey, chicken, primate, e.g., monkey, and fish (aquaculture species), e.g. salmon. Thus, the invention can also be used to treat diseases or disorders in human or non-human subjects.

In some embodiments, a SNA described herein is administered in one dose to treat a subject with SMA in an effective amount to increase expression levels of SMN over a baseline level in the subject in order to treat the disorder. As used herein, a baseline level is the level of SMN in the subject prior to treatment with a SNA described herein. In some embodiments, a subject having SMA is administered at least two doses of a SNA, in an effective amount to increase expression levels of SMN over a baseline level in the subject in order to treat the disorder. In some embodiments, the second dose is administered about 3 months, 6 months, 9 months, one year, 15 months, 18 months, 21 months or two years after the first dose.

As used herein, “pharmaceutically acceptable carrier or diluent” refers to any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In some embodiments, a pharmaceutically acceptable carrier or diluent is sterile water; sterile saline; or sterile buffer solution.

As used herein, “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein, “pharmaceutical composition” means a mixture of substances suitable for administering to a subject. For example, a pharmaceutical composition may comprise an antisense compound and a sterile aqueous solution. In some embodiments, a pharmaceutical composition shows activity in free uptake assay in certain cell lines.

For use in therapy, an effective amount of the SNAs or structures can be administered to a subject by any mode that delivers the SNAs to the desired cell. Administering pharmaceutical compositions may be accomplished by any means known to the skilled artisan. Routes of administration include but are not limited to oral, parenteral, intramuscular, intravenous, intrathecal, subcutaneous, mucosal, intranasal, sublingual, intratracheal, inhalation, ocular, vaginal, dermal or rectal administration, and by direct injection.

In some embodiments, the intrathecal administration is through a lumbar puncture. (See e.g., Astrid et al. European Journal of Paediatric Neurology (2018) 22(1):122-7 and Haché et al. Journal of Child Neurology 31.7 (2016):899-906, the contents of which are incorporated by reference in their entirety).

In some embodiments, any of the SNAs or structures described herein are delivered intrathecally (IT). In some embodiments, any of the SNAs or structures described herein are in a formulation that is compatible with intrathecal administration. Non-limiting examples of formulations that are compatible with intrathecal administration include artificial cerebral spinal fluid (aCSF); 100 mM sodium phosphate, 150 mM NaCl, 0.001% P 80; 10 mM citrate, 150 mM NaCl; 5% dextran in saline (hyperbaric solution); 0.75% or 7.5% glucose; paraben (methyl—and propylparabens); glycerin (50%); isotonic mannitol in normal saline; EDTA; DepoFoam; PEG suspension 2.5% PEG (3400); and 0.9% NaCl with 2.5% PEG and 0.025% polysorbate 80. Other formulations that are known to one of ordinary skill in the art are also contemplated herein.

The term “effective amount” is used interchangeably with the term “therapeutically effective amount” and refers to the amount of at least one SNA or structure described herein, at dosages and for periods of time necessary to achieve the desired therapeutic result, for example, to reduce or stop at least one symptom of SMA, for example a symptom of decreased muscle mass, known as muscle wasting, in the subject. For example, an effective amount using the methods as disclosed herein would be considered as the amount sufficient to reduce a symptom of SMA by at least 10%. An effective amount as used herein would also include an amount sufficient to prevent or delay the development of a symptom of the disease, alter the course of a symptom disease (for example but not limited to, slow the progression of a symptom of the disease), or reverse a symptom of the disease. Accordingly, the term “effective amount” or “therapeutically effective amount” as used herein refers to the amount of a pharmaceutical composition described herein to alleviate at least one symptom of SMA. Stated another way, “therapeutically effective amount” of an antisense oligonucleotide SNA as disclosed herein is the amount of SNA which exerts a beneficial effect on, for example, the symptoms of SMA. The dosage administered, as single or multiple doses, to an individual will vary depending upon a variety of factors, including pharmacokinetic properties of the muscarinic acetylcholine receptor inhibitor, the route of administration, conditions and characteristics (sex, age, body weight, health, size) of subjects, extent of symptoms, concurrent treatments, frequency of treatment and the effect desired. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. In general, the phrases “therapeutically-effective” and “effective for the treatment, prevention, or inhibition”, are intended to qualify the antisense oligonucleotide SNA as disclosed herein which will achieve the goal of reduction in the severity of at least one symptom of SMA.

In some embodiments, any of the SNAs or structures described herein are administered to a subject having SMA in an effective amount to increase expression levels of SMN2 protein or mRNA over a baseline level of SMN2 protein or mRNA in the CNS of the subject to treat SMA, wherein the effective amount of SNA is less than 12 mg/dose. In some embodiments, the effective amount of SNA is less than 11.5 mg/dose, 11 mg/dose, 10.5 mg/dose, 10 mg/dose, 9.5 mg/dose, 9 mg/dose, 8.5 mg/dose, 8 mg/dose, 7.5 mg/dose, 7 mg/dose, 6.5 mg/dose, 6 mg/dose, 5.5 mg/dose, 5 mg/dose, 4.5 mg/dose, 3.5 mg/dose, 3 mg/dose, 2.5 mg/dose, 2 mg/dose, 1.5 mg/dose, 1 mg/dose, 0.5 mg/dose, or 0.1 mg/dose.

In some embodiments, any of the SNAs or structures described herein are administered to a subject having SMA in an effective amount to increase expression levels of SMN2 protein or mRNA over a baseline level of SMN2 protein or mRNA in the CNS of the subject to treat SMA, wherein the effective amount of SNA is less than 12 mg/kg of body weight. In some embodiments, the effective amount of SNA is less than 11.5 mg/kg of body weight, 11 mg/kg of body weight, 10.5 mg/kg of body weight, 10 mg/kg of body weight, 9.5 mg/kg of body weight, 9 mg/kg of body weight, 8.5 mg/kg of body weight, 8 mg/kg of body weight, 7.5 mg/kg of body weight, 7 mg/kg of body weight, 6.5 mg/kg of body weight, 6 mg/kg of body weight, 5.5 mg/kg of body weight, 5 mg/kg of body weight, 4.5 mg/kg of body weight, 3.5 mg/kg of body weight, 3 mg/kg of body weight, 2.5 mg/kg of body weight, 2 mg/kg of body weight, 1.5 mg/kg of body weight, 1 mg/kg of body weight, 0.5 mg/kg of body weight, or 0.1 mg/kg of body weight.

In some embodiments, any of the SNAs or structures described herein are administered to a subject having SMA in an effective amount to increase expression levels of SMN2 protein or mRNA over a baseline level of SMN2 protein or mRNA in the CNS of the subject to treat SMA, wherein the effective amount of SNA is more than 12 mg/dose. In some embodiments, the effective amount of SNA is more than 12.5 mg/dose, 13 mg/dose, 13.5 mg/dose, 14 mg/dose, 14.5 mg/dose, 15 mg/dose, 15.5 mg/dose, 16 mg/dose, 16.5 mg/dose, 17 mg/dose, 17.5 mg/dose, 18 mg/dose, 18.5 mg/dose, 19 mg/dose, 19.5 mg/dose, 20 mg/dose, 22 mg/dose, 24 mg/dose, 26 mg/dose, 28 mg/dose, 30 mg/dose, 40 mg/dose, 50 mg/dose, 60 mg/dose, 70 mg/dose, 80 mg/dose, 90 mg/dose, 100 mg/dose, 500 mg/dose, or 1000 mg/dose.

In some embodiments, any of the SNAs or structures described herein are administered to a subject having SMA in an effective amount to increase expression levels of SMN2 protein or mRNA over a baseline level of SMN2 protein or mRNA in the CNS of the subject to treat SMA, wherein the effective amount of SNA is more than 12 mg/kg of body weight. In some embodiments, the effective amount of SNA is more than 12.5 mg/kg of body weight, 13 mg/kg of body weight, 13.5 mg/kg of body weight, 14 mg/kg of body weight, 14.5 mg/kg of body weight, 15 mg/kg of body weight, 15.5 mg/kg of body weight, 16 mg/kg of body weight, 16.5 mg/kg of body weight, 17 mg/kg of body weight, 17.5 mg/kg of body weight, 18 mg/kg of body weight, 18.5 mg/kg of body weight, 19 mg/kg of body weight, 19.5 mg/kg of body weight, 20 mg/kg of body weight, 22 mg/kg of body weight, 24 mg/kg of body weight, 26 mg/kg of body weight, 28 mg/kg of body weight, 30 mg/kg of body weight, 40 mg/kg of body weight, 50 mg/kg of body weight, 60 mg/kg of body weight, 70 mg/kg of body weight, 80 mg/kg of body weight, 90 mg/kg of body weight, 100 mg/kg of body weight, 500 mg/kg of body weight, or 1000 mg/kg of body weight.

In some embodiments, any of the SNAs or structures described herein are administered to a subject having SMA in an effective amount to increase expression levels of SMN2 protein or mRNA over a baseline level of SMN2 protein or mRNA in the CNS of the subject to treat SMA, wherein the effective amount of SNA is or about 0.1 mg/dose, 0.2 mg/dose, 0.3 mg/dose, 0.4 mg/dose, 0.5 mg/dose, 0.6 mg/dose, 0.7 mg/dose, 0.8 mg/dose, 0.9 mg/dose, 1 mg/dose, 1.5 mg/dose, 2 mg/dose, 2.5 mg/dose, 3 mg/dose, 3.5 mg/dose, 4 mg/dose, 4.5 mg/dose, 5 mg/dose, 5.5 mg/dose, 6 mg/dose, 6.5 mg/dose, 7 mg/dose, 7.5 mg/dose, 8 mg/dose, 8.5 mg/dose, 9 mg/dose, 9.5 mg/dose, 10 mg/dose, 10.5 mg/dose, 11 mg/dose, 11.5 mg/dose, 12 mg/dose, 12.5 mg/dose, 13 mg/dose, 13.5 mg/dose, 14 mg/dose, 14.5 mg/dose, 15 mg/dose, 15.5 mg/dose, 16 mg/dose, 16.5 mg/dose, 17 mg/dose, 17.5 mg/dose, 18 mg/dose, 18.5 mg/dose, 19 mg/dose, 19.5 mg/dose, 20 mg/dose, 20.5 mg/dose, 21 mg/dose, 21.5 mg/dose, 22 mg/dose, 23 mg/dose, 24 mg/dose, 25 mg/dose, 26 mg/dose, 27 mg/dose, 28 mg/dose, 29 mg/dose, 30 mg/dose, 31 mg/dose, 32 mg/dose, 33 mg/dose, 34 mg/dose, 35 mg/dose, 36 mg/dose, 37 mg/dose, 38 mg/dose, 39 mg/dose, 40 mg/dose, 45 mg/dose, 50 mg/dose, 55 mg/dose, 60 mg/dose, 65 mg/dose, 70 mg/dose, 75 mg/dose, 80 mg/dose, 85 mg/dose, 90 mg/dose, 95 mg/dose, 100 mg/dose, 500 mg/dose, 1000 mg/dose or any range there of or combination thereof.

some embodiments, any of the SNAs or structures described herein are administered to a subject having SMA in an effective amount to increase expression levels of SMN2 protein or mRNA over a baseline level of SMN2 protein or mRNA in the CNS of the subject to treat SMA, wherein the effective amount of SNA is or about 0.1 mg/kg of body weight, 0.2 mg/kg of body weight, 0.3 mg/kg of body weight, 0.4 mg/kg of body weight, 0.5 mg/kg of body weight, 0.6 mg/kg of body weight, 0.7 mg/kg of body weight, 0.8 mg/kg of body weight, 0.9 mg/kg of body weight, 1 mg/kg of body weight, 1.5 mg/kg of body weight, 2 mg/kg of body weight, 2.5 mg/kg of body weight, 3 mg/kg of body weight, 3.5 mg/kg of body weight, 4 mg/kg of body weight, 4.5 mg/kg of body weight, 5 mg/kg of body weight, 5.5 mg/kg of body weight, 6 mg/kg of body weight, 6.5 mg/kg of body weight, 7 mg/kg of body weight, 7.5 mg/kg of body weight, 8 mg/kg of body weight, 8.5 mg/kg of body weight, 9 mg/kg of body weight, 9.5 mg/kg of body weight, 10 mg/kg of body weight, 10.5 mg/kg of body weight, 11 mg/kg of body weight, 11.5 mg/kg of body weight, 12 mg/kg of body weight, 12.5 mg/kg of body weight, 13 mg/kg of body weight, 13.5 mg/kg of body weight, 14 mg/kg of body weight, 14.5 mg/kg of body weight, 15 mg/kg of body weight, 15.5 mg/kg of body weight, 16 mg/kg of body weight, 16.5 mg/kg of body weight, 17 mg/kg of body weight, 17.5 mg/kg of body weight, 18 mg/kg of body weight, 18.5 mg/kg of body weight, 19 mg/kg of body weight, 19.5 mg/kg of body weight, 20 mg/kg of body weight, 20.5 mg/kg of body weight, 21 mg/kg of body weight, 21.5 mg/kg of body weight, 22 mg/kg of body weight, 23 mg/kg of body weight, 24 mg/kg of body weight, 25 mg/kg of body weight, 26 mg/kg of body weight, 27 mg/kg of body weight, 28 mg/kg of body weight, 29 mg/kg of body weight, 30 mg/kg of body weight, 31 mg/kg of body weight, 32 mg/kg of body weight, 33 mg/kg of body weight, 34 mg/kg of body weight, 35 mg/kg of body weight, 36 mg/kg of body weight, 37 mg/kg of body weight, 38 mg/kg of body weight, 39 mg/kg of body weight, 40 mg/kg of body weight, 45 mg/kg of body weight, 50 mg/kg of body weight, 55 mg/kg of body weight, 60 mg/kg of body weight, 65 mg/kg of body weight, 70 mg/kg of body weight, 75 mg/kg of body weight, 80 mg/kg of body weight, 85 mg/kg of body weight, 90 mg/kg of body weight, 95 mg/kg of body weight, 100 mg/kg of body weight, 500 mg/kg of body weight, 1000 mg/kg of body weight or any range there of or combination thereof.

In some embodiments, at least two doses of any of the SNAs or structures described herein are administered to a subject having SMA in an effective amount to increase expression levels of SMN2 protein or mRNA over a baseline level. In some embodiments, the second dose is administered about one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 27 days, 28 days, 29 days, 30 days, 31 days after the first dose. In some embodiments, the second dose is administered 15 days to about three months after the first dose. In some embodiments, the second dose is administered about 2 weeks, 3 weeks, 4 weeks, 5 weeks, 6 weeks, 7 weeks, 8 weeks, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years after administration of the first dose.

In some embodiments, two or more doses of a SNA or a structure disclosed herein are administered at intervals of or about one day, two days, three days, four days, five days, six days, seven days, eight days, nine days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 27 days, 28 days, 29 days, 30 days, 31 days, 2 weeks, 3 weeks, 4 weeks, 5 weeks, 6 weeks, 7 weeks, 8 weeks, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 12 months, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years or more than 10 years, or any ranges or combinations thereof.

In an embodiment, a second dose of SNA or structure is administered one week to three weeks after the first dose of SNA or structure, a third dose of SNA or structure is administered one week to three weeks after the second dose of SNA or structure, a fourth dose is administered two weeks to six weeks after the third dose of SNA or structure, a fifth and subsequent doses of SNA or structure are administered between two and six months after the preceding dose. In some embodiments, all the SNA doses or structures are administered at the same or substantially the same time intervals. As disclosed herein, substantially the same time intervals refers to administration within three days of each other. In some embodiments, at least two of the SNA or structure doses are administered at the same time interval and any remaining SNA or structure doses at different time intervals, such as at any combination of the time intervals disclosed herein.

In some embodiments, an effective amount refers to the amount that is able to deliver about 2% to about 150% more therapeutic oligonucleotides to one or more tissues or regions of the body of the subject than administration of a linear therapeutic oligonucleotide that is not in an SNA format. In some embodiments, a SNA delivers about 2% to about 500%, about 2% to about 450%, about 2% to about 400%, about 2% to about 350%, about 2% to about 300%, about 2% to about 250%, about 2% to about 200%, about 2% to about 175%, about 2% to about 160%, about 2% to about 150%, about 2% to about 140%, about 2% to about 130%, about 2% to about 120%, about 2% to about 110%, about 2% to about 100%, about 2% to about 95%, about 2% to about 90% about 2% to about 85% to about 2% to about 80%, about 2% to about 75%, about 2% to about 70%, about 2% to about 65%, about 2% to about 60%, about 2% to about 55%, about 2% to about 50%, about 2% to about 45% to about 2% to about 40%, about 2% to about 35%, about 2% to about 30%, about 2% to about 25%, about 2% to about 20%, about 2% to about 15%, about 2% to about 10%, about 2% to about 5%, about 10% to about 500%, about 10% to about 450%, about 10% to about 400%, about 10% to about 350%, about 10% to about 300%, about 10% to about 250%, about 10% to about 200%, about 10% to about 175%, about 10% to about 160%, about 10% to about 150%, about 10% to about 140%, about 10% to about 130%, about 10% to about 120%, about 10% to about 110%, about 10% to about 100%, about 10% to about 95%, about 10% to about 90% about 10% to about 85% to about 10% to about 80%, about 10% to about 75%, about 10% to about 70%, about 10% to about 65%, about 10% to about 60%, about 10% to about 55%, about 10% to about 50%, about 10% to about 45% to about 10% to about 40%, about 10% to about 35%, about 10% to about 30%, about 10% to about 25%, about 10% to about 20%, about 10% to about 15%, about 10% to about 10%, about 10% to about 5% more therapeutic oligonucleotides to one or more tissues or regions of the body of the subject than administration of a linear therapeutic oligonucleotide that is not in an SNA format.

In some embodiments, any of the SNAs or structures described herein are administered in an effective amount to deliver a stable level of the therapeutic oligonucleotides to the CNS of the subject. In some embodiments, the stable level of the therapeutic oligonucleotides is achieved when at least 50% of the therapeutic oligonucleotides are present in one or more tissues or one or more regions of the CNS of the subject within seven days of administration of the SNA or structure to the subject, relative to the amount of therapeutic oligonucleotides present in the tissue of the CNS within one hour of administration of the SNA or the structure to the subject. In some embodiments, the stable level of the therapeutic oligonucleotides is achieved when at least 10%, 15%, 20%, 25%, 30%, 35%, 40%, 45%, 50%, 55%, 60%, 65%, 70%, 75%, 80%, 85%, 90%, 95%, or 100% of the therapeutic oligonucleotides are present in one or more tissues or one or more regions of the CNS of the subject within 6 hours, 18 hours, 24 hours, 48 hours, 72 hours, 4 days, 5 days, 6 days, 7 days, 8 days, 9 days, 10 days, 12 days, 14 days, 60 days, 18 days, 20 days, 22 days, 24 days, 26 days, 28 days, 30 days, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 1 year, 1.5 years, 2 years, 2.5 years, 3 years, 3.5 years, 4 years, 4.5 years, 5 years, 6 years, 7 years, 8 years, 9 years, or 10 years of administration of the SNA or the structure to the subject, relative to the amount of therapeutic oligonucleotides present in the tissue of the CNS within 1 hour, 3 hours, 6 hours, 12 hours, or 24 hours of administration of the SNA or the structure to the subject.

In some embodiments less than 50% of the oligonucleotides or therapeutic oligonucleotides in any of the SNA described herein are detectable within six hours of administration to the subject in one or both kidneys of the subject. In some embodiments, less than 100%, 95%, 90%, 85%, 80%, 75%, 70%, 65%, 60%, 55%, 50%, 45%, 40%, 35%, 30%, 25%, 20%, 15%, 10%, 5%, or 1% of the oligonucleotides or therapeutic oligonucleotides in any of the SNA described herein are detectable within 30 min., 1 hour, 2 hours, 3 hours, 4 hours, 5 hours, 6 hours, 12 hours, 18 hours, 24 hours, 48 hours, 72 hours, 4 days, 5 days, 6 days, 7 days, 8 days, 9 days, 10 days, 12 days, 14 days, 16 days, 18 days, 20 days, 22 days, 24 days, 26 days, 28 days, 30 days, 1.5 months, 2 months, 2.5 months, 3 months, 3.5 months, 4 months, 4.5 months, 5 months, 5.5 months, 6 months, 6.5 months, 7 months, 7.5 months, 8 months, 8.5 months, 9 months, 9.5 months, 10 months, 10.5 months, 11 months, 11.5 months, 1 year, 1.5 years, 2 years, 2.5 years, 3 years, 3.5 years, 4 years, 4.5 years, 5 years, 6 years, 7 years, 8 years, 9 years, or 10 years of administration to the subject in one or both kidneys of the subject.

In some embodiments, the duration of the method for treating a disease or disorder with a SNA or structure disclosed herein is for three months, for six months, for nine months, for one year, for 1.5 years, for two years, for 2.5 years, for 3 years, for 3.5 years, for 4 years, for 4.5 years, for 5 years, for 5.5 years, for 6 years, for 6.5 years, for 7 years, for 7.5 years, for 8 years, for 8.5 years, for 9 years, for 9.5 years, for 10 years, for 15 years, for 20 years or more than 20 years.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. The phrases “systemic administration,” “administered systemically”, “peripheral administration” and “administered peripherally” as used herein mean the administration of a pharmaceutical composition comprising at least an muscarinic acetylcholine receptor inhibitor as disclosed herein such that it enters the animal's system and, thus, is subject to metabolism and other like processes, for example, subcutaneous administration.

In some embodiments, a SNA or structure described herein is administered to a cell in vitro or is administered to a subject in order for the SNA to come into contact with a cell of the subject in vivo. Non-limiting examples of a cell contemplated herein include a fibroblast, epithelial, endothelial, neuronal, adipose, cardiac, skeletal muscle, immune cells, hepatic, splenic, lung, circulating blood, gastrointestinal, renal, bone marrow, or pancreatic cell. The differentiated cell can be a primary cell isolated from any somatic tissue including, but not limited to brain, liver, lung, gut, stomach, intestine, fat, muscle, uterus, skin, spleen, endocrine organ, bone, etc.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with SMA. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a malignant disease, diminishment of extent of a malignant disease, stabilized (i.e., not worsening) state of a malignant disease, delay or slowing of progression of a malignant disease, amelioration or palliation of the malignant disease state, and remission (whether partial or total), whether detectable or undetectable. The term “treatment” of a disease also includes providing relief from the symptoms or side-effects of the disease (including palliative treatment).

The terms “significantly different than,” “statistically significant,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i. e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., synaptic function. The dosage can be determined by a physician and adjusted, as necessary, to suit observed effects of the treatment.

As used herein, “ameliorates symptoms and/or defects” is improving any defect or symptom associated with SMA. As compared with an equivalent untreated control or with an equivalent receiving linear or free antisense oligonucleotide of the same sequence as the antisense oligonucleotide in the SNA, such reduction is by at least 5%, 10%, 20%, 40%, 50%, 60%, 80%, 90%, 95%, 99% or more as measured by any standard technique.

In some embodiments, administration to a subject of an antisense oligonucleotide in a SNA disclosed herein results in achieving milestones, such as the ability to sit unassisted, stand or walk, in other words, improved motor function, sooner compared to a subject receiving the corresponding free or linear antisense oligonucleotide or compared to a subject receiving control treatment (e.g., no treatment, placebo, etc.). Non-limiting examples of methods to measure milestones, such as motor milestone response and (Children's Hospital of Philadelphia Infant Test of Neuromuscular Disorders (CHOP-INTEND, are readily known and available to one of ordinary skill in the art. Other methods for measuring milestones, such as motor milestones, and survival, known to one of ordinary skill in the art are also contemplated herein. Non-limiting examples of SMA symptom management include (1) Orthopaedic treatment. Weak spine muscles may lead to development of kyphosis, scoliosis and other orthopaedic problems. Spine fusion is sometimes performed in people with SMA1 and SMA2 once they reach the age of 8-10 to relieve the pressure of a deformed spine on the lungs. People with SMA might also benefit greatly from various forms of physiotherapy and occupational therapy. (2) Mobility support. Orthotic devices can be used to support the body and to aid walking. For example, orthotics such as AFO's (ankle foot orthosis) are used to stabilize the foot and to aid gait, TLSO's (thoracic lumbar sacral orthosis) are used to stabilize the torso. Assistive technologies may help in managing movement and daily activity, and greatly increase the quality of life. (3) Respiratory care and treatment. Respiratory system requires utmost attention in SMA as once weakened it never fully recovers. Weakened pulmonary muscles in people with SMA1 and SMA2 can make breathing more difficult and pose a risk of hypoxiation, especially in sleep when muscles are more relaxed. Impaired cough reflex poses a constant risk of respiratory infection and pneumonia. Non-invasive ventilation (BiPAP) is frequently used and tracheostomy may be sometimes performed in more severe cases; both methods of ventilation prolong survival in a comparable degree, although tracheostomy prevents speech development. (4) Nutritional therapy. Difficulties in jaw opening, chewing and swallowing food might put people with SMA at risk of malnutrition. A feeding tube or gastrostomy can be necessary in SMA1 and people with more SMA2. Additionally, metabolic abnormalities resulting from SMA impair (3-oxidation of fatty acids in muscles and can lead to organic acidemia and consequent muscle damage, especially when fasting. It is suggested that people with SMA, especially those with more severe forms of the disease, reduce intake of fat and avoid prolonged fasting (i.e., eat more frequently than healthy people). (5) Cardiology treatment. Although the heart is not a matter of routine concern, a link between SMA and certain heart conditions has been suggested. (6) Mental health treatment. SMA children do not differ from the general population in their behaviour; their cognitive development can be slightly faster, and certain aspects of their intelligence are above the average.

In another aspect, the present invention is directed to a kit including one or more of the components of a SNA or a structure previously discussed. A “kit,” as used herein, typically defines a package or an assembly including one or more of the compositions of the invention, and/or other compositions associated with the invention, for example, as previously described. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions that may be associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

In some embodiments, a kit associated with the invention includes one or more lipid cores. A kit can also include one or more oligonucleotides. A kit can also include one or more anchors or linkers.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the use of the compositions, for example, for a particular use, e.g., to a sample. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoting” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

All references, including patent documents, disclosed herein are incorporated by reference in their entirety.

The genomic nucleic acid sequence, pre-mRNA nucleic acid sequence, mRNA nucleic acid sequence and amino acid sequence of SMN2 are well known to one of ordinary skill in the art. Non-limiting examples include:

Homo sapiens genomic SMN2 nucleic acid sequence
NCBI Ref. Seq.: NG 008728.1
(SEQ ID NO: 12)
CCACAAATGT GGGAGGGCGA TAACCACTCG TAGAAAGCGT GAGAAGTTAC TACAAGCGGT
CCTCCCGGCC ACCGTACTGT TCCGCTCCCA GAAGCCCCGG GCGGCGGAAG TCGTCACTCT
TAAGAAGGGA CGGGGCCCCA CGCTGCGCAC CCGCGGGTTT GCTATGGCGA TGAGCAGCGG
CGGCAGTGGT GGCGGCGTCC CGGAGCAGGA GGATTCCGTG CTGTTCCGGC GCGGCACAGG
CCAGGTGAGG TCGCAGCCAG TGCAGTCTCC CTATTAGCGC TCTCAGCACC CTTCTTCCGG
CCCAACTCTC CTTCCGCAGC CTCGGGACAG CATCAAGTCG ATCCGCTCAC TGGAGTTGTG
GTCCGCGTTT TTCTACGTCT TTTCCCACTC CGTTCCCTGC GAACCACATC CGCAAGCTCC
TTCCTCGAGC AGTTTGGGCT CCTTGATAGC GTTGAGTGGA GGCCCTGCCG CGACTTGGCA
GTAGCTTATT TTGTTCACTC CTCTCTGGCT GGTGTGGGGG AGGTGGGGGC ATTAGGCCAG
GGTGAAGCAG GGGAACCACT TAGGAGTCTG TTAAGATGAT CTGAACTTCA GAACAAGATG
TTATTAACAG AGTGAAAGTA TTTGGATTCT GGGTATATTT TGAAATCGGA GGCAACAGGT
TTTTCAGATA GATTCGATAA CGGAGGTTAT CCTGAATAGT TGAAAAGATA AAGTTGCCTT
TTGCTGAGGT GGGAAAGAGA AGATTGCCAG TAGAGCAGGT TTCTCAGGAG TTCAGTCTTG
GGCATAGCAT GGTAGGGGTG AATTTGGCTG GAGTGAGTTG GAGAGTAGGA GAAGAGAAAT
CCAAGGCAAC ATTTGACCAG CCTGGGCAAC ATAGTGTGAC TCCGAGTCTG CAAAAATTAG
ACGGGTGTTG TGGTGCGCGT CTGTGGTCTC AGCTACCTGG AAGGTTCAGG CCTTGGAAGG
CTCAGGGAGG TGGAGGCTGC AGTGATCTGT GATTGCGCCT CTGCACTCCA GCCTGGGCGA
CAGAGCCAGA CCCTGTCTTA AAACAAAATA AACGGCCGGG CGCGGTGGCT CAAGCCTGTA
ATCCCAGCAC TTTGGGAGGC CGAGGCGGCC GGATCACAAG GTCAGGAGAT CGAGACCATC
CTGGCTAACA CGGTGAAACC CCGTCTCTAC TACAAATACA AAAAATTAGC CGGGCGTGGT
GACGGGCGCC TGTAGTCCCA GCTACTCGGG AGGCTGAGGC AGGAGAATGT CATGAAGCCG
GGAGGCGGAG CTTGCAGTGA GCCGAGATCG CGCCACTGCA CTCCAGCCTG GGCGATAGAG
CAAGACTCCG TCTCAAATAA ATAAATAAAT AAATAAATAA ATAATAAAAA CATCGGTAGG
CATATTTCAA GGAATTCTAT TTAAAAAAAA TTTTTTTAGA GACAAGTTCG CTCTCTGTGG
CCCAGGCTGG AGTACAGTGG CATGATCCTA GCCCATGGCA GCGTTGATCT CTTGGCCTCA
AGCGACCCTC CTTTGGAGTC GCTGGGCCTA AAGGAGTGAG CCACCACGAA ATTTTATTAT
AAATGGAGGG TAGAGAAATT GGGCAATAAA TGGAGGGGGA AGTGAGTTAA GAGGAATTTT
AATTATGTGT GTGTGGTTTT AAAAGAGGGG GGTCTTGCTC TGTTGCCCAG GCTGCTGGGG
TGCCAGTGGC GCAATCATGA ATCACTACAG CCTTGGACTC CTGGCCTCAA GCTATCCTCC
CACCTCTGCC TCCCAAAGTA CTGGGATTAC TAGTGTGAGC CACTGCACTA AGATAGGAGC
AACATGTTTC AGCATGTTTG TGGGTTGATA GGAAAGATGA GAATGGGAAA GTTGATGTCG
GAAAGAAGAC AATGGCTAGA GCAATGTCCT AGAGTAGGTA AGAAGGGATG GATTTGGCCT
TTGTTGGAAA CATTAGCGGT TCTTTTGGTG ACAGCTATAT AGTTAACACA TCTATGATAC
GTGAATGGGC AGATAGGATG GCAGGAGATT TTGAAAGTTC TCTTGATTCT TACTGTTCTC
TTAGTGAAAG AAGCAAGGTT ATCAGCTAGA AGCTGGGATG GGAGAGGAAA GAGAAGATGG
GAAGTAGATA GTTCTTTAGA AGAGTGGGCA AGGGTTGGAC TAGGGAAGTT TAGTGGAAAT
ATTGCTAGGC AACATAAAGA GCCTACTTGA GATTCGTGGT CATGAGTTGA AGGAGACCAG
ACAGCAAGAT TGTGTATGAG GGCACCCACA GAGTAAATGG AGAGTTGAAA TTAATGCAGT
TGTGATTTTA CCACGTGGAT ATGAAGAAGT GAGGGGGAGA AGTACAAAGG AGTTCTCTTA
ATGATTGACC ATGGAATTTA AGCTGGCTAA GAAAGGAAGT GAGAGGCCGG GCGCGGTGGC
TCACGCCTGT AATCCCAGCA CTTTGGGAGA CTGAGGTGGG TGGATTACCT GAGGTCAGGA
GTTTGAGACC AACCTGGCCG ATATGGCGAA ACCCCATCTC TAATAAAAAT ACAGAAAAAT
TAGCCGGGAA TGGTGGCAGG TGCCTGTAAT CCCAGCTACT CAAGAGGCTG TGGCAGGAGT
ATCCCTTGGA CCCAGGAGGT GGAGGTTGCA GTGAGCCGAG ATCACGCCAC TGTACTCCAG
CCTGGACGAT ATAGTGAGAC TTCACCTCAA AAAAAAAAAA AAAGAAAGGA AGTGAGGATT
TTAAGACCCT GAGAGACAGT TTAAAAAGTG GGAGGATCGG CCGGGCGCTG TGGCTGACAC
CTGTAATCCC AGCACTTTGG GAGGCCGAGT TGGGCAGATC ACAAGGTCAG GAGTTCGAGA
CCAGCCTGGC CAATATGGTG AAACCTTGTC TCTACTAAAA ATACAAAAAT TAGCCGGGCA
TGGTGTCACG TGTCTATAAT CCCAGCTACT CGGGAGGCTG AGGCAGAAAA ATTGCTTGAA
CCTGGGAGGC AGAGGTTGCA GACAGCTGAG ATCACTCCAT TGCACTCCAG CCTGGGCAAC
AAGAGCAAAA CTTTGTCTTT AAAAAAAAAA AAAAAAAAAG AATACAAAAA TTAGCCGGGC
GTGGTGGCGC GTGCCTATAA TCCCAGCTAC TTGGGAGGCT GAGGCAGGAG AATCAGTTGA
ACACGGGAGG CGAGGTTTGC AGTGAGCCGA GATTGCGCCA CTGCACTCCA GCCTGGGCGA
CAGAGCAGGA CTCCTCTTGG AAAAAAAAAA TTAGCTGGGC ATGGTGGCAG GTGCCTGTAG
TCTCAGCTAC TAGGGAGGCT GAGGCAGGAA AATCACTTGA ACCCGGGATG TGGAGTTTGC
AGTGACCCGA GATCGTGCCA CTGTACTCCA TCCTGGGCGA CAAAATGAGA CTCTGCCTCA
AAAAAAAAAA AAAAAAAAAG TGGGAGGATC AATGTACTGC CAGTCCTAAT GAAGTGGAAT
GATTGTCCCC ATCAAATCAC TAGTAGGAGT AAGTTGCAGA GCCTAGAAGG TGATGGTTAA
GAGAGTGGGA TTCTTGAAAC TGCATTTATG GAGAGGTTGT GGTTATTGGT TATAATAAAT
AAATACAGTT GAAGTGAGTG AGTAGCTGAG ATTTGGGGAT GTATCAGTTC ATTCTTACAC
TGCTACAAAG ACATACCTGA GACCAGGTAT TTATAAAGAT AAGAGGTTTA ATCAGCTCAC
AGTTCTGCTG CCTGTACAGG CTTCTCTTGT GGAGGCCTAA GGAAACTTAC AGTCATGGTG
GAAGGTGAAG GGGAAACAAG CACAGTCTTC ACATGGCCAG CAGGAGAGAG AGAGAAGGGG
GAAGTGCTAC ATACTTTAAA ACAACCAGAT CTTGTGAGAA CGCTTATCAG GAAACAGCAC
TTGGGGATGG TGCTAAATCA TTAGAAATCA CCCCCATGAT CCAGTCGCCT CCTACCATGC
CCACCTCCAA CACTGGGGAT CACAATTCAG CATGAGATTT GGGTAGGAAC ACAGAGCTGC
ACCACATCAG AGGATGTACA AGATTGTGGT GGAGAGGAGT TTAGAGACCT GCAAATATAG
GGTAATTGAA GGGATCATCT ACATGGATAT TTAAATCACC AAAAATTATG ACAGGAGTAG
TGTTGGAGAG AGAACTGCGA TGTAAACATT AAGGAATGAG GAAGAGTGAC TCGGTAGGCT
GTAGGTGACT GCAATAGGAA ACGATAATAG ACTGTGAGTC TGGTGACAAG ATTTTCCTTC
TTTCTTTTTT TCCCCCCCCC CGAGACAGGG CCTCTTTTTG TTGCCCAGGT GGGAGTGCAG
TGGCGCGATC ACGGCTCACT ACAACCTCCT CCCAAGCTCA AGGGATTCTC CCACTTCAGC
CTCTCAAGTA GCTGGAACTA CAGGTGCTGA CCACCATGCC TGGCTACTTT TTGTCAGGAT
TTTCAAGGCT GGGAATTTTG AGAGGGGAAT GGAGGAGAAT AATCTGAAAG TGCAAGTAAG
GAGCAGGGAA GATTTCTTTT TTCTTTTTTT TTTTTTTTTT TGAGTCGGAG TCTGGCTCAG
TCGCCCAGGC TGGAGTGCAG TGGCGAGATC TCCGCTCACT GCAAGCTCCG CCTCCCGTGT
TCACGCCATT CTCCTCCTTC AGCCTCCCGA GTAGCTGGGA CTACAGGCGC CCGCCACCAC
GCCCAGCTAA TTGTTTTTTT GTATTTTTAG TAGAGACGGG GTTTCACCGT GTTAGCCAGG
ATGGTCTCAA TCTCCTGACT TTGTGATCCG CCCACCCCGG CCTCCCAAAG CGCTTGGGAT
TACAGGCGTG AGCCACCGCG CCAGCCAGAG CAGGGAAGAT TTCTTCCCCA CATCTCCAGT
AGGTACAGTG ATATGAAGTG TGTGGAGGAG AAAAGAGGAA ACATCTATCA TTTGAGATGG
CTGCGAAAGG AAAAGGCATC CTCAGGGAGC TAGATTTTAC TTAGAGCAAG AAATGAAGGG
ATGATTCAGA GGTTAAAAGA GTGGATTTTA TGAATTACTC AAGGGAGCAC AGTGGAAGTT
TCAGGAAGTG GTAGGAGAAG GTAGAAGATG GCAGGGTGTT GGGAATAATT TGAGAAATCT
GAGCTACTGG AAATGACTGA GAATCAGATA TAAAGGCAGT CCTGGTGGTC CGTTCTGGCT
GCCGTTGCTG TGTAACGAAT CTGCCAAAAC TTAGTGGCTT GAAACAACAA AGAACATTTT
ATTATCTCTC ATTGTTTCTG TGGGTTAGGA ATTTGTGAGA GCCGTGCTGG GCAGTTTTCG
TGCGGCTGTC TCGTGGTTGC ACCTACATAG TTGCTAGAGC TACAGTAGCT GGGGACTGAG
CAGCTAGGGA TTGGCAGGCT ATCTCTTTTT TTCATGTAGT CTCATGAAGA TTTCTTTATG
TGGTTTCAAT GTGTGGGCTG GTTTGGATTT CCTTATAGCA TGGTGGCCTC AGTTGGATTG
CTGTTTTGTG ATCCTTTTCA TCCCTCCTTG TCCTGTCCCC AGACAACCAC TGATCTACTT
TCTGTCACCA TAGATTAGCC TGCATTTTTA AGAATTTTTA TAAACGTGGA ATGATAGAGT
ACCTTTTTTG TCACGTTTCT TTTATTTATC ATAGCTATTT TGATTTTCAT CCATTTTATT
GCTGAGTAGT ATCCCATTGC ATGTATATAC TATACTGTAT TCATTCGCTT GCTTGTGAAC
ATTTGGGCTT TTTCCAGTTT GGGACTGTTA ACAAGTAGAG CCACTATGAA TATTAGTGTA
TAAGACTTCA TATAGCCAAG GCTGGCAGAT CGCTTGAGCC CAGGAGTTTG AGACCAGCCT
GGGAAACATG GTGAAACCTC TATTTTTATT TTAAAATCAA AAATTAAAAA TTTTCTATAA
AAAATTTTAA AGAAGACTTT GTATAGACAT ACGCTTTCAT TTTTCTTGAG TGAATACTTA
GGTCTCAGGG TAGATGTATT TTAAGTCTTT AAGGAGCTGT CAAACTCTTC CTCAAAGTGG
TGGTTGTACC ATGTTACTTT TTAATATAAC AGAGATTAAT TGAGCAAAGA AAAATTCAAA
AGTTGGACAG CCCCCACAAC TAAATAGGTT CAGAACAGCT CCCCCATTTT GCATTTTGAC
CAGCAATGTA TGAAAGTTCC ATTTGCTCAG TGTCCCTGCA AACACCTGGT ATGGTCAGTC
TTTTTAATTT TAGGCATTAT AATAGATATA GTGGCTTCTT GTGATTTTAA TTAGCATTTC
CTAATGACCA GTGCTGCTGT TGATCATTTC ATGAGTGTAT TTGCCATCCG TATATCTTTT
TTGGTGAAGT GTCTATTCAA ATCATTTGGG TTTTTTTTTT TTTTGTTTTT TTTTTTTGGA
GACAGTGTCT CACTCTGTCA CCCAGGCTGT TGTGCAGTGG TGCAATCACA CAGCCTACTG
CAGCCTCCAC CTCCTGCGCT CAGTCTTCTT GTCTCAGCCT TCTGAGTAGC TGAAATTACG
AGCACACGCC ACAATGCCTG GCTAATTTTT TAAAATTTTG TAGAAACAAG GTCTCATTAT
GTTGCCTGGG CTTGTCGTGA ACTCCTGGGC TCAAGCAATC TTCCTGCCTC AGCCTCCCAA
AGATTGGGAT TGCAAGTATG AGCCACTGCA CCCGGCCAAC TTACCCATCT TTTAATTGAA
TTTTTTTGTT GTTGAGGTTT GAGAGTTCTT CATGTTTGCT GGGTACAATA TCTTTATCAG
ATAGGTAACT TGCATGTATT TTCTCCCGGT TTACACTTTG GTTTTTCATT TTGTTAACAA
CGTCTTTTTA AGAACAGAAA ATCTTAATTT TGCTGAAATC TAATTTTTCA GTTTTTTCTT
TGATGGTTTT GAGAGAGGAG GTAAAAAAAG ACTAGGTAAG CCGATAGTTA GACAGAGTCC
TCGGTAGAAC TTCCCTTCTA ACAAAAAGCA GCCCAAGAAA TCACTTCTCT TCTAACAAGG
AGCAGCCTGG AAGATCGGGC TGTAAACATG TATAAGGAAG CAGCTCTGGC ACAGAGGGGG
AGCTTCCTGG GTAATCAGCA AGCTTCACAT ACGTAAGGTG GGTATGTGAA GTAAACACAG
TATGTGAAGT AAACACAGTG GACCTTAGTA CATACTCAGA TAAGGAAGCT GGAAGCTTGC
ATGTTGTGAG TTGTTGGGGT TGCCTGCAGC TGCACGGAGA GAAAGGGGTA CCTGGGGCCA
GGCATGTCCA CCATGGTGGC TCCACCTCCC CTTATTTAGC ACATGCACAA TAGGAAAGAG
ATAAGCAATG TGGAGTAGCT CAGGCCAAGG ACCTGCCTGC ATAATAAAAG GTTGGGGTGG
GGGATGCCAG AGATTCACGC TCTGTGCAGA TGGCAACACC TGGTCCTAAC TGGTTTTTTG
CTCCCTATGT GTAGATAAGC TACCCCCTTC CCATTAGCTC ATTTATAAAA ATGCTTGCAT
TTCACTGTGG AATGGGAACT CTTTTCAGGA CCTCTCTCTG CAGGAGAGAG CTAGTCTCTT
TCTTTTGCCT ATTAAACTTC TGCTCTAGCC TCACACCCTT GGTGTGTCAG CGTCCTTGAT
TTCCTCAGCG TGAGACCAAG AACCTCGGGT GCCACCCCAG GCAACAAGGC CATTTCAGTT
TGTTCTTTTG TTATAGGCAA TCCATGATCA CAGATTTTTC TCTCTTTTTT TTTTTTACAC
AGTTTAGAGT TTTAGTTTTA CACTTAGGTC TGTAATCCAT TTTGTATTAA TTCTTATATG
TGGCTCAGTG TAGGTGGAAA TTTGGTTTGT TTTTGCATAA GGATTTCCAA TAGTTTTACC
ACCATTTCTT GAAACTACTA TGCTTTCTCT ATTAAACCAC ATTTGTAACT TTAGTTAAAA
TCAGTCACAT ATATCACAGG GCTATTTCTG ACTCTCAATT CTGTTACATT GTCTATTAGT
GTATATTGAT GTCAGTACTA CACTTTTAAT TACTATTGCT TCAGGGTATG TCTTGTAAAC
CAAAAATAAA ATTATAGGCC CCCCCCGCCC CTGCACAACC AACTGAATGG ACCCATCCTC
TCAGCCAAGG GCATTCCAAA ATTAACCTGA AAAACTAGTT CAAGCCATGA TGGGAAGGGG
GAGTTGGACA TGTCTCATCA CACCCTACTA CCTTTTGGAA TTACTGATAG AACAGACTCT
TAAAGTCTGA AAAGAAACAT TTACAACCTA CCCTCTCTGA AGCCTGCTAC CTGGGAGCTT
CATCTGCATG ATAAAACCTT GGTCTCCACA ACCCCTTATG GTAACCCAAA CATTCCTTTC
TGTTGATAAT AACTCTTTCA ACTAGTTGCC AATTAGAAAA TCTTTAAATC TTCCTATGAC
CTAGAAACCT CCCTACCCCC ACTTTGAGTT GTCCTGCCTT TCCTGACAGA ACTCATGTAC
ATCTTACATA TATTGATTGA TGCCTCATGT CTCCCTAAAA TGTATAAAAC AAAGCTGTAC
CCCACCACCT TGGGGACATG TCATCAGGAC CTCCTGTGGC TGTGTCATAG GAGCGTCTTT
AACTTTGGCA AAATAAACTT TCTAAATTGA TTGAAACCTG TCTTAGCTAC TTCTGGTTTA
CAGTCTTAAA GTTAGATAAT GTAAATTGTC CAGCTTTGGT TTATTTTTGT CCTTAGTAGT
TCCATATAAA TTTTAGAATC AGCTTTTCAA TTTAATACAC TACTTTCCTC TTAGATCCAC
AATTAAATAT ATTTGATGCT AACAATTCTG TTTTATGTTT TTCGTTTTTT TTTTTTGAGA
CAAGAGTTTC GCTCTTGTTG CCCAGGCTGG AGTGCAGTGG CGCGATCTTG GCTCACCACA
ACCTCCACCT CCCAGGTTCA AGCAATTCTT CTGCCTCAGC CTCCCGAGTA GCTGGGATTA
CAGGCATGCG CCACCACGCC CGGCTAATTT TGTATTTTTA GTAGAGACGG GGTTTCACCA
TGTTGATCAG GCTGGTCTTG AACTCCTGAC CTCAGGTGAT CCACCCACCT CGGCCTCCCA
AAGTGTTGGG ATTACAGGCG TGAACCACCA TGCCTGGCCA GTTCTGTTAT TTTTAAAACC
CAAGTTTCCC TGGTCATATC TTGGTTGGAT GAAGCGTATT TTCAATAGAT TACCCTGGAA
AGGCTAGTGA GTACGGTATT CTTCTACATT TTAGACTTTT CTTAGTCTTG CTACTTCAAG
GACAGCTAGG CTGCATATAA AATTCTTGGC TCATACTTTT TCCCCATAAA TTTCTATGAG
AAAGTCTAAT GATAACTGAT TTTCTTTATT TTGTAACTTA GTCTTTTTGC TTAGAGGCTC
TCTGAGGATG GGAGGGGGTT CTTCCTCCCA TCCCTAGGAA TTTTTCTTTT TTTTAAATTC
CTAATCACTA GACCACCAGG AAGATTGTTT GTTTTGTTTT GTTTTTATTC TTCAGGGACC
CCATTTATAC ATACGTTAAA TAAATACTGT TTGCCAATGT ATCAACCATT TTGCTTCTTA
TTTATTTTTG TTCCTTTGGT TCTTTTTCAT GGCTTTGCTT TGGTGCTCCT TAGATTTTCA
GTCAGATGTA TTTGTCCTTG GGTACCTTGT AATCAGTATT ACCTTTTCTT CTGTCGCTTT
GTTTTCTGTT CGTTTTGAAA TTACTTGTTT CCTGGTCTGG CAATAACAGT TGAGATATGA
GGAGTTTGAG CTGCCATCTG TCTATGTATC TTGCTTTAAG ACTGCACTCT TCTATTGATA
TCACTGGCCT TGATTTTGTG ATTTCTTTAT TTCTTCAGGA CCACCCTTCA TTTTCTACTG
TTTGCTTCCT TTTTTTTTGA GATGGAGTCT CACTCTGTCA CTCAGGCTGG AGTGCAGTGA
TCTTGGCTCA TTGCAACCTC TGCCTCCCGG GTTCCAGCAA TTCTCCTGCC TCAGCCTCCC
AAGTATCTGG GACTACAGGT GTGCACCACC ATGCCCGGCT AAGTTTTGTA TTTTTAATAG
AGACGGGGTT TTGCCACATT GGCAGGCTGG TCTCAAACTC CTGATGTCAA GTGATCCACC
CACCCCACCC ACCTCTGCAT CCCAAAGTGC TGGGATTACA GGAATGAGCT GCCGTGCCCA
GCCTCCCCCC TACCCCCCTT TTTTTCTTTC GAGACAGAGA TTATAGGTGT GAGCCACTGG
ACCCAGCCTG TTTTTATTCC TTTTACCAAA TCTCCAAGGA ATATCTTCCC TTCCAAGTGC
GAATGTAACC TTAAGTCAGT TAACCTCTTT GTGATTACTT TTCTTATCTG CAAAGTGACT
TAATGATCTT AAGTACTTTT TTTTTTTGAG ACAGGGTCTC ACTGTCACCC TGGCTGGAGT
GCAGTGGCAC GATCTCTGAT CTCCACTCAC TGCAATCTCC TCTTCCCTGG TTCAAGCGGC
CCTCCCACCT TAGCCTTCTG GGTAGCTGGG ACTACAGATG TGAACCACCA CGCCCAGCTA
ATTTTTGTAC TTTTTGTAGA GATGGGGTTT TGCCATGTTG CCCAGGCTGG GATTATTAAG
TACTTTTTAT CATACAGCAA GATTGACATT TTATATTGGA ATACATTTGT CTCTATATAA
CGGAGATTAA CAGGAAAATG ACAAGCCTGG GTGCGGTGGC TCATGCCTGT AATCCCAGCA
CTTTGGGAGG CTGAGGTGGG AGGATCACTT GAGGTCAGGA GTTCGAGACC AGTTTTGCCA
AGATGATGAA AGCCCATGTC TACTAAAAAT ACAAAAATTA GCCCAGCTTG ATGGTGGGCG
CCTATAATCC CAGCTATTTG AGAGACTGAG GCAGGAGAAT CACTTGAACC TGGGCAGCAG
AGGTTGCAGT GAGCCGAGAT CATGCCACTG CACTCCAGCC TGGGTGGCAT AGCGAGACTC
TTGTCTCAAG AGAAAACAAA ACAAAACAAA AAAAAAACAG GAAAATGACA AAAAGTAATA
TTACAACTCA GTGAATTTTA TAACAAACTT TTTTGGAATT CATTGACTAA TACTATACCA
AATCCAAAAT ACTCTCTAGT ATACCAAATC CAACTCTACC CTATAGTATA AATTGGATTC
TATTTGGACT TGTCTCACTA ATCCCTCATA CAGTGTGTTT TATTTTTTAT TGAAGTAAAA
AAATTTGTCA TTTTAACCAT TTTTAAGTAT ATAGTTCAGT AATATTAAGT ATGTTCATGT
TGTTGCGCAA TAGATCTTCG GAAGTTTTTC GTCTTGCAAC CTGAAACTCT ACCCATTAGC
AAATTCCCAT TTCTCCTTAC ACTTAGCCCT TGGTAATCAT CATTCTTTTT TTTTTTTTTT
TGAGATGGAG TTTTACTCTT GTTGCCCAGG CTGGAGTGCA ATGGTGCAAT CTCGACTCAC
CACAACCTCC GCCTCCCAGG TTCAAGCAAT TCTACCTCAG CCTCCCGAGT AGCTGGGATT
ACAGTCATGC ACCACCACGC CCGGCTAATT TTGTATTTTT AGTAGAGAAG GGGTTTCTCC
ATGTTGAGGC TGGTCTCGAA CTCCTGACCT CAGGTGATCT GCCCACCTCG GCCTCCCAAA
GTGCTGGGAT TACAGGCGTG AGCCACTGCG CCTGGCCCAT TCTTTCTAAT TCTATAAATT
TGACTACTTA GTTACCTTAC ATAAATAAAT TCTTATAGTT AGTGTTATTT TTGCTTCCAT
GCCTTTTTTG TTGTTGTTCA TGCTCTTACT TGGAATGCGT TCTATTTTGT CTACCTATGC
ACATCCTGTT GGGTTTTTTT TTTTTTTGGG GGTTTTTTTT GTTTTTTTTT GTTTTTTTTT
CCCAGACAAG GTCTCAATTT GTTACCCAGG CTGGAGTGCA GCGGCGCCAT CTCCACTCAC
TGCATCCTCA ACTTCCTGGG CCCAGGTGAT CCTCTCGCCT CAGCCCCTGC AGGTAGCTGG
GACTATAGGC ATGTGCCACC ATGCCCAGCT AAATTTGGTT TTTTTGTTTG TTTGTTTTTG
AGACAGAGTC TCACTCTGTC ACCCAGGCTG GAGTGCAGTG GCACAATCTC AGCTCACTGC
AATCTCTGCC GCCCGGGTTC AAGTGATTCT CCTGCCTCAG CCTCCCAAGC AGCTGGGATT
ACAGGTGACT GCCACCACGC CAGCTAAGTT TTGTAGTTTT AGTAGAGATG GGGTTTCACC
TTGTTGGCCA TGCTGGTCTC GAACTCCTGA CCTCGTGATC TGCCTGCTTC TGCCTCCCAA
AGTGCTGGAA TTACAGGCAT GAGCCACCAC GCCCGGCCAG AATTTTTGTA TTTTTAGTAG
ACACAAGGTT CTTACCCTGT TGCCTAGGCT GGTCTGGAAG TCCTGGACTC AAGCAATTCA
CCTGCCTTGG CCTCCCAAAA TGCTGGGATT ACAAGCCACC ATGCCCGGCC TAAATCCTGT
TGTTTTGTTT TGTTTTATTT TGTTTTGTTT TGTTTTGTTT GTTTTTTGAG ACAGAGTCTC
GCTATGTCTC TCAGGCTGTA GTGCAGTGGC GCGATCTTGG CTCACTGCCA CCTCTGCCTC
CCAGGTTCAA GTGATTCTCC TGCCTCAGCC TCCCAAGTAG CTGGGATTAC AGGCATGTGC
TACTATGTCC GGCTAATTTT TGTATTTTTA GTAGAGACAG GGTTTCACCA TGTTGGCCAG
GCTGGTCTCG AACTCCTGAC CTCGTGATCC ACCCACCTCG GCCACCCAAA GTGCTGGGAT
TACAGGCGTG AGTGGTTTTT ATTTCTTAGG CCGGTTTCCT CCATATGATC TTGCAGTAGA
CATTAATTTC TTTCCTTTTT AATTAAAATA CTGTTTGTAT TTCACATTTT GATGTTTGTT
AAGATTTGTT TTATATTGTT TTTTGTTTTG TCTTGTGTGA TAGTCTTAAA TCCCTAGTTA
GATAATAACT GGAGAGTACC ATGTTTCTAT ATATCTCTCA GTGACTTGCA CAGTGCTAGC
AGATAGTGCT AAAAAATTAT TTATTATTAT TATTATTTTG TTATTGTTGT TGTTGTTGTT
AGACAGGGTC TTCCTCTGTC ACCCAGGCTA GAGGGCAATG GGATGATCAT AGCTTACTGC
AGCCTCCAAC AACTGGGCTC ATGTAATTCT CCTGCCTCAG CTTCCCAAGT AGCTGGGATT
ACAGGCATGA GCCACCATGT CTGGACAAAA ATATTTCCAG GTGCAGTGGC TCATGCCTGT
AATTCCCACA CTTGGGAGGC CGAGCGAGGC TGGAGGATCA CTTGAGCCTA GGAGTTCAAG
ACCAGCTTGG CTAAGATGGC GAGACCCCGT CCCTACAAAA AATTTTAAAA ACTAGCCAGG
CATGGTGGCA TGCACCTATA TTCCCAACTA CTCAGTGGGC TGAGGTGGGA GGGTCATTTG
AACACAGGAA TTTGAGGGGA GAAAAAAAGA AGAGAGAAAG AGAAGTGAAG GAAGGAAGAA
AGGAAGGAGG GAGGGAGAGA AGAAAGAAAC GAAAGAAAGG AAAAGAAAAG GAAGGAAAGA
AAATTGGTAC CAGGAAAGCA GGAAAGGGAA ATGGAAGTAA AAAAATAATA ATAATAATAA
AATGAAAATT GGTTAGTCAC TATTAACAAT TTGTATCCTT ATAATCTGGA AACATTATAA
TTTCAAAAGA AAAAATATTC TTTGGATCAT AGGTTCTGAG GTCAGAACAG CATTCCCGTA
GTCTAGATGA AGTCAAGTTT TATCTGATCT TAATTGAAAT AAATATAGCT GGCCTTGAAC
AAATCTACTC ATGGTATGTG GATAGGAATT AAATTGTAGG GGCATTCACT TGATGGCATT
CATTCTTAGA ACATTTACCT ATGTCTAGCT TTTGGAGTAA AGTCACATAA CCTCTAACCA
GGTAAGTTTC CTGTGGCTTT ATTTAGGATT TTAAATACTC ATTTTCAGTG TAATTTTGTT
ATGTGTGGAT TAAGATGACT CTTGGTACTA ACATACATTT TCTGATTAAA CCTATCTGAA
CATGAGTTGT TTTTATTTCT TACCCTTTCC AGAGCGATGA TTCTGACATT TGGGATGATA
CAGCACTGAT AAAAGCATAT GATAAAGCTG TGGCTTCATT TAAGGTATGA AATGCTTGCT
TAGTCGTTTT CTTATTTTCT CGTTATTCAT TTGGAAAGGA ATTGATAACA TACGATAAAG
TGTTAAAGTA CATGTTATTC AGTTTTCATT TTGAAGATTA GATGGTAGTA TGAGTTAGTT
AAATCAGGTG ATATCCTCCT TTAGAAGTTG ATAGCCTATA TATGTCATCC TTTGTGGAGG
CAATTTAAAT AAAATTTAAA ACATTTATTC CTGGCTGGGT ATGGTGGCTC ACTCCTGTAA
TCCCAGCACT TTGAGAGGCT GAGGCGGGTG GATCACCTGA GGTCAGGAGT TTGAGACCAG
CCTGGCCAAC ATGGTGAAAC CCCGTCTTTA CTAAAAATAC AAAAATTAGC CAAGCATGGT
GGCACGTGCC TGTAATCCCA GCTGCTTGGG ACACTGAGGC AGGAGAATTG CTTGAACCTG
GGGGGCAGAG GTTGCAATGA TTGCACCACT GCACTCCAGC CTGGGCGATA GAGTGAGACT
CCATCTCAGA AAACGAACAA ACAATGTATT CCTTTTAGTA TTTTTACATT GTATCAAACT
ATGGAAGTCC TCTAATTGAG ATTAATAAGA AAAAGACAAT CTGAATTATA ATTTTAAACA
TTTAACAAGC ATGTAGTAAA ATAATGATGA AGATAAATAG CATTAGTACA GCAATTAATA
TTTGTAGCAT GCTGACAGTG CTCTGTGTGC GTTTCATATA TTAAATTACT CTAATCATCC
CAAATCCTGT AAGTTGGGTA TCAATTCAAG TGTTCCTATT GGGTAGGAAT ATACAGTTCT
TTTAGGAAAT GTAGTATGGT TCTGTGTCTC AAACAGGACA CTTACACAGT TGGCCAACAT
CATCACCTTC TCCATTCTCT GAGATGTTTA GTCTTACTGA GCACTAAATA TGGGTCATCA
ATAGTCCAGA CTACCTTGAG CAAACAATAG TCCAGACTAC CTTGAGCAAA CAGAGCATAT
ACTCATACAG TGTATAAAGA GCACCAAGCA TACAGATTTC ATGTCTTTCT CATAGTTACT
CTTGTAACAT GAGCTAAAGA TCAGACCTCT ATGTCACCTT TGTAACTGAT TTCTAGATTT
TTTTTTTTTT TTGAGATGGG GTCTTGCCCT GTCACCCAGG CTGGAGTGTA GTGGCGTGAT
CATGCCTCAT TGGAGCCTTC AACTCATGAG CTCAAACAAT CCTCCTACCT CAGCTTCCTG
AGTAGTTGGG ACCACAGGTG TGTGCCACCA CACCCAGCTC ATTTTTGTAT TCTTTGTAGA
GATGCAGTCT CACCCTGTTG CCCACGCTGG CCTGGAACTC CTGAGCTCAA AAGATCCCTC
CGCCTTGACC TTCCAAAGTG CTGGGATTAC AAGCATGAAC CACTGCACCC GGCCTAGATT
TTTAAATGTG CTTTCCAGTA TACACTGAAA CTAGAAGTCG ACTAAAGAAT TACCAAGAGA
ATTCTATAAA ATAGAGATTG AAATGGGGCT CGATGTGGGA TGGGTTGGTG ATATTGCAGG
GAGAAGTAAT CTGAGTAAAG GAGGAAAAGA ACTGATTTGG GAAAACGATA GTTTTAGTAG
TGAGTTTGAG TATGAATTAA GTTGAGATTG AATTTGAATT AAGTTGAGGT TGAATATGAA
TTAAGTTGAG GTTGAGTTTG AGGTATGAAT TAAGATGTGA AATTGATCAT TGGAAATGTT
AGATTGAGAA AAGTCACAGC TGGATTAATA GCTTCAGAAG TGTGTTTGCA GACAGTTGCA
ACTAAAGTAA TAAGAATAGA TGGCCTTGGC CGGGCGCGGT GGCTCACGCC TGTAATCCCA
GTACTTTGGG AGGCTGAGGC GAGCAAATCA CGAGGTCAGG AGTTCAAGAC CAGCCTGGCC
CACATGGTGA AACCCCGTCT TTATTAAAAA TACAAAAATT AGCTGTGCAC AGTGGTGCAC
GCCTGTAATC CCAGCTACTC GGGAGGCTGA GACAGGAGAA TCGCTTGAAC CTGGGAGGTG
GAGGTTGCAG TGAGCTGAGA TCAGTGTGAC TGCACTCCAG CCCGGTGACA GAGTGAGACT
CTGTGTAAAA AAATAAAATA AATAAAATAA TGGCCGTAAG CAAGTAAAGA AGGATGGCCA
GCTCTTATTG GGAATGCCTA AATCTAAGGC TTGATCAGAA GTAATGAAAC CGTTGGGGCC
CTACATTGCT ATGACATCCA AAGGGCCATG AATATCAGGA AGAAAGATAA TTAACAGGGT
CTAATGTTAC AGAGAGGTTG AGAGCAAGGA GATTTGATTA AAAGGGTCTT TAGAGCTGAT
GTCAGGTGTA TGATGCCTTT AAGAGCAGTT TTTATAGTGC AGGGGGTGGT CAAAAGAGAA
AATAGGTGCT TTCTGAGGTG ACGGAGCCTT GAGACTAGCT TATAGTAGTA ACTGGGTTAT
GTCGTGACTT TTATTCTGTG CACCACCCTG TAACATGTAC ATTTTTATTC CTATTTTCGT
AGCATGCTCT AAAGAATGGT GACATTTGTG AAACTTCGGG TAAACCAAAA ACCACACCTA
AAAGAAAACC TGCTAAGAAG AATAAAAGCC AAAAGAAGAA TACTGCAGCT TCCTTACAAC
AGGTTATTTT AAAATGTTGA GATTTAACTT CAAAGGATGT CTCATTAGTC CTTATTTAAT
AGTGTAAAAT GTCTTTAACT TAAGTGATTA GTACAGTGTT TCTATTGACA TATACTTATA
CAACTTCAAA AACAACTATT AAATTTTCTG TTATTTAGGA ACATGCATAT TAGTCATGAA
AGTATAAAGA ATTAGATGGG AATGATAAAT GCTAAAATCA GGACATGTGT TCCATTTGTG
AATGGAAGGC AGGGAGAAGG TGCCGTTTGG AAGGAGTACC CAAGAGCCGT AAGCTGAATT
GGCAGTGTTT TACATCTTAA GCTGAGAGAT AGATTTTTTT TTCCCCTTTT TCTTTAAAAA
CTCTAAAACT GTTAATTCCA AGGAACCCAG AAGTCTAGGT AGATTATTTC TGCTAGTTAA
AAGCAGTAGT CCTGAAAGCT GAATATTTTG GTGTCTTTTG AGCCAACTTT AGTTTCATCA
TTACCAAGGG GGAAGAGAGC TAACAGTTGA TGAGCACTTG CTCTAGGCCA GTCCAGAGTG
CTGGGCACCA TACGCATTTT ATCTCCCTCC CGCTATTCAC AACAAATATG GGAGGTAGTT
TATATTATAG CCATCTAATA AGATGGGGAA ACTAAGACTC AAAGAGATTC AGAAACTTGT
CCATGATTAT AAATGTAAGA GAGTTGGAAT TCAGATTTAT GTATTTAGAC CCCAAGCCTT
TCTCATTACA TCATTTTGCC TTCCAAATCT CTACCCTCTA TCCTTCACCT CCCCACTGAT
CAAAACGAGA TGATAGTTTG CCCTCTTCAA AAGAAATGTG TGCATGTATA TATCTTTGAT
TTCTTTTGTA GTGGAAAGTT GGGGACAAAT GTTCTGCCAT TTGGTCAGAA GACGGTTGCA
TTTACCCAGC TACCATTGCT TCAATTGATT TTAAGAGAGA AACCTGTGTT GTGGTTTACA
CTGGATATGG AAATAGAGAG GAGCAAAATC TGTCCGATCT ACTTTCCCCA ATCTGTGAAG
TAGCTAATAA TATAGAACAA AATGCTCAAG AGGTAAGGAT ACAAAAAAAA AAAAATTCAA
TTTCTGGAAG CAGAGACTAG ATGAGAAACT GTTAAACAGT ATACACAGTT GTCAGTTTGA
TCCACCGAGG CATTAATTTT TTCTTAATCA CACCCTTATA ACAAAAACCT GCATATTTTT
TCTTTTTAAA GAATGAAAAT GAAAGCCAAG TTTCAACAGA TGAAAGTGAG AACTCCAGGT
CTCCTGGAAA TAAATCAGAT AACATCAAGC CCAAATCTGC TCCATGGAAC TCTTTTCTCC
CTCCACCACC CCCCATGCCA GGGCCAAGAC TGGGACCAGG AAAGGTAAAC CTTCTATGAA
AGTTTTCCAG AAAATAGTTA ATGTCGGGAC ATTTAACCTC TCTGTTAACT AATTTGTAGC
TCTCCCATGA AACTTTTGTA GCTTAAATAC ACAAGAATTT TTTGAAAAGG AAATAAGATA
ATGATGCAAA ATAGTTAATT TTTTAAAAAA ATGTTAGACA CTGCAGTGGA TGCAACAAAA
TACTTTATAT GAAAGATTTA TCCAGTTAAC TTTTGTGGAG TATTAGGTAT TAGACTAATA
ATTAGCACAC TTACTTAAGT TAGAAAGTAT AATAATGCGC CGGACGCGGT AGCTCACGCC
TGTAATCCCA GCACTTTGGG AGGCCAAGGT GGGCGGATCA CAAGGTCAGG AGATCGAGAC
CATCCTGGCT AACACGGTGA AACCCCATCT CTACTGAAAA TACAAAAAAA TTTGCCGGGC
GTGATGGCGG GCACCTGTAG TCCCAGCTAC TCGGGAGGCT GAGGCAGGAG GATGGTGTGA
ACCCCGGAGG CAGAGCTTGC AGTGAGTCAA GATCGTGCCA CTGCACTCCA ACCTGGGCGA
CAGAATGAGA CTCCATCTCA AACAAAAAAA CAAAACAAAA CAAAAAAAAG TGTAATAATA
ATTTATCATT AGCTGGATGA TATGCTGTTG TTTCCCATGT CACCTGTATA AGATATGTAA
AATAAGAACA CATTATTTAC ATCTAATATA GATAAAATCC TGAGGCGCTC TCAGATTGTT
TTGTAGAGTT CAAATGTAAA TATTGTTTTC ATTTATGGTC CTTTTGGTTA TAAGTAACAG
AAATCAACTC TAAAAAGATT TTTATTATAG GTTAGATTAT GTCATGGAAC CTTAAGGCTT
GTCCCTTTCT AGTTCTTTTG TGTAAAGCGG TGATTTCTTC CATGGAGGGA ATGGTATTTA
GGCAATTTTT TTTTTTTTTT CGAGATGGAG TCTTGCTCTG TCGCTCAGGC TGGAGTGCAG
TGGCACCATT TCAGCTCACT GCAACTTCCA CCTCCTGGGT TCAAGTGATT CTCCTGCTTC
AGCCTCCCAA GTAGCTGAGA TTACAGGCAC CCGCCACCAC ACCCGGCTTA TTTTGTATTT
TTAGTAGAGA TGGGGTTTCA CCATGTTGGC CAGGCTGGTC TTGAACTCCT GACCTCAAGT
GATCTCCCCA CCTTGGCCTT CCAAAGTGCT AGGATTACAG GCGCCTAGCC TAGGCAGTCA
TTTTCAAAAA ACAAGCATGA CTCACCAAAA GTTTTAAGAT TTTCTGTGAT AATGTTCTTA
TTGAGGCTTA CATTATATTA CAGTTTCTTG AATCTAAAAT GATGTACCCT CTTAGAATAT
ATACATCATG CTTCATTGGT CTCAGGGGGC TGATTTTTAT AAGGAGAGAT TTGCTAGTTT
TCACAATATG TCCTCTAAGT TGGCATGTAT AGCTAAACAG GCTTTCATAA AAATATACAA
TTTAGTTAAT GAAATTTGGG ATATAGTCTT TTATGATTGA AATAATTTTG CTAAATAGAC
TGTCTCTGAT TTATTAGGTA ATCACCACTC TTATTTTGTT TTACTTCCTT AATGTCTACA
TAGAAAGGAA ATGAGAAAAA TCCAGAGGTT GTCATTTGAC TTATGAGTCT GTTTGACTTC
AGGATTTGGT ACATGAAATT TCACTTAATC TTTTTGATAT GTATAAAACA AATATTCTGG
GTAATTATTT TTATCCTTTT GGTTTTGAGT CCTTTTTATT CCTATCATAT TGAAATTGGT
AAGTTAATTT TCCTTTGAAA TATTCCTTAT AGCCAGGTCT AAAATTCAAT GGCCCACCAC
CGCCACCGCC ACCACCACCA CCCCACTTAC TATCATGCTG GCTGCCTCCA TTTCCTTCTG
GACCACCAGT AAGTAAAAAA GAGTATAGGT TAGATTTTGC TTTCACATAC AATTTGATAA
TTAGCAGAAT AGAGGATTGT AAAATGTCAT TGTAGAACAT CCCTTGGGCC AGATTCTAAT
GGGTAGAAAT TTGAACTAAA CCTCTGGGTT TTGTTTGTTT TTAATGCCTT TCTGTTACCC
AGATGCAGTG CTCTTGTAGT CCCAAGTCTA AGCTCTAGGT TGCCTTCTTT CCTGGCAGAA
GTTGGTGTCT ATGCCATAAG GAGGTAGTTC CTGTTAGAAG GGATTTAATT ATACCTTATA
TAAGGAATTA GTGTTTGCCC TTCTAGGTAT AGTTGGATGT TAGCTTCTGA TGTAAACTGG
ATTTCTTTTT CTTTCTCTCT CTTTTTTTTT TTTTGTTTTG GAGGCAGAGT TTTGCCCTTG
TACCCCAGGC TGGAGTGCAG TGGTGTGATC TCAGCTCACA GCAACCTCCG CCTCCTGGGT
TCAAGCAATT CTGCCTCGGC CTCCCAAGTA GCTGGGATTA CAGGCGACTG CCACCACACC
CGGCTAATTT TTGTTTTATT AGTAGAGATG GGGTTTCACC ATGTTGGCCA GACTGATCTT
GAACTCCTGA CCTCAGGTGA TCCACCCGCC TTGGCCTCCC AAAGCGCTGG GATTACAGGC
GTGAGCTGCC GCACCCAGCT GTAAACTGGA TTTCTAATGG TAGATTTTTA GGTATTAACA
ATAGATAAAA AGATACTTTT TGGCATACTG TGTATTGGGA TGGGGTTAGA ACAGGTGTTC
TACCCAAGAC ATTTACTTAA AATCGCCCTC GAAATGCTAT GTGAGCTGTG TGTGTGTGTG
TGTGTGTGTG TGTATTAAGG AAAAGCATGA AAGTATTTAT GCTTGATTTT TTTTTTTTAC
TCATAGCTTC ATAGTGGAAC AGATACATAG TCTAAATCAA AATGTTTAAA CTTTTTATGT
CACTTGCTGT CTTTTCGTCC TCGTTAAATT TAATTTTGTT GGTCTTTTGT TGTTATTGGT
TGGTTTTCTC CAAATGCTAG CTATGTTAAG AAATTTAAGG CCAGGTACAG TGGCTCATGC
CTGTAATCCC GGCATTTTAG AAGGCTGAGG CAGGAGGATC ACTTGAGCTC AGGAGTTTGA
GACCAGTCTG GGCAACATAG CAAGACCTCG TCTTTGTTTA GGGGAAAAAA AAGAAATTTA
AGTAGGAGAT TATATAAGCA AAAATACAAT TAATTTCCAG CATTCACTAT ATAATATAAA
TCTCCAGACT TTACTTTTTT GTTTACTGGA TATAAACAAT ATCTTTTTCT GTCTCCAGAT
AATTCCCCCA CCACCTCCCA TATGTCCAGA TTCTCTTGAT GATGCTGATG CTTTGGGAAG
TATGTTAATT TCATGGTACA TGAGTGGCTA TCATACTGGC TATTATATGG TAAGTAATCA
CTCAGCATCT TTTCCTGACA ATTTTTTTGT AGTTATGTGA CTTTGTTTTG TAAATTTATA
AAATACTACT TGCTTCTCTC TTTATATTAC TAAAAAATAA AAATAAAAAA ATACAACTGT
CTGAGGCTTA AATTACTCTT GCATTGTCCC TAAGTATAAT TTTAGTTAAT TTTAAAAAGC
TTTCATGCTA TTGTTAGATT ATTTTGATTA TACACTTTTG AATTGAAATT ATACTTTTTC
TAAATAATGT TTTAATCTCT GATTTGAAAT TGATTGTAGG GAATGGAAAA GATGGGATAA
TTTTTCATAA ATGAAAAATG AAATTCTTTT TTTTTTTTTT TTTTTTTTGA GACGGAGTCT
TGCTCTGTTG CCCAGGCTGG AGTGCAATGG CGTGATCTTG GCTCACAGCA AGCTCTGCCT
CCTGGATTCA CGCCATTCTC CTGCCTCAGC CTCAGAGGTA GCTGGGACTA CAGGTGCCTG
CCACCACGCC TGTCTAATTT TTTGTATTTT TTTGTAAAGA CAGGGTTTCA CTGTGTTAGC
CAGGATGGTC TCAATCTCCT GACCCCGTGA TCCACCCGCC TCGGCCTTCC AAGAGAAATG
AAATTTTTTT AATGCACAAA GATCTGGGGT AATGTGTACC ACATTGAACC TTGGGGAGTA
TGGCTTCAAA CTTGTCACTT TATACGTTAG TCTCCTACGG ACATGTTCTA TTGTATTTTA
GTCAGAACAT TTAAAATTAT TTTATTTTAT TTTATTTTTT TTTTTTTTTT GAGACGGAGT
CTCGCTCTGT CACCCAGGCT GGAGTACAGT GGCGCAGTCT CGGCTCACTG CAAGCTCCGC
CTCCCGGGTT CACGCCATTC TCCTGCCTCA GCCTCTCCGA GTAGCTGGGA CTACAGGCGC
CCGCCACCAC GCCCGGCTAA TTTTTTTTTA TTTTTAGTAG AGACGGGGTT TCACCGTGGT
CTCGATCTCC TGACCTCGTG ATCCACCCGC CTCGGCCTCC CAAAGTGCTG GGATTACAAG
CGTGAGCCAC CGCGCCCGGC CTAAAATTAT TTTTAAAAGT AAGCTCTTGT GCCCTGCTAA
AATTATGATG TGATATTGTA GGCACTTGTA TTTTTAGTAA ATTAATATAG AAGAAACAAC
TGACTTAAAG GTGTATGTTT TTAAATGTAT CATCTGTGTG TGCCCCCATT AATATTCTTA
TTTAAAAGTT AAGGCCAGAC ATGGTGGCTT ACAACTGTAA TCCCAACAGT TTGTGAGGCC
GAGGCAGGCA GATCACTTGA GGTCAGGAGT TTGAGACCAG CCTGGCCAAC ATGATGAAAC
CTTGTCTCTA CTAAAAATAC CAAAAAAAAT TTAGCCAGGC ATGGTGGCAC ATGCCTGTAA
TCCGAGCTAC TTGGGAGGCT GTGGCAGGAA AATTGCTTTA ATCTGGGAGG CAGAGGTTGC
AGTGAGTTGA GATTGTGCCA CTGCACTCCA CCCTTGGTGA CAGAGTGAGA TTCCATCTCA
AAAAAAGAAA AAGGCCTGGC ACGGTGGCTC ACACCTATAA TCCCAGTACT TTGGGAGGTA
GAGGCAGGTG GATCACTTGA GGTTAGGAGT TCAGGACCAG CCTGGCCAAC ATGGTGACTA
CTCCATTTCT ACTAAATACA CAAAACTTAG CCCAGTGGCG GGCAGTTGTA ATCCCAGCTA
CTTGAGAGGT TGAGGCAGGA GAATCACTTG AACCTGGGAG GCAGAGGTTG CAGTGAGCCG
AGATCACACC GCTGCACTCT AGCCTGGCCA ACAGAGTGAG AATTTGCGGA GGGAAAAAAA
AGTCACGCTT CAGTTGTTGT AGTATAACCT TGGTATATTG TATGTATCAT GAATTCCTCA
TTTTAATGAC CAAAAAGTAA TAAATCAACA GCTTGTAATT TGTTTTGAGA TCAGTTATCT
GACTGTAACA CTGTAGGCTT TTGTGTTTTT TAAATTATGA AATATTTGAA AAAAATACAT
AATGTATATA TAAAGTATTG GTATAATTTA TGTTCTAAAT AACTTTCTTG AGAAATAATT
CACATGGTGT GCAGTTTACC TTTGAAAGTA TACAAGTTGG CTGGGCACAA TGGCTCACGC
CTGTAATCCC AGCACTTTGG GAGGCCAGGG CAGGTGGATC ACGAGGTCAG GAGATCGAGA
CCATCCTGGC TAACATGGTG AAACCCCGTC TCTACTAAAA GTACAAAAAC AAATTAGCCG
GGCATGTTGG CGGGCACCTT TTGTCCCAGC TGCTCGGGAG GCTGAGGCAG GAGAGTGGCG
TGAACCCAGG AGGTGGAGCT TGCAGTGAGC CGAGATTGTG CCAGTGCACT CCAGCCTGGG
CGACAGAGCG AGACTCTGTC TCAAAAAATA AAATAAAAAA GAAAGTATAC AAGTCAGTGG
TTTTGGTTTT CAGTTATGCA ACCATCACTA CAATTTAAGA ACATTTTCAT CACCCCAAAA
AGAAACCCTG TTACCTTCAT TTTCCCCAGC CCTAGGCAGT CAGTACACTT TCTGTCTCTA
TGAATTTGTC TATTTTAGAT ATTATATATA AACGGAATTA TACGATATGT GGTCTTTTGT
GTCTGGCTTC TTTCACTTAG CATGCTATTT TCAAGATTCA TCCATGCTGT AGAATGCACC
AGTACTGCAT TCCTTCTTAT TGCTGAATAT TCTGTTGTTT GGTTATATCA CATTTTATCC
ATTCATCAGT TCATGGACAT TTAGGTTGTT TTTATTTTTG GGCTATAATG AATAATGTTG
CTATGAACAT TCGTTTGTGT TCTTTTTGTT TTTTTGGTTT TTTGGGTTTT TTTTGTTTTG
TTTTTGTTTT TGAGACAGTC TTGCTCTGTC TCCTAAGCTG GAGTGCAGTG GCATGATCTT
GGCTTACTGC AAGCTCTGCC TCCCGGGTTC ACACCATTCT CCTGCCTCAG CCCGACAAGT
AGCTGGGACT ACAGGCGTGT GCCACCATGC ACGGCTAATT TTTTGTATTT TTAGTAGAGA
TGGGGTTTCA CCGTGTTAGC CAGGATGGTC TCGATCTCCT GACCTCGTGA TCTGCCTGCC
TAGGCCTCCC AAAGTGCTGG GATTACAGGC GTGAGCCACT GCACCTGGCC TTAAGTGTTT
TTAATACGTC ATTGCCTTAA GCTAACAATT CTTAACCTTT GTTCTACTGA AGCCACGTGG
TTGAGATAGG CTCTGAGTCT AGCTTTTAAC CTCTATCTTT TTGTCTTAGA AATCTAAGCA
GAATGCAAAT GACTAAGAAT AATGTTGTTG AAATAACATA AAATAGGTTA TAACTTTGAT
ACTCATTAGT AACAAATCTT TCAATACATC TTACGGTCTG TTAGGTGTAG ATTAGTAATG
AAGTGGGAAG CCACTGCAAG CTAGTATACA TGTAGGGAAA GATAGAAAGC ATTGAAGCCA
GAAGAGAGAC AGAGGACATT TGGGCTAGAT CTGACAAGAA AAACAAATGT TTTAGTATTA
ATTTTTGACT TTAAATTTTT TTTTTATTTA GTGAATACTG GTGTTTAATG GTCTCATTTT
AATAAGTATG ACACAGGTAG TTTAAGGTCA TATATTTTAT TTGATGAAAA TAAGGTATAG
GCCGGGCACG GTGGCTCACA CCTGTAATCC CAGCACTTTG GGAGGCCGAG GCAGGCGGAT
CACCTGAGGT CGGGAGTTAG AGACTAGCCT CAACATGGAG AAACCCCGTC TCTACTAAAA
AAAATACAAA ATTAGGCGGG CGTGGTGGTG CATGCCTGTA ATCCCAGCTA CTCAGGAGGC
TGAGGCAGGA GAATTGCTTG AACCTGGGAG GTGGAGGTTG CGGTGAGCCG AGATCACCTC
ATTGCACTCC AGCCTGGGCA ACAAGAGCAA AACTCCATCT CAAAAAAAAA AAAATAAGGT
ATAAGCGGGC TCAGGAACAT CATTGGACAT ACTGAAAGAA GAAAAATCAG CTGGGCGCAG
TGGCTCACGC CGGTAATCCC AACACTTTGG GAGGCCAAGG CAGGCGAATC ACCTGAAGTC
GGGAGTTCCA GATCAGCCTG ACCAACATGG AGAAACCCTG TCTCTACTAA AAATACAAAA
CTAGCCGGGC ATGGTGGCGC ATGCCTGTAA TCCCAGCTAC TTGGGAGGCT GAGGCAGGAG
AATTGCTTGA ACCGAGAAGG CGGAGGTTGC GGTGAGCCAA GATTGCACCA TTGCACTCCA
GCCTGGGCAA CAAGAGCGAA ACTCCGTCTC AAAAAAAAAA GGAAGAAAAA TATTTTTTTA
AATTAATTAG TTTATTTATT TTTTAAGATG GAGTTTTGCC CTGTCACCCA GGCTGGGGTG
CAATGGTGCA ATCTCGGCTC ACTGCAACCT CCGCCTCCTG GGTTCAAGTG ATTCTCCTGC
CTCAGCTTCC CGAGTAGCTG TGATTACAGC CATATGCCAC CACGCCCAGC CAGTTTTGTG
TTTTGTTTTG TTTTTTGTTT TTTTTTTTTG AGAGGGTGTC TTGCTCTGTC CCCCAAGCTG
GAGTGCAGCG GCGCGATCTT GGCTCACTGC AAGCTCTGCC TCCCAGGTTC ACACCATTCT
CTTGCCTCAG CCTCCCGAGT AGCTGGGACT ACAGGTGCCC GCCACCACAC CCGGCTAATT
TTTTTGTGTT TTTAGTAGAG ATGGGGTTTC ACTGTGTTAG CCAGGATGGT CTCGATCTCC
TGACCTTTTG ATCCACCCGC CTCAGCCTCC CCAAGTGCTG GGATTATAGG CGTGAGCCAC
TGTGCCCGGC CTAGTCTTGT ATTTTTAGTA GAGTCGGGAT TTCTCCATGT TGGTCAGGCT
GTTCTCCAAA TCCGACCTCA GGTGATCCGC CCGCCTTGGC CTCCAAAAGT GCAAGGCAAG
GCATTACAGG CATGAGCCAC TGTGACCGGC AATGTTTTTA AATTTTTTAC ATTTAAATTT
TATTTTTTAG AGACCAGGTC TCACTCTATT GCTCAGGCTG GAGTGCAAGG GCACATTCAC
AGCTCACTGC AGCCTTGACC TCCAGGGCTC AAGCAGTCCT CTCACCTCAG TTTCCCGAGT
AGCTGGGACT ACAGTGATAA TGCCACTGCA CCTGGCTAAT TTTTATTTTT ATTTATTTAT
TTTTTTTTGA GACAGAGTCT TGCTCTGTCA CCCAGGCTGG AGTGCAGTGG TGTAAATCTC
AGCTCACTGC AGCCTCCGCC TCCTGGGTTC AAGTGATTCT CCTGCCTCAA CCTCCCAAGT
AGCTGGGATT AGAGGTCCCC ACCACCATGC CTGGCTAATT TTTTGTACTT TCAGTAGAAA
CGGGGTTTTG CCATGTTGGC CAGGCTGTTC TCGAACTCCT GAGCTCAGGT GATCCAACTG
TCTCGGCCTC CCAAAGTGCT GGGATTACAG GCGTGAGCCA CTGTGCCTAG CCTGAGCCAC
CACGCCGGCC TAATTTTTAA ATTTTTTGTA GAGACAGGGT CTCATTATGT TGCCCAGGGT
GGTGTCAAGC TCCAGGTCTC AAGTGATCCC CCTACCTCCG CCTCCCAAAG TTGTGGGATT
GTAGGCATGA GCCACTGCAA GAAAACCTTA ACTGCAGCCT AATAATTGTT TTCTTTGGGA
TAACTTTTAA AGTACATTAA AAGACTATCA ACTTAATTTC TGATCATATT TTGTTGAATA
AAATAAGTAA AATGTCTTGT GAAACAAAAT GCTTTTTAAC ATCCATATAA AGCTATCTAT
ATATAGCTAT CTATATCTAT ATAGCTATTT TTTTTAACTT CCTTTATTTT CCTTACAGGG
TTTTAGACAA AATCAAAAAG AAGGAAGGTG CTCACATTCC TTAAATTAAG GAGTAAGTCT
GCCAGCATTA TGAAAGTGAA TCTTACTTTT GTAAAACTTT ATGGTTTGTG GAAAACAAAT
GTTTTTGAAC ATTTAAAAAG TTCAGATGTT AGAAAGTTGA AAGGTTAATG TAAAACAATC
AATATTAAAG AATTTTGATG CCAAAACTAT TAGATAAAAG GTTAATCTAC ATCCCTACTA
GAATTCTCAT ACTTAACTGG TTGGTTGTGT GGAAGAAACA TACTTTCACA ATAAAGAGCT
TTAGGATATG ATGCCATTTT ATATCACTAG TAGGCAGACC AGCAGACTTT TTTTTATTGT
GATATGGGAT AACCTAGGCA TACTGCACTG TACACTCTGA CATATGAAGT GCTCTAGTCA
AGTTTAACTG GTGTCCACAG AGGACATGGT TTAACTGGAA TTCGTCAAGC CTCTGGTTCT
AATTTCTCAT TTGCAGGAAA TGCTGGCATA GAGCAGCACT AAATGACACC ACTAAAGAAA
CGATCAGACA GATCTGGAAT GTGAAGCGTT ATAGAAGATA ACTGGCCTCA TTTCTTCAAA
ATATCAAGTG TTGGGAAAGA AAAAAGGAAG TGGAATGGGT AACTCTTCTT GATTAAAAGT
TATGTAATAA CCAAATGCAA TGTGAAATAT TTTACTGGAC TCTATTTTGA AAAACCATCT
GTAAAAGACT GAGGTGGGGG TGGGAGGCCA GCACGGTGGT GAGGCAGTTG AGAAAATTTG
AATGTGGATT AGATTTTGAA TGATATTGGA TAATTATTGG TAATTTTATG AGCTGTGAGA
AGGGTGTTGT AGTTTATAAA AGACTGTCTT AATTTGCATA CTTAAGCATT TAGGAATGAA
GTGTTAGAGT GTCTTAAAAT GTTTCAAATG GTTTAACAAA ATGTATGTGA GGCGTATGTG
GCAAAATGTT ACAGAATCTA ACTGGTGGAC ATGGCTGTTC ATTGTACTGT TTTTTTCTAT
CTTCTATATG TTTAAAAGTA TATAATAAAA ATATTTAATT TTTTTTTAAA TTA
Homo sapiens SMN2 pre-mRNA nucleic acid sequence
(SEQ ID NO: 64)
CCACAAAUGU GGGAGGGCGA UAACCACUCG UAGAAAGCGU GAGAAGUUAC UACAAGCGGU
CCUCCCGGCC ACCGUACUGU UCCGCUCCCA GAAGCCCCGG GCGGCGGAAG UCGUCACUCU
UAAGAAGGGA CGGGGCCCCA CGCUGCGCAC CCGCGGGUUU GCUAUGGCGA UGAGCAGCGG
CGGCAGUGGU GGCGGCGUCC CGGAGCAGGA GGAUUCCGUG CUGUUCCGGC GCGGCACAGG
CCAGGUGAGG UCGCAGCCAG UGCAGUCUCC CUAUUAGCGC UCUCAGCACC CUUCUUCCGG
CCCAACUCUC CUUCCGCAGC CUCGGGACAG CAUCAAGUCG AUCCGCUCAC UGGAGUUGUG
GUCCGCGUUU UUCUACGUCU UUUCCCACUC CGUUCCCUGC GAACCACAUC CGCAAGCUCC
UUCCUCGAGC AGUUUGGGCU CCUUGAUAGC GUUGAGUGGA GGCCCUGCCG CGACUUGGCA
GUAGCUUAUU UUGUUCACUC CUCUCUGGCU GGUGUGGGGG AGGUGGGGGC AUUAGGCCAG
GGUGAAGCAG GGGAACCACU UAGGAGUCUG UUAAGAUGAU CUGAACUUCA GAACAAGAUG
UUAUUAACAG AGUGAAAGUA UUUGGAUUCU GGGUAUAUUU UGAAAUCGGA GGCAACAGGU
UUUUCAGAUA GAUUCGAUAA CGGAGGUUAU CCUGAAUAGU UGAAAAGAUA AAGUUGCCUU
UUGCUGAGGU GGGAAAGAGA AGAUUGCCAG UAGAGCAGGU UUCUCAGGAG UUCAGUCUUG
GGCAUAGCAU GGUAGGGGUG AAUUUGGCUG GAGUGAGUUG GAGAGUAGGA GAAGAGAAAU
CCAAGGCAAC AUUUGACCAG CCUGGGCAAC AUAGUGUGAC UCCGAGUCUG CAAAAAUUAG
ACGGGUGUUG UGGUGCGCGU CUGUGGUCUC AGCUACCUGG AAGGUUCAGG CCUUGGAAGG
CUCAGGGAGG UGGAGGCUGC AGUGAUCUGU GAUUGCGCCU CUGCACUCCA GCCUGGGCGA
CAGAGCCAGA CCCUGUCUUA AAACAAAAUA AACGGCCGGG CGCGGUGGCU CAAGCCUGUA
AUCCCAGCAC UUUGGGAGGC CGAGGCGGCC GGAUCACAAG GUCAGGAGAU CGAGACCAUC
CUGGCUAACA CGGUGAAACC CCGUCUCUAC UACAAAUACA AAAAAUUAGC CGGGCGUGGU
GACGGGCGCC UGUAGUCCCA GCUACUCGGG AGGCUGAGGC AGGAGAAUGU CAUGAAGCCG
GGAGGCGGAG CUUGCAGUGA GCCGAGAUCG CGCCACUGCA CUCCAGCCUG GGCGAUAGAG
CAAGACUCCG UCUCAAAUAA AUAAAUAAAU AAAUAAAUAA AUAAUAAAAA CAUCGGUAGG
CAUAUUUCAA GGAAUUCUAU UUAAAAAAAA UUUUUUUAGA GACAAGUUCG CUCUCUGUGG
CCCAGGCUGG AGUACAGUGG CAUGAUCCUA GCCCAUGGCA GCGUUGAUCU CUUGGCCUCA
AGCGACCCUC CUUUGGAGUC GCUGGGCCUA AAGGAGUGAG CCACCACGAA AUUUUAUUAU
AAAUGGAGGG UAGAGAAAUU GGGCAAUAAA UGGAGGGGGA AGUGAGUUAA GAGGAAUUUU
AAUUAUGUGU GUGUGGUUUU AAAAGAGGGG GGUCUUGCUC UGUUGCCCAG GCUGCUGGGG
UGCCAGUGGC GCAAUCAUGA AUCACUACAG CCUUGGACUC CUGGCCUCAA GCUAUCCUCC
CACCUCUGCC UCCCAAAGUA CUGGGAUUAC UAGUGUGAGC CACUGCACUA AGAUAGGAGC
AACAUGUUUC AGCAUGUUUG UGGGUUGAUA GGAAAGAUGA GAAUGGGAAA GUUGAUGUCG
GAAAGAAGAC AAUGGCUAGA GCAAUGUCCU AGAGUAGGUA AGAAGGGAUG GAUUUGGCCU
UUGUUGGAAA CAUUAGCGGU UCUUUUGGUG ACAGCUAUAU AGUUAACACA UCUAUGAUAC
GUGAAUGGGC AGAUAGGAUG GCAGGAGAUU UUGAAAGUUC UCUUGAUUCU UACUGUUCUC
UUAGUGAAAG AAGCAAGGUU AUCAGCUAGA AGCUGGGAUG GGAGAGGAAA GAGAAGAUGG
GAAGUAGAUA GUUCUUUAGA AGAGUGGGCA AGGGUUGGAC UAGGGAAGUU UAGUGGAAAU
AUUGCUAGGC AACAUAAAGA GCCUACUUGA GAUUCGUGGU CAUGAGUUGA AGGAGACCAG
ACAGCAAGAU UGUGUAUGAG GGCACCCACA GAGUAAAUGG AGAGUUGAAA UUAAUGCAGU
UGUGAUUUUA CCACGUGGAU AUGAAGAAGU GAGGGGGAGA AGUACAAAGG AGUUCUCUUA
AUGAUUGACC AUGGAAUUUA AGCUGGCUAA GAAAGGAAGU GAGAGGCCGG GCGCGGUGGC
UCACGCCUGU AAUCCCAGCA CUUUGGGAGA CUGAGGUGGG UGGAUUACCU GAGGUCAGGA
GUUUGAGACC AACCUGGCCG AUAUGGCGAA ACCCCAUCUC UAAUAAAAAU ACAGAAAAAU
UAGCCGGGAA UGGUGGCAGG UGCCUGUAAU CCCAGCUACU CAAGAGGCUG UGGCAGGAGU
AUCCCUUGGA CCCAGGAGGU GGAGGUUGCA GUGAGCCGAG AUCACGCCAC UGUACUCCAG
CCUGGACGAU AUAGUGAGAC UUCACCUCAA AAAAAAAAAA AAAGAAAGGA AGUGAGGAUU
UUAAGACCCU GAGAGACAGU UUAAAAAGUG GGAGGAUCGG CCGGGCGCUG UGGCUGACAC
CUGUAAUCCC AGCACUUUGG GAGGCCGAGU UGGGCAGAUC ACAAGGUCAG GAGUUCGAGA
CCAGCCUGGC CAAUAUGGUG AAACCUUGUC UCUACUAAAA AUACAAAAAU UAGCCGGGCA
UGGUGUCACG UGUCUAUAAU CCCAGCUACU CGGGAGGCUG AGGCAGAAAA AUUGCUUGAA
CCUGGGAGGC AGAGGUUGCA GACAGCUGAG AUCACUCCAU UGCACUCCAG CCUGGGCAAC
AAGAGCAAAA CUUUGUCUUU AAAAAAAAAA AAAAAAAAAG AAUACAAAAA UUAGCCGGGC
GUGGUGGCGC GUGCCUAUAA UCCCAGCUAC UUGGGAGGCU GAGGCAGGAG AAUCAGUUGA
ACACGGGAGG CGAGGUUUGC AGUGAGCCGA GAUUGCGCCA CUGCACUCCA GCCUGGGCGA
CAGAGCAGGA CUCCUCUUGG AAAAAAAAAA UUAGCUGGGC AUGGUGGCAG GUGCCUGUAG
UCUCAGCUAC UAGGGAGGCU GAGGCAGGAA AAUCACUUGA ACCCGGGAUG UGGAGUUUGC
AGUGACCCGA GAUCGUGCCA CUGUACUCCA UCCUGGGCGA CAAAAUGAGA CUCUGCCUCA
AAAAAAAAAA AAAAAAAAAG UGGGAGGAUC AAUGUACUGC CAGUCCUAAU GAAGUGGAAU
GAUUGUCCCC AUCAAAUCAC UAGUAGGAGU AAGUUGCAGA GCCUAGAAGG UGAUGGUUAA
GAGAGUGGGA UUCUUGAAAC UGCAUUUAUG GAGAGGUUGU GGUUAUUGGU UAUAAUAAAU
AAAUACAGUU GAAGUGAGUG AGUAGCUGAG AUUUGGGGAU GUAUCAGUUC AUUCUUACAC
UGCUACAAAG ACAUACCUGA GACCAGGUAU UUAUAAAGAU AAGAGGUUUA AUCAGCUCAC
AGUUCUGCUG CCUGUACAGG CUUCUCUUGU GGAGGCCUAA GGAAACUUAC AGUCAUGGUG
GAAGGUGAAG GGGAAACAAG CACAGUCUUC ACAUGGCCAG CAGGAGAGAG AGAGAAGGGG
GAAGUGCUAC AUACUUUAAA ACAACCAGAU CUUGUGAGAA CGCUUAUCAG GAAACAGCAC
UUGGGGAUGG UGCUAAAUCA UUAGAAAUCA CCCCCAUGAU CCAGUCGCCU CCUACCAUGC
CCACCUCCAA CACUGGGGAU CACAAUUCAG CAUGAGAUUU GGGUAGGAAC ACAGAGCUGC
ACCACAUCAG AGGAUGUACA AGAUUGUGGU GGAGAGGAGU UUAGAGACCU GCAAAUAUAG
GGUAAUUGAA GGGAUCAUCU ACAUGGAUAU UUAAAUCACC AAAAAUUAUG ACAGGAGUAG
UGUUGGAGAG AGAACUGCGA UGUAAACAUU AAGGAAUGAG GAAGAGUGAC UCGGUAGGCU
GUAGGUGACU GCAAUAGGAA ACGAUAAUAG ACUGUGAGUC UGGUGACAAG AUUUUCCUUC
UUUCUUUUUU UCCCCCCCCC CGAGACAGGG CCUCUUUUUG UUGCCCAGGU GGGAGUGCAG
UGGCGCGAUC ACGGCUCACU ACAACCUCCU CCCAAGCUCA AGGGAUUCUC CCACUUCAGC
CUCUCAAGUA GCUGGAACUA CAGGUGCUGA CCACCAUGCC UGGCUACUUU UUGUCAGGAU
UUUCAAGGCU GGGAAUUUUG AGAGGGGAAU GGAGGAGAAU AAUCUGAAAG UGCAAGUAAG
GAGCAGGGAA GAUUUCUUUU UUCUUUUUUU UUUUUUUUUU UGAGUCGGAG UCUGGCUCAG
UCGCCCAGGC UGGAGUGCAG UGGCGAGAUC UCCGCUCACU GCAAGCUCCG CCUCCCGUGU
UCACGCCAUU CUCCUCCUUC AGCCUCCCGA GUAGCUGGGA CUACAGGCGC CCGCCACCAC
GCCCAGCUAA UUGUUUUUUU GUAUUUUUAG UAGAGACGGG GUUUCACCGU GUUAGCCAGG
AUGGUCUCAA UCUCCUGACU UUGUGAUCCG CCCACCCCGG CCUCCCAAAG CGCUUGGGAU
UACAGGCGUG AGCCACCGCG CCAGCCAGAG CAGGGAAGAU UUCUUCCCCA CAUCUCCAGU
AGGUACAGUG AUAUGAAGUG UGUGGAGGAG AAAAGAGGAA ACAUCUAUCA UUUGAGAUGG
CUGCGAAAGG AAAAGGCAUC CUCAGGGAGC UAGAUUUUAC UUAGAGCAAG AAAUGAAGGG
AUGAUUCAGA GGUUAAAAGA GUGGAUUUUA UGAAUUACUC AAGGGAGCAC AGUGGAAGUU
UCAGGAAGUG GUAGGAGAAG GUAGAAGAUG GCAGGGUGUU GGGAAUAAUU UGAGAAAUCU
GAGCUACUGG AAAUGACUGA GAAUCAGAUA UAAAGGCAGU CCUGGUGGUC CGUUCUGGCU
GCCGUUGCUG UGUAACGAAU CUGCCAAAAC UUAGUGGCUU GAAACAACAA AGAACAUUUU
AUUAUCUCUC AUUGUUUCUG UGGGUUAGGA AUUUGUGAGA GCCGUGCUGG GCAGUUUUCG
UGCGGCUGUC UCGUGGUUGC ACCUACAUAG UUGCUAGAGC UACAGUAGCU GGGGACUGAG
CAGCUAGGGA UUGGCAGGCU AUCUCUUUUU UUCAUGUAGU CUCAUGAAGA UUUCUUUAUG
UGGUUUCAAU GUGUGGGCUG GUUUGGAUUU CCUUAUAGCA UGGUGGCCUC AGUUGGAUUG
CUGUUUUGUG AUCCUUUUCA UCCCUCCUUG UCCUGUCCCC AGACAACCAC UGAUCUACUU
UCUGUCACCA UAGAUUAGCC UGCAUUUUUA AGAAUUUUUA UAAACGUGGA AUGAUAGAGU
ACCUUUUUUG UCACGUUUCU UUUAUUUAUC AUAGCUAUUU UGAUUUUCAU CCAUUUUAUU
GCUGAGUAGU AUCCCAUUGC AUGUAUAUAC UAUACUGUAU UCAUUCGCUU GCUUGUGAAC
AUUUGGGCUU UUUCCAGUUU GGGACUGUUA ACAAGUAGAG CCACUAUGAA UAUUAGUGUA
UAAGACUUCA UAUAGCCAAG GCUGGCAGAU CGCUUGAGCC CAGGAGUUUG AGACCAGCCU
GGGAAACAUG GUGAAACCUC UAUUUUUAUU UUAAAAUCAA AAAUUAAAAA UUUUCUAUAA
AAAAUUUUAA AGAAGACUUU GUAUAGACAU ACGCUUUCAU UUUUCUUGAG UGAAUACUUA
GGUCUCAGGG UAGAUGUAUU UUAAGUCUUU AAGGAGCUGU CAAACUCUUC CUCAAAGUGG
UGGUUGUACC AUGUUACUUU UUAAUAUAAC AGAGAUUAAU UGAGCAAAGA AAAAUUCAAA
AGUUGGACAG CCCCCACAAC UAAAUAGGUU CAGAACAGCU CCCCCAUUUU GCAUUUUGAC
CAGCAAUGUA UGAAAGUUCC AUUUGCUCAG UGUCCCUGCA AACACCUGGU AUGGUCAGUC
UUUUUAAUUU UAGGCAUUAU AAUAGAUAUA GUGGCUUCUU GUGAUUUUAA UUAGCAUUUC
CUAAUGACCA GUGCUGCUGU UGAUCAUUUC AUGAGUGUAU UUGCCAUCCG UAUAUCUUUU
UUGGUGAAGU GUCUAUUCAA AUCAUUUGGG UUUUUUUUUU UUUUGUUUUU UUUUUUUGGA
GACAGUGUCU CACUCUGUCA CCCAGGCUGU UGUGCAGUGG UGCAAUCACA CAGCCUACUG
CAGCCUCCAC CUCCUGCGCU CAGUCUUCUU GUCUCAGCCU UCUGAGUAGC UGAAAUUACG
AGCACACGCC ACAAUGCCUG GCUAAUUUUU UAAAAUUUUG UAGAAACAAG GUCUCAUUAU
GUUGCCUGGG CUUGUCGUGA ACUCCUGGGC UCAAGCAAUC UUCCUGCCUC AGCCUCCCAA
AGAUUGGGAU UGCAAGUAUG AGCCACUGCA CCCGGCCAAC UUACCCAUCU UUUAAUUGAA
UUUUUUUGUU GUUGAGGUUU GAGAGUUCUU CAUGUUUGCU GGGUACAAUA UCUUUAUCAG
AUAGGUAACU UGCAUGUAUU UUCUCCCGGU UUACACUUUG GUUUUUCAUU UUGUUAACAA
CGUCUUUUUA AGAACAGAAA AUCUUAAUUU UGCUGAAAUC UAAUUUUUCA GUUUUUUCUU
UGAUGGUUUU GAGAGAGGAG GUAAAAAAAG ACUAGGUAAG CCGAUAGUUA GACAGAGUCC
UCGGUAGAAC UUCCCUUCUA ACAAAAAGCA GCCCAAGAAA UCACUUCUCU UCUAACAAGG
AGCAGCCUGG AAGAUCGGGC UGUAAACAUG UAUAAGGAAG CAGCUCUGGC ACAGAGGGGG
AGCUUCCUGG GUAAUCAGCA AGCUUCACAU ACGUAAGGUG GGUAUGUGAA GUAAACACAG
UAUGUGAAGU AAACACAGUG GACCUUAGUA CAUACUCAGA UAAGGAAGCU GGAAGCUUGC
AUGUUGUGAG UUGUUGGGGU UGCCUGCAGC UGCACGGAGA GAAAGGGGUA CCUGGGGCCA
GGCAUGUCCA CCAUGGUGGC UCCACCUCCC CUUAUUUAGC ACAUGCACAA UAGGAAAGAG
AUAAGCAAUG UGGAGUAGCU CAGGCCAAGG ACCUGCCUGC AUAAUAAAAG GUUGGGGUGG
GGGAUGCCAG AGAUUCACGC UCUGUGCAGA UGGCAACACC UGGUCCUAAC UGGUUUUUUG
CUCCCUAUGU GUAGAUAAGC UACCCCCUUC CCAUUAGCUC AUUUAUAAAA AUGCUUGCAU
UUCACUGUGG AAUGGGAACU CUUUUCAGGA CCUCUCUCUG CAGGAGAGAG CUAGUCUCUU
UCUUUUGCCU AUUAAACUUC UGCUCUAGCC UCACACCCUU GGUGUGUCAG CGUCCUUGAU
UUCCUCAGCG UGAGACCAAG AACCUCGGGU GCCACCCCAG GCAACAAGGC CAUUUCAGUU
UGUUCUUUUG UUAUAGGCAA UCCAUGAUCA CAGAUUUUUC UCUCUUUUUU UUUUUUACAC
AGUUUAGAGU UUUAGUUUUA CACUUAGGUC UGUAAUCCAU UUUGUAUUAA UUCUUAUAUG
UGGCUCAGUG UAGGUGGAAA UUUGGUUUGU UUUUGCAUAA GGAUUUCCAA UAGUUUUACC
ACCAUUUCUU GAAACUACUA UGCUUUCUCU AUUAAACCAC AUUUGUAACU UUAGUUAAAA
UCAGUCACAU AUAUCACAGG GCUAUUUCUG ACUCUCAAUU CUGUUACAUU GUCUAUUAGU
GUAUAUUGAU GUCAGUACUA CACUUUUAAU UACUAUUGCU UCAGGGUAUG UCUUGUAAAC
CAAAAAUAAA AUUAUAGGCC CCCCCCGCCC CUGCACAACC AACUGAAUGG ACCCAUCCUC
UCAGCCAAGG GCAUUCCAAA AUUAACCUGA AAAACUAGUU CAAGCCAUGA UGGGAAGGGG
GAGUUGGACA UGUCUCAUCA CACCCUACUA CCUUUUGGAA UUACUGAUAG AACAGACUCU
UAAAGUCUGA AAAGAAACAU UUACAACCUA CCCUCUCUGA AGCCUGCUAC CUGGGAGCUU
CAUCUGCAUG AUAAAACCUU GGUCUCCACA ACCCCUUAUG GUAACCCAAA CAUUCCUUUC
UGUUGAUAAU AACUCUUUCA ACUAGUUGCC AAUUAGAAAA UCUUUAAAUC UUCCUAUGAC
CUAGAAACCU CCCUACCCCC ACUUUGAGUU GUCCUGCCUU UCCUGACAGA ACUCAUGUAC
AUCUUACAUA UAUUGAUUGA UGCCUCAUGU CUCCCUAAAA UGUAUAAAAC AAAGCUGUAC
CCCACCACCU UGGGGACAUG UCAUCAGGAC CUCCUGUGGC UGUGUCAUAG GAGCGUCUUU
AACUUUGGCA AAAUAAACUU UCUAAAUUGA UUGAAACCUG UCUUAGCUAC UUCUGGUUUA
CAGUCUUAAA GUUAGAUAAU GUAAAUUGUC CAGCUUUGGU UUAUUUUUGU CCUUAGUAGU
UCCAUAUAAA UUUUAGAAUC AGCUUUUCAA UUUAAUACAC UACUUUCCUC UUAGAUCCAC
AAUUAAAUAU AUUUGAUGCU AACAAUUCUG UUUUAUGUUU UUCGUUUUUU UUUUUUGAGA
CAAGAGUUUC GCUCUUGUUG CCCAGGCUGG AGUGCAGUGG CGCGAUCUUG GCUCACCACA
ACCUCCACCU CCCAGGUUCA AGCAAUUCUU CUGCCUCAGC CUCCCGAGUA GCUGGGAUUA
CAGGCAUGCG CCACCACGCC CGGCUAAUUU UGUAUUUUUA GUAGAGACGG GGUUUCACCA
UGUUGAUCAG GCUGGUCUUG AACUCCUGAC CUCAGGUGAU CCACCCACCU CGGCCUCCCA
AAGUGUUGGG AUUACAGGCG UGAACCACCA UGCCUGGCCA GUUCUGUUAU UUUUAAAACC
CAAGUUUCCC UGGUCAUAUC UUGGUUGGAU GAAGCGUAUU UUCAAUAGAU UACCCUGGAA
AGGCUAGUGA GUACGGUAUU CUUCUACAUU UUAGACUUUU CUUAGUCUUG CUACUUCAAG
GACAGCUAGG CUGCAUAUAA AAUUCUUGGC UCAUACUUUU UCCCCAUAAA UUUCUAUGAG
AAAGUCUAAU GAUAACUGAU UUUCUUUAUU UUGUAACUUA GUCUUUUUGC UUAGAGGCUC
UCUGAGGAUG GGAGGGGGUU CUUCCUCCCA UCCCUAGGAA UUUUUCUUUU UUUUAAAUUC
CUAAUCACUA GACCACCAGG AAGAUUGUUU GUUUUGUUUU GUUUUUAUUC UUCAGGGACC
CCAUUUAUAC AUACGUUAAA UAAAUACUGU UUGCCAAUGU AUCAACCAUU UUGCUUCUUA
UUUAUUUUUG UUCCUUUGGU UCUUUUUCAU GGCUUUGCUU UGGUGCUCCU UAGAUUUUCA
GUCAGAUGUA UUUGUCCUUG GGUACCUUGU AAUCAGUAUU ACCUUUUCUU CUGUCGCUUU
GUUUUCUGUU CGUUUUGAAA UUACUUGUUU CCUGGUCUGG CAAUAACAGU UGAGAUAUGA
GGAGUUUGAG CUGCCAUCUG UCUAUGUAUC UUGCUUUAAG ACUGCACUCU UCUAUUGAUA
UCACUGGCCU UGAUUUUGUG AUUUCUUUAU UUCUUCAGGA CCACCCUUCA UUUUCUACUG
UUUGCUUCCU UUUUUUUUGA GAUGGAGUCU CACUCUGUCA CUCAGGCUGG AGUGCAGUGA
UCUUGGCUCA UUGCAACCUC UGCCUCCCGG GUUCCAGCAA UUCUCCUGCC UCAGCCUCCC
AAGUAUCUGG GACUACAGGU GUGCACCACC AUGCCCGGCU AAGUUUUGUA UUUUUAAUAG
AGACGGGGUU UUGCCACAUU GGCAGGCUGG UCUCAAACUC CUGAUGUCAA GUGAUCCACC
CACCCCACCC ACCUCUGCAU CCCAAAGUGC UGGGAUUACA GGAAUGAGCU GCCGUGCCCA
GCCUCCCCCC UACCCCCCUU UUUUUCUUUC GAGACAGAGA UUAUAGGUGU GAGCCACUGG
ACCCAGCCUG UUUUUAUUCC UUUUACCAAA UCUCCAAGGA AUAUCUUCCC UUCCAAGUGC
GAAUGUAACC UUAAGUCAGU UAACCUCUUU GUGAUUACUU UUCUUAUCUG CAAAGUGACU
UAAUGAUCUU AAGUACUUUU UUUUUUUGAG ACAGGGUCUC ACUGUCACCC UGGCUGGAGU
GCAGUGGCAC GAUCUCUGAU CUCCACUCAC UGCAAUCUCC UCUUCCCUGG UUCAAGCGGC
CCUCCCACCU UAGCCUUCUG GGUAGCUGGG ACUACAGAUG UGAACCACCA CGCCCAGCUA
AUUUUUGUAC UUUUUGUAGA GAUGGGGUUU UGCCAUGUUG CCCAGGCUGG GAUUAUUAAG
UACUUUUUAU CAUACAGCAA GAUUGACAUU UUAUAUUGGA AUACAUUUGU CUCUAUAUAA
CGGAGAUUAA CAGGAAAAUG ACAAGCCUGG GUGCGGUGGC UCAUGCCUGU AAUCCCAGCA
CUUUGGGAGG CUGAGGUGGG AGGAUCACUU GAGGUCAGGA GUUCGAGACC AGUUUUGCCA
AGAUGAUGAA AGCCCAUGUC UACUAAAAAU ACAAAAAUUA GCCCAGCUUG AUGGUGGGCG
CCUAUAAUCC CAGCUAUUUG AGAGACUGAG GCAGGAGAAU CACUUGAACC UGGGCAGCAG
AGGUUGCAGU GAGCCGAGAU CAUGCCACUG CACUCCAGCC UGGGUGGCAU AGCGAGACUC
UUGUCUCAAG AGAAAACAAA ACAAAACAAA AAAAAAACAG GAAAAUGACA AAAAGUAAUA
UUACAACUCA GUGAAUUUUA UAACAAACUU UUUUGGAAUU CAUUGACUAA UACUAUACCA
AAUCCAAAAU ACUCUCUAGU AUACCAAAUC CAACUCUACC CUAUAGUAUA AAUUGGAUUC
UAUUUGGACU UGUCUCACUA AUCCCUCAUA CAGUGUGUUU UAUUUUUUAU UGAAGUAAAA
AAAUUUGUCA UUUUAACCAU UUUUAAGUAU AUAGUUCAGU AAUAUUAAGU AUGUUCAUGU
UGUUGCGCAA UAGAUCUUCG GAAGUUUUUC GUCUUGCAAC CUGAAACUCU ACCCAUUAGC
AAAUUCCCAU UUCUCCUUAC ACUUAGCCCU UGGUAAUCAU CAUUCUUUUU UUUUUUUUUU
UGAGAUGGAG UUUUACUCUU GUUGCCCAGG CUGGAGUGCA AUGGUGCAAU CUCGACUCAC
CACAACCUCC GCCUCCCAGG UUCAAGCAAU UCUACCUCAG CCUCCCGAGU AGCUGGGAUU
ACAGUCAUGC ACCACCACGC CCGGCUAAUU UUGUAUUUUU AGUAGAGAAG GGGUUUCUCC
AUGUUGAGGC UGGUCUCGAA CUCCUGACCU CAGGUGAUCU GCCCACCUCG GCCUCCCAAA
GUGCUGGGAU UACAGGCGUG AGCCACUGCG CCUGGCCCAU UCUUUCUAAU UCUAUAAAUU
UGACUACUUA GUUACCUUAC AUAAAUAAAU UCUUAUAGUU AGUGUUAUUU UUGCUUCCAU
GCCUUUUUUG UUGUUGUUCA UGCUCUUACU UGGAAUGCGU UCUAUUUUGU CUACCUAUGC
ACAUCCUGUU GGGUUUUUUU UUUUUUUGGG GGUUUUUUUU GUUUUUUUUU GUUUUUUUUU
CCCAGACAAG GUCUCAAUUU GUUACCCAGG CUGGAGUGCA GCGGCGCCAU CUCCACUCAC
UGCAUCCUCA ACUUCCUGGG CCCAGGUGAU CCUCUCGCCU CAGCCCCUGC AGGUAGCUGG
GACUAUAGGC AUGUGCCACC AUGCCCAGCU AAAUUUGGUU UUUUUGUUUG UUUGUUUUUG
AGACAGAGUC UCACUCUGUC ACCCAGGCUG GAGUGCAGUG GCACAAUCUC AGCUCACUGC
AAUCUCUGCC GCCCGGGUUC AAGUGAUUCU CCUGCCUCAG CCUCCCAAGC AGCUGGGAUU
ACAGGUGACU GCCACCACGC CAGCUAAGUU UUGUAGUUUU AGUAGAGAUG GGGUUUCACC
UUGUUGGCCA UGCUGGUCUC GAACUCCUGA CCUCGUGAUC UGCCUGCUUC UGCCUCCCAA
AGUGCUGGAA UUACAGGCAU GAGCCACCAC GCCCGGCCAG AAUUUUUGUA UUUUUAGUAG
ACACAAGGUU CUUACCCUGU UGCCUAGGCU GGUCUGGAAG UCCUGGACUC AAGCAAUUCA
CCUGCCUUGG CCUCCCAAAA UGCUGGGAUU ACAAGCCACC AUGCCCGGCC UAAAUCCUGU
UGUUUUGUUU UGUUUUAUUU UGUUUUGUUU UGUUUUGUUU GUUUUUUGAG ACAGAGUCUC
GCUAUGUCUC UCAGGCUGUA GUGCAGUGGC GCGAUCUUGG CUCACUGCCA CCUCUGCCUC
CCAGGUUCAA GUGAUUCUCC UGCCUCAGCC UCCCAAGUAG CUGGGAUUAC AGGCAUGUGC
UACUAUGUCC GGCUAAUUUU UGUAUUUUUA GUAGAGACAG GGUUUCACCA UGUUGGCCAG
GCUGGUCUCG AACUCCUGAC CUCGUGAUCC ACCCACCUCG GCCACCCAAA GUGCUGGGAU
UACAGGCGUG AGUGGUUUUU AUUUCUUAGG CCGGUUUCCU CCAUAUGAUC UUGCAGUAGA
CAUUAAUUUC UUUCCUUUUU AAUUAAAAUA CUGUUUGUAU UUCACAUUUU GAUGUUUGUU
AAGAUUUGUU UUAUAUUGUU UUUUGUUUUG UCUUGUGUGA UAGUCUUAAA UCCCUAGUUA
GAUAAUAACU GGAGAGUACC AUGUUUCUAU AUAUCUCUCA GUGACUUGCA CAGUGCUAGC
AGAUAGUGCU AAAAAAUUAU UUAUUAUUAU UAUUAUUUUG UUAUUGUUGU UGUUGUUGUU
AGACAGGGUC UUCCUCUGUC ACCCAGGCUA GAGGGCAAUG GGAUGAUCAU AGCUUACUGC
AGCCUCCAAC AACUGGGCUC AUGUAAUUCU CCUGCCUCAG CUUCCCAAGU AGCUGGGAUU
ACAGGCAUGA GCCACCAUGU CUGGACAAAA AUAUUUCCAG GUGCAGUGGC UCAUGCCUGU
AAUUCCCACA CUUGGGAGGC CGAGCGAGGC UGGAGGAUCA CUUGAGCCUA GGAGUUCAAG
ACCAGCUUGG CUAAGAUGGC GAGACCCCGU CCCUACAAAA AAUUUUAAAA ACUAGCCAGG
CAUGGUGGCA UGCACCUAUA UUCCCAACUA CUCAGUGGGC UGAGGUGGGA GGGUCAUUUG
AACACAGGAA UUUGAGGGGA GAAAAAAAGA AGAGAGAAAG AGAAGUGAAG GAAGGAAGAA
AGGAAGGAGG GAGGGAGAGA AGAAAGAAAC GAAAGAAAGG AAAAGAAAAG GAAGGAAAGA
AAAUUGGUAC CAGGAAAGCA GGAAAGGGAA AUGGAAGUAA AAAAAUAAUA AUAAUAAUAA
AAUGAAAAUU GGUUAGUCAC UAUUAACAAU UUGUAUCCUU AUAAUCUGGA AACAUUAUAA
UUUCAAAAGA AAAAAUAUUC UUUGGAUCAU AGGUUCUGAG GUCAGAACAG CAUUCCCGUA
GUCUAGAUGA AGUCAAGUUU UAUCUGAUCU UAAUUGAAAU AAAUAUAGCU GGCCUUGAAC
AAAUCUACUC AUGGUAUGUG GAUAGGAAUU AAAUUGUAGG GGCAUUCACU UGAUGGCAUU
CAUUCUUAGA ACAUUUACCU AUGUCUAGCU UUUGGAGUAA AGUCACAUAA CCUCUAACCA
GGUAAGUUUC CUGUGGCUUU AUUUAGGAUU UUAAAUACUC AUUUUCAGUG UAAUUUUGUU
AUGUGUGGAU UAAGAUGACU CUUGGUACUA ACAUACAUUU UCUGAUUAAA CCUAUCUGAA
CAUGAGUUGU UUUUAUUUCU UACCCUUUCC AGAGCGAUGA UUCUGACAUU UGGGAUGAUA
CAGCACUGAU AAAAGCAUAU GAUAAAGCUG UGGCUUCAUU UAAGGUAUGA AAUGCUUGCU
UAGUCGUUUU CUUAUUUUCU CGUUAUUCAU UUGGAAAGGA AUUGAUAACA UACGAUAAAG
UGUUAAAGUA CAUGUUAUUC AGUUUUCAUU UUGAAGAUUA GAUGGUAGUA UGAGUUAGUU
AAAUCAGGUG AUAUCCUCCU UUAGAAGUUG AUAGCCUAUA UAUGUCAUCC UUUGUGGAGG
CAAUUUAAAU AAAAUUUAAA ACAUUUAUUC CUGGCUGGGU AUGGUGGCUC ACUCCUGUAA
UCCCAGCACU UUGAGAGGCU GAGGCGGGUG GAUCACCUGA GGUCAGGAGU UUGAGACCAG
CCUGGCCAAC AUGGUGAAAC CCCGUCUUUA CUAAAAAUAC AAAAAUUAGC CAAGCAUGGU
GGCACGUGCC UGUAAUCCCA GCUGCUUGGG ACACUGAGGC AGGAGAAUUG CUUGAACCUG
GGGGGCAGAG GUUGCAAUGA UUGCACCACU GCACUCCAGC CUGGGCGAUA GAGUGAGACU
CCAUCUCAGA AAACGAACAA ACAAUGUAUU CCUUUUAGUA UUUUUACAUU GUAUCAAACU
AUGGAAGUCC UCUAAUUGAG AUUAAUAAGA AAAAGACAAU CUGAAUUAUA AUUUUAAACA
UUUAACAAGC AUGUAGUAAA AUAAUGAUGA AGAUAAAUAG CAUUAGUACA GCAAUUAAUA
UUUGUAGCAU GCUGACAGUG CUCUGUGUGC GUUUCAUAUA UUAAAUUACU CUAAUCAUCC
CAAAUCCUGU AAGUUGGGUA UCAAUUCAAG UGUUCCUAUU GGGUAGGAAU AUACAGUUCU
UUUAGGAAAU GUAGUAUGGU UCUGUGUCUC AAACAGGACA CUUACACAGU UGGCCAACAU
CAUCACCUUC UCCAUUCUCU GAGAUGUUUA GUCUUACUGA GCACUAAAUA UGGGUCAUCA
AUAGUCCAGA CUACCUUGAG CAAACAAUAG UCCAGACUAC CUUGAGCAAA CAGAGCAUAU
ACUCAUACAG UGUAUAAAGA GCACCAAGCA UACAGAUUUC AUGUCUUUCU CAUAGUUACU
CUUGUAACAU GAGCUAAAGA UCAGACCUCU AUGUCACCUU UGUAACUGAU UUCUAGAUUU
UUUUUUUUUU UUGAGAUGGG GUCUUGCCCU GUCACCCAGG CUGGAGUGUA GUGGCGUGAU
CAUGCCUCAU UGGAGCCUUC AACUCAUGAG CUCAAACAAU CCUCCUACCU CAGCUUCCUG
AGUAGUUGGG ACCACAGGUG UGUGCCACCA CACCCAGCUC AUUUUUGUAU UCUUUGUAGA
GAUGCAGUCU CACCCUGUUG CCCACGCUGG CCUGGAACUC CUGAGCUCAA AAGAUCCCUC
CGCCUUGACC UUCCAAAGUG CUGGGAUUAC AAGCAUGAAC CACUGCACCC GGCCUAGAUU
UUUAAAUGUG CUUUCCAGUA UACACUGAAA CUAGAAGUCG ACUAAAGAAU UACCAAGAGA
AUUCUAUAAA AUAGAGAUUG AAAUGGGGCU CGAUGUGGGA UGGGUUGGUG AUAUUGCAGG
GAGAAGUAAU CUGAGUAAAG GAGGAAAAGA ACUGAUUUGG GAAAACGAUA GUUUUAGUAG
UGAGUUUGAG UAUGAAUUAA GUUGAGAUUG AAUUUGAAUU AAGUUGAGGU UGAAUAUGAA
UUAAGUUGAG GUUGAGUUUG AGGUAUGAAU UAAGAUGUGA AAUUGAUCAU UGGAAAUGUU
AGAUUGAGAA AAGUCACAGC UGGAUUAAUA GCUUCAGAAG UGUGUUUGCA GACAGUUGCA
ACUAAAGUAA UAAGAAUAGA UGGCCUUGGC CGGGCGCGGU GGCUCACGCC UGUAAUCCCA
GUACUUUGGG AGGCUGAGGC GAGCAAAUCA CGAGGUCAGG AGUUCAAGAC CAGCCUGGCC
CACAUGGUGA AACCCCGUCU UUAUUAAAAA UACAAAAAUU AGCUGUGCAC AGUGGUGCAC
GCCUGUAAUC CCAGCUACUC GGGAGGCUGA GACAGGAGAA UCGCUUGAAC CUGGGAGGUG
GAGGUUGCAG UGAGCUGAGA UCAGUGUGAC UGCACUCCAG CCCGGUGACA GAGUGAGACU
CUGUGUAAAA AAAUAAAAUA AAUAAAAUAA UGGCCGUAAG CAAGUAAAGA AGGAUGGCCA
GCUCUUAUUG GGAAUGCCUA AAUCUAAGGC UUGAUCAGAA GUAAUGAAAC CGUUGGGGCC
CUACAUUGCU AUGACAUCCA AAGGGCCAUG AAUAUCAGGA AGAAAGAUAA UUAACAGGGU
CUAAUGUUAC AGAGAGGUUG AGAGCAAGGA GAUUUGAUUA AAAGGGUCUU UAGAGCUGAU
GUCAGGUGUA UGAUGCCUUU AAGAGCAGUU UUUAUAGUGC AGGGGGUGGU CAAAAGAGAA
AAUAGGUGCU UUCUGAGGUG ACGGAGCCUU GAGACUAGCU UAUAGUAGUA ACUGGGUUAU
GUCGUGACUU UUAUUCUGUG CACCACCCUG UAACAUGUAC AUUUUUAUUC CUAUUUUCGU
AGCAUGCUCU AAAGAAUGGU GACAUUUGUG AAACUUCGGG UAAACCAAAA ACCACACCUA
AAAGAAAACC UGCUAAGAAG AAUAAAAGCC AAAAGAAGAA UACUGCAGCU UCCUUACAAC
AGGUUAUUUU AAAAUGUUGA GAUUUAACUU CAAAGGAUGU CUCAUUAGUC CUUAUUUAAU
AGUGUAAAAU GUCUUUAACU UAAGUGAUUA GUACAGUGUU UCUAUUGACA UAUACUUAUA
CAACUUCAAA AACAACUAUU AAAUUUUCUG UUAUUUAGGA ACAUGCAUAU UAGUCAUGAA
AGUAUAAAGA AUUAGAUGGG AAUGAUAAAU GCUAAAAUCA GGACAUGUGU UCCAUUUGUG
AAUGGAAGGC AGGGAGAAGG UGCCGUUUGG AAGGAGUACC CAAGAGCCGU AAGCUGAAUU
GGCAGUGUUU UACAUCUUAA GCUGAGAGAU AGAUUUUUUU UUCCCCUUUU UCUUUAAAAA
CUCUAAAACU GUUAAUUCCA AGGAACCCAG AAGUCUAGGU AGAUUAUUUC UGCUAGUUAA
AAGCAGUAGU CCUGAAAGCU GAAUAUUUUG GUGUCUUUUG AGCCAACUUU AGUUUCAUCA
UUACCAAGGG GGAAGAGAGC UAACAGUUGA UGAGCACUUG CUCUAGGCCA GUCCAGAGUG
CUGGGCACCA UACGCAUUUU AUCUCCCUCC CGCUAUUCAC AACAAAUAUG GGAGGUAGUU
UAUAUUAUAG CCAUCUAAUA AGAUGGGGAA ACUAAGACUC AAAGAGAUUC AGAAACUUGU
CCAUGAUUAU AAAUGUAAGA GAGUUGGAAU UCAGAUUUAU GUAUUUAGAC CCCAAGCCUU
UCUCAUUACA UCAUUUUGCC UUCCAAAUCU CUACCCUCUA UCCUUCACCU CCCCACUGAU
CAAAACGAGA UGAUAGUUUG CCCUCUUCAA AAGAAAUGUG UGCAUGUAUA UAUCUUUGAU
UUCUUUUGUA GUGGAAAGUU GGGGACAAAU GUUCUGCCAU UUGGUCAGAA GACGGUUGCA
UUUACCCAGC UACCAUUGCU UCAAUUGAUU UUAAGAGAGA AACCUGUGUU GUGGUUUACA
CUGGAUAUGG AAAUAGAGAG GAGCAAAAUC UGUCCGAUCU ACUUUCCCCA AUCUGUGAAG
UAGCUAAUAA UAUAGAACAA AAUGCUCAAG AGGUAAGGAU ACAAAAAAAA AAAAAUUCAA
UUUCUGGAAG CAGAGACUAG AUGAGAAACU GUUAAACAGU AUACACAGUU GUCAGUUUGA
UCCACCGAGG CAUUAAUUUU UUCUUAAUCA CACCCUUAUA ACAAAAACCU GCAUAUUUUU
UCUUUUUAAA GAAUGAAAAU GAAAGCCAAG UUUCAACAGA UGAAAGUGAG AACUCCAGGU
CUCCUGGAAA UAAAUCAGAU AACAUCAAGC CCAAAUCUGC UCCAUGGAAC UCUUUUCUCC
CUCCACCACC CCCCAUGCCA GGGCCAAGAC UGGGACCAGG AAAGGUAAAC CUUCUAUGAA
AGUUUUCCAG AAAAUAGUUA AUGUCGGGAC AUUUAACCUC UCUGUUAACU AAUUUGUAGC
UCUCCCAUGA AACUUUUGUA GCUUAAAUAC ACAAGAAUUU UUUGAAAAGG AAAUAAGAUA
AUGAUGCAAA AUAGUUAAUU UUUUAAAAAA AUGUUAGACA CUGCAGUGGA UGCAACAAAA
UACUUUAUAU GAAAGAUUUA UCCAGUUAAC UUUUGUGGAG UAUUAGGUAU UAGACUAAUA
AUUAGCACAC UUACUUAAGU UAGAAAGUAU AAUAAUGCGC CGGACGCGGU AGCUCACGCC
UGUAAUCCCA GCACUUUGGG AGGCCAAGGU GGGCGGAUCA CAAGGUCAGG AGAUCGAGAC
CAUCCUGGCU AACACGGUGA AACCCCAUCU CUACUGAAAA UACAAAAAAA UUUGCCGGGC
GUGAUGGCGG GCACCUGUAG UCCCAGCUAC UCGGGAGGCU GAGGCAGGAG GAUGGUGUGA
ACCCCGGAGG CAGAGCUUGC AGUGAGUCAA GAUCGUGCCA CUGCACUCCA ACCUGGGCGA
CAGAAUGAGA CUCCAUCUCA AACAAAAAAA CAAAACAAAA CAAAAAAAAG UGUAAUAAUA
AUUUAUCAUU AGCUGGAUGA UAUGCUGUUG UUUCCCAUGU CACCUGUAUA AGAUAUGUAA
AAUAAGAACA CAUUAUUUAC AUCUAAUAUA GAUAAAAUCC UGAGGCGCUC UCAGAUUGUU
UUGUAGAGUU CAAAUGUAAA UAUUGUUUUC AUUUAUGGUC CUUUUGGUUA UAAGUAACAG
AAAUCAACUC UAAAAAGAUU UUUAUUAUAG GUUAGAUUAU GUCAUGGAAC CUUAAGGCUU
GUCCCUUUCU AGUUCUUUUG UGUAAAGCGG UGAUUUCUUC CAUGGAGGGA AUGGUAUUUA
GGCAAUUUUU UUUUUUUUUU CGAGAUGGAG UCUUGCUCUG UCGCUCAGGC UGGAGUGCAG
UGGCACCAUU UCAGCUCACU GCAACUUCCA CCUCCUGGGU UCAAGUGAUU CUCCUGCUUC
AGCCUCCCAA GUAGCUGAGA UUACAGGCAC CCGCCACCAC ACCCGGCUUA UUUUGUAUUU
UUAGUAGAGA UGGGGUUUCA CCAUGUUGGC CAGGCUGGUC UUGAACUCCU GACCUCAAGU
GAUCUCCCCA CCUUGGCCUU CCAAAGUGCU AGGAUUACAG GCGCCUAGCC UAGGCAGUCA
UUUUCAAAAA ACAAGCAUGA CUCACCAAAA GUUUUAAGAU UUUCUGUGAU AAUGUUCUUA
UUGAGGCUUA CAUUAUAUUA CAGUUUCUUG AAUCUAAAAU GAUGUACCCU CUUAGAAUAU
AUACAUCAUG CUUCAUUGGU CUCAGGGGGC UGAUUUUUAU AAGGAGAGAU UUGCUAGUUU
UCACAAUAUG UCCUCUAAGU UGGCAUGUAU AGCUAAACAG GCUUUCAUAA AAAUAUACAA
UUUAGUUAAU GAAAUUUGGG AUAUAGUCUU UUAUGAUUGA AAUAAUUUUG CUAAAUAGAC
UGUCUCUGAU UUAUUAGGUA AUCACCACUC UUAUUUUGUU UUACUUCCUU AAUGUCUACA
UAGAAAGGAA AUGAGAAAAA UCCAGAGGUU GUCAUUUGAC UUAUGAGUCU GUUUGACUUC
AGGAUUUGGU ACAUGAAAUU UCACUUAAUC UUUUUGAUAU GUAUAAAACA AAUAUUCUGG
GUAAUUAUUU UUAUCCUUUU GGUUUUGAGU CCUUUUUAUU CCUAUCAUAU UGAAAUUGGU
AAGUUAAUUU UCCUUUGAAA UAUUCCUUAU AGCCAGGUCU AAAAUUCAAU GGCCCACCAC
CGCCACCGCC ACCACCACCA CCCCACUUAC UAUCAUGCUG GCUGCCUCCA UUUCCUUCUG
GACCACCAGU AAGUAAAAAA GAGUAUAGGU UAGAUUUUGC UUUCACAUAC AAUUUGAUAA
UUAGCAGAAU AGAGGAUUGU AAAAUGUCAU UGUAGAACAU CCCUUGGGCC AGAUUCUAAU
GGGUAGAAAU UUGAACUAAA CCUCUGGGUU UUGUUUGUUU UUAAUGCCUU UCUGUUACCC
AGAUGCAGUG CUCUUGUAGU CCCAAGUCUA AGCUCUAGGU UGCCUUCUUU CCUGGCAGAA
GUUGGUGUCU AUGCCAUAAG GAGGUAGUUC CUGUUAGAAG GGAUUUAAUU AUACCUUAUA
UAAGGAAUUA GUGUUUGCCC UUCUAGGUAU AGUUGGAUGU UAGCUUCUGA UGUAAACUGG
AUUUCUUUUU CUUUCUCUCU CUUUUUUUUU UUUUGUUUUG GAGGCAGAGU UUUGCCCUUG
UACCCCAGGC UGGAGUGCAG UGGUGUGAUC UCAGCUCACA GCAACCUCCG CCUCCUGGGU
UCAAGCAAUU CUGCCUCGGC CUCCCAAGUA GCUGGGAUUA CAGGCGACUG CCACCACACC
CGGCUAAUUU UUGUUUUAUU AGUAGAGAUG GGGUUUCACC AUGUUGGCCA GACUGAUCUU
GAACUCCUGA CCUCAGGUGA UCCACCCGCC UUGGCCUCCC AAAGCGCUGG GAUUACAGGC
GUGAGCUGCC GCACCCAGCU GUAAACUGGA UUUCUAAUGG UAGAUUUUUA GGUAUUAACA
AUAGAUAAAA AGAUACUUUU UGGCAUACUG UGUAUUGGGA UGGGGUUAGA ACAGGUGUUC
UACCCAAGAC AUUUACUUAA AAUCGCCCUC GAAAUGCUAU GUGAGCUGUG UGUGUGUGUG
UGUGUGUGUG UGUAUUAAGG AAAAGCAUGA AAGUAUUUAU GCUUGAUUUU UUUUUUUUAC
UCAUAGCUUC AUAGUGGAAC AGAUACAUAG UCUAAAUCAA AAUGUUUAAA CUUUUUAUGU
CACUUGCUGU CUUUUCGUCC UCGUUAAAUU UAAUUUUGUU GGUCUUUUGU UGUUAUUGGU
UGGUUUUCUC CAAAUGCUAG CUAUGUUAAG AAAUUUAAGG CCAGGUACAG UGGCUCAUGC
CUGUAAUCCC GGCAUUUUAG AAGGCUGAGG CAGGAGGAUC ACUUGAGCUC AGGAGUUUGA
GACCAGUCUG GGCAACAUAG CAAGACCUCG UCUUUGUUUA GGGGAAAAAA AAGAAAUUUA
AGUAGGAGAU UAUAUAAGCA AAAAUACAAU UAAUUUCCAG CAUUCACUAU AUAAUAUAAA
UCUCCAGACU UUACUUUUUU GUUUACUGGA UAUAAACAAU AUCUUUUUCU GUCUCCAGAU
AAUUCCCCCA CCACCUCCCA UAUGUCCAGA UUCUCUUGAU GAUGCUGAUG CUUUGGGAAG
UAUGUUAAUU UCAUGGUACA UGAGUGGCUA UCAUACUGGC UAUUAUAUGG UAAGUAAUCA
CUCAGCAUCU UUUCCUGACA AUUUUUUUGU AGUUAUGUGA CUUUGUUUUG UAAAUUUAUA
AAAUACUACU UGCUUCUCUC UUUAUAUUAC UAAAAAAUAA AAAUAAAAAA AUACAACUGU
CUGAGGCUUA AAUUACUCUU GCAUUGUCCC UAAGUAUAAU UUUAGUUAAU UUUAAAAAGC
UUUCAUGCUA UUGUUAGAUU AUUUUGAUUA UACACUUUUG AAUUGAAAUU AUACUUUUUC
UAAAUAAUGU UUUAAUCUCU GAUUUGAAAU UGAUUGUAGG GAAUGGAAAA GAUGGGAUAA
UUUUUCAUAA AUGAAAAAUG AAAUUCUUUU UUUUUUUUUU UUUUUUUUGA GACGGAGUCU
UGCUCUGUUG CCCAGGCUGG AGUGCAAUGG CGUGAUCUUG GCUCACAGCA AGCUCUGCCU
CCUGGAUUCA CGCCAUUCUC CUGCCUCAGC CUCAGAGGUA GCUGGGACUA CAGGUGCCUG
CCACCACGCC UGUCUAAUUU UUUGUAUUUU UUUGUAAAGA CAGGGUUUCA CUGUGUUAGC
CAGGAUGGUC UCAAUCUCCU GACCCCGUGA UCCACCCGCC UCGGCCUUCC AAGAGAAAUG
AAAUUUUUUU AAUGCACAAA GAUCUGGGGU AAUGUGUACC ACAUUGAACC UUGGGGAGUA
UGGCUUCAAA CUUGUCACUU UAUACGUUAG UCUCCUACGG ACAUGUUCUA UUGUAUUUUA
GUCAGAACAU UUAAAAUUAU UUUAUUUUAU UUUAUUUUUU UUUUUUUUUU GAGACGGAGU
CUCGCUCUGU CACCCAGGCU GGAGUACAGU GGCGCAGUCU CGGCUCACUG CAAGCUCCGC
CUCCCGGGUU CACGCCAUUC UCCUGCCUCA GCCUCUCCGA GUAGCUGGGA CUACAGGCGC
CCGCCACCAC GCCCGGCUAA UUUUUUUUUA UUUUUAGUAG AGACGGGGUU UCACCGUGGU
CUCGAUCUCC UGACCUCGUG AUCCACCCGC CUCGGCCUCC CAAAGUGCUG GGAUUACAAG
CGUGAGCCAC CGCGCCCGGC CUAAAAUUAU UUUUAAAAGU AAGCUCUUGU GCCCUGCUAA
AAUUAUGAUG UGAUAUUGUA GGCACUUGUA UUUUUAGUAA AUUAAUAUAG AAGAAACAAC
UGACUUAAAG GUGUAUGUUU UUAAAUGUAU CAUCUGUGUG UGCCCCCAUU AAUAUUCUUA
UUUAAAAGUU AAGGCCAGAC AUGGUGGCUU ACAACUGUAA UCCCAACAGU UUGUGAGGCC
GAGGCAGGCA GAUCACUUGA GGUCAGGAGU UUGAGACCAG CCUGGCCAAC AUGAUGAAAC
CUUGUCUCUA CUAAAAAUAC CAAAAAAAAU UUAGCCAGGC AUGGUGGCAC AUGCCUGUAA
UCCGAGCUAC UUGGGAGGCU GUGGCAGGAA AAUUGCUUUA AUCUGGGAGG CAGAGGUUGC
AGUGAGUUGA GAUUGUGCCA CUGCACUCCA CCCUUGGUGA CAGAGUGAGA UUCCAUCUCA
AAAAAAGAAA AAGGCCUGGC ACGGUGGCUC ACACCUAUAA UCCCAGUACU UUGGGAGGUA
GAGGCAGGUG GAUCACUUGA GGUUAGGAGU UCAGGACCAG CCUGGCCAAC AUGGUGACUA
CUCCAUUUCU ACUAAAUACA CAAAACUUAG CCCAGUGGCG GGCAGUUGUA AUCCCAGCUA
CUUGAGAGGU UGAGGCAGGA GAAUCACUUG AACCUGGGAG GCAGAGGUUG CAGUGAGCCG
AGAUCACACC GCUGCACUCU AGCCUGGCCA ACAGAGUGAG AAUUUGCGGA GGGAAAAAAA
AGUCACGCUU CAGUUGUUGU AGUAUAACCU UGGUAUAUUG UAUGUAUCAU GAAUUCCUCA
UUUUAAUGAC CAAAAAGUAA UAAAUCAACA GCUUGUAAUU UGUUUUGAGA UCAGUUAUCU
GACUGUAACA CUGUAGGCUU UUGUGUUUUU UAAAUUAUGA AAUAUUUGAA AAAAAUACAU
AAUGUAUAUA UAAAGUAUUG GUAUAAUUUA UGUUCUAAAU AACUUUCUUG AGAAAUAAUU
CACAUGGUGU GCAGUUUACC UUUGAAAGUA UACAAGUUGG CUGGGCACAA UGGCUCACGC
CUGUAAUCCC AGCACUUUGG GAGGCCAGGG CAGGUGGAUC ACGAGGUCAG GAGAUCGAGA
CCAUCCUGGC UAACAUGGUG AAACCCCGUC UCUACUAAAA GUACAAAAAC AAAUUAGCCG
GGCAUGUUGG CGGGCACCUU UUGUCCCAGC UGCUCGGGAG GCUGAGGCAG GAGAGUGGCG
UGAACCCAGG AGGUGGAGCU UGCAGUGAGC CGAGAUUGUG CCAGUGCACU CCAGCCUGGG
CGACAGAGCG AGACUCUGUC UCAAAAAAUA AAAUAAAAAA GAAAGUAUAC AAGUCAGUGG
UUUUGGUUUU CAGUUAUGCA ACCAUCACUA CAAUUUAAGA ACAUUUUCAU CACCCCAAAA
AGAAACCCUG UUACCUUCAU UUUCCCCAGC CCUAGGCAGU CAGUACACUU UCUGUCUCUA
UGAAUUUGUC UAUUUUAGAU AUUAUAUAUA AACGGAAUUA UACGAUAUGU GGUCUUUUGU
GUCUGGCUUC UUUCACUUAG CAUGCUAUUU UCAAGAUUCA UCCAUGCUGU AGAAUGCACC
AGUACUGCAU UCCUUCUUAU UGCUGAAUAU UCUGUUGUUU GGUUAUAUCA CAUUUUAUCC
AUUCAUCAGU UCAUGGACAU UUAGGUUGUU UUUAUUUUUG GGCUAUAAUG AAUAAUGUUG
CUAUGAACAU UCGUUUGUGU UCUUUUUGUU UUUUUGGUUU UUUGGGUUUU UUUUGUUUUG
UUUUUGUUUU UGAGACAGUC UUGCUCUGUC UCCUAAGCUG GAGUGCAGUG GCAUGAUCUU
GGCUUACUGC AAGCUCUGCC UCCCGGGUUC ACACCAUUCU CCUGCCUCAG CCCGACAAGU
AGCUGGGACU ACAGGCGUGU GCCACCAUGC ACGGCUAAUU UUUUGUAUUU UUAGUAGAGA
UGGGGUUUCA CCGUGUUAGC CAGGAUGGUC UCGAUCUCCU GACCUCGUGA UCUGCCUGCC
UAGGCCUCCC AAAGUGCUGG GAUUACAGGC GUGAGCCACU GCACCUGGCC UUAAGUGUUU
UUAAUACGUC AUUGCCUUAA GCUAACAAUU CUUAACCUUU GUUCUACUGA AGCCACGUGG
UUGAGAUAGG CUCUGAGUCU AGCUUUUAAC CUCUAUCUUU UUGUCUUAGA AAUCUAAGCA
GAAUGCAAAU GACUAAGAAU AAUGUUGUUG AAAUAACAUA AAAUAGGUUA UAACUUUGAU
ACUCAUUAGU AACAAAUCUU UCAAUACAUC UUACGGUCUG UUAGGUGUAG AUUAGUAAUG
AAGUGGGAAG CCACUGCAAG CUAGUAUACA UGUAGGGAAA GAUAGAAAGC AUUGAAGCCA
GAAGAGAGAC AGAGGACAUU UGGGCUAGAU CUGACAAGAA AAACAAAUGU UUUAGUAUUA
AUUUUUGACU UUAAAUUUUU UUUUUAUUUA GUGAAUACUG GUGUUUAAUG GUCUCAUUUU
AAUAAGUAUG ACACAGGUAG UUUAAGGUCA UAUAUUUUAU UUGAUGAAAA UAAGGUAUAG
GCCGGGCACG GUGGCUCACA CCUGUAAUCC CAGCACUUUG GGAGGCCGAG GCAGGCGGAU
CACCUGAGGU CGGGAGUUAG AGACUAGCCU CAACAUGGAG AAACCCCGUC UCUACUAAAA
AAAAUACAAA AUUAGGCGGG CGUGGUGGUG CAUGCCUGUA AUCCCAGCUA CUCAGGAGGC
UGAGGCAGGA GAAUUGCUUG AACCUGGGAG GUGGAGGUUG CGGUGAGCCG AGAUCACCUC
AUUGCACUCC AGCCUGGGCA ACAAGAGCAA AACUCCAUCU CAAAAAAAAA AAAAUAAGGU
AUAAGCGGGC UCAGGAACAU CAUUGGACAU ACUGAAAGAA GAAAAAUCAG CUGGGCGCAG
UGGCUCACGC CGGUAAUCCC AACACUUUGG GAGGCCAAGG CAGGCGAAUC ACCUGAAGUC
GGGAGUUCCA GAUCAGCCUG ACCAACAUGG AGAAACCCUG UCUCUACUAA AAAUACAAAA
CUAGCCGGGC AUGGUGGCGC AUGCCUGUAA UCCCAGCUAC UUGGGAGGCU GAGGCAGGAG
AAUUGCUUGA ACCGAGAAGG CGGAGGUUGC GGUGAGCCAA GAUUGCACCA UUGCACUCCA
GCCUGGGCAA CAAGAGCGAA ACUCCGUCUC AAAAAAAAAA GGAAGAAAAA UAUUUUUUUA
AAUUAAUUAG UUUAUUUAUU UUUUAAGAUG GAGUUUUGCC CUGUCACCCA GGCUGGGGUG
CAAUGGUGCA AUCUCGGCUC ACUGCAACCU CCGCCUCCUG GGUUCAAGUG AUUCUCCUGC
CUCAGCUUCC CGAGUAGCUG UGAUUACAGC CAUAUGCCAC CACGCCCAGC CAGUUUUGUG
UUUUGUUUUG UUUUUUGUUU UUUUUUUUUG AGAGGGUGUC UUGCUCUGUC CCCCAAGCUG
GAGUGCAGCG GCGCGAUCUU GGCUCACUGC AAGCUCUGCC UCCCAGGUUC ACACCAUUCU
CUUGCCUCAG CCUCCCGAGU AGCUGGGACU ACAGGUGCCC GCCACCACAC CCGGCUAAUU
UUUUUGUGUU UUUAGUAGAG AUGGGGUUUC ACUGUGUUAG CCAGGAUGGU CUCGAUCUCC
UGACCUUUUG AUCCACCCGC CUCAGCCUCC CCAAGUGCUG GGAUUAUAGG CGUGAGCCAC
UGUGCCCGGC CUAGUCUUGU AUUUUUAGUA GAGUCGGGAU UUCUCCAUGU UGGUCAGGCU
GUUCUCCAAA UCCGACCUCA GGUGAUCCGC CCGCCUUGGC CUCCAAAAGU GCAAGGCAAG
GCAUUACAGG CAUGAGCCAC UGUGACCGGC AAUGUUUUUA AAUUUUUUAC AUUUAAAUUU
UAUUUUUUAG AGACCAGGUC UCACUCUAUU GCUCAGGCUG GAGUGCAAGG GCACAUUCAC
AGCUCACUGC AGCCUUGACC UCCAGGGCUC AAGCAGUCCU CUCACCUCAG UUUCCCGAGU
AGCUGGGACU ACAGUGAUAA UGCCACUGCA CCUGGCUAAU UUUUAUUUUU AUUUAUUUAU
UUUUUUUUGA GACAGAGUCU UGCUCUGUCA CCCAGGCUGG AGUGCAGUGG UGUAAAUCUC
AGCUCACUGC AGCCUCCGCC UCCUGGGUUC AAGUGAUUCU CCUGCCUCAA CCUCCCAAGU
AGCUGGGAUU AGAGGUCCCC ACCACCAUGC CUGGCUAAUU UUUUGUACUU UCAGUAGAAA
CGGGGUUUUG CCAUGUUGGC CAGGCUGUUC UCGAACUCCU GAGCUCAGGU GAUCCAACUG
UCUCGGCCUC CCAAAGUGCU GGGAUUACAG GCGUGAGCCA CUGUGCCUAG CCUGAGCCAC
CACGCCGGCC UAAUUUUUAA AUUUUUUGUA GAGACAGGGU CUCAUUAUGU UGCCCAGGGU
GGUGUCAAGC UCCAGGUCUC AAGUGAUCCC CCUACCUCCG CCUCCCAAAG UUGUGGGAUU
GUAGGCAUGA GCCACUGCAA GAAAACCUUA ACUGCAGCCU AAUAAUUGUU UUCUUUGGGA
UAACUUUUAA AGUACAUUAA AAGACUAUCA ACUUAAUUUC UGAUCAUAUU UUGUUGAAUA
AAAUAAGUAA AAUGUCUUGU GAAACAAAAU GCUUUUUAAC AUCCAUAUAA AGCUAUCUAU
AUAUAGCUAU CUAUAUCUAU AUAGCUAUUU UUUUUAACUU CCUUUAUUUU CCUUACAGGG
UUUUAGACAA AAUCAAAAAG AAGGAAGGUG CUCACAUUCC UUAAAUUAAG GAGUAAGUCU
GCCAGCAUUA UGAAAGUGAA UCUUACUUUU GUAAAACUUU AUGGUUUGUG GAAAACAAAU
GUUUUUGAAC AUUUAAAAAG UUCAGAUGUU AGAAAGUUGA AAGGUUAAUG UAAAACAAUC
AAUAUUAAAG AAUUUUGAUG CCAAAACUAU UAGAUAAAAG GUUAAUCUAC AUCCCUACUA
GAAUUCUCAU ACUUAACUGG UUGGUUGUGU GGAAGAAACA UACUUUCACA AUAAAGAGCU
UUAGGAUAUG AUGCCAUUUU AUAUCACUAG UAGGCAGACC AGCAGACUUU UUUUUAUUGU
GAUAUGGGAU AACCUAGGCA UACUGCACUG UACACUCUGA CAUAUGAAGU GCUCUAGUCA
AGUUUAACUG GUGUCCACAG AGGACAUGGU UUAACUGGAA UUCGUCAAGC CUCUGGUUCU
AAUUUCUCAU UUGCAGGAAA UGCUGGCAUA GAGCAGCACU AAAUGACACC ACUAAAGAAA
CGAUCAGACA GAUCUGGAAU GUGAAGCGUU AUAGAAGAUA ACUGGCCUCA UUUCUUCAAA
AUAUCAAGUG UUGGGAAAGA AAAAAGGAAG UGGAAUGGGU AACUCUUCUU GAUUAAAAGU
UAUGUAAUAA CCAAAUGCAA UGUGAAAUAU UUUACUGGAC UCUAUUUUGA AAAACCAUCU
GUAAAAGACU GAGGUGGGGG UGGGAGGCCA GCACGGUGGU GAGGCAGUUG AGAAAAUUUG
AAUGUGGAUU AGAUUUUGAA UGAUAUUGGA UAAUUAUUGG UAAUUUUAUG AGCUGUGAGA
AGGGUGUUGU AGUUUAUAAA AGACUGUCUU AAUUUGCAUA CUUAAGCAUU UAGGAAUGAA
GUGUUAGAGU GUCUUAAAAU GUUUCAAAUG GUUUAACAAA AUGUAUGUGA GGCGUAUGUG
GCAAAAUGUU ACAGAAUCUA ACUGGUGGAC AUGGCUGUUC AUUGUACUGU UUUUUUCUAU
CUUCUAUAUG UUUAAAAGUA UAUAAUAAAA AUAUUUAAUU UUUUUUUAAA UUA
Homo sapiens SMN2 mRNA nucleic acid sequence
NCBI Ref. Seq.: NM 017411.3
(SEQ ID NO: 13)
CCACAAATGT GGGAGGGCGA TAACCACTCG TAGAAAGCGT GAGAAGTTAC TACAAGCGGT
CCTCCCGGCC ACCGTACTGT TCCGCTCCCA GAAGCCCCGG GCGGCGGAAG TCGTCACTCT
TAAGAAGGGA CGGGGCCCCA CGCTGCGCAC CCGCGGGTTT GCTATGGCGA TGAGCAGCGG
CGGCAGTGGT GGCGGCGTCC CGGAGCAGGA GGATTCCGTG CTGTTCCGGC GCGGCACAGG
CCAGAGCGAT GATTCTGACA TTTGGGATGA TACAGCACTG ATAAAAGCAT ATGATAAAGC
TGTGGCTTCA TTTAAGCATG CTCTAAAGAA TGGTGACATT TGTGAAACTT CGGGTAAACC
AAAAACCACA CCTAAAAGAA AACCTGCTAA GAAGAATAAA AGCCAAAAGA AGAATACTGC
AGCTTCCTTA CAACAGTGGA AAGTTGGGGA CAAATGTTCT GCCATTTGGT CAGAAGACGG
TTGCATTTAC CCAGCTACCA TTGCTTCAAT TGATTTTAAG AGAGAAACCT GTGTTGTGGT
TTACACTGGA TATGGAAATA GAGAGGAGCA AAATCTGTCC GATCTACTTT CCCCAATCTG
TGAAGTAGCT AATAATATAG AACAAAATGC TCAAGAGAAT GAAAATGAAA GCCAAGTTTC
AACAGATGAA AGTGAGAACT CCAGGTCTCC TGGAAATAAA TCAGATAACA TCAAGCCCAA
ATCTGCTCCA TGGAACTCTT TTCTCCCTCC ACCACCCCCC ATGCCAGGGC CAAGACTGGG
ACCAGGAAAG CCAGGTCTAA AATTCAATGG CCCACCACCG CCACCGCCAC CACCACCACC
CCACTTACTA TCATGCTGGC TGCCTCCATT TCCTTCTGGA CCACCAATAA TTCCCCCACC
ACCTCCCATA TGTCCAGATT CTCTTGATGA TGCTGATGCT TTGGGAAGTA TGTTAATTTC
ATGGTACATG AGTGGCTATC ATACTGGCTA TTATATGGGT TTTAGACAAA ATCAAAAAGA
AGGAAGGTGC TCACATTCCT TAAATTAAGG AGAAATGCTG GCATAGAGCA GCACTAAATG
ACACCACTAA AGAAACGATC AGACAGATCT GGAATGTGAA GCGTTATAGA AGATAACTGG
CCTCATTTCT TCAAAATATC AAGTGTTGGG AAAGAAAAAA GGAAGTGGAA TGGGTAACTC
TTCTTGATTA AAAGTTATGT AATAACCAAA TGCAATGTGA AATATTTTAC TGGACTCTAT
TTTGAAAAAC CATCTGTAAA AGACTGAGGT GGGGGTGGGA GGCCAGCACG GTGGTGAGGC
AGTTGAGAAA ATTTGAATGT GGATTAGATT TTGAATGATA TTGGATAATT ATTGGTAATT
TTATGAGCTG TGAGAAGGGT GTTGTAGTTT ATAAAAGACT GTCTTAATTT GCATACTTAA
GCATTTAGGA ATGAAGTGTT AGAGTGTCTT AAAATGTTTC AAATGGTTTA ACAAAATGTA
TGTGAGGCGT ATGTGGCAAA ATGTTACAGA ATCTAACTGG TGGACATGGC TGTTCATTGT
ACTGTTTTTT TCTATCTTCT ATATGTTTAA AAGTATATAA TAAAAATATT TAATTTTTTT
TTAAATTAAA AAAA
Homo sapiens SMN2 amino acid sequence
NCBI Ref. Seq.: NP 059107.1; UniProtKB: Q16637
(SEQ ID NO: 14)
MAMSSGGSGG GVPEQEDSVL FRRGTGQSDD SDIWDDTALI KAYDKAVASF KHALKNGDIC
ETSGKPKTTP KRKPAKKNKS QKKNTAASLQ QWKVGDKCSA IWSEDGCIYP ATIASIDFKR
ETCVVVYTGY GNREEQNLSD LLSPICEVAN NIEQNAQENE NESQVSTDES ENSRSPGNKS
DNIKPKSAPW NSFLPPPPPM PGPRLGPGKP GLKFNGPPPP PPPPPPHLLS CWLPPFPSGP
PIIPPPPPIC PDSLDDADAL GSMLISWYMS GYHTGYYMGF RQNQKEGRCS HSLN
Long Non-coding RNA sequence
GenBank accession #BC045789.1
(SEQ ID NO: 161)
AGTAATTAAA AGTGTAGTAC TGACATCAAT ATACACTAAT AGACAATGTA ACAGAATTGA
GAGTCAGAAA TAGCCCTGTG ATATATGTGA CTGATTTTAA CTAAAGTTAC AAATGTGGTT
TAATAGAGAA AGCATAGTAG TTTCAAGAAA TGGTGGTAAA ACTATTGGAA ATCCTTATGC
AAAAACAAAC CAAATTTCCA CCTACACTGA GCCACATATA AGAATTAATA CAAAATGGAT
TACAGACCTA AGTGTAAAAC TAAAACTCTA AACTGTGTAA AAAAAAAAAA GAGAGAAAAA
TCTGTGATCA TGGATTGCCT ATAACAAAAG AACAAACTGA AATGGCCTTG TTGCCTGGGG
TGGCACCCGA GGTTCTTGGT CTCACGCTGA GGAAATCAAG GACGCTGACA CACCAAGGGT
GTGAGGCTAG AGCAGAAGTT TAATAGGCAA AAGAAAGAGA CTAGCTCTCT CCTGCAGAGA
GAGGTCCTGA AAAGAGTTCC CATTCCACAG TGAAATGCAA GCATTTTTAT AAATGAGCTA
ATGGGAAGGG GGTAGCTTAT CTACACATAG GGAGCAAAAA ACCAGTTAGG ACCAGGTGTT
GCCATCTGCA CAGAGCGTGA ATCTCTGGCA TCCCCCACCC CAACCTTTTA TTATGCAGGC
AGGTCCTTGG CCTGAGCTAC TCCACATTGC TTATCTCTTT CCTATTGTGC ATGTGCTAAA
TAAGGGGAGG TGGAGCCACC ATGGTGGACA TGCCTGGCCC CAGGTACCCC TTTCTCTCCG
TGCAGCTGCA GGCAACCCCA ACAACTCACA ACATGCAAGC TTCCAGCTTC CTTATCTGAG
TATGTACTAA GGTCCACTGT GTTTACTTCA CATACTGTGT TTACTTCACA TACCCACCTT
ACGTATGTGA AGCTTGCTGA TTACCCAGGA AGCTCCCCCT CTGTGCCAGA GCTGCTTCCT
TATACATGTT TACAGCCCGA TCTTCCAGGC TGCTCCTTGT TAGAAGAGAA GTGATTTCTT
GGGCTGCTTT TTGTTAGAAG GGAAGTTCTA CCGAGGACTC TGTCTAACTA TCGGCTTACC
TAGTCTTTTT TTACCTCCTC TCTCAAAACC ATCAAAGAAA AAACTGAAAA ATTAGATTTC
AGCAAAATTA AGATTTTCTG TTCTTAAAAA GACGTTGTTA ACAAAATGAA AAACCAAAGT
GTAAACCGGG AGAAAATACA TGCAAGTTAC CTATCTGATA AAGATATTGT ACCCAGCAAA
CATGAAGAAC TCTCAAACCT CAACAACAAA AAAATTCAAT TAAAAGATGG GTAAGTTGGC
CGGGTGCAGT GGCTCATACT TGCAATCCCA ATCTTTGGGA GGCTGAGGCA GGAAGATTGC
TTGAGCCCAG GAGTTCACGA CAAGCCCAGG CAACATAATG AGACCTTGTT TCTACAAAAT
TTTAAAAAAT TAGCCAGGCA TTGTGGCGTG TGCTCGTAAT TTCAGCTACT CAGAAGGCTG
AGACAAGAAG ACTGAGCGCA GGAGGTGGAG GCTGCAGTAG GCTGTGTGAT TGCACCACTG
CACAACAGCC TGGGTGACAG AGTGAGACAC TGTCTCCAAA AAAAAAAAAA AAA

As used herein, “SMN2 pre-mRNA” refers to an RNA sequence, including all exons, introns, and untranslated regions, transcribed from DNA encoding human SMN2.

As used herein, “intronic splicing silencer N1” or “ISS-N1” refers to an intronic splice silencing domain in intron 7 of the SMN2 gene or pre-mRNA (see e.g., Singh et al., Mol Cell Biol (2006) 26(4):1333-46). Splicing of a critical exon of human Survival Motor Neuron is regulated by a unique silencer element located in the last intron.

In some embodiments, ISS-N1 comprises the nucleic acid sequence:

(SEQ ID NO: 15)
CCAGCAUUAUGAAAG

In some embodiments, the SMN2 pre-mRNA is targeted with one or more of the exemplary oligonucleotides disclosed in Tables 2-6 below in one or more SNAs. Unless indicated otherwise, the sequences contain phosphodiester internucleotide linkages.

TABLE 2

| | SEQ | |
|---|---|---|
| 2′-OMOE | ID | |
| Chemistry | NO | Sequence |
| ASO 10-27- | 65 | moeT*/5-Me-moeC/moeA/5-Me-moeC/moeTmoeTmoeT/5-Me- |
| MOE-PS- | | moeC/moeAmoeTmoeAmoeAmoeTmoeG/5-Me- |
| Chol SMN- | | moeC/moeTmoeG*moeG/iSp18//iSp18//3CholTEG/ |
| 2PS | | |
| ASO 10-27- | 66 | moeT*/5-Me-moeC/moeA/5-Me-moeC/moeTmoeTmoeT/5-Me- |
| MOE-PS- | | moeC/*moeAmoeTmoeAmoeAmoeTmoeG*/5-Me- |
| Chol SMN- | | moeC/moeTmoeGmoeG/iSp18//iSp18//3CholTEG/ |
| 3PS | | |
| ASO 10-27- | 67 | moeT*/5-Me-moeC/moeA/5-Me-moeC/moeTmoeTmoeT/5-Me- |
| MOE-PS- | | moeC/*moeA*moeTmoeAmoeAmoeT*moeG*/5-Me- |
| Chol SMN- | | moeC/moeTmoeGmoeG/iSp18//iSp18//3CholTEG/ |
| 5PS | | |
| ASO 10-27- | 68 | moeT*/5-Me-moeC/moeA/5-me-moeC/moeTmoeTmoeT/5- |
| MOE-PS- | | MemoeC/*moeA*moeT*moeA*moeA*moeT*moeG*/5MemoeC/ |
| Chol SMN- | | moeTmoeGmoeG/iSp18//iSp18//3CholTEG/ |
| 8PS | | |
| 5′-Chol ASO | 69 | /5CholTEG//iSp18//iSp18/moeT*/5-Me-moeC/*moeA*/5- |
| 10-27 MOE | | Me-moeC/*moeT*moeT*moeT*/5-Me- |
| PS SMN | | moeC/*moeA*moeT*moeA*moeA*moeT*moeG*/ |
| | | 5-Me-moeC/*moeT*moeG*moeG |

*= phosphorothioate internucleotide linkage;
moe = 2′-O-(2-methoxyethyl);
iSp18 = internal hexaethyleneglycol spacer;
5-Me-moeC = 5′methyl C with moe modification;
3CholTEG = 3′-Cholesteryl-TEG;
5CholTEG = 5′Cholesteryl-TEG;
TEG = tetraethylene glycol

TABLE 3

| 2′-O-Me | |
|---|---|
| Chemistry | Sequence |
| ASO 10-27- | mU*mC*mA*mC*mU*mU*mU*mC*mA*mU*mA* |
| Me-PS- | mA*mU*mG*mC*mU*mG*mG/iSp18//iSp18// |
| Chol SMN | 3CholTEG/ (SEQ ID NO: 70) |
| ASO 10-27- | mUmCmAmCmUmUmUmCmAmUmAmAmUmGmCmUmGmG/ |
| Me-Chol | iSp18//iSp18//3CholTEG/ (SEQ ID NO: 71) |
| SMN all | |
| PO | |
| ASO 10-27- | mT*/5-Me-MC/*mA*/5-Me-mC/*mT*mT*mT*/ |
| Me-PS- | 5-Me-mC/*mA*mT*mA*mA*mT*mG*/5-Me-mC/ |
| Chol SMN | *mT*mG*mG/iSp18//iSp18//3CholTEG/ |
| (5MeC and | (SEQ ID NO: 72) |
| 5MeU) | |
| Linear | mT*/5-Me-MC/*mA*/5-Me-mC/*mT*mT*mT*/ |
| ASO 10-27- | 5-Me-mC/*mA*mT*mA*mA*mT*mG*/5- |
| Me-PS- | Me-mC/*mT*mG*mG (SEQ ID NO: 73) |
| SMN (5 | |
| MeC and | |
| 5MeU) | |
| Linear | mUmCmAmCmUmUmUmCmAmUmAmAmUmGmCmUmGmG |
| ASO 10-27- | (SEQ ID NO: 74) |
| Me-SMN | |
| all PO | |

*= phosphorothioate internucleotide linkage;
m = 2′-O-methyl;
iSp18 = internal hexaethyleneglycol spacer;
5-Me-mC = 5′methyl C with 2′-O-methyl modification;
3CholTEG = 3′-Cholesteryl-TEG;
TEG = tetraethylene glycol

TABLE 4

| Morpholino | |
|---|---|
| Chemistry | Sequence |
| Morpholino | 5′-TCACTTTCATAATGCTGG-CholEG-3′ |
| Chol SMN | (SEQ ID NO: 75) |
| oligo | |
| Linear | 5′-TCACTTTCATAATGCTGG-3′ |
| Morpholino | (SEQ ID NO: 76) |
| oligo | |

CholEG = Cholesterol with ethylene glycol spacer; all bases in the above two sequences contain morpholino sugar modification

TABLE 5

| ENA Chemistry | Sequence |
|---|---|
| ASO 10-27- | enT*/5-Me-enC/*enA*/5-Me-enC/ |
| ENA-PS- | *enT*enT*enT*/5-Me-enC/*enA* |
| Chol SMN | enT*enA*enA*enT*enG*/5-Me-enC/ |
| | *enT*enG*enG/isp18//isp18// |
| | 3CholTEG/ (SEQ ID NO: 77) |
| Linear ASO | enT*/5-Me-enC/*enA*/5-Me-enC/ |
| 10-27-ENA- | *enT*enT*enT*/5-Me-enC/*enA |
| PS-SMN | *enT*enA*enA*enT*enG*/5-Me-enC/ |
| | *enT*enG*enG (SEQ ID NO: 78) |

*= phosphorothioate internucleotide linkage;
en = 2′-O,4′-C-ethylene-bridged sugar modification;
iSp18 = internal hexaethyleneglycol spacer;
5-Me-enC = 5′methyl C with en modification;
3CholTEG = 3′-Cholesteryl-TEG;
TEG = tetraethylene glycol

TABLE 6

| LNA Chemistry | Sequence |
|---|---|
| ASO 10-27- | lnT*/5-Me-lnC/*lnA*/5-Me-lnC/ |
| LNA-PS-Chol | *lnT*lnT*lnT*/5-Me-lnC/*lnA*lnT |
| SMN | *lnA*lnA*lnT*lnG*/5-Me-lnC/*lnT |
| | *lnG*lnG/isp18//isp18//3CholTEG/ |
| | (SEQ ID NO: 79) |
| Linear ASO | lnT*/5-Me-lnC/*lnA*/5-Me-lnC/ |
| 10-27-LNA- | *lnT*lnT*lnT*/5-Me-lnC/*lnA*lnT |
| PS-SMN | *lnA*lnA*lnT*lnG*/5-Me-lnC/ |
| | *lnT*lnG*lnG (SEQ ID NO: 80) |

*= phosphorothioate internucleotide linkage;
ln = LNA sugar modification;
iSp18 = internal hexaethyleneglycol spacer;
5-Me-InC = 5′methyl C with LNA sugar modification;
3CholTEG = 3′-Cholesteryl-TEG;
TEG = tetraethylene glycol

The term “percent identical” refers to sequence identity between two amino acid sequences or between two nucleotide sequences of oligonucleotides disclosed herein, such as antisense oligonucleotides. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASP AR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

In order that the invention described herein may be more fully understood, the following examples are set forth. The examples described in this application are offered to illustrate the compounds, pharmaceutical compositions, and methods provided herein and are not to be construed in any way as limiting their scope.

### EXAMPLES

### Example 1. SMN2-Targeted SNA Increases Expression of SMN2 mRNA and Protein for Treatment of Spinal Muscular Atrophy

Based on these unique properties of SNAs, SNAs have been developed targeting mRNA for down regulation of gene expression and TLR9 protein to activate the immune system. Antisense SNAs for dermal diseases and TLR9 agonist SNAs for immuno-oncology applications are in clinical development. A linear and a SNA version of Spinraza were compared for their effect on the inclusion of exon 7 in SMN2 mRNA in SMA patient-derived fibroblasts. The results show that in patient-derived fibroblasts, SNA version of Spinraza yields greater expression of exon 7 included SMN2 mRNA and protein compared with the linear version of Spinraza currently used to treat SMA patients.

Methods

Linear oligonucleotides (linear ASO) and 3′-cholesterol attached linear oligonucleotides via two hexaethyleneglycol (spacer18) moieties for SNA were synthesized with 2′-methoxyethyl (2′-MOE) and phosphorothioate (PS) backbone modification. The oligonucleotide sequence is the same as that of Spinraza. SNAs (SNA-ASO) were prepared by loading 3′-cholesterol attached oligonucleotides onto DOPC liposomes at a ratio of 30 oligonucleotide molecules per 20 nm liposome.

Oligo sequence:
(SEQ ID NO: 1)
5′ - TCA CTT TCA TAA TGC TGG - (Spacer 18)₂ -
3CholTEG

SMA patient fibroblast cells (GM03813C, GM09677C and GM00232D) were obtained from Coriell Institute for Medical Research. Cells were cultured in DMEM medium containing 10% FBS and 100 U/ml penicillin and 100 μg/ml streptomycin. Linear and SNA ASOs were added to the cell cultures without transfecting agents and incubated for 48 hours or 72 hours. Then the cells were collected at 48 hours for mRNA extraction and at 72 hours for protein isolation. The levels of SMN2 mRNA, SMNΔ7 mRNA, and total SMN mRNAs were measured by qPCR using the following set of probes and primers. SMN2 mRNA and SMNΔ7 mRNA primers were obtained from IDT and the probes were from Thermo Fisher Scientific, and the commercially available primers and probes for total SMN mRNA were purchased from Life Technologies (cat #Hs00165806_ml). SMN2 mRNA forward primer: 5′-GCTG ATGCTTTGGG AAGTATGTTA-3′ (SEQ ID NO: 2), SMN2 mRNA reverse primer: 5′-CACCTTCCTTCTTTTTGATTTTGTC-3′ (SEQ ID NO: 3), SMN2 mRNA probe: 5′-6FAM-TACATGAGTGGCTATCATACTT-MGBNFQ-3′ (SEQ ID NO: 4), SMNΔ7 mRNA forward primer: 5′-TGGACCACCAATAATTCCCC-3′ (SEQ ID NO: 5), SMNΔ7 mRNA reverse primer: 5′-ATGCCAGCATTT CCATATAATAGCC-3′ (SEQ ID NO: 6) and SMNΔ7 mRNA probe: 5′-6FAM-TACATGAGTGGCTATCATACT-MGBNFQ-3′ (SEQ ID NO: 7). The levels of SMN2 protein were measured by Western blotting using SMN antibody obtained from BD Biosciences (cat #610646) and the control GRP94 protein by the Grp94 (9G10) antibody obtained from Enzo Lifesciences (cat #ADI-SPA-850). The fold increase of SMN2 mRNA over SMNΔ7 mRNA was calculated by dividing the values of % SMN2 mRNA expression with % SMNΔ7 mRNA expression.

Results

ASO-SNAs and Linear ASOs targeting ISS-N1 site of the SMN2 mRNA were tested at various concentrations in three different SMA patient-derived fibroblasts. In addition, phenylbutyrate (PBA, a known small molecule compound, positive control) and negative controls (control SNA and control linear) were included in the assays for comparison.

The results are included in FIG. 1A (Full-length SMN mRNA) and 1B (Δ7 SMN mRNA) (pPBA not shown). The results showed that ASO-SNA treatment led to greater inclusion of exon 7 in SMN2 mRNA compared with linear ASO. ASO-SNA treatment resulted in up to 45-fold increase in the inclusion of exon 7 over SMNΔ7 mRNA depending on the source of fibroblasts. Whereas, linear ASO resulted in about 2.5-fold higher inclusion of exon 7 over SMNΔ7 mRNA.

Next, the upregulation of SMN2 protein was measured by ASO-SNA and linear ASO at 72 hours by Western blotting. GM09677C were treated with SNAs for 72 hours and, then assessed by western blot and qRT-PCR. ASO-SNA treatment resulted in greater expression of SMN2 protein compared with linear ASO in GM09677C (FIGS. 2A and 2B), which is consistent with the results from the mRNA levels from above. FIG. 2A Western blot showing total SMN protein and loading control GRP94. GRP94 protein loading control was detected with ADI-SPA-850-F (Enzo Life Sciences). SMN was detected with VMA00249 (Bio-Rad). FIG. 2B is a densitometric quantification of SMN western blot (solid bars) and qRT-PCR of full-length SMN mRNA (hashed bars) from identically treated wells. SMN qRT-PCR was performed on SMA patient fibroblasts (GM09677C) that were plated in 96-well plates and treated in triplicate with SNAs in complete media. After cell lysis, cDNA was derived from extracted RNA and assessed by qRT-PCR with technical duplicates for each sample. Full-length SMN2 was measured relative to GAPDH.

Conclusion

ASO-SNA treatment of SMA patient-derived fibroblasts facilitates increased of exon 7 inclusion and SMN2 protein expression compared with the same sequence of linear ASO (Spinraza). Previous studies have shown that oligonucleotides in SNA format are taken up by cells to a greater extent than linear oligonucleotides and function as potent antisense agents at mRNA level in the cytoplasm to down regulate gene expression. The current results are the first demonstration of SNAs interacting with pre-mRNA in the nucleus facilitating exon 7 inclusion in SMN2 mRNA in SMA patient-derived fibroblasts.

Thus, these in vitro studies showed that SNAs are several fold more potent in generating exon7 included SMN2 mRNA and full-length protein compared with linear oligo.

### Example 2. SMN2-Targeted Antisense Spherical Nucleic Acid (SNA) Treatment of SMA in a Mouse Model

The constructs were tested in vivo in a mouse model to evaluate the potency of SMN2-targeted SNA in comparison with a linear MOE-ASO. Tolerability of SNA compounds can be evaluated by intrathecal (IT) or intracerebroventricular injection (ICV). Spinraza is administered to patients using IT administration so one ideal comparison will involve IT administration in mouse models. It would be a great improvement to be able to deliver the therapeutic SNA into central nervous system using other administration modalities, such subcutaneous, intramuscular, intravenous, oral, ophthalmic, topical delivery in the ear, such as ear drops or similar forms, transtympanic administration, etc. These other administration routes are less invasive compared to intrathecal administration and may improve patient comfort. Spinraza is administered in 5 mL volume (2.4 mg/mL); in mice this volume would be much smaller, on the order of a few microliters. In animal models, survival and other parameters such as SMN mRNA and protein levels, might be sufficient especially for modelling severe SMA. Electromyograms (EMG) can also be recorded for compound muscle action potential (CMAP) as well as motor unit number estimation. These parameters are reduced in SMA. If SMN levels are normalized by therapeutic interventions, these values have been observed to recover. In human clinical trials, CMAP is observed to correlate well with motor function and has the potential value as a relevant surrogate for disease status. This is one of the only measures that can be made in humans and mouse models.

It has previously been shown that morpholino antisense treatment directed at the negative regulatory ISS-N1 in SMN2 results in increased incorporation of SMN2 exon7 and increased levels of SMN protein. It has further been shown that the second hnRNP A1 site at −85-109 in intron7 can also be blocked to give an equivalent level of SMN to blocking ISS-N1. However due to delivery to critical cells the latter therapy was not as effective when used as a morpholino as ISS-N1. The blocking of ISS-N1 as well as −85-109 results in increased survival and function of SMA model mice. In addition, there is significant recovery of the electrophysiologic function. The latter is critical as regards SMA treatment in humans as in SMA there is clear decrement of motor neuron function in human and no critical evidence for a role of the periphery. Indeed, patients treated early using either antisense oligonucleotide or gene therapy show remarkable improvement in phenotype achieving milestones never observed in SMA patients.

An ASO targeting ISS-N1 site of SMN2 mRNA (−10-27) with MOE chemistry (Spinraza) has been recently approved by the FDA. When using an intrathecal delivery system in human SMA the antisense oligonucleotide is showing good effect when treatment is given pre-symptomatically. In preclinical work, the MOE-ASO did show toxicity in mice and the MOE-ASO could not be used at the same concentration as the morpholino. Passini et al used a single dose of MOE-ASO via intracerebral ventricular (ICV) injection up to 8 μg and obtained a survival improvement from 14 days to 23 days, Hua et al used a ICV dose of 20 μg with no adverse effect and in a different SMA animal model had an increased survival from 10 days to 16 days. Hua et al also obtained further improvement by giving the ASO into the periphery. This contrasts with the morpholino data that showed survival beyond 100 days in the delta? SMA mice whereas mice without treatment lived for 13 days.

Methods

Linear oligonucleotides (linear ASO) and 3′-cholesterol attached linear oligonucleotides via two hexaethyleneglycol (spacer18) moieties for SNA were synthesized with 2′-methoxyethyl (2′-MOE) and phosphorothioate (PS) backbone modification. The oligonucleotide sequence is the same as that of Spinraza. SNAs (SNA-ASO) were prepared by loading 3′-cholesterol attached oligonucleotides onto DOPC liposomes at a ratio of 30 oligonucleotide molecules per 20 nm liposome particle.

Compounds were administered to mice by intracerebro-ventricular injections as described previously (P. N. Porensky, et al, Hum. Mol. Genet. 21, 1625-1638, 2012). Briefly, P0 pup was cryo-anesthetized and hand-mounted over a back-light to visualize the intersection of the coronal and sagittal cranial sutures (bregma). A fine-drawn capillary needle with injection assembly was inserted 1 mm lateral and 1 mm posterior to bregma, and then tunneled 1 mm deep to the skin edge (approximating) ipsilateral lateral ventricle. An opaque tracer (Evans Blue, 0.04%) was added to the reagent to visualize the borders of the lateral ventricle after injection of 2 or 3 μl of SNA-ASO or linear ASO. A single dose of SNA-ASO or linear ASO at 10, 20 or 30 μg dose/mouse administered by ICV at age P0. Following administration of compounds, mouse survival and body weights were recorded.

Spinal cords of SMA mice treated with 30 μg dose of SNA-ASO on P0 and untreated control mice were collected on P10 and measured full-length SMN2 mRNA transcript by digital droplet PCR as described previously by P. N. Porensky, et al, Hum. Mol. Genet. 21, 1625-1638, 2012. Table 7 outlines the compounds used given along with an examplary animal number.

TABLE 7
Protocol for the study of SMN2 antisense SNA and MOE-ASO (linear oligo) in SMN^{-/-} SMN2 D7 SMA mice

| Compound | Volume/dossing | Dose/animal | Phenotype | Genotype | Animals/group | Outcome |
|---|---|---|---|---|---|---|
| -10-27 linear oligo | 2 ul once | 10 ug | SMA | Tox trial | 5 | Tox |
| -10-27 linear oligo | 2 ul once | 20 ug | SMA | Tox trial | 5 | Tox |
| -10-27 SNA | 2 ul once | 10 ug | SMA | Tox trial | 5 | Tox |
| -10-27 SNA | 2 ul once | 20 ug | SMA | Tox trial | 5 | Tox |
| -10-27 linear oligo | 2 ul once | 10 ug | SMA | Smn^{-/-} SMN2 D7 | 13 | Survival/EMG |
| -10-27 linear oligo | 2 ul once | 20 ug | SMA | Smn^{-/-} SMN2 D7 | 13 | Survival/EMG |
| -10-27 linear oligo | 3 ul once | 30 ug | SMA | Smn^{-/-} SMN2 D7 | 13 | Survival/EMG |
| -10-27 SNA | 2 ul once | 10 ug | SMA | Smn^{-/-} SMN2 D7 | 13 | Survival/EMG |
| -10-27 SNA | 2 ul once | 20 ug | SMA | Smn^{-/-} SMN2 D7 | 13 | Survival/EMG |
| -10-27 SNA | 3 ul once | 30 ug | SMA | Smn^{-/-} SMN2 D7 | 13 | Survival/EMG |
| ISS-N1 PMO oligo | 2 ul once | 40 ug | SMA | Smn^{-/-} SMN2 D7 | 13 | Survival/EMG |
| Control SNA | 3 ul once | 30 ug | SMA | Smn^{-/-} SMN2 D7 | 13 | Survival/EMG |
| RNA testing | 2 ul | | SMA | Smn^{-/-} SMN2 D7 | 5 × 6 = 30 | ddPCR |
| SMN protein | 2 ul | | SMA | Smn^{-/-} SMN2 D7 | 5 × 6 | Western |
| RNA testing | 2 ul | | Carrier | Smn +/− SMN2 D7 | 5 × 6 = 30 | ddPCR |
| SMN protein | 2 ul | | Carrier | Smn +/− SMN2 D7 | 5 × 6 | Western |

The pharmacodynamic activity of the compounds is followed by survival of mice in each group compared with untreated mice. In a previous study, morpholino ASO prolonged the Smn^{-/-} SMN2 D7 mice survival over 100 days, which serves as a reference for the current study. Further, the EMGs will be recorded for muscle action potential (CMAP) as well as motor unit number estimation. Both these parameters are reduced in SMA at 6 days and beyond. When SMN levels are corrected due to the action of the test compounds, these values recover and when mice live out can reach normal levels. This is an important measure as it shows that the motor neuron has recovered and the muscle is innervated correctly. It is one of the only measures that can be made in man and mouse and is altered in human SMA.

The measures of SMN protein and RNA give a measure of the increased incorporation of SMN exon7 and the amount of SMN protein. In the cases of the carrier mice tested only the human SMN is detected thus the increase can be seen on a background where no cell loss is occurring.

Results

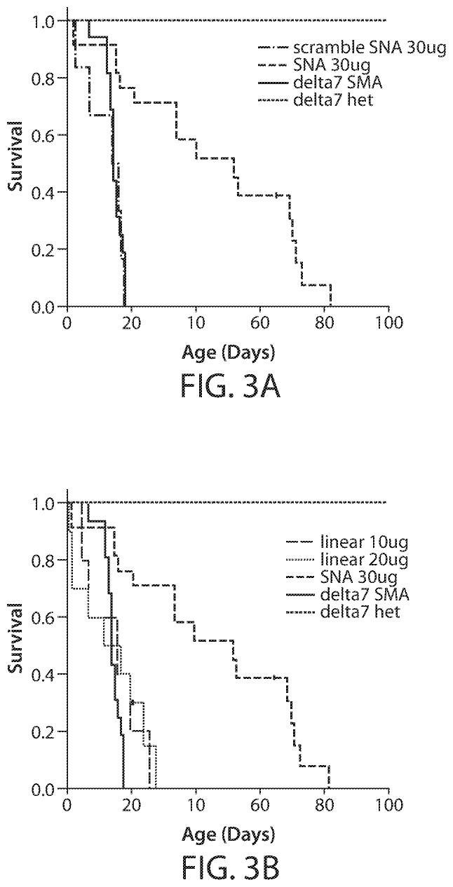

A single dose of SNA-ASO or linear ASO was injected to mice on P0 at 10, 20 or 30 μg. The Kaplan-Meier survival plots of SMA mice treated with SNA-ASO and linear ASO and untreated mice are shown in FIGS. 3A and 3B. Mice were genotyped at P0 (day of birth) and injected via Intracerebroventricular injection (ICV) on P0. The recorder of events was blinded to genotype and treatment. Control untreated mice died within 18 days with a median survival of about 14 days. Mice treated with linear ASO showed a median survival of 16, 17 and 2 days at 10, 20 and 30 μg doses, respectively with a maximal survival prolongation of about 28 days. SNA-ASO treatment lead to increased survival of SMA mice at all dose levels compared with linear ASO. The median survival of SNA-ASO treated mice was 26, 69 and 70 days at 10, 20, and 30 μg doses, respectively. The survival of SMA mice was prolonged up to about 117 days in 20 μg SNA-ASO dose group and the mice in 30 μg dose group have not reached end point. These results clearly demonstrate that SNA-ASO prolongs survival of SMA mice to a greater extent than linear ASO. Additionally, early death of mice in 30 μg dose linear ASO group suggest possible toxicity. These results suggest that SNA-ASO treatment is safe and well tolerated up to 30 μg dose level in SMA mice.

FIG. 3A shows Δ7SMA mice treated with the 30 μg dose Nusinersen-SNA had increased survival to a maximum of 82 days while scramble SNA has no effect on survival. FIG. 3B shows that linear Nusinersen improved survival of Δ7 SMA mice to a maximum of 28 days. The data is also summarized in the table below.

| | | Mean | Maximum | |
|---|---|---|---|---|
| Treatment with | # of | survival | survival | |
| Nusinersen | mice | (days) | (days) | Log rank p value |
| Linear 10 μg | 5 | 14.8 ± 4.0 | 26 | NS |
| Linear 20 μg | 10 | 14.0 ± 3.0 | 28 | NS (censored) |
| Linear 30 μg | 5 | 2.2 ± 0.1 | 2 | Toxicity |
| SNA 10 μg | 8 | 25.7 ± 3.3 | 40 | 0.00064 |
| SNA 20 μg | 9 | 57.0 ± 14.0 | 115 | .002 censored |
| SNA 30 μg | 23 | 45.6 ± 6.1 | 82 | 0.000017 censored |
| Scrambled SNA | 6 | 12.5 ± 2.5 | 18 | NS |
| 30 μg | | | | |
| untreated Δ75MA | 16 | 14.3 ± 0.7 | 18 | Tested against |

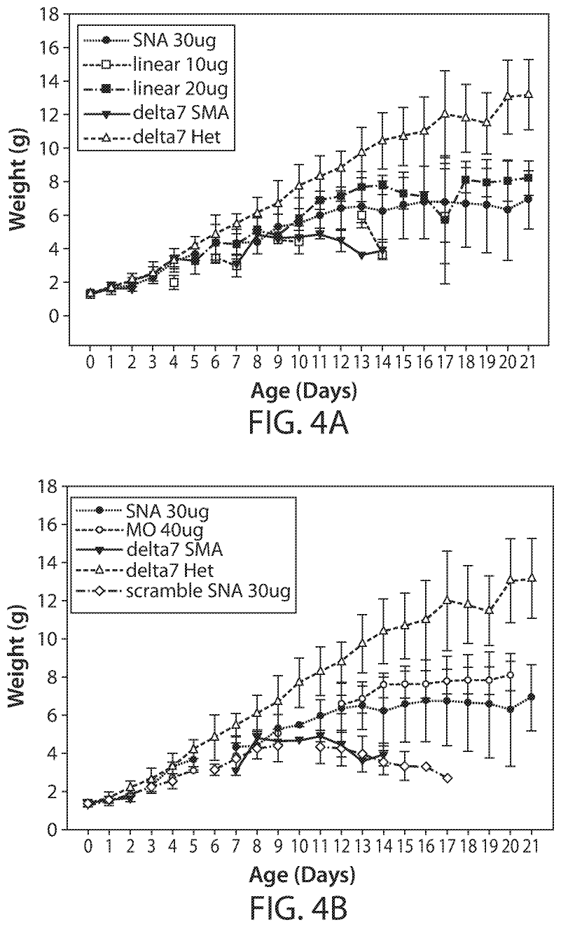

Phenotypic changes, including weight changes, on the treated mice were assessed. Weight curves to 21 days of age in treated and untreated control mice are shown in FIGS. 4A and 4B. Mice were weighed each day. FIG. 4A shows that weights are similar in Δ7SMA mice treated with linear or Nusinersen-SNA treated mice. FIG. 4B shows that weights are similar in Δ7SMA mice treated with morpholino to ISS-N1 or Nusinersen-SNA. The scramble-SNA did not alter the weight of the Δ7SMA mice.

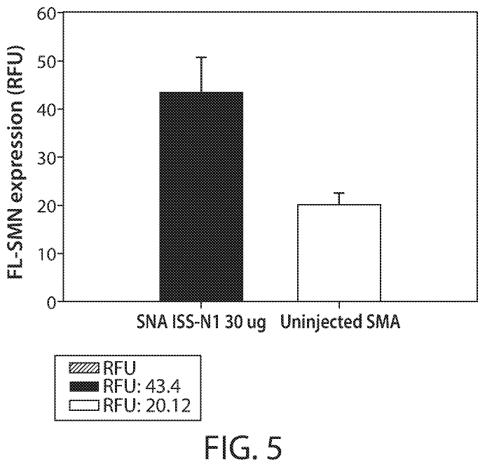

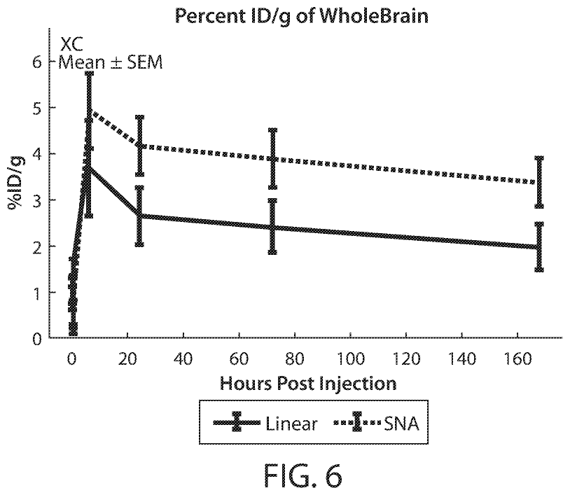

To examine if the treatment of SMA mice with SNA-ASO lead to increased levels of SMN2 full-length mRNA transcript, spinal cords were collected on P10 from mice treated with 30 μg SNA-ASO and untreated control mice, and measured SMN2 mRNA transcript levels by digital droplet PCR. The results shown in FIG. 5 demonstrate that SNA-ASO treatment increased the full-length SMN2 mRNA transcript in SMA mice compared with untreated mice on P10.

Thus, treatment of SMA mice with a single ICV dose of ASO-SNA increased exon 7 inclusion. Moreover, the treatment of SMA mice with ASO-SNA resulted in increased median survival of up to 69/70 days with a prolongation of survival beyond 100 days compared with linear ASO. Further the SNA-ASOs are safe and well tolerated in SMA mice compared with linear ASO. These animal model studies support delivery of SNA to CNS and for neuromuscular disease treatment.

The SNAs increased uptake of MOE Nusinersen in cell models lacking SMN1 but containing SMN2, resulting in increased amounts of full-length mRNA and SMN protein from SMN2. Additionally, SNAs when delivered to CSF in the Δ7SMA mouse model allow increased dosing of Nusinersen and increased efficacy with prolonged survival of SMA mice. SNAs when delivered to CSF in the Δ7SMA mouse model also have increased full-length SMN mRNA levels in spinal cord tissue. In view of these data demonstrating the enhanced use of SNA relative to Nusinersen, the therapeutic utility of the SNA is substantial.

Additional experiments for further analysis include: Performing EMG, compound muscle action potential (CMAP) and motor unit number estimation (MUNE) to assess the extent of motor neuron correction and determining Nusinersen-SNA bio-distribution and SMN levels in all treatment groups using ELISA and Western blot.

### Example 3. Comparative Analysis of ¹²⁵I-Oligonucleotides by SPECT/CT Imaging in Sprague Dawley Rats

The examples above illustrate that, compared to linear nusinersen, the SNA version of nusinersen has superior splice modulating activity in cell culture in SMA-patient derived fibroblasts. The examples above also illustrate that, in mouse models of SMA, in comparison to linear nusinersen, the SNA version of nusinersen increases median survival and has lower toxicity at higher doses.

The central nervous system (CNS) distribution of intrathecally administrated oligonucleotides (linear ASO and SNA-ASO) was characterized using single-photon emission computed tomography combined with computed tomography (SPECT/CT) imaging in Sprague Dawley rats. Linear ASO and 3′-cholesterol attached linear oligonucleotides via two hexaethyleneglycol (spacer18) moieties for SNA were synthesized with 2′-methoxyethyl (2′-MOE) and phosphorothioate (PS) backbone modifications as described below. The oligonucleotide sequence is the same as that of Spinraza. The oligonucleotides were further modified on the 5′ terminus with amino modified to enable eventual attachment of iodine-125 radio-label element. SNAs (SNA-ASO) were prepared by loading 3′-cholesterol attached oligonucleotides onto DOPC liposomes at a ratio of 30 oligonucleotide molecules per 20 nm liposome particle. The oligonucleotides in both linear ASO and SNA-ASO groups were labeled with iodine-125. The radio-labeled compounds were injected into SD rats (up to 3 rats per group) and whole body SPECT/CT was performed at 0, 0.25, 0.5, 0.75, 6, 24, 72 and 168 hours after injection. 0 hours after injection is essentially immediately after injection of the radio-labeled compounds. Each rat received 180 μg of radio-labeled oligonucleotide in single bolus injection via intrathecal administration in the lower lumbar region, around the 6th lumbar vertebra.

| | SEQ | |
|---|---|---|
| | ID | |
| Compound | NO | Sequence |
| Linear | 162 | /5AmMC6/*moeT*/5-Me-moeC/*moeA*/5-Me- |
| ASO | | moeC/*moeT*moeT*moeT*/5-Me- |
| | | moeC/*moeA*moeT*moeA*moeA*moeT*moeG*/ |
| | | 5-Me-moeC/*moeT*moeG*moeG |
| SNA-ASO | 163 | /5AmMC6/*moeT*/5-Me-moeC/*moeA*/5-Me- |
| | | moeC/*moeT*moeT*moeT*/5-Me- |
| | | moeC/*moeA*moeT*moeA*moeA*moeT*moeG*/ |
| | | 5-Me-moeC/*moeT*moeG*moeG/isp18/// |
| | | isp18//3CholTEG |

*= phosphorothioate internucleotide linkage;
moe = 2′-O-(2-methoxyethyl);
iSp18 = internal hexaethylenegylcol spacer;
5-Me-moeC = 5′methyl C with moe modification;
3CholTEG = 3′-Cholesteryl-TEG;
5CholTEG = 5′Cholesteryl-TEG;
TEG = tetraethylene glycol;
5AmMC6 = 5′-Amino-Modifier C6

The SPECT/CT image analyses show that there is a profound difference between the distribution and persistence of linear ASO compared with SNA-ASO. The linear ASO rapidly distributes from the site of administration in lower lumbar region to the other areas of the spinal cord. Within 1-6 hours, noticeable amount of signal from the iodine label is present in the brain as well. Over the course of the 7-day monitoring period, starting as early as 6-hours post-administration, the oligonucleotide signal is decreasing in many regions of the CNS, and is being observed via the kidneys.

By contrast, SNA-ASO distributed away from the site of administration relatively slowly. During the first hour, oligonucleotide is detectable in the spinal cord but not in the brain. Starting at 6 hours post-administration, high amount of oligonucleotide is present in the brain along with the spinal cord. This strong signal remains present in the brain and parts of the spinal cord through the 7-day monitoring period. Less SNA-ASO is observed in the kidney, which likely indicates a relatively slow clearance rate of SNA-ASO from the CNS. Overall, SNA-ASO is persistent in the CNS longer and at higher levels compared to linear ASO.

The whole body images were further analyzed to determine percent of injected dose per gram of tissue present in various regions of interest. Regions of interest, including 13 regions of rat brain, were placed onto each image using automated software tools or approximate anotomical location. Fixed volume regions of interest were used for regions with limited signal. Iodine-125 levels were measured, converted to units of activity, decay corrected and corrected for background radiation. The values were converted to percent injected dose per gram of tissue. Comparison between linear ASO and SNA-ASO shows that following intrathecal injection, SNA delivers approximately 34-71% more oligonucleotide to the whole brain compared to linear ASO. For various regions of the brain, generally linear ASO shows higher oligonucleotide levels at early time points, usually at 0 hours. Unexpectedly, SNA generally shows higher oligonucleotide levels at later time points, typically starting at 6 hours but often earlier for many regions of the brain. Regions with relatively higher distribution for SNA ASO include amygdala (approximately 41-75% higher), basal ganglia (approximately 26-37% higher), cerebellum (approximately 25-78% higher), corpus callosum (approximately 6-149% higher), cortex (approximately 14-73% higher), hippocampus (approximately 6-102% higher), hypothalamus (approximately 41-72% higher), midbrain (approximately 32-73% higher), olfactory (approximately 61-102% higher), ventricles (approximately 8-79% higher), septal area (approximately 19% higher), thalamus (approximately 2-92% higher), and white matter (approximately 27-72% higher). Similar data are also observed in the cervical and thoracic CSF where SNA shows approximately 7-77% and 92-103% higher distribution respectively. In the lumbar CSF, where the compounds are administered, SNA ASO shows higher distribution at nearly all time points (approximately 27-59% higher). Surprisingly, the linear ASO appears at high levels in the kidneys whereas SNA ASO shows high levels in the liver, and superficial and deep cervical lymph nodes, which indicates distinctly different distribution and clearance profiles for SNA ASO compared to linear ASO.

The longer persistence of SNA-ASO suggests that SNA-based therapy could be administered less frequently compared to linear ASO. Since the examples also illustrate that SNA version of nusinersen is not toxic at high doses, in contrast to linear nusinersen which has high toxicity at 30 μg dose in SMA mouse model, higher absolute amount of therapy can also be administered. The combination of higher persistence and lower toxicity can potentially further reducing dosing frequency. The higher distribution in various regions of the brain could enable intrathecal administration of therapies that target diseases of regions of the brain that are quite distal from site of administration.

The subjects were male Sprague Dawley rats (n=9 injected; n=6^{b} on study). The modalities were whole body SPECT/CT. Image agents were formulated with artificial cerebrospinal fluid (aCSF) for intrathecal (IT) injection. The test article is ¹²⁵I-ASO 10-27-MOE-PS SNA, spherical nucleic acid (SNA) composed of an oligonucleotide labeled with iodine-125. The control article is ¹²⁵I-ASO 10-27-MOE-PS, linear ASO labeled with iodine-125. The study design is summarized in Table 8.

TABLE 8

| | | Radiolabeled Test Article | | | | | | |
|---|---|---|---|---|---|---|---|---|
| | | | Dose | | Dose | | Gamma | |
| | Number of | Test | Level | Dose | Radioactivity | SPECT/CT | Counting | Acquisition |
| Group | Animals/Sex | Article | (μg) | Volume, Route | (μCi) | Timepoints | Samples | Timeline |
| Group 1 | 3M | ¹²⁵I-ASO | ~180 | 30 μE + 40 μE | 263.5 ± 94.5 | 0-1, 6, 24, | Whole | Aug. 28, 2018- |
| | | Linear | | flush aCSF, IT | | 72, and | Blood | Sep. 4, 2018 |
| Group 2 | 3M | ¹²⁵I-ASO | ~180 | 30 μE + 40 μE | 241.1 ± 65.0 | 168 hours | Plasma | Sep. 4, 2018- |
| | | SNA | | flush aCSF, IT | | | | Sep. 11, 2018^{a} |

^{a}Imaging dates for animal A4007 (Linear ASO Group). Animal maintains same imaging timepoints as first cohort.
^{b}Only five animals will be used in quantitative analysis.

### Analysis Methods

### Image Analysis

SPECT images were co-registered to CT images and resampled to uniform voxel sizes (0.3 mm³). Regions of Interest (ROIs) were defined using various methods in VivoQuant software. Invicro's 13-region rat brain atlas was placed automatically onto each image using the 3D Brain Atlas Tool in VivoQuant. Fixed volume ellipsoidal ROIs were placed in the center of the liver, kidneys and spleen to encompass areas of representative concentration for each respective region. The superficial and deep cervical lymph nodes, and thyroid were identified using the SPECT. For subjects with limited SPECT activity in these regions, ROIs were placed in the approximate anatomical location using the CT for reference. Fixed volume spherical ROIs were placed in the left and right sides of each of these regions. The CSF was defined using connected thresholding and then split into three regions based on identification of vertebrae: lumbar, thoracic, cervical.

### Gamma Counting

1000 μL aliquots of blood and plasma were collected at 168 hours, placed in tubes and assayed for radioactivity in a gamma counter. The measured count rate, counts per minute (CPM), was converted to units of activity (μCi) using an efficiency value of 0.673 counts per decay for ¹²⁵I. Activities were decay corrected from the time of measurement to the time of injection and corrected for background radiation.

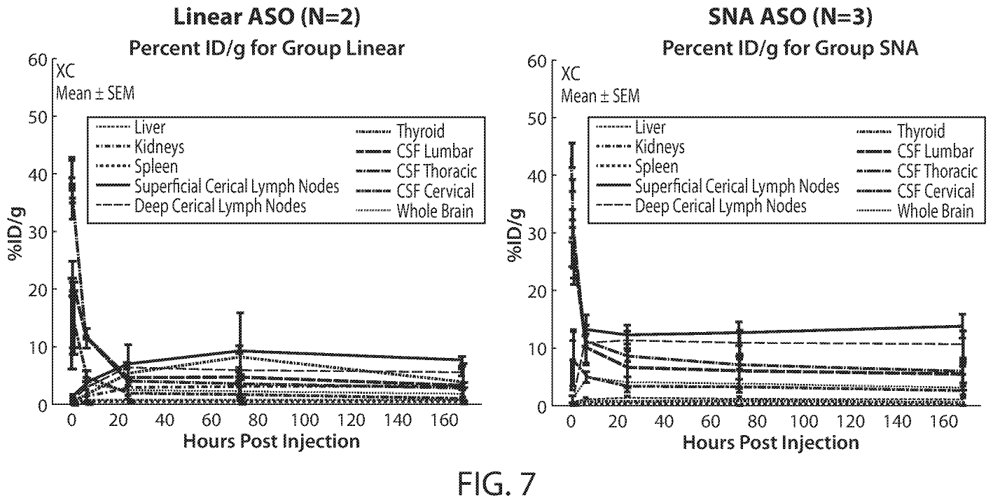

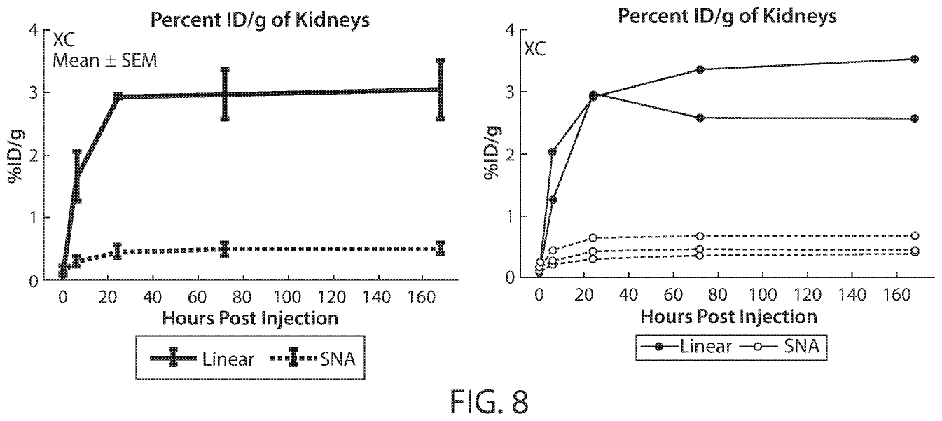

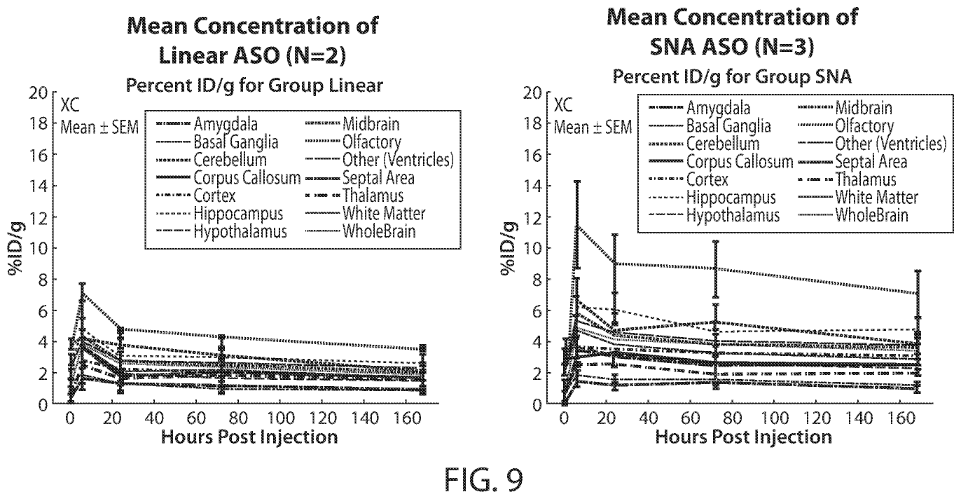

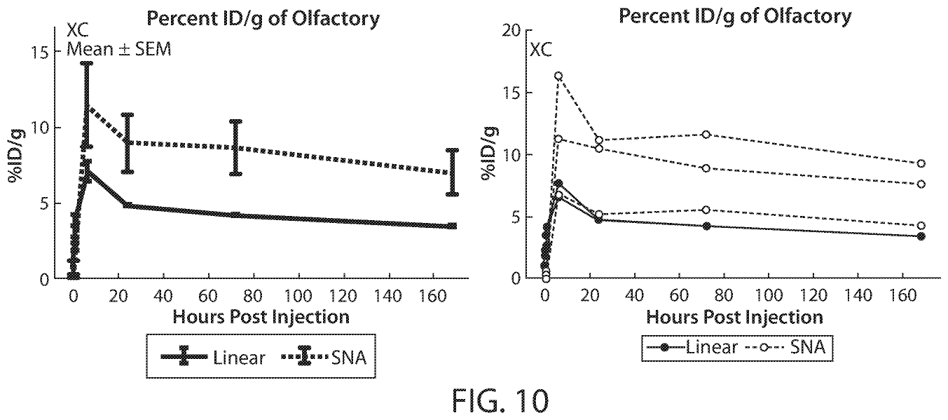

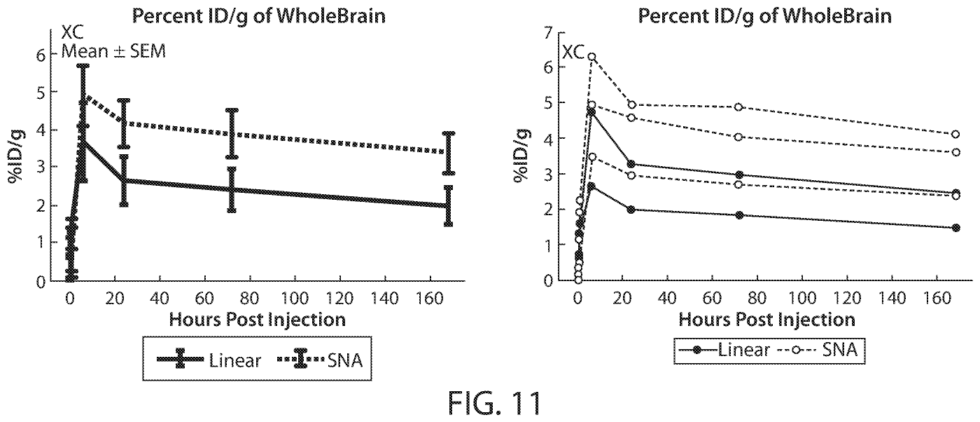

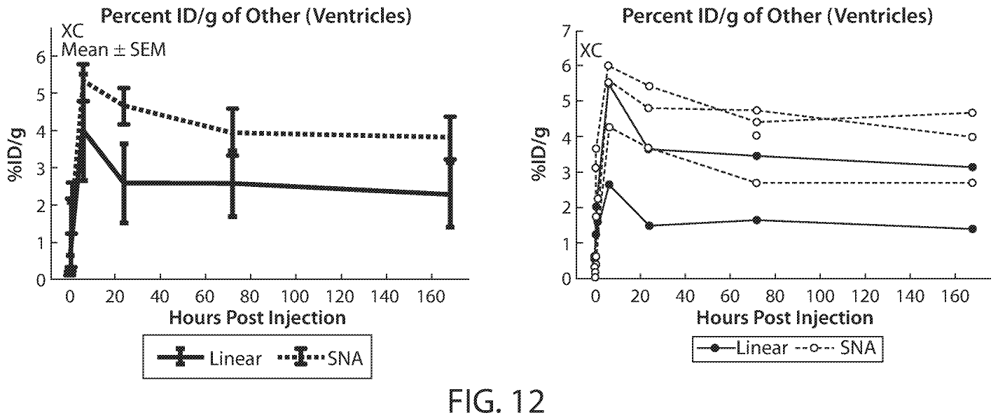

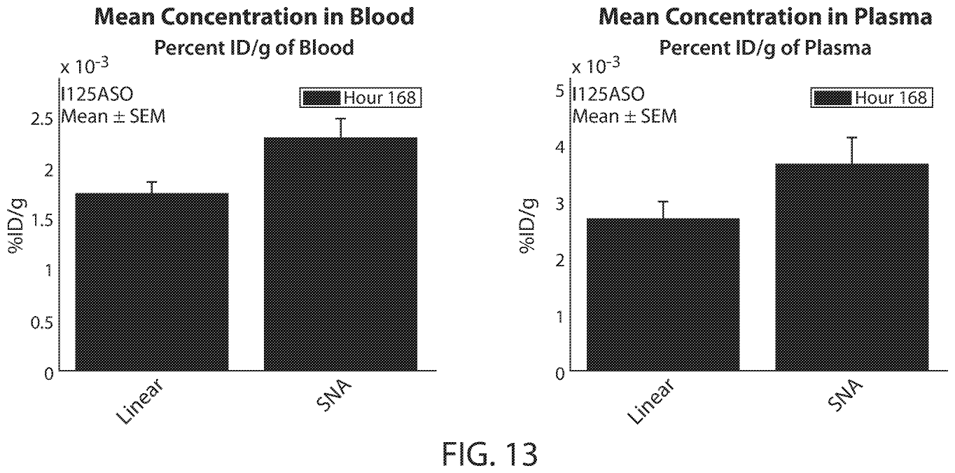

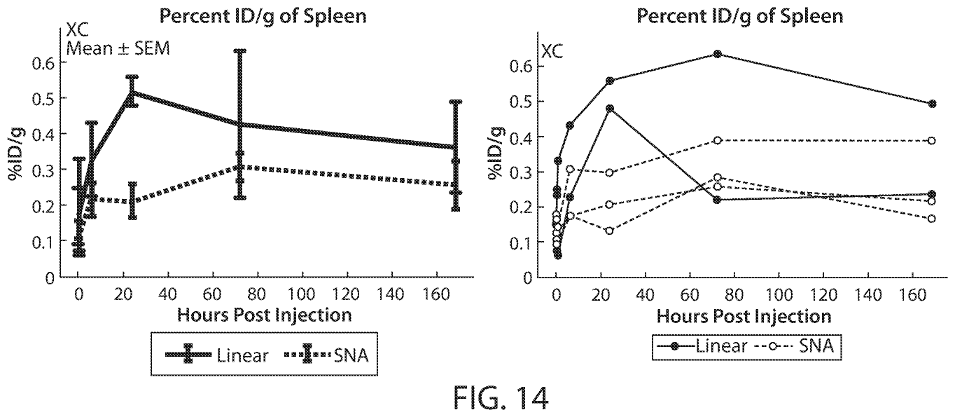

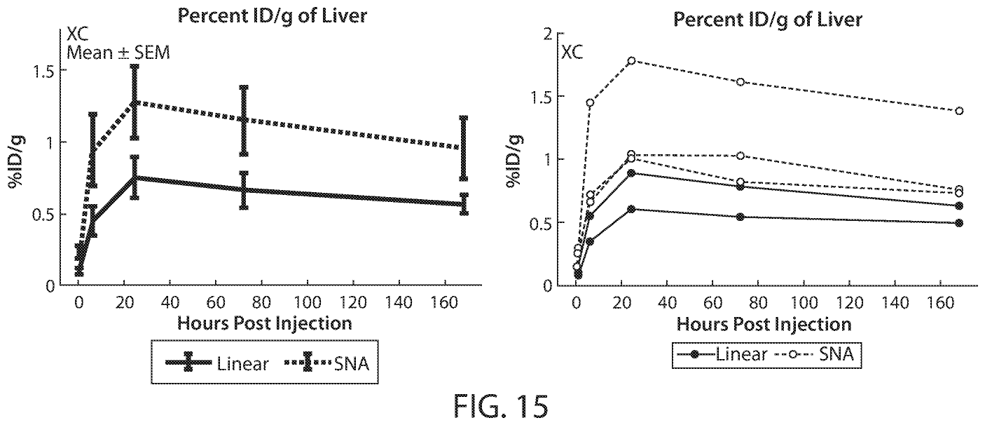

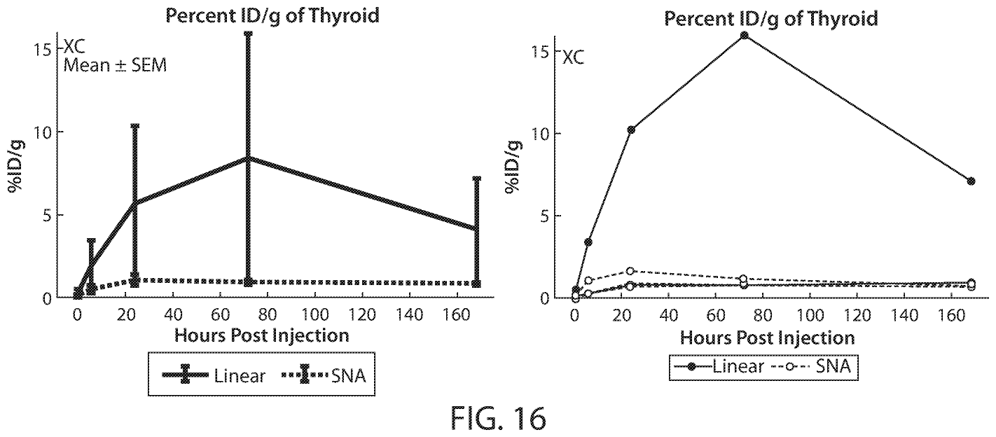

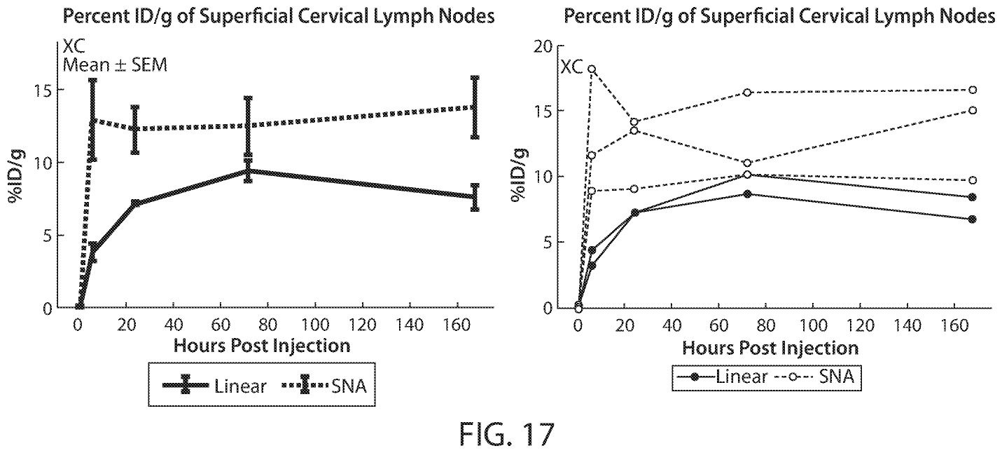

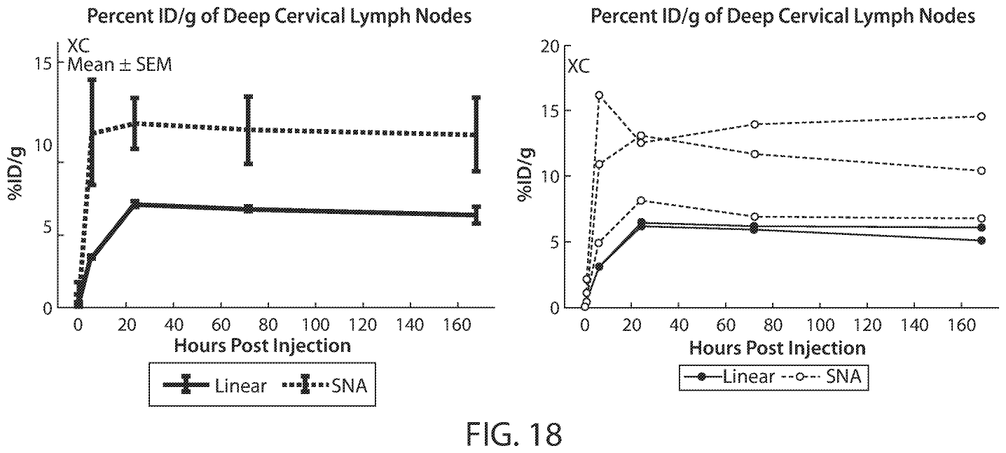

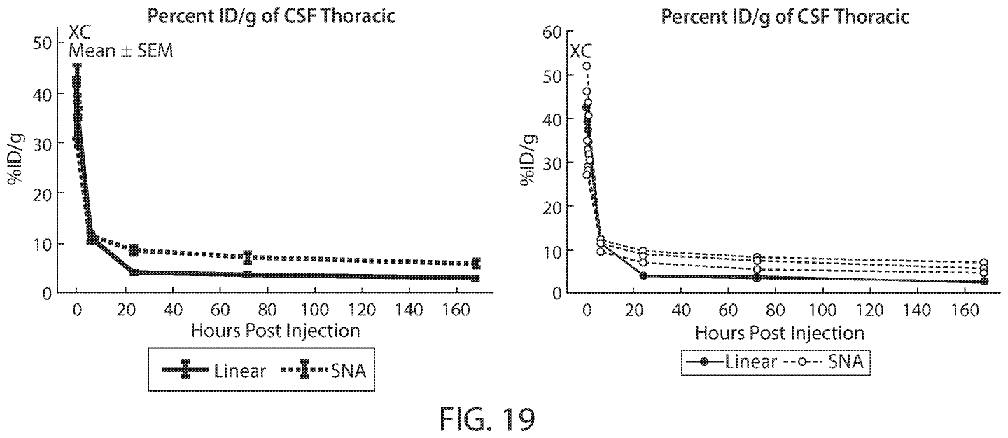

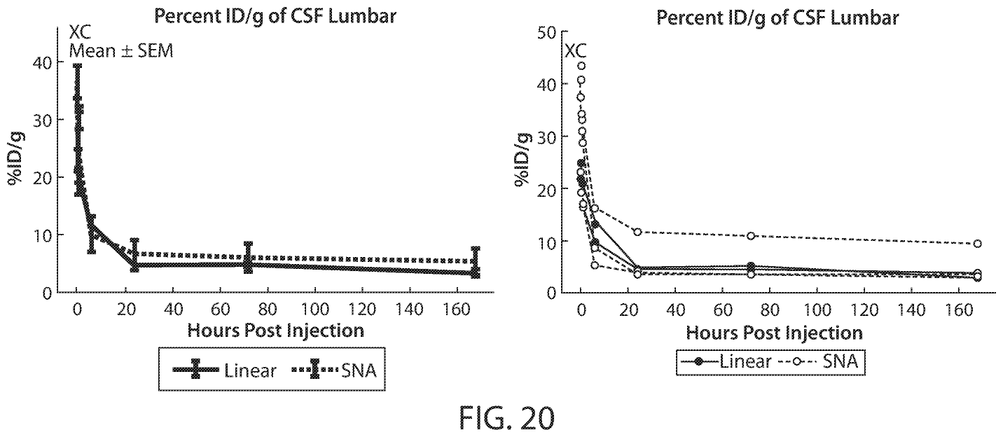

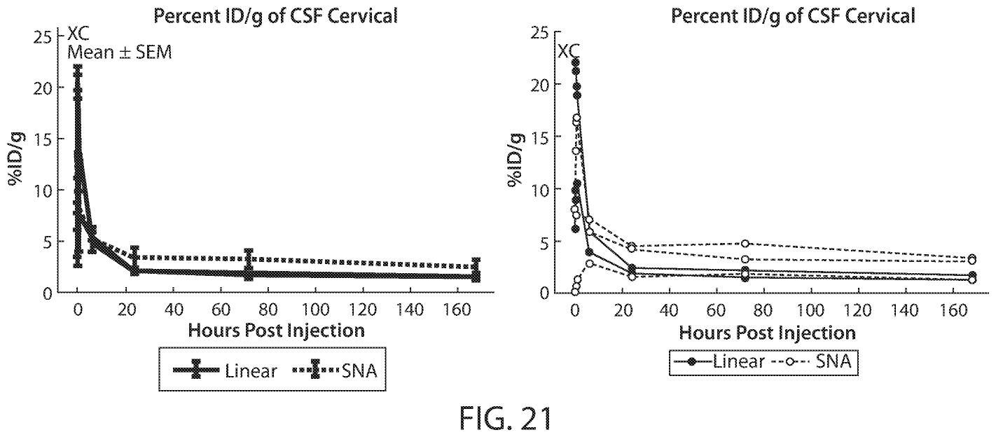

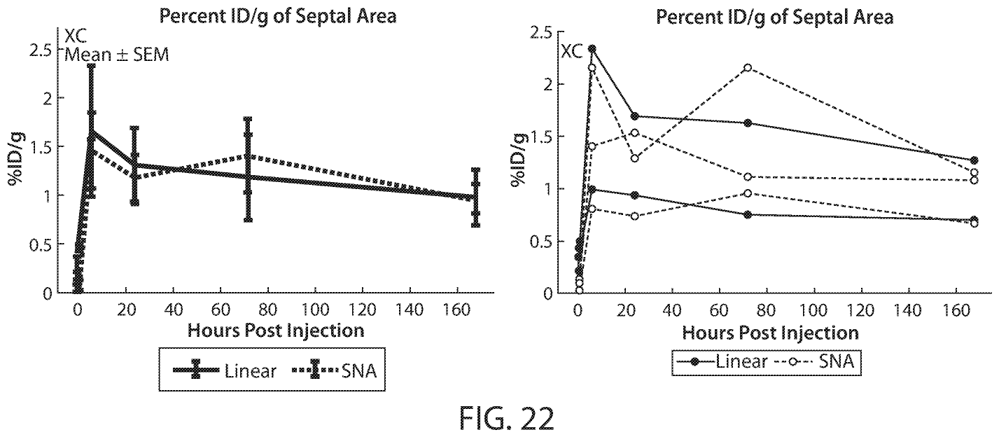

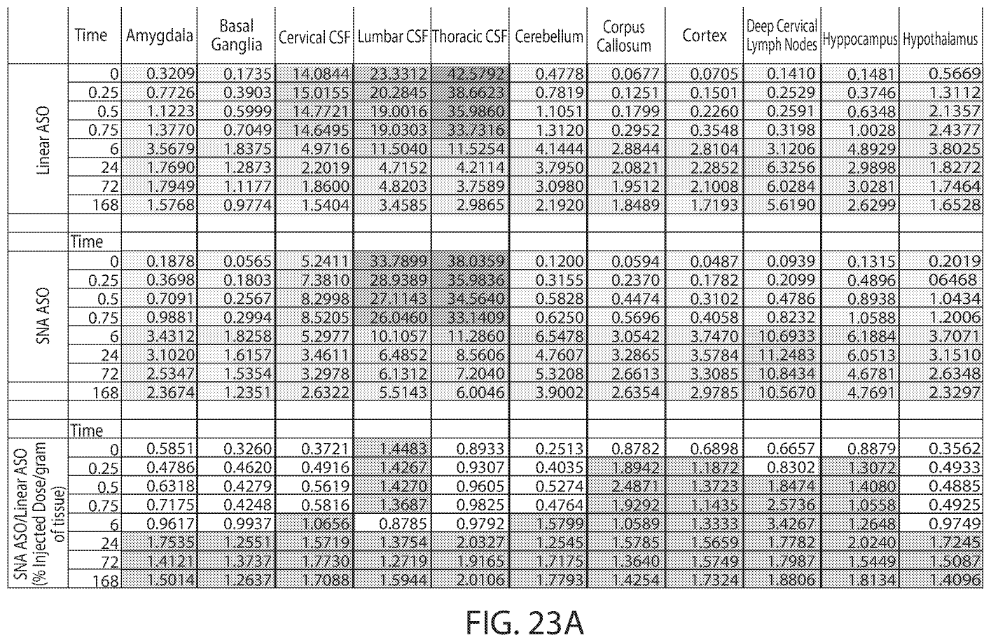

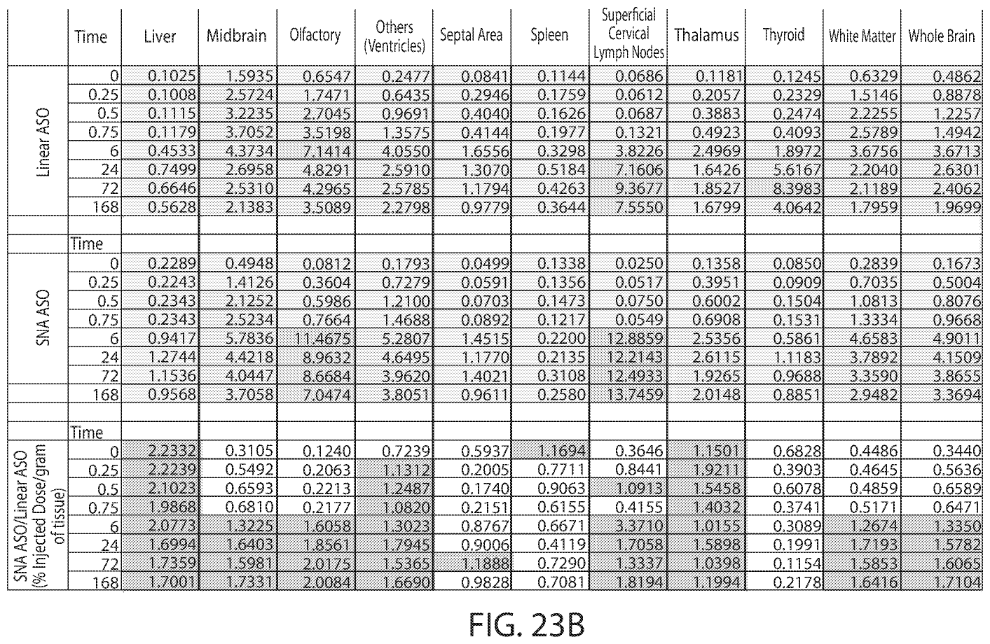

The concentration (% ID/g) of ¹²⁵I-Linear ASO was significantly greater than ¹²⁵I-SNA ASO in the kidneys at 6, 24, 72, 148 h (FIG. 7). The concentration (% ID/g) of ¹²⁵I-Linear ASO was significantly greater than ¹²⁵I-SNA ASO in the kidneys at 6, 24, 72, 148 h (p<0.05, IS t-test) (FIG. 8). Graphs of further results are shown in FIGS. 9-14.

### Example 4. Effects of SNA-ASO, Gold (Au)-SNAs and Linear Oligonucleotides Comprising the Sequence of Spinraza on SMN2 and SMN2Δ7 Levels in Fibroblasts

### Methods

Linear oligonucleotides with the same sequence as spinraza were synthesized with 2′-methoxyethyl (2′-MOE) and phosphorothioate (PS) backbone modifications. These oligonucleotides contained 3′ cholesterol, distearyl, monothiol, or dithiol modifications attached via hexaethyleneglycol (spacer18) moieties. Nonsense control sequences were also synthesized to compare efficacy. Table 9 contains information on oligonucleotide sequence and modifications. SNAs (SNA-ASO) were prepared by loading oligonucleotides containing 3′ cholesterol or distearyl onto DOPC liposomes. Oligonucleotides containing monothiol and dithiol modifications were functionalized onto gold nanoparticles to produce gold SNAs (Au-SNA). SNA core size and oligonucleotide loading densities per particle are described in Table 10.

TABLE 9

| 2′-OMOE | SEQ | |
|---|---|---|
| Chemistry | ID NO | Sequence |
| ASO 10-27- | 164 | moeT*/5-Me-moeC/*moeA*/5-Me- |
| MOE-PS-Chol | | moeC/*moeT*moeT*moeT*/5-Me- |
| | | moeC/*moeA*moeT*moeA*moeA*moeT*moeG*/5-Me- |
| | | moeC/*moeT*moeG*moeG/isp18//isp18//3CholTEG/ |
| SMN Control- | 165 | moeT*moeG*moeT*moeA*moeT*/5-Me-moeC/*moeT*/5-Me- |
| 1-MOE-PS- | | moeC/*moeA*moeT*moeT*moeG*moeT*moeA*moeG/isp18// |
| Chol | | isp18//3CholTEG/ |
| ASO 10-27- | 166 | moeT*/5-Me-moeC/*moeA*/5-Me- |
| MOE-PS | | moeC/*moeT*moeT*moeT*/5-Me- |
| Distearyl | | moeC/*moeA*moeT*moeA*moeA*moeT*moeG*/5-Me- |
| | | moeC/*moeT*moeG*moeG/iSP18//iSP18//branch//STA/ |
| SMN Control- | 167 | moeT*moeG*moeT*moeA*moeT*/5-Me-moeC/*moeT*/5-Me- |
| MOE-PS- | | moeC/*moeA*moeT*moeT*moeG*moeT*moeA*moeG/iSp18// |
| Distearyl | | iSp18//branch//STA/ |
| ASO 10-27- | 168 | moeT*/5-Me-moeC/*moeA*/5-Me- |
| MOE-PS | | moeC/*moeT*moeT*moeT*/5-Me- |
| Monothiol | | moeC/*moeA*moeT*moeA*moeA*moeT*moeG*/5-Me- |
| | | moeC/*moeT*moeG*moeG/iSP18//iSP18//3ThioMC6-D/ |
| SMN Control- | 169 | moeT*moeG*moeT*moeA*moeT*/5-Me-moeC/*moeT*/5-Me- |
| MOE-PS | | moeC/*moeA*moeT*moeT*moeG*moeT*moeA*moeG/iSp18// |
| Monothiol | | iSp18//3ThioMC6-D/ |
| ASO 10-27- | 170 | moeT*/5-Me-moeC/*moeA*/5-Me- |
| MOE-PS | | moeC/*moeT*moeT*moeT*/5-Me- |
| Dithiol | | moeC/*moeA*moeT*moeA*moeA*moeT*moeG*/5-Me- |
| | | moeC/*moeT*moeG*moeG/iSP18//iSP18//3SerinolDTPA/ |
| SMN Control- | 171 | moeT*moeG*moeT*moeA*moeT*/5-Me-moeC/*moeT*/5-Me- |
| MOE-PS | | moeC/*moeA*moeT*moeT*moeG*moeT*moeA*moeG/iSp18// |
| Dithiol | | iSp18//3SerinolDTPA/ |

*= phosphorothioate internucleotide linkage;
moe = 2′-O-(2-methoxyethyl);
iSp18 = internal hexaethyleneglycol spacer;
5-Me-moeC = 5′methyl C with moe modification;
3CholTEG = 3′-Cholesteryl-TEG;
5CholTEG = 5′Cholesteryl-TEG;
TEG = tetraethylene glycol;
branch = symmetrical branching;
STA = stearyl;
3ThioMC6-D = Monothiol;
3SerinolDTPA = Dithiol serinol

TABLE 10

| | SNA Core | Oligonucleotides |
|---|---|---|
| SNA Oligonucleotide | (core diameter in nm) | per Core |
| ASO 10-27-MOE-PS-Chol | DOPC (20) | 30 |
| SMN Control-MOE-PS-Chol | DOPC (20) | 30 |
| ASO 10-27-MOE-PS Distearyl | DOPC (20) | 30 |
| SMN Control-MOE-PS- | DOPC (20) | 30 |
| Distearyl | | |
| ASO 10-27-MOE-PS Monothiol | Gold (13) | 198 |
| SMN Control-MOE-PS | Gold (13) | 197 |
| Monothiol | | |
| ASO 10-27-MOE-PS Dithiol | Gold (13) | 147 |
| SMN Control-MOE-PS Dithiol | Gold (13) | 155 |

SMA patient fibroblast cells (GM09677C) were obtained from Coriell Institute for Medical Research and cultured in EMEM medium containing 15% FBS. Fibroblasts were plated in a 96-well plate at a density of 10,000 cells per well. SNA-ASOs, Au—SNAs or linear cholesterol/distearyl oligonucleotides were added to the culture media in triplicate. After 48 hours of treatment the cells were collected for mRNA extraction. The levels of SMN2, SMN2Δ7, and total SMN2 mRNAs were measured by RT-PCR using assays from ThermoFisher Scientific. SMN2 mRNA, SMNΔ7 mRNA primer and probe sequences were: SMN2 mRNA forward primer: 5′-GCTG ATGCTTTGGG AAGTATGTTA-3′ (SEQ ID NO: 2), SMN2 mRNA reverse primer: 5′-CACCTTCCTTCTTTTTGATTTTGTC-3′ (SEQ ID NO: 3), SMN2 mRNA probe: 5′-6FAM-TACATGAGTGGCTATCATACTT-MGBNFQ-3′ (SEQ ID NO: 4), SMN2Δ7 mRNA forward primer: 5′-TGGACCACCAATAATTCCCC-3′ (SEQ ID NO: 5), SMN2Δ7 mRNA reverse primer: 5′-ATGCCAGCATTT CCATATAATAGCC-3′ (SEQ ID NO: 6) and SMN2Δ7 mRNA probe: 5′-6FAM-TACATGAGTGGCTATCATACT-MGBNFQ-3′ (SEQ ID NO: 7). Total SMN2 mRNAs were measured using a commercial gene expression assay (cat #Hs00165806_ml). Fold changes in SMN2 and SMN2Δ7 transcripts were calculated and normalized to untreated fibroblasts expression levels.

### Results

SNA-ASO, Au—SNAs and linear oligonucleotides consisting of the spinraza or control sequence were tested in SMA patient fibroblasts. SNAs were tested at 5, 1 and 0.2 μM, while linear cholesterol or distearyl oligonucleotides were tested at 1 μM. Fibroblasts were treated for 48 hours prior to processing.

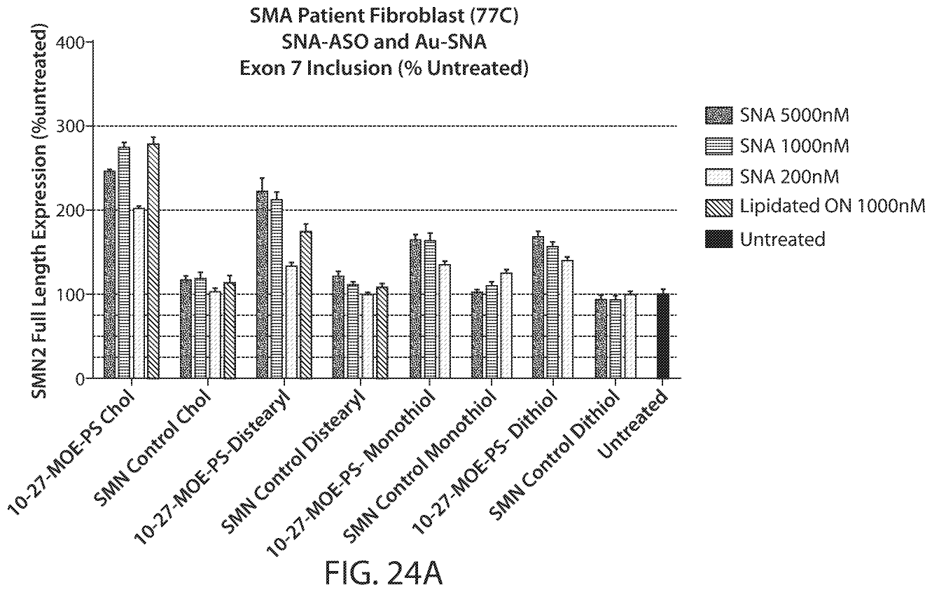

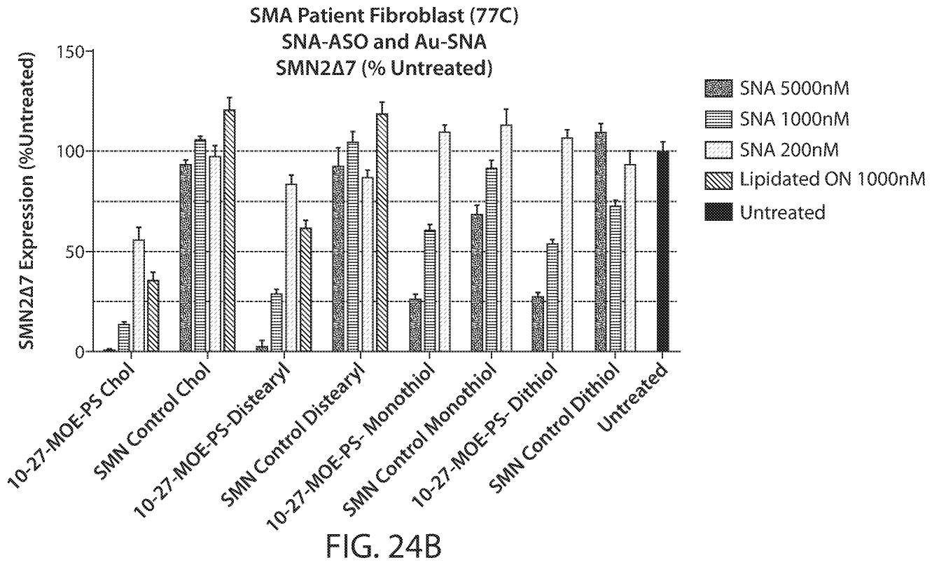

Data are included in FIG. 24A (SNA-ASO and Au-SNA: Full-length SMN2 mRNA) and FIG. 24B (SNA-ASO and Au-SNA: Δ7 SMN2 mRNA). All SNAs that contained the spinraza sequence showed SMN2 exon 7 inclusion and an associated SMN247 transcript reduction. In general, SNA-ASO outperformed Au-SNAs but compound efficacy varied. SNAs with 3′ distearyl or cholesterol showed approximately a 2-2.5-fold increase in full-length SMN2 mRNA relative to untreated at the highest concentration. In comparison, monothiol and dithiol Au-SNAs only produced a 1.5-fold increase. SNA-ASOs also showed greater reduction in SMN247 mRNA as expected. Linear versions of the cholesterol/distearyl oligonucleotides caused SMN2 exon 7 inclusion and Δ7 reduction, but showed reduced activity compared to SNAs. This is evident in FIG. 24B where greater SMN247 reduction was seen with the SNA compared to the linear oligonucleotides at 1 μM.

### Conclusion

It was previously shown that SNA-ASOs containing the spinraza sequence modified at the 3′ end with cholesterol were able to cause SMN2 exon 7 inclusion in patient fibroblasts. In the current study, a SNA-ASO containing the distearyl-modified oligonucleotide and two different Au-SNAs consisting of oligonucleotides covalently attached to gold nanoparticles also showed splice-switching activity. SNAs with distearyl or thiol modifications had different efficacies but the SNA with the cholesterol-modified oligonucleotide outperformed both. Differences in the bond strength between the oligonucleotide modification and SNA core may have played a role. This is the first indication that liposomal SNAs containing distearyl-modified oligonucleotides and gold SNAs are able to target the ISS-N1 region of the SMN2 pre-mRNA in the nucleus. Cholesterol-modified oligonucleotide consisting spinraza sequence also showed similar level of full-length SMN2 expression as the same oligonucleotide in SNA format. Surprisingly, the SNA version showed greater reduction in the Δ7 variant of SMN2 mRNA compared to cholesterol-modified oligonucleotide.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1. A spherical nucleic acid (SNA), comprising
a core and an antisense oligonucleotide comprised of 8 to 50 linked nucleosides in length targeted to a regulatory site of Survival of Motor Neuron 2 (SMN2) pre-mRNA, and wherein the antisense oligonucleotide is attached to the core and forms an oligonucleotide shell.

2. The SNA of claim 1, wherein the core has a minimal number mean diameter of about 8 nm.

3. The SNA of claim 1, wherein the core has a minimal number mean diameter of about 10 nm.

4. The SNA of claim 1, wherein the core has a minimal number mean diameter of about 15 nm.

5. The SNA of claim 1, wherein the core has a number mean diameter of about 10 nm to about 50 nm.

6. The SNA of claim 1, wherein the core has a number mean diameter of about 20 nm to about 25 nm.

7. The SNA of claim 1, wherein the core has a number mean diameter of about 20 nm.

8. The SNA of claim 1, wherein the core has a number mean diameter of about 10 nm to about 15 nm.

9. The SNA of claim 1, wherein the core has a number mean diameter of about 13 nm.

10. The SNA of claim 1, wherein the regulatory site is a ISS-N1 site.

11. The SNA of claim 1, wherein the regulatory site is a E1 site, a 3′ splice site of exon 8 site or a ISS+100 site.

12. The SNA of claim 1, wherein the core is a lipid bilayer containing core or liposomal core and the antisense oligonucleotide is attached to the lipid bilayer containing core or liposomal core.

13. The SNA of any one of claims 1-11, wherein the core is a metal core.

14. The SNA of any one of claims 1-11, wherein the core is a gold core.

15. The SNA of claim 14, wherein the antisense oligonucleotide is attached to the gold core through a covalent interaction.

16. The SNA of claim 1 or 12, wherein the antisense oligonucleotide is 18 nucleotides in length.

17. The SNA of claim 1 or 12, wherein the ISS-N1 site of the SMN2 pre-mRNA comprises a nucleic acid sequence of SEQ ID NO: 15.

18. The SNA of any one of claims 1-17, wherein less than all of the internucleoside linkages are phosphodiester.

19. The SNA of any one of claims 1-17, wherein the antisense oligonucleotide has phosphorothioate internucleoside linkages.

20. The SNA of claim 19, wherein less than all of the internucleoside linkages are phosphorothioate.

21. The SNA of any one of claims 1-20, wherein the antisense oligonucleotide has 2′O methyl modifications.

22. The SNA of claim 21, wherein less than all of the nucleotides include a 2′O methyl modification.

23. The SNA of any one of claims 1-22, wherein the antisense oligonucleotide is comprised of 18 to 21 linked nucleosides.

24. The SNA of any one of claims 16-23, wherein the antisense oligonucleotides of the oligonucleotide shell are directly attached to the lipid bilayer containing core.

25. The SNA of any one of claims 16-23, wherein the antisense oligonucleotides of the oligonucleotide shell are indirectly attached to the lipid bilayer containing core through a linker.

26. The SNA of claim 25, wherein the linker comprises a molecular species at the 3′ or 5′ termini of the antisense oligonucleotide, wherein the molecular species is positioned in a core and the antisense oligonucleotide extends radially from the core.

27. The SNA of claim 26, wherein the molecular species is linked to the antisense oligonucleotide at the 5′ end of the antisense oligonucleotide.

28. The SNA of claim 26, wherein the molecular species is a hydrophobic group.

29. The SNA of claim 28, wherein the hydrophobic group is selected from the group consisting of cholesterol, a cholesteryl or modified cholesteryl residue, a stearyl, a distearyl, tocopherol, adamantine, dihydrotesterone, long chain alkyl, long chain alkenyl, long chain alkynyl, olely-lithocholic, cholenic, oleoyl-cholenic, decane, dodecane, docosahexaenoyl, palmityl, C6-palmityl, heptadecyl, myrisityl, arachidyl, stearyl, behenyl, linoleyl, bile acids, cholic acid or taurocholic acid, deoxycholate, oleyl litocholic acid, oleoyl cholenic acid, glycolipids, phospholipids, sphingolipids, isoprenoids, such as steroids, vitamins, such as vitamin E, fatty acids either saturated or unsaturated, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen.

30. The SNA of claim 28, wherein the hydrophobic group is cholesterol.

31. The SNA of claim 28, wherein the hydrophobic group is distearyl.

32. The SNA of any one of claims 26-30, wherein the linker moiety comprises a non-nucleotidic linker moiety linked to the molecular species.

33. The SNA of claim 32, wherein the non-nucleotidic linker moiety is selected from the group consisting of an abasic residue (dSpacer), oligoethyleneglycol, triethyleneglycol, hexaethyleneglycol, polyethylene glycol, alkane-diol, or butanediol.

34. The SNA of claim 32, wherein the non-nucleotidic linker moiety is a double linker.

35. The SNA of claim 34, wherein the double linker is two oligoethyleneglycols.

36. The SNA of claim 35, wherein the two oligoethyleneglycols are triethyleneglycol.

37. The SNA of claim 35, wherein the two oligoethyleneglycols are hexaethyleneglycol.

38. The SNA of claim 34, wherein the double linker is two alkane-diols.

39. The SNA of claim 34, wherein the two alkane-diols are butanediol.

40. The SNA of any one of claims 34-39, wherein the double linker is linked in the center by a phosphodiester, phosphorothioate, methylphosphonate, or amide linkage.

41. The SNA of claim 32, wherein the non-nucleotidic linker moiety is a triple linker.

42. The SNA of claim 41, wherein the triple linker is three oligoethyleneglycols.

43. The SNA of claim 42, wherein the three oligoethyleneglycols are triethyleneglycol.

44. The SNA of claim 42, wherein the three oligoethyleneglycols are hexaethyleneglycol.

45. The SNA of claim 41, wherein the triple linker is three alkane-diols.

46. The SNA of claim 45, wherein the three alkane-diols are butanediol.

47. The SNA of any one of claims 41-46, wherein the triple linker is linked in between each single linker by a phosphodiester, phosphorothioate, methylphosphonate, or amide linkage.

48. The SNA of any one of claims 1-47, wherein the antisense oligonucleotides comprise the entire SNA such that no other structural components are part of the nanostructure and wherein the antisense oligonucleotide includes a molecular species and non-nucleotidic linker moiety that form the core, with the antisense oligonucleotides extending radially from the core.

49. The SNA of claim 48, wherein the SNA is free of lipids, polymers or solid cores.

50. The SNA of any one of claims 1-49, wherein the oligonucleotide shell has a density of 5-1,000 oligonucleotides per SNA.

51. The SNA of any one of claims 1-49, wherein the oligonucleotide shell has a density of 100-1,000 oligonucleotides per SNA.

52. The SNA of any one of claims 1-49, wherein the oligonucleotide shell has a density of 500-1,000 oligonucleotides per SNA.

53. The SNA of claim 12, wherein the lipid bilayer containing core is comprised of one or more lipids selected from: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof.

54. The SNA of claim 12, wherein the lipid bilayer containing core or liposomal core is comprised of DOPC.

55. The SNA of claim 54, wherein the ratio of number of oligonucleotide molecules to the diameter of the lipid bilayer containing core or liposomal core of DOPC in nm is 30:20.

56. The SNA of any one of claims 1-55 wherein the antisense oligonucleotide comprises or consists of
(SEQ ID NO: 1)
5′- TCA CTT TCA TAA TGC TGG - (Spacer 18)₂ -
3CholTEG.

57. A method for treating a subject having spinal muscular atrophy (SMA), comprising
administering to a subject having SMA a spherical nucleic acid (SNA) of any one of claims 1-56, in an effective amount to increase expression levels of SMN2 protein over a baseline level in the subject in order to treat the disorder.

58. The method of claim 57, wherein the baseline level is the level of SMN2 protein in the subject prior to treatment with the SNA.

59. The method of claim 58, wherein the baseline level is the level of SMN2 protein in a subject having SMA and treated with a linear antisense oligonucleotide targeted to ISS-N1 site of SMN2 pre-mRNA.

60. The method of claim 57, wherein the SNA is delivered by a route selected from the group consisting of intrathecal, oral, nasal, sublingual, intravenous, subcutaneous, mucosal, respiratory, direct injection, and dermally.

61. A method for treating a subject having spinal muscular atrophy (SMA), comprising
administering to a subject having SMA at least two doses of a spherical nucleic acid (SNA), in an effective amount to increase expression levels of Survival of Motor Neuron 2 (SMN2) protein over a baseline level in the subject in order to treat the disorder, wherein the second dose is administered about 3 months to 2 years after the first dose, and wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and wherein the antisense oligonucleotides are attached to the core and thus form an oligonucleotide shell.

62. The SNA of claim 61, wherein the regulatory site is a ISS-N1 site.

63. The SNA of claim 61, wherein the regulatory site is a E1 site.

64. A method of enhancing a level of exon 7-containing SMN2 mRNA relative to exon-deleted Survival of Motor Neuron 2 (SMN2) mRNA in a cell, comprising contacting the cell with an spherical nucleic acid (SNA) comprising a core and an antisense oligonucleotide comprised of 10 to 40 linked nucleosides in length targeted to a region of SMN2 pre-mRNA, such that the level of exon 7-containing SMN2 mRNA relative to exon 7-deleted SMN2 mRNA in the cell is enhanced.

65. The SNA of claim 64, wherein the core is a lipid bilayer containing core or liposomal core and the antisense oligonucleotide is attached to the lipid bilayer containing core or liposomal core.

66. The SNA of claim 64, wherein the core is a metal core.

67. The SNA of claim 64, wherein the core is a gold core.

68. The SNA of claim 67, wherein the antisense oligonucleotide is attached to the gold core through a covalent interaction.

69. The SNA of any one of claims 64-68, wherein the cell is a cell in connective tissue.

70. The SNA of any one of claims 64-68, wherein the cell is a spinal motor neuron.

71. The method of claim 58, wherein the antisense oligonucleotide comprises a sequence which is complementary to a portion of intron 7 of the SMN2 gene or the SMN2 pre-mRNA.

72. A spherical nucleic acid (SNA), comprising
a core and an antisense oligonucleotide comprised of 8 to 50 linked nucleosides in length targeted to a regulator of splicing of Survival of Motor Neuron 2 (SMN2) pre-mRNA, and wherein the antisense oligonucleotide is attached to the core and forms an oligonucleotide shell.

73. The SNA of claim 72, wherein the regulator of splicing of SMN2 pre-mRNA regulates inclusion of exon 7 in the SMN2 mRNA.

74. The SNA of claim 72, wherein the regulator of splicing of SMN2 pre-mRNA is an RNA binding protein.

75. The SNA of claim 74, wherein the RNA binding protein is RBM10.

76. The SNA of claim 72 or 73, wherein the regulator of splicing of SMN2 pre-mRNA is a serine/arginine (SR) splicing factor or a heterogeneous ribonucleoprotein (hnRNP) protein.

77. The SNA of claim 76, wherein the SR splicing factor is SRSF1, SRSF2, SRSF3, SRSF4, SRSF5, SRSF6, SRSF7 or SRSF11.

78. The SNA of claim 76, wherein the hnRNP protein is hnRNPA1, hnRNP A2B1, hnRNP C or hnRNP U.

79. The SNA of claim 72 or 73, wherein the regulator of splicing of SMN2 pre-mRNA is HuR/ELAVL1, Puf60, Sam68, SF1, SON, U2AF35 or ZIS2/ZNF265.

80. The SNA of any one of claims 1-20, wherein the antisense oligonucleotide has 2′O (2-methoxyethyl) modifications.

81. The SNA of claim 80, wherein less than all of the nucleotides include a 2′O (2-methoxyethyl) modification.

82. The SNA of any one of claims 1-20, wherein the antisense oligonucleotide has LNA modifications.

83. The SNA of claim 82 wherein less than all of the nucleotides include a LNA modification.

84. The SNA of any one of claims 1-20, wherein the antisense oligonucleotide has morpholino modifications.

85. The SNA of claim 84, wherein less than all of the nucleotides include a morpholino modification.

86. A method for treating a subject having spinal muscular atrophy (SMA), comprising
administering to a subject having SMA a spherical nucleic acid (SNA) comprising a core and an antisense oligonucleotide comprised of 8 to 50 linked nucleosides in length targeted to a regulator of splicing of Survival of Motor Neuron 2 (SMN2) pre-mRNA, and wherein the antisense oligonucleotide is attached to the core and forms an oligonucleotide shell, in an effective amount to increase expression levels of SMN2 protein over a baseline level in the subject in order to treat the disorder.

87. The method of claim 86, wherein the baseline level is the level of SMN2 protein in the subject prior to treatment with the SNA.

88. The method of claim 86 wherein the baseline level is the level of SMN2 protein in a subject having SMA and treated with a linear antisense oligonucleotide targeted to ISS-N1 site of SMN2 pre-mRNA.

89. The method of claim 86, wherein the subject has an increased survival rate relative to an average survival rate of a subject treated with a linear antisense oligonucleotide targeted to ISS-N1 site of SMN2 pre-mRNA.

90. The method of any one of claims 86-89, wherein the subject is administered a dose of oligonucleotide of greater than 12 mg/5 ml.

91. The method of any one of claims 86-89, wherein the subject is administered a dose of oligonucleotide of 15-20 mg/5 ml.

92. A spherical nucleic acid (SNA), comprising
a core and a first antisense oligonucleotide comprised of 8 to 50 linked nucleosides in length targeted to a regulatory site of Survival of Motor Neuron 2 (SMN2) pre-mRNA, and a second antisense oligonucleotide comprised of 8 to 50 linked nucleosides in length targeted to a region of a lncRNA, and wherein the antisense oligonucleotides are attached to the core and form an oligonucleotide shell.

93. The SNA of claim 92, wherein the core has a minimal number mean diameter of about 8 nm.

94. The SNA of claim 92, wherein the core has a minimal number mean diameter of about 10 nm.

95. The SNA of claim 92, wherein the core has a minimal number mean diameter of about 15 nm.

96. The SNA of claim 92, wherein the core has a number mean diameter of about 10 nm to about 50 nm.

97. The SNA of claim 92, wherein the core has a number mean diameter of about 20 nm to about 25 nm.

98. The SNA of claim 92, wherein the core has a number mean diameter of about 20 nm.

99. The SNA of claim 92, wherein the core is a lipid bilayer containing core and the antisense oligonucleotide is attached to the lipid bilayer containing core.

100. The SNA of any one of claims 92-99, wherein the lncRNA is SMN-AS1.

101. The SNA of any one of claims 92-99, wherein the second antisense oligonucleotide is selected from SEQ ID NO: 81 to SEQ ID NO: 160.

102. The SNA of any one of claims 92-99, wherein the second antisense oligonucleotide is selected from oligonucleotides having 85%, 90%, 95%, 96%, 97%, 98%, or 99% sequence identity with oligonucleotides of SEQ ID NO: 81 to SEQ ID NO: 160.

103. The SNA of any one of claims 92-99, wherein the second antisense oligonucleotide has a 5-10-5 MOE gapmer design, wherein the central gap segment comprises of ten 2′-deoxynucleosides and is flanked by wing segments on the 5′ direction and the 3′ direction comprising five nucleosides each.

104. The SNA of claim 103, wherein each nucleoside in the 5′ wing segment and/or each nucleoside in the 3′ wing segment has a 2′-MOE modification.

105. The SNA of claim 103, wherein the internucleoside linkages throughout each gapmer are phosphorothioate (P═S) linkages.

106. The SNA of claim 104, wherein the gapmers have mixed backbone, including phosphorothioate and phosphodiester internucleotide linkages.

107. The SNA of claim 104, wherein one or more or all cytosine residues throughout each gapmer are 5-methylcytosines.

108. A method of increasing expression of full length SMN2 mRNA in a cell comprising contacting the cell with the SNA from any one of claims 92-107.

109. A method of increasing expression of full length SMN2 mRNA in a cell comprising, contacting the cell with an spherical nucleic acid (SNA) comprising a core and an antisense oligonucleotide comprised of 10 to 40 linked nucleosides in length targeted to a region of SMN2 pre-mRNA and another SNA comprising a core and an antisense oligonucleotide comprised of 10 to 40 linked nucleosides in length targeted to a region of SMN-AS1.

110. The SNA of claim 109, wherein the core is a lipid bilayer containing core or liposome core and the antisense oligonucleotide is attached to the lipid bilayer containing core or liposomal core.

111. The SNA of claim 109, wherein the core is a metal core.

112. The SNA of claim 109, wherein the core is a gold core.

113. The SNA of claim 112, wherein the antisense oligonucleotide is attached to the gold core through a covalent interaction.

114. The SNA of any one of claims 109-113, wherein the cell is a cell in connective tissue.

115. The SNA of any one of claims 109-113, wherein the cell is a spinal motor neuron.

116. The SNA of claim 26, wherein the molecular species is linked to the antisense oligonucleotide at the 3′ end of the antisense oligonucleotide.

117. A method for delivering a stable level of therapeutic oligonucleotides to a central nervous system (CNS) of a subject, wherein the method comprises
administering to the subject a spherical nucleic acid (SNA) wherein the SNA comprises a core and therapeutic oligonucleotides comprised of 10 to 60 linked nucleosides in length, wherein the therapeutic oligonucleotides are attached to the core and thus form an oligonucleotide shell,
wherein the SNA is administered in an effective amount to deliver a stable level of the therapeutic oligonucleotide to the CNS of the subject
wherein the stable level of the therapeutic oligonucleotides is achieved when at least 50% of the therapeutic oligonucleotides are present in a tissue of the CNS within seven days of administration of the SNA to the subject, relative to the amount of therapeutic oligonucleotides present in the tissue of the CNS within one hour of administration of the SNA to the subject.

118. The method of claim 117, wherein the SNA is administered intrathecally (IT).

119. The method of claim 117 or 118, wherein the SNA is administered in the lower lumbar region.

120. The method of any one of claims 117-119, wherein the SNA is IT-administered through a lumbar puncture.

121. The method of any one of claims 117-120, wherein the subject is a mammal.

122. The method of any one of claims 117-120, wherein the subject is a rat.

123. The method of any one of claims 117-120, wherein the subject is a human.

124. The method of any one of claims 117-123, wherein a stable level is achieved when at least 50% of the therapeutic oligonucleotides are present in a tissue of the CNS within three days of administration of the SNA to the subject, relative to the amount of therapeutic oligonucleotides present in the tissue of the CNS within one hour of administration of the SNA to the subject.

125. The method of any one of claims 117-123, wherein a stable level is achieved when at least 50% of the therapeutic oligonucleotides are present in a tissue of the CNS within 48 hours of administration of the SNA to the subject, relative to the amount of therapeutic oligonucleotides present in the tissue of the CNS within one hour of administration of the SNA to the subject.

126. The method of any one of claims 117-123, wherein a stable level is achieved when at least 50% of the therapeutic oligonucleotides are present in a tissue of the CNS within 24 hours of administration of the SNA to the subject, relative to the amount of therapeutic oligonucleotides present in the tissue of the CNS within one hour of administration of the SNA to the subject.

127. The method of any one of claims 117-126, wherein the therapeutic oligonucleotide is an antisense oligonucleotide comprised of 10 to 40 linked nucleosides in length targeted to a regulatory site of Survival of Motor Neuron 2 (SMN2) pre-mRNA.

128. The method of claim 127, wherein the level of exon 7-containing SMN2 mRNA relative to exon 7-deleted SMN2 mRNA in the subject is enhanced.

129. The method of any one of claims 117-128, wherein less than 50% of the therapeutic oligonucleotides are detectable within six hours of administration to the subject in one or both kidneys of the subject.

130. The method of any one of claims 117-128, wherein less than 40% of the therapeutic oligonucleotides are detectable within six hours of administration to the subject in one or both kidneys of the subject.

131. The method of any one of claims 117-128, wherein less than 30% of the therapeutic oligonucleotides are detectable within six hours of administration to the subject in one or both kidneys of the subject.

131. The method of any one of claims 117-128, wherein less than 20% of the therapeutic oligonucleotides are detectable within six hours of administration to the subject in one or both kidneys of the subject.

132. The method of any one of claims 117-128, wherein less than 10% of the therapeutic oligonucleotides are detectable within six hours of administration to the subject in one or both kidneys of the subject.

133. The method of any one of claims 117-128, wherein less than 5% of the therapeutic oligonucleotides are detectable within six hours of administration to the subject in one or both kidneys of the subject.

134. The method of any one of claims 117-134, using the SNA of any one of claim 1-50, 80-95 or 98.

135. The method of any one of claims 117-135, wherein the SNA is in a formulation and wherein the formulation comprises artificial cerebral spinal fluid (aCSF).

136. A method for delivering a stable level of therapeutic oligonucleotides to a central nervous system (CNS) of a subject having spinal muscular atrophy (SMA), wherein the method comprises
administering to a subject having SMA a spherical nucleic acid (SNA) in an effective amount to deliver therapeutic oligonucleotides to the brain of the subject,
wherein the administration of SNA delivers about 2% to about 150% more therapeutic oligonucleotides to one or more tissues or regions of the CNS of the subject than administration of linear therapeutic oligonucleotides that are not in a SNA,
wherein the SNA comprises a core and therapeutic oligonucleotides comprised of 10 to 60 linked nucleosides in length, wherein the therapeutic oligonucleotides are attached to the core and thus form an oligonucleotide shell.

138. The method of claim 137, wherein the one or more tissues or regions of the CNS is one or more regions of the brain.

139. The method of claim 138, wherein the one or more regions of the brain is selected from the group consisting of the amygdala, basal ganglia, cerebellum, corpus callosum, cortex, hippocampus, hypothalamus, midbrain, olfactory region, one or more ventricles, septal area, white matter and thalamus.

140. The method of claim 137, wherein the one or more tissues or regions of the CNS are the cervical cerebral spinal fluid (CSF) or thoracic CSF.

141. The method of any one of claims 137-140, wherein the therapeutic oligonucleotides in the SNA and the linear therapeutic oligonucleotides that are not in a SNA have different routes of distribution and clearance.

142. A method for treating a subject having spinal muscular atrophy (SMA), the method comprising
administering to the subject having SMA a spherical nucleic acid (SNA) in an effective amount to increase the expression level of survival of motor neuron 2 (SMN2) protein over a baseline level of SMN2 protein in the central nervous system (CNS) of the subject to treat SMA,
wherein the effective amount of SNA is greater than 12 mg/dose, and
wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and
wherein the antisense oligonucleotides are attached to the core and thus form an oligonucleotide shell.

143. A method for treating a subject having spinal muscular atrophy (SMA), the method comprising
administering to the subject having SMA a spherical nucleic acid (SNA) in an effective amount to increase the expression level of survival of motor neuron 2 (SMN2) protein over a baseline level of SMN2 protein in the central nervous system (CNS) of the subject to treat SMA,
wherein the effective amount of SNA is less than 12 mg/dose, and
wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and
wherein the antisense oligonucleotides are attached to the core and thus form an oligonucleotide shell.

144. A method for treating a subject having spinal muscular atrophy (SMA), comprising
administering to a subject having SMA at least two doses of a spherical nucleic acid (SNA) in an effective amount to increase expression levels of survival of motor neuron 2 (SMN2) protein over a baseline level in the subject in order to treat SMA, wherein the second dose is administered about 15 days to about three months after the first dose, and
wherein the SNA comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced, and wherein the antisense oligonucleotides are attached to the core and thus form an oligonucleotide shell.

145. The method of claim 61, wherein the second dose is administered about two years after the first dose.

146. The method of claim 61, wherein the second dose is administered about 1.5 years after the first dose.

147. The method of claim 61, wherein the second dose is administered about one year after the first dose.

148. The method of claim 61, wherein the second dose is administered about six months after the first dose.

149. The method of claim 61, wherein the second dose is administered about four months after the first dose.

150. The method of claim 144, wherein the second dose is administered about three months after the first dose.

151. The method of claim 144, wherein the second dose is administered about two months after the first dose.

152. The method of claim 144, wherein the second dose is administered about one month after the first dose.

153. A structure, comprising an antisense oligonucleotide comprised of 8 to 50 linked nucleosides in length targeted to a regulatory site of Survival of Motor Neuron 2 (SMN2) pre-mRNA and a linker comprising a molecular species at the 3′-end or the 5′-end of the antisense oligonucleotide, wherein the linker comprises two oligoethylene glycols.

154. The structure of claim 153, wherein the oligoethylene glycol is a hexaethylene glycol.

155. A structure, comprising an antisense oligonucleotide comprising the nucleotide sequence 5′-TCACTTTCATAATGCTGG-3′ (SEQ ID NO: 172) or the nucleotide sequence 5′-Tes mCes Aes mCes Tes Tes Tes mCes Aes Tes Aes Aes Tes Ges mCes Tes Ges Ge-3′ (SEQ ID NO: 16) and a linker at the 3′-end or the 5′-end of the antisense oligonucleotide comprising two oligoethylene glycols and a cholesterol.

156. The structure of claim 155, wherein the oligoethylene glycol is a hexaethylene glycol.

157. A structure, comprising an antisense oligonucleotide comprising or consisting of the nucleotide sequence 5′-TCA CTT TCA TAA TGC TGG-(Spacer 18)2-3CholTEG (SEQ ID NO: 1) or the nucleotide sequence moeT*/5-Me-moeC/*moeA*/5-Me-moeC/*moeT*moeT*moeT*/5-Me-moeC/*moeA*moeT*moeA*moeA*moeT*moeG*/5-Me-moeC/*moeT*moeG*moeG/isp18//isp18//3CholTEG/(SEQ ID NO: 164).

158. A structure, comprising an antisense oligonucleotide comprised of 8 to 50 linked nucleosides in length targeted to a regulatory site of Survival of Motor Neuron 2 (SMN2) pre-mRNA and a linker comprising a molecular species at the 3′-end or the 5′-end of the antisense oligonucleotide, wherein the molecular species is a hydrophobic group comprising a stearyl.

159. The structure of claim 158, wherein the stearyl is a distearyl.

160. A method for treating a subject having spinal muscular atrophy (SMA), comprising
administering to a subject having SMA a structure of any one of claims 153-158 in an effective amount to increase expression levels of SMN2 protein over a baseline level in the subject in order to treat the disorder.

161. A method for treating a subject having spinal muscular atrophy (SMA), comprising
administering to a subject having SMA at least two doses of a structure in an effective amount to increase expression levels of Survival of Motor Neuron 2 (SMN2) protein over a baseline level in the subject in order to treat the disorder, wherein the second dose is administered about 3 months to 2 years after the first dose, and wherein the structure comprises a core and an antisense oligonucleotide comprised of 10 to 40 linked nucleosides in length targeted to a regulatory site of SMN2 pre-mRNA, such that a level of exon 7-containing SMN2 mRNA relative to exon 7-deleted SMN2 mRNA in the subject is enhanced.

162. A method of enhancing a level of exon 7-containing SMN2 mRNA relative to exon-deleted Survival of Motor Neuron 2 (SMN2) mRNA in a cell, comprising contacting the cell with a structure of claims 153-158, such that the level of exon 7-containing SMN2 mRNA relative to exon 7-deleted SMN2 mRNA in the cell is enhanced.

163. A structure, comprising
an antisense oligonucleotide comprised of 8 to 50 linked nucleosides in length targeted to a region of a lncRNA, wherein the structure comprises a linker.

164. A method of increasing expression of full length SMN2 mRNA in a cell comprising, contacting the cell with structure comprising an antisense oligonucleotide comprised of 10 to 40 linked nucleosides in length targeted to a region of SMN2 pre-mRNA and contacting the cell with another structure comprising an antisense oligonucleotide comprised of 10 to 40 linked nucleosides in length targeted to a region of SMN-AS1.

165. A method for delivering a stable level of therapeutic oligonucleotides to a central nervous system (CNS) of a subject, wherein the method comprises
administering to the subject the structure of any one of claims 153-158 in an effective amount to deliver a stable level of the therapeutic oligonucleotide to the CNS of the subject
wherein the stable level of the therapeutic oligonucleotides is achieved when at least 50% of the therapeutic oligonucleotides are present in a tissue of the CNS within seven days of administration of the structure to the subject, relative to the amount of therapeutic oligonucleotides present in the tissue of the CNS within one hour of administration of the structure to the subject.

166. A method for treating a subject having spinal muscular atrophy (SMA), the method comprising
administering to the subject having SMA the structure of any one of claims 153-158 in an effective amount to increase the expression level of survival of motor neuron 2 (SMN2) protein over a baseline level of SMN2 protein in the central nervous system (CNS) of the subject to treat SMA,
wherein the effective amount of structure is greater than 12 mg/dose, such that a level of exon 7-containing SMN2 mRNA relative to exon 7-deleted SMN2 mRNA in the subject is enhanced.

167. A method for treating a subject having spinal muscular atrophy (SMA), the method comprising
administering to the subject having SMA a structure of any one of claims 153-158 in an effective amount to increase the expression level of survival of motor neuron 2 (SMN2) protein over a baseline level of SMN2 protein in the central nervous system (CNS) of the subject to treat SMA,
wherein the effective amount of structure is less than 12 mg/dose, such that a level of exon 7-containing SMN2 mRNA relative to exon 7-deleted SMN2 mRNA in the subject is enhanced.

168. A method for treating a subject having spinal muscular atrophy (SMA), comprising
administering to a subject having SMA at least two doses of a structure of any one of claims 153-158 in an effective amount to increase expression levels of survival of motor neuron 2 (SMN2) protein over a baseline level in the subject in order to treat SMA, wherein the second dose is administered about 15 days to about three months after the first dose, such that a level of exon 7-containing SMN2 mRNA relative to exon 7-deleted SMN2 mRNA in the subject is enhanced.


SEQUENCE LISTING (173 sequences)

SEQ ID NO: 1 - DNA, 18 bp, Artificial Sequence
  Synthetic Polynucleotide
  misc_feature (18)..(18): modified by /iSp18//iSp18//3CholTEG/
tcactttcataatgctgg

SEQ ID NO: 2 - DNA, 24 bp, Artificial Sequence
  Synthetic Polynucleotide
gctgatgctttgggaagtatgtta

SEQ ID NO: 3 - DNA, 25 bp, Artificial Sequence
  Synthetic Polynucleotide
caccttccttctttttgattttgtc

SEQ ID NO: 4 - DNA, 22 bp, Artificial Sequence
  Synthetic Polynucleotide
  misc_feature (22)..(22): modified by MGBNFQ
tacatgagtggctatcatactt

SEQ ID NO: 5 - DNA, 20 bp, Artificial Sequence
  Synthetic Polynucleotide
tggaccaccaataattcccc

SEQ ID NO: 6 - DNA, 25 bp, Artificial Sequence
  Synthetic Polynucleotide
atgccagcatttccatataatagcc

SEQ ID NO: 7 - DNA, 21 bp, Artificial Sequence
  Synthetic Polynucleotide
  misc_feature (1)..(1): modified by 6FAM
  misc_feature (21)..(21): modified by MGBNFQ
tacatgagtggctatcatact

SEQ ID NO: 8-9 - skipped (2 entries)

SEQ ID NO: 10 - DNA, 77 bp, Artificial Sequence
  Synthetic Polynucleotide
ctatatatagatagctttatatggatgttaaaaagcattttgtttcacaagacattttacttattttattcaacaaa

SEQ ID NO: 11 - skipped

SEQ ID NO: 12 - DNA, 28073 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccaggtgaggtcgcagccagtgcagtctccctattagcgctctcagcacccttcttccggcccaactctccttccgcagcctcgggacagcatcaagtcgatccgctcactggagttgtggtccgcgtttttctacgtcttttcccactccgttccctgcgaaccacatccgcaagctccttcctcgagcagtttgggctccttgatagcgttgagtggaggccctgccgcgacttggcagtagcttattttgttcactcctctctggctggtgtgggggaggtgggggcattaggccagggtgaagcaggggaaccacttaggagtctgttaagatgatctgaacttcagaacaagatgttattaacagagtgaaagtatttggattctgggtatattttgaaatcggaggcaacaggtttttcagatagattcgataacggaggttatcctgaatagttgaaaagataaagttgccttttgctgaggtgggaaagagaagattgccagtagagcaggtttctcaggagttcagtcttgggcatagcatggtaggggtgaatttggctggagtgagttggagagtaggagaagagaaatccaaggcaacatttgaccagcctgggcaacatagtgtgactccgagtctgcaaaaattagacgggtgttgtggtgcgcgtctgtggtctcagctacctggaaggttcaggccttggaaggctcagggaggtggaggctgcagtgatctgtgattgcgcctctgcactccagcctgggcgacagagccagaccctgtcttaaaacaaaataaacggccgggcgcggtggctcaagcctgtaatcccagcactttgggaggccgaggcggccggatcacaaggtcaggagatcgagaccatcctggctaacacggtgaaaccccgtctctactacaaatacaaaaaattagccgggcgtggtgacgggcgcctgtagtcccagctactcgggaggctgaggcaggagaatgtcatgaagccgggaggcggagcttgcagtgagccgagatcgcgccactgcactccagcctgggcgatagagcaagactccgtctcaaataaataaataaataaataaataaataataaaaacatcggtaggcatatttcaaggaattctatttaaaaaaaatttttttagagacaagttcgctctctgtggcccaggctggagtacagtggcatgatcctagcccatggcagcgttgatctcttggcctcaagcgaccctcctttggagtcgctgggcctaaaggagtgagccaccacgaaattttattataaatggagggtagagaaattgggcaataaatggagggggaagtgagttaagaggaattttaattatgtgtgtgtggttttaaaagaggggggtcttgctctgttgcccaggctgctggggtgccagtggcgcaatcatgaatcactacagccttggactcctggcctcaagctatcctcccacctctgcctcccaaagtactgggattactagtgtgagccactgcactaagataggagcaacatgtttcagcatgtttgtgggttgataggaaagatgagaatgggaaagttgatgtcggaaagaagacaatggctagagcaatgtcctagagtaggtaagaagggatggatttggcctttgttggaaacattagcggttcttttggtgacagctatatagttaacacatctatgatacgtgaatgggcagataggatggcaggagattttgaaagttctcttgattcttactgttctcttagtgaaagaagcaaggttatcagctagaagctgggatgggagaggaaagagaagatgggaagtagatagttctttagaagagtgggcaagggttggactagggaagtttagtggaaatattgctaggcaacataaagagcctacttgagattcgtggtcatgagttgaaggagaccagacagcaagattgtgtatgagggcacccacagagtaaatggagagttgaaattaatgcagttgtgattttaccacgtggatatgaagaagtgagggggagaagtacaaaggagttctcttaatgattgaccatggaatttaagctggctaagaaaggaagtgagaggccgggcgcggtggctcacgcctgtaatcccagcactttgggagactgaggtgggtggattacctgaggtcaggagtttgagaccaacctggccgatatggcgaaaccccatctctaataaaaatacagaaaaattagccgggaatggtggcaggtgcctgtaatcccagctactcaagaggctgtggcaggagtatcccttggacccaggaggtggaggttgcagtgagccgagatcacgccactgtactccagcctggacgatatagtgagacttcacctcaaaaaaaaaaaaaaagaaaggaagtgaggattttaagaccctgagagacagtttaaaaagtgggaggatcggccgggcgctgtggctgacacctgtaatcccagcactttgggaggccgagttgggcagatcacaaggtcaggagttcgagaccagcctggccaatatggtgaaaccttgtctctactaaaaatacaaaaattagccgggcatggtgtcacgtgtctataatcccagctactcgggaggctgaggcagaaaaattgcttgaacctgggaggcagaggttgcagacagctgagatcactccattgcactccagcctgggcaacaagagcaaaactttgtctttaaaaaaaaaaaaaaaaaaagaatacaaaaattagccgggcgtggtggcgcgtgcctataatcccagctacttgggaggctgaggcaggagaatcagttgaacacgggaggcgaggtttgcagtgagccgagattgcgccactgcactccagcctgggcgacagagcaggactcctcttggaaaaaaaaaattagctgggcatggtggcaggtgcctgtagtctcagctactagggaggctgaggcaggaaaatcacttgaacccgggatgtggagtttgcagtgacccgagatcgtgccactgtactccatcctgggcgacaaaatgagactctgcctcaaaaaaaaaaaaaaaaaaaagtgggaggatcaatgtactgccagtcctaatgaagtggaatgattgtccccatcaaatcactagtaggagtaagttgcagagcctagaaggtgatggttaagagagtgggattcttgaaactgcatttatggagaggttgtggttattggttataataaataaatacagttgaagtgagtgagtagctgagatttggggatgtatcagttcattcttacactgctacaaagacatacctgagaccaggtatttataaagataagaggtttaatcagctcacagttctgctgcctgtacaggcttctcttgtggaggcctaaggaaacttacagtcatggtggaaggtgaaggggaaacaagcacagtcttcacatggccagcaggagagagagagaagggggaagtgctacatactttaaaacaaccagatcttgtgagaacgcttatcaggaaacagcacttggggatggtgctaaatcattagaaatcacccccatgatccagtcgcctcctaccatgcccacctccaacactggggatcacaattcagcatgagatttgggtaggaacacagagctgcaccacatcagaggatgtacaagattgtggtggagaggagtttagagacctgcaaatatagggtaattgaagggatcatctacatggatatttaaatcaccaaaaattatgacaggagtagtgttggagagagaactgcgatgtaaacattaaggaatgaggaagagtgactcggtaggctgtaggtgactgcaataggaaacgataatagactgtgagtctggtgacaagattttccttctttctttttttccccccccccgagacagggcctctttttgttgcccaggtgggagtgcagtggcgcgatcacggctcactacaacctcctcccaagctcaagggattctcccacttcagcctctcaagtagctggaactacaggtgctgaccaccatgcctggctactttttgtcaggattttcaaggctgggaattttgagaggggaatggaggagaataatctgaaagtgcaagtaaggagcagggaagatttcttttttcttttttttttttttttttgagtcggagtctggctcagtcgcccaggctggagtgcagtggcgagatctccgctcactgcaagctccgcctcccgtgttcacgccattctcctccttcagcctcccgagtagctgggactacaggcgcccgccaccacgcccagctaattgtttttttgtatttttagtagagacggggtttcaccgtgttagccaggatggtctcaatctcctgactttgtgatccgcccaccccggcctcccaaagcgcttgggattacaggcgtgagccaccgcgccagccagagcagggaagatttcttccccacatctccagtaggtacagtgatatgaagtgtgtggaggagaaaagaggaaacatctatcatttgagatggctgcgaaaggaaaaggcatcctcagggagctagattttacttagagcaagaaatgaagggatgattcagaggttaaaagagtggattttatgaattactcaagggagcacagtggaagtttcaggaagtggtaggagaaggtagaagatggcagggtgttgggaataatttgagaaatctgagctactggaaatgactgagaatcagatataaaggcagtcctggtggtccgttctggctgccgttgctgtgtaacgaatctgccaaaacttagtggcttgaaacaacaaagaacattttattatctctcattgtttctgtgggttaggaatttgtgagagccgtgctgggcagttttcgtgcggctgtctcgtggttgcacctacatagttgctagagctacagtagctggggactgagcagctagggattggcaggctatctctttttttcatgtagtctcatgaagatttctttatgtggtttcaatgtgtgggctggtttggatttccttatagcatggtggcctcagttggattgctgttttgtgatccttttcatccctccttgtcctgtccccagacaaccactgatctactttctgtcaccatagattagcctgcatttttaagaatttttataaacgtggaatgatagagtaccttttttgtcacgtttcttttatttatcatagctattttgattttcatccattttattgctgagtagtatcccattgcatgtatatactatactgtattcattcgcttgcttgtgaacatttgggctttttccagtttgggactgttaacaagtagagccactatgaatattagtgtataagacttcatatagccaaggctggcagatcgcttgagcccaggagtttgagaccagcctgggaaacatggtgaaacctctatttttattttaaaatcaaaaattaaaaattttctataaaaaattttaaagaagactttgtatagacatacgctttcatttttcttgagtgaatacttaggtctcagggtagatgtattttaagtctttaaggagctgtcaaactcttcctcaaagtggtggttgtaccatgttactttttaatataacagagattaattgagcaaagaaaaattcaaaagttggacagcccccacaactaaataggttcagaacagctcccccattttgcattttgaccagcaatgtatgaaagttccatttgctcagtgtccctgcaaacacctggtatggtcagtctttttaattttaggcattataatagatatagtggcttcttgtgattttaattagcatttcctaatgaccagtgctgctgttgatcatttcatgagtgtatttgccatccgtatatcttttttggtgaagtgtctattcaaatcatttgggttttttttttttttgttttttttttttggagacagtgtctcactctgtcacccaggctgttgtgcagtggtgcaatcacacagcctactgcagcctccacctcctgcgctcagtcttcttgtctcagccttctgagtagctgaaattacgagcacacgccacaatgcctggctaattttttaaaattttgtagaaacaaggtctcattatgttgcctgggcttgtcgtgaactcctgggctcaagcaatcttcctgcctcagcctcccaaagattgggattgcaagtatgagccactgcacccggccaacttacccatcttttaattgaatttttttgttgttgaggtttgagagttcttcatgtttgctgggtacaatatctttatcagataggtaacttgcatgtattttctcccggtttacactttggtttttcattttgttaacaacgtctttttaagaacagaaaatcttaattttgctgaaatctaatttttcagttttttctttgatggttttgagagaggaggtaaaaaaagactaggtaagccgatagttagacagagtcctcggtagaacttcccttctaacaaaaagcagcccaagaaatcacttctcttctaacaaggagcagcctggaagatcgggctgtaaacatgtataaggaagcagctctggcacagagggggagcttcctgggtaatcagcaagcttcacatacgtaaggtgggtatgtgaagtaaacacagtatgtgaagtaaacacagtggaccttagtacatactcagataaggaagctggaagcttgcatgttgtgagttgttggggttgcctgcagctgcacggagagaaaggggtacctggggccaggcatgtccaccatggtggctccacctccccttatttagcacatgcacaataggaaagagataagcaatgtggagtagctcaggccaaggacctgcctgcataataaaaggttggggtgggggatgccagagattcacgctctgtgcagatggcaacacctggtcctaactggttttttgctccctatgtgtagataagctacccccttcccattagctcatttataaaaatgcttgcatttcactgtggaatgggaactcttttcaggacctctctctgcaggagagagctagtctctttcttttgcctattaaacttctgctctagcctcacacccttggtgtgtcagcgtccttgatttcctcagcgtgagaccaagaacctcgggtgccaccccaggcaacaaggccatttcagtttgttcttttgttataggcaatccatgatcacagatttttctctcttttttttttttacacagtttagagttttagttttacacttaggtctgtaatccattttgtattaattcttatatgtggctcagtgtaggtggaaatttggtttgtttttgcataaggatttccaatagttttaccaccatttcttgaaactactatgctttctctattaaaccacatttgtaactttagttaaaatcagtcacatatatcacagggctatttctgactctcaattctgttacattgtctattagtgtatattgatgtcagtactacacttttaattactattgcttcagggtatgtcttgtaaaccaaaaataaaattataggccccccccgcccctgcacaaccaactgaatggacccatcctctcagccaagggcattccaaaattaacctgaaaaactagttcaagccatgatgggaagggggagttggacatgtctcatcacaccctactaccttttggaattactgatagaacagactcttaaagtctgaaaagaaacatttacaacctaccctctctgaagcctgctacctgggagcttcatctgcatgataaaaccttggtctccacaaccccttatggtaacccaaacattcctttctgttgataataactctttcaactagttgccaattagaaaatctttaaatcttcctatgacctagaaacctccctacccccactttgagttgtcctgcctttcctgacagaactcatgtacatcttacatatattgattgatgcctcatgtctccctaaaatgtataaaacaaagctgtaccccaccaccttggggacatgtcatcaggacctcctgtggctgtgtcataggagcgtctttaactttggcaaaataaactttctaaattgattgaaacctgtcttagctacttctggtttacagtcttaaagttagataatgtaaattgtccagctttggtttatttttgtccttagtagttccatataaattttagaatcagcttttcaatttaatacactactttcctcttagatccacaattaaatatatttgatgctaacaattctgttttatgtttttcgttttttttttttgagacaagagtttcgctcttgttgcccaggctggagtgcagtggcgcgatcttggctcaccacaacctccacctcccaggttcaagcaattcttctgcctcagcctcccgagtagctgggattacaggcatgcgccaccacgcccggctaattttgtatttttagtagagacggggtttcaccatgttgatcaggctggtcttgaactcctgacctcaggtgatccacccacctcggcctcccaaagtgttgggattacaggcgtgaaccaccatgcctggccagttctgttatttttaaaacccaagtttccctggtcatatcttggttggatgaagcgtattttcaatagattaccctggaaaggctagtgagtacggtattcttctacattttagacttttcttagtcttgctacttcaaggacagctaggctgcatataaaattcttggctcatactttttccccataaatttctatgagaaagtctaatgataactgattttctttattttgtaacttagtctttttgcttagaggctctctgaggatgggagggggttcttcctcccatccctaggaatttttcttttttttaaattcctaatcactagaccaccaggaagattgtttgttttgttttgtttttattcttcagggaccccatttatacatacgttaaataaatactgtttgccaatgtatcaaccattttgcttcttatttatttttgttcctttggttctttttcatggctttgctttggtgctccttagattttcagtcagatgtatttgtccttgggtaccttgtaatcagtattaccttttcttctgtcgctttgttttctgttcgttttgaaattacttgtttcctggtctggcaataacagttgagatatgaggagtttgagctgccatctgtctatgtatcttgctttaagactgcactcttctattgatatcactggccttgattttgtgatttctttatttcttcaggaccacccttcattttctactgtttgcttcctttttttttgagatggagtctcactctgtcactcaggctggagtgcagtgatcttggctcattgcaacctctgcctcccgggttccagcaattctcctgcctcagcctcccaagtatctgggactacaggtgtgcaccaccatgcccggctaagttttgtatttttaatagagacggggttttgccacattggcaggctggtctcaaactcctgatgtcaagtgatccacccaccccacccacctctgcatcccaaagtgctgggattacaggaatgagctgccgtgcccagcctcccccctacccccctttttttctttcgagacagagattataggtgtgagccactggacccagcctgtttttattccttttaccaaatctccaaggaatatcttcccttccaagtgcgaatgtaaccttaagtcagttaacctctttgtgattacttttcttatctgcaaagtgacttaatgatcttaagtactttttttttttgagacagggtctcactgtcaccctggctggagtgcagtggcacgatctctgatctccactcactgcaatctcctcttccctggttcaagcggccctcccaccttagccttctgggtagctgggactacagatgtgaaccaccacgcccagctaatttttgtactttttgtagagatggggttttgccatgttgcccaggctgggattattaagtactttttatcatacagcaagattgacattttatattggaatacatttgtctctatataacggagattaacaggaaaatgacaagcctgggtgcggtggctcatgcctgtaatcccagcactttgggaggctgaggtgggaggatcacttgaggtcaggagttcgagaccagttttgccaagatgatgaaagcccatgtctactaaaaatacaaaaattagcccagcttgatggtgggcgcctataatcccagctatttgagagactgaggcaggagaatcacttgaacctgggcagcagaggttgcagtgagccgagatcatgccactgcactccagcctgggtggcatagcgagactcttgtctcaagagaaaacaaaacaaaacaaaaaaaaaacaggaaaatgacaaaaagtaatattacaactcagtgaattttataacaaacttttttggaattcattgactaatactataccaaatccaaaatactctctagtataccaaatccaactctaccctatagtataaattggattctatttggacttgtctcactaatccctcatacagtgtgttttattttttattgaagtaaaaaaatttgtcattttaaccatttttaagtatatagttcagtaatattaagtatgttcatgttgttgcgcaatagatcttcggaagtttttcgtcttgcaacctgaaactctacccattagcaaattcccatttctccttacacttagcccttggtaatcatcattcttttttttttttttttgagatggagttttactcttgttgcccaggctggagtgcaatggtgcaatctcgactcaccacaacctccgcctcccaggttcaagcaattctacctcagcctcccgagtagctgggattacagtcatgcaccaccacgcccggctaattttgtatttttagtagagaaggggtttctccatgttgaggctggtctcgaactcctgacctcaggtgatctgcccacctcggcctcccaaagtgctgggattacaggcgtgagccactgcgcctggcccattctttctaattctataaatttgactacttagttaccttacataaataaattcttatagttagtgttatttttgcttccatgccttttttgttgttgttcatgctcttacttggaatgcgttctattttgtctacctatgcacatcctgttgggttttttttttttttgggggttttttttgtttttttttgtttttttttcccagacaaggtctcaatttgttacccaggctggagtgcagcggcgccatctccactcactgcatcctcaacttcctgggcccaggtgatcctctcgcctcagcccctgcaggtagctgggactataggcatgtgccaccatgcccagctaaatttggtttttttgtttgtttgtttttgagacagagtctcactctgtcacccaggctggagtgcagtggcacaatctcagctcactgcaatctctgccgcccgggttcaagtgattctcctgcctcagcctcccaagcagctgggattacaggtgactgccaccacgccagctaagttttgtagttttagtagagatggggtttcaccttgttggccatgctggtctcgaactcctgacctcgtgatctgcctgcttctgcctcccaaagtgctggaattacaggcatgagccaccacgcccggccagaatttttgtatttttagtagacacaaggttcttaccctgttgcctaggctggtctggaagtcctggactcaagcaattcacctgccttggcctcccaaaatgctgggattacaagccaccatgcccggcctaaatcctgttgttttgttttgttttattttgttttgttttgttttgtttgttttttgagacagagtctcgctatgtctctcaggctgtagtgcagtggcgcgatcttggctcactgccacctctgcctcccaggttcaagtgattctcctgcctcagcctcccaagtagctgggattacaggcatgtgctactatgtccggctaatttttgtatttttagtagagacagggtttcaccatgttggccaggctggtctcgaactcctgacctcgtgatccacccacctcggccacccaaagtgctgggattacaggcgtgagtggtttttatttcttaggccggtttcctccatatgatcttgcagtagacattaatttctttcctttttaattaaaatactgtttgtatttcacattttgatgtttgttaagatttgttttatattgttttttgttttgtcttgtgtgatagtcttaaatccctagttagataataactggagagtaccatgtttctatatatctctcagtgacttgcacagtgctagcagatagtgctaaaaaattatttattattattattattttgttattgttgttgttgttgttagacagggtcttcctctgtcacccaggctagagggcaatgggatgatcatagcttactgcagcctccaacaactgggctcatgtaattctcctgcctcagcttcccaagtagctgggattacaggcatgagccaccatgtctggacaaaaatatttccaggtgcagtggctcatgcctgtaattcccacacttgggaggccgagcgaggctggaggatcacttgagcctaggagttcaagaccagcttggctaagatggcgagaccccgtccctacaaaaaattttaaaaactagccaggcatggtggcatgcacctatattcccaactactcagtgggctgaggtgggagggtcatttgaacacaggaatttgaggggagaaaaaaagaagagagaaagagaagtgaaggaaggaagaaaggaaggagggagggagagaagaaagaaacgaaagaaaggaaaagaaaaggaaggaaagaaaattggtaccaggaaagcaggaaagggaaatggaagtaaaaaaataataataataataaaatgaaaattggttagtcactattaacaatttgtatccttataatctggaaacattataatttcaaaagaaaaaatattctttggatcataggttctgaggtcagaacagcattcccgtagtctagatgaagtcaagttttatctgatcttaattgaaataaatatagctggccttgaacaaatctactcatggtatgtggataggaattaaattgtaggggcattcacttgatggcattcattcttagaacatttacctatgtctagcttttggagtaaagtcacataacctctaaccaggtaagtttcctgtggctttatttaggattttaaatactcattttcagtgtaattttgttatgtgtggattaagatgactcttggtactaacatacattttctgattaaacctatctgaacatgagttgtttttatttcttaccctttccagagcgatgattctgacatttgggatgatacagcactgataaaagcatatgataaagctgtggcttcatttaaggtatgaaatgcttgcttagtcgttttcttattttctcgttattcatttggaaaggaattgataacatacgataaagtgttaaagtacatgttattcagttttcattttgaagattagatggtagtatgagttagttaaatcaggtgatatcctcctttagaagttgatagcctatatatgtcatcctttgtggaggcaatttaaataaaatttaaaacatttattcctggctgggtatggtggctcactcctgtaatcccagcactttgagaggctgaggcgggtggatcacctgaggtcaggagtttgagaccagcctggccaacatggtgaaaccccgtctttactaaaaatacaaaaattagccaagcatggtggcacgtgcctgtaatcccagctgcttgggacactgaggcaggagaattgcttgaacctggggggcagaggttgcaatgattgcaccactgcactccagcctgggcgatagagtgagactccatctcagaaaacgaacaaacaatgtattccttttagtatttttacattgtatcaaactatggaagtcctctaattgagattaataagaaaaagacaatctgaattataattttaaacatttaacaagcatgtagtaaaataatgatgaagataaatagcattagtacagcaattaatatttgtagcatgctgacagtgctctgtgtgcgtttcatatattaaattactctaatcatcccaaatcctgtaagttgggtatcaattcaagtgttcctattgggtaggaatatacagttcttttaggaaatgtagtatggttctgtgtctcaaacaggacacttacacagttggccaacatcatcaccttctccattctctgagatgtttagtcttactgagcactaaatatgggtcatcaatagtccagactaccttgagcaaacaatagtccagactaccttgagcaaacagagcatatactcatacagtgtataaagagcaccaagcatacagatttcatgtctttctcatagttactcttgtaacatgagctaaagatcagacctctatgtcacctttgtaactgatttctagatttttttttttttttgagatggggtcttgccctgtcacccaggctggagtgtagtggcgtgatcatgcctcattggagccttcaactcatgagctcaaacaatcctcctacctcagcttcctgagtagttgggaccacaggtgtgtgccaccacacccagctcatttttgtattctttgtagagatgcagtctcaccctgttgcccacgctggcctggaactcctgagctcaaaagatccctccgccttgaccttccaaagtgctgggattacaagcatgaaccactgcacccggcctagatttttaaatgtgctttccagtatacactgaaactagaagtcgactaaagaattaccaagagaattctataaaatagagattgaaatggggctcgatgtgggatgggttggtgatattgcagggagaagtaatctgagtaaaggaggaaaagaactgatttgggaaaacgatagttttagtagtgagtttgagtatgaattaagttgagattgaatttgaattaagttgaggttgaatatgaattaagttgaggttgagtttgaggtatgaattaagatgtgaaattgatcattggaaatgttagattgagaaaagtcacagctggattaatagcttcagaagtgtgtttgcagacagttgcaactaaagtaataagaatagatggccttggccgggcgcggtggctcacgcctgtaatcccagtactttgggaggctgaggcgagcaaatcacgaggtcaggagttcaagaccagcctggcccacatggtgaaaccccgtctttattaaaaatacaaaaattagctgtgcacagtggtgcacgcctgtaatcccagctactcgggaggctgagacaggagaatcgcttgaacctgggaggtggaggttgcagtgagctgagatcagtgtgactgcactccagcccggtgacagagtgagactctgtgtaaaaaaataaaataaataaaataatggccgtaagcaagtaaagaaggatggccagctcttattgggaatgcctaaatctaaggcttgatcagaagtaatgaaaccgttggggccctacattgctatgacatccaaagggccatgaatatcaggaagaaagataattaacagggtctaatgttacagagaggttgagagcaaggagatttgattaaaagggtctttagagctgatgtcaggtgtatgatgcctttaagagcagtttttatagtgcagggggtggtcaaaagagaaaataggtgctttctgaggtgacggagccttgagactagcttatagtagtaactgggttatgtcgtgacttttattctgtgcaccaccctgtaacatgtacatttttattcctattttcgtagcatgctctaaagaatggtgacatttgtgaaacttcgggtaaaccaaaaaccacacctaaaagaaaacctgctaagaagaataaaagccaaaagaagaatactgcagcttccttacaacaggttattttaaaatgttgagatttaacttcaaaggatgtctcattagtccttatttaatagtgtaaaatgtctttaacttaagtgattagtacagtgtttctattgacatatacttatacaacttcaaaaacaactattaaattttctgttatttaggaacatgcatattagtcatgaaagtataaagaattagatgggaatgataaatgctaaaatcaggacatgtgttccatttgtgaatggaaggcagggagaaggtgccgtttggaaggagtacccaagagccgtaagctgaattggcagtgttttacatcttaagctgagagatagatttttttttcccctttttctttaaaaactctaaaactgttaattccaaggaacccagaagtctaggtagattatttctgctagttaaaagcagtagtcctgaaagctgaatattttggtgtcttttgagccaactttagtttcatcattaccaagggggaagagagctaacagttgatgagcacttgctctaggccagtccagagtgctgggcaccatacgcattttatctccctcccgctattcacaacaaatatgggaggtagtttatattatagccatctaataagatggggaaactaagactcaaagagattcagaaacttgtccatgattataaatgtaagagagttggaattcagatttatgtatttagaccccaagcctttctcattacatcattttgccttccaaatctctaccctctatccttcacctccccactgatcaaaacgagatgatagtttgccctcttcaaaagaaatgtgtgcatgtatatatctttgatttcttttgtagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagaggtaaggatacaaaaaaaaaaaaattcaatttctggaagcagagactagatgagaaactgttaaacagtatacacagttgtcagtttgatccaccgaggcattaattttttcttaatcacacccttataacaaaaacctgcatattttttctttttaaagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaaggtaaaccttctatgaaagttttccagaaaatagttaatgtcgggacatttaacctctctgttaactaatttgtagctctcccatgaaacttttgtagcttaaatacacaagaattttttgaaaaggaaataagataatgatgcaaaatagttaattttttaaaaaaatgttagacactgcagtggatgcaacaaaatactttatatgaaagatttatccagttaacttttgtggagtattaggtattagactaataattagcacacttacttaagttagaaagtataataatgcgccggacgcggtagctcacgcctgtaatcccagcactttgggaggccaaggtgggcggatcacaaggtcaggagatcgagaccatcctggctaacacggtgaaaccccatctctactgaaaatacaaaaaaatttgccgggcgtgatggcgggcacctgtagtcccagctactcgggaggctgaggcaggaggatggtgtgaaccccggaggcagagcttgcagtgagtcaagatcgtgccactgcactccaacctgggcgacagaatgagactccatctcaaacaaaaaaacaaaacaaaacaaaaaaaagtgtaataataatttatcattagctggatgatatgctgttgtttcccatgtcacctgtataagatatgtaaaataagaacacattatttacatctaatatagataaaatcctgaggcgctctcagattgttttgtagagttcaaatgtaaatattgttttcatttatggtccttttggttataagtaacagaaatcaactctaaaaagatttttattataggttagattatgtcatggaaccttaaggcttgtccctttctagttcttttgtgtaaagcggtgatttcttccatggagggaatggtatttaggcaatttttttttttttttcgagatggagtcttgctctgtcgctcaggctggagtgcagtggcaccatttcagctcactgcaacttccacctcctgggttcaagtgattctcctgcttcagcctcccaagtagctgagattacaggcacccgccaccacacccggcttattttgtatttttagtagagatggggtttcaccatgttggccaggctggtcttgaactcctgacctcaagtgatctccccaccttggccttccaaagtgctaggattacaggcgcctagcctaggcagtcattttcaaaaaacaagcatgactcaccaaaagttttaagattttctgtgataatgttcttattgaggcttacattatattacagtttcttgaatctaaaatgatgtaccctcttagaatatatacatcatgcttcattggtctcagggggctgatttttataaggagagatttgctagttttcacaatatgtcctctaagttggcatgtatagctaaacaggctttcataaaaatatacaatttagttaatgaaatttgggatatagtcttttatgattgaaataattttgctaaatagactgtctctgatttattaggtaatcaccactcttattttgttttacttccttaatgtctacatagaaaggaaatgagaaaaatccagaggttgtcatttgacttatgagtctgtttgacttcaggatttggtacatgaaatttcacttaatctttttgatatgtataaaacaaatattctgggtaattatttttatccttttggttttgagtcctttttattcctatcatattgaaattggtaagttaattttcctttgaaatattccttatagccaggtctaaaattcaatggcccaccaccgccaccgccaccaccaccaccccacttactatcatgctggctgcctccatttccttctggaccaccagtaagtaaaaaagagtataggttagattttgctttcacatacaatttgataattagcagaatagaggattgtaaaatgtcattgtagaacatcccttgggccagattctaatgggtagaaatttgaactaaacctctgggttttgtttgtttttaatgcctttctgttacccagatgcagtgctcttgtagtcccaagtctaagctctaggttgccttctttcctggcagaagttggtgtctatgccataaggaggtagttcctgttagaagggatttaattataccttatataaggaattagtgtttgcccttctaggtatagttggatgttagcttctgatgtaaactggatttctttttctttctctctctttttttttttttgttttggaggcagagttttgcccttgtaccccaggctggagtgcagtggtgtgatctcagctcacagcaacctccgcctcctgggttcaagcaattctgcctcggcctcccaagtagctgggattacaggcgactgccaccacacccggctaatttttgttttattagtagagatggggtttcaccatgttggccagactgatcttgaactcctgacctcaggtgatccacccgccttggcctcccaaagcgctgggattacaggcgtgagctgccgcacccagctgtaaactggatttctaatggtagatttttaggtattaacaatagataaaaagatactttttggcatactgtgtattgggatggggttagaacaggtgttctacccaagacatttacttaaaatcgccctcgaaatgctatgtgagctgtgtgtgtgtgtgtgtgtgtgtgtgtattaaggaaaagcatgaaagtatttatgcttgattttttttttttactcatagcttcatagtggaacagatacatagtctaaatcaaaatgtttaaactttttatgtcacttgctgtcttttcgtcctcgttaaatttaattttgttggtcttttgttgttattggttggttttctccaaatgctagctatgttaagaaatttaaggccaggtacagtggctcatgcctgtaatcccggcattttagaaggctgaggcaggaggatcacttgagctcaggagtttgagaccagtctgggcaacatagcaagacctcgtctttgtttaggggaaaaaaaagaaatttaagtaggagattatataagcaaaaatacaattaatttccagcattcactatataatataaatctccagactttacttttttgtttactggatataaacaatatctttttctgtctccagataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctgtctaattttttgtatttttttgtaaagacagggtttcactgtgttagccaggatggtctcaatctcctgaccccgtgatccacccgcctcggccttccaagagaaatgaaatttttttaatgcacaaagatctggggtaatgtgtaccacattgaaccttggggagtatggcttcaaacttgtcactttatacgttagtctcctacggacatgttctattgtattttagtcagaacatttaaaattattttattttattttattttttttttttttttgagacggagtctcgctctgtcacccaggctggagtacagtggcgcagtctcggctcactgcaagctccgcctcccgggttcacgccattctcctgcctcagcctctccgagtagctgggactacaggcgcccgccaccacgcccggctaatttttttttatttttagtagagacggggtttcaccgtggtctcgatctcctgacctcgtgatccacccgcctcggcctcccaaagtgctgggattacaagcgtgagccaccgcgcccggcctaaaattatttttaaaagtaagctcttgtgccctgctaaaattatgatgtgatattgtaggcacttgtatttttagtaaattaatatagaagaaacaactgacttaaaggtgtatgtttttaaatgtatcatctgtgtgtgcccccattaatattcttatttaaaagttaaggccagacatggtggcttacaactgtaatcccaacagtttgtgaggccgaggcaggcagatcacttgaggtcaggagtttgagaccagcctggccaacatgatgaaaccttgtctctactaaaaataccaaaaaaaatttagccaggcatggtggcacatgcctgtaatccgagctacttgggaggctgtggcaggaaaattgctttaatctgggaggcagaggttgcagtgagttgagattgtgccactgcactccacccttggtgacagagtgagattccatctcaaaaaaagaaaaaggcctggcacggtggctcacacctataatcccagtactttgggaggtagaggcaggtggatcacttgaggttaggagttcaggaccagcctggccaacatggtgactactccatttctactaaatacacaaaacttagcccagtggcgggcagttgtaatcccagctacttgagaggttgaggcaggagaatcacttgaacctgggaggcagaggttgcagtgagccgagatcacaccgctgcactctagcctggccaacagagtgagaatttgcggagggaaaaaaaagtcacgcttcagttgttgtagtataaccttggtatattgtatgtatcatgaattcctcattttaatgaccaaaaagtaataaatcaacagcttgtaatttgttttgagatcagttatctgactgtaacactgtaggcttttgtgttttttaaattatgaaatatttgaaaaaaatacataatgtatatataaagtattggtataatttatgttctaaataactttcttgagaaataattcacatggtgtgcagtttacctttgaaagtatacaagttggctgggcacaatggctcacgcctgtaatcccagcactttgggaggccagggcaggtggatcacgaggtcaggagatcgagaccatcctggctaacatggtgaaaccccgtctctactaaaagtacaaaaacaaattagccgggcatgttggcgggcaccttttgtcccagctgctcgggaggctgaggcaggagagtggcgtgaacccaggaggtggagcttgcagtgagccgagattgtgccagtgcactccagcctgggcgacagagcgagactctgtctcaaaaaataaaataaaaaagaaagtatacaagtcagtggttttggttttcagttatgcaaccatcactacaatttaagaacattttcatcaccccaaaaagaaaccctgttaccttcattttccccagccctaggcagtcagtacactttctgtctctatgaatttgtctattttagatattatatataaacggaattatacgatatgtggtcttttgtgtctggcttctttcacttagcatgctattttcaagattcatccatgctgtagaatgcaccagtactgcattccttcttattgctgaatattctgttgtttggttatatcacattttatccattcatcagttcatggacatttaggttgtttttatttttgggctataatgaataatgttgctatgaacattcgtttgtgttctttttgtttttttggttttttgggttttttttgttttgtttttgtttttgagacagtcttgctctgtctcctaagctggagtgcagtggcatgatcttggcttactgcaagctctgcctcccgggttcacaccattctcctgcctcagcccgacaagtagctgggactacaggcgtgtgccaccatgcacggctaattttttgtatttttagtagagatggggtttcaccgtgttagccaggatggtctcgatctcctgacctcgtgatctgcctgcctaggcctcccaaagtgctgggattacaggcgtgagccactgcacctggccttaagtgtttttaatacgtcattgccttaagctaacaattcttaacctttgttctactgaagccacgtggttgagataggctctgagtctagcttttaacctctatctttttgtcttagaaatctaagcagaatgcaaatgactaagaataatgttgttgaaataacataaaataggttataactttgatactcattagtaacaaatctttcaatacatcttacggtctgttaggtgtagattagtaatgaagtgggaagccactgcaagctagtatacatgtagggaaagatagaaagcattgaagccagaagagagacagaggacatttgggctagatctgacaagaaaaacaaatgttttagtattaatttttgactttaaattttttttttatttagtgaatactggtgtttaatggtctcattttaataagtatgacacaggtagtttaaggtcatatattttatttgatgaaaataaggtataggccgggcacggtggctcacacctgtaatcccagcactttgggaggccgaggcaggcggatcacctgaggtcgggagttagagactagcctcaacatggagaaaccccgtctctactaaaaaaaatacaaaattaggcgggcgtggtggtgcatgcctgtaatcccagctactcaggaggctgaggcaggagaattgcttgaacctgggaggtggaggttgcggtgagccgagatcacctcattgcactccagcctgggcaacaagagcaaaactccatctcaaaaaaaaaaaaataaggtataagcgggctcaggaacatcattggacatactgaaagaagaaaaatcagctgggcgcagtggctcacgccggtaatcccaacactttgggaggccaaggcaggcgaatcacctgaagtcgggagttccagatcagcctgaccaacatggagaaaccctgtctctactaaaaatacaaaactagccgggcatggtggcgcatgcctgtaatcccagctacttgggaggctgaggcaggagaattgcttgaaccgagaaggcggaggttgcggtgagccaagattgcaccattgcactccagcctgggcaacaagagcgaaactccgtctcaaaaaaaaaaggaagaaaaatatttttttaaattaattagtttatttattttttaagatggagttttgccctgtcacccaggctggggtgcaatggtgcaatctcggctcactgcaacctccgcctcctgggttcaagtgattctcctgcctcagcttcccgagtagctgtgattacagccatatgccaccacgcccagccagttttgtgttttgttttgttttttgttttttttttttgagagggtgtcttgctctgtcccccaagctggagtgcagcggcgcgatcttggctcactgcaagctctgcctcccaggttcacaccattctcttgcctcagcctcccgagtagctgggactacaggtgcccgccaccacacccggctaatttttttgtgtttttagtagagatggggtttcactgtgttagccaggatggtctcgatctcctgaccttttgatccacccgcctcagcctccccaagtgctgggattataggcgtgagccactgtgcccggcctagtcttgtatttttagtagagtcgggatttctccatgttggtcaggctgttctccaaatccgacctcaggtgatccgcccgccttggcctccaaaagtgcaaggcaaggcattacaggcatgagccactgtgaccggcaatgtttttaaattttttacatttaaattttattttttagagaccaggtctcactctattgctcaggctggagtgcaagggcacattcacagctcactgcagccttgacctccagggctcaagcagtcctctcacctcagtttcccgagtagctgggactacagtgataatgccactgcacctggctaatttttatttttatttatttatttttttttgagacagagtcttgctctgtcacccaggctggagtgcagtggtgtaaatctcagctcactgcagcctccgcctcctgggttcaagtgattctcctgcctcaacctcccaagtagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaatta

SEQ ID NO: 13 - DNA, 1634 bp, Homo sapiens
ccacaaatgtgggagggcgataaccactcgtagaaagcgtgagaagttactacaagcggtcctcccggccaccgtactgttccgctcccagaagccccgggcggcggaagtcgtcactcttaagaagggacggggccccacgctgcgcacccgcgggtttgctatggcgatgagcagcggcggcagtggtggcggcgtcccggagcaggaggattccgtgctgttccggcgcggcacaggccagagcgatgattctgacatttgggatgatacagcactgataaaagcatatgataaagctgtggcttcatttaagcatgctctaaagaatggtgacatttgtgaaacttcgggtaaaccaaaaaccacacctaaaagaaaacctgctaagaagaataaaagccaaaagaagaatactgcagcttccttacaacagtggaaagttggggacaaatgttctgccatttggtcagaagacggttgcatttacccagctaccattgcttcaattgattttaagagagaaacctgtgttgtggtttacactggatatggaaatagagaggagcaaaatctgtccgatctactttccccaatctgtgaagtagctaataatatagaacaaaatgctcaagagaatgaaaatgaaagccaagtttcaacagatgaaagtgagaactccaggtctcctggaaataaatcagataacatcaagcccaaatctgctccatggaactcttttctccctccaccaccccccatgccagggccaagactgggaccaggaaagccaggtctaaaattcaatggcccaccaccgccaccgccaccaccaccaccccacttactatcatgctggctgcctccatttccttctggaccaccaataattcccccaccacctcccatatgtccagattctcttgatgatgctgatgctttgggaagtatgttaatttcatggtacatgagtggctatcatactggctattatatgggttttagacaaaatcaaaaagaaggaaggtgctcacattccttaaattaaggagaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtggcaaaatgttacagaatctaactggtggacatggctgttcattgtactgtttttttctatcttctatatgtttaaaagtatataataaaaatatttaatttttttttaaattaaaaaaa

SEQ ID NO: 14 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 15 - RNA, 15 nt, Artificial Sequence
  Synthetic Polynucleotide
ccagcauuaugaaag

SEQ ID NO: 16 - DNA, 18 bp, Artificial Sequence
  Synthetic Polynucleotide
  modified_base (1)..(18): 2′-O-(2-methoxyethyl) modification
  misc_feature (1)..(18): phosphorothioate linkage between the indicated positions
  modified_base (2)..(2): 5-methyl cytosine
  modified_base (4)..(4): 5-methyl cytosine
  modified_base (8)..(8): 5-methyl cytosine
  modified_base (15)..(15): 5-methyl cytosine
tcactttcataatgctgg

SEQ ID NO: 17 - RNA, 26 nt, Artificial Sequence
  Synthetic Polynucleotide
cuauauauagauaguuauucaacaaa

SEQ ID NO: 18 - RNA, 19 nt, Artificial Sequence
  Synthetic Polynucleotide
aaggugucgaugcacuaca

SEQ ID NO: 19 - RNA, 19 nt, Artificial Sequence
  Synthetic Polynucleotide
gcauuguaacgccuaucga

SEQ ID NO: 20 - RNA, 18 nt, Artificial Sequence
  Synthetic Polynucleotide
gcagaugaacucgggaug

SEQ ID NO: 21 - RNA, 19 nt, Artificial Sequence
  Synthetic Polynucleotide
caucaccgaguucaucugc

SEQ ID NO: 22 - DNA, 21 bp, Artificial Sequence
  Synthetic Polynucleotide
ccgcacucguucucgaucutt

SEQ ID NO: 23 - RNA, 21 nt, Artificial Sequence
  Synthetic Polynucleotide
agaucgagaacgagugcggga

SEQ ID NO: 24 - DNA, 20 bp, Artificial Sequence
  Synthetic Polynucleotide
gcuagauggaagaacacuat

SEQ ID NO: 25 - DNA, 21 bp, Artificial Sequence
  Synthetic Polynucleotide
uaguguucuuccaucuagctc

SEQ ID NO: 26 - DNA, 21 bp, Artificial Sequence
  Synthetic Polynucleotide
ggacugccuccaaguggaatt

SEQ ID NO: 27 - RNA, 21 nt, Artificial Sequence
  Synthetic Polynucleotide
uuccacuuggaggcaguccag

SEQ ID NO: 28 - DNA, 21 bp, Artificial Sequence
  Synthetic Polynucleotide
ccucgaaaugauagacgaatt

SEQ ID NO: 29 - DNA, 21 bp, Artificial Sequence
  Synthetic Polynucleotide
uucgucuaucauuucgaggtt

SEQ ID NO: 30 - DNA, 21 bp, Artificial Sequence
  Synthetic Polynucleotide
gcauaggguugacugauaatt

SEQ ID NO: 31 - DNA, 21 bp, Artificial Sequence
  Synthetic Polynucleotide
uuaucagucaacccuaugctc

SEQ ID NO: 32 - DNA, 21 bp, Artificial Sequence
  Synthetic Polynucleotide
cgacgucccuuugauccaatt

SEQ ID NO: 33 - RNA, 21 nt, Artificial Sequence
  Synthetic Polynucleotide
uuggaucaaagggacgucggg

SEQ ID NO: 34 - DNA, 21 bp, Artificial Sequence
  Synthetic Polynucleotide
ggauaccucuaguaaagaatt

SEQ ID NO: 35 - RNA, 21 nt, Artificial Sequence
  Synthetic Polynucleotide
uucuuuacuagagguauccga

SEQ ID NO: 36 - DNA, 21 bp, Artificial Sequence
  Synthetic Polynucleotide
ggauuauuuaauaacauuatt

SEQ ID NO: 37 - RNA, 21 nt, Artificial Sequence
  Synthetic Polynucleotide
uaauguuauuaaauaauccaa

SEQ ID NO: 38 - DNA, 21 bp, Artificial Sequence
  Synthetic Polynucleotide
cgucagcguguaucaggaatt

SEQ ID NO: 39 - RNA, 19 nt, Artificial Sequence
  Synthetic Polynucleotide
uuccugauacacgcugacg

SEQ ID NO: 40 - DNA, 21 bp, Artificial Sequence
  Synthetic Polynucleotide
ggccgugguaguuacucaatt

SEQ ID NO: 41 - RNA, 21 nt, Artificial Sequence
  Synthetic Polynucleotide
uugaguaacuaccacggccag

SEQ ID NO: 42 - DNA, 17 bp, Artificial Sequence
  Synthetic Polynucleotide
cagagtggttgtctctg

SEQ ID NO: 43 - DNA, 18 bp, Artificial Sequence
  Synthetic Polynucleotide
ctccacgacaccagtgcc

SEQ ID NO: 44 - DNA, 17 bp, Artificial Sequence
  Synthetic Polynucleotide
ggacgccggagccgcag

SEQ ID NO: 45 - DNA, 18 bp, Artificial Sequence
  Synthetic Polynucleotide
gagatcgagaacgagtgc

SEQ ID NO: 46 - DNA, 17 bp, Artificial Sequence
  Synthetic Polynucleotide
atgcatcgtgattcctg

SEQ ID NO: 47 - DNA, 17 bp, Artificial Sequence
  Synthetic Polynucleotide
ctgcgacgaggtggagg

SEQ ID NO: 48 - DNA, 17 bp, Artificial Sequence
  Synthetic Polynucleotide
gttacggttctggacgc

SEQ ID NO: 49 - DNA, 16 bp, Artificial Sequence
  Synthetic Polynucleotide
gctccgggagcgggag

SEQ ID NO: 50 - DNA, 19 bp, Artificial Sequence
  Synthetic Polynucleotide
gatccaagggatgcagatg

SEQ ID NO: 51 - DNA, 20 bp, Artificial Sequence
  Synthetic Polynucleotide
ctatcatttcgaggtctgcg

SEQ ID NO: 52 - DNA, 17 bp, Artificial Sequence
  Synthetic Polynucleotide
gtggatacagcagtcgg

SEQ ID NO: 53 - DNA, 18 bp, Artificial Sequence
  Synthetic Polynucleotide
ctggatctgcttccagag

SEQ ID NO: 54 - DNA, 19 bp, Artificial Sequence
  Synthetic Polynucleotide
ggtctagatcacattctcg

SEQ ID NO: 55 - DNA, 18 bp, Artificial Sequence
  Synthetic Polynucleotide
ccagacctagatcttctg

SEQ ID NO: 56 - DNA, 18 bp, Artificial Sequence
  Synthetic Polynucleotide
caggagcgagaacccgag

SEQ ID NO: 57 - DNA, 19 bp, Artificial Sequence
  Synthetic Polynucleotide
cttctgcatatggtacgac

SEQ ID NO: 58-59 - skipped (2 entries)

SEQ ID NO: 60 - DNA, 19 bp, Artificial Sequence
  Synthetic Polynucleotide
gttacccagcacgtgtacc

SEQ ID NO: 61 - DNA, 18 bp, Artificial Sequence
  Synthetic Polynucleotide
ggcctgaaggtcatctcc

SEQ ID NO: 62 - DNA, 21 bp, Artificial Sequence
  Synthetic Polynucleotide
gagtacattgaagagaacaag

SEQ ID NO: 63 - DNA, 21 bp, Artificial Sequence
  Synthetic Polynucleotide
cactgtgtcatcgaagtgttc

SEQ ID NO: 64 - RNA, 28073 nt, Artificial Sequence
  Synthetic Polynucleotide
ccacaaaugugggagggcgauaaccacucguagaaagcgugagaaguuacuacaagcgguccucccggccaccguacuguuccgcucccagaagccccgggcggcggaagucgucacucuuaagaagggacggggccccacgcugcgcacccgcggguuugcuauggcgaugagcagcggcggcagugguggcggcgucccggagcaggaggauuccgugcuguuccggcgcggcacaggccaggugaggucgcagccagugcagucucccuauuagcgcucucagcacccuucuuccggcccaacucuccuuccgcagccucgggacagcaucaagucgauccgcucacuggaguugugguccgcguuuuucuacgucuuuucccacuccguucccugcgaaccacauccgcaagcuccuuccucgagcaguuugggcuccuugauagcguugaguggaggcccugccgcgacuuggcaguagcuuauuuuguucacuccucucuggcuggugugggggaggugggggcauuaggccagggugaagcaggggaaccacuuaggagucuguuaagaugaucugaacuucagaacaagauguuauuaacagagugaaaguauuuggauucuggguauauuuugaaaucggaggcaacagguuuuucagauagauucgauaacggagguuauccugaauaguugaaaagauaaaguugccuuuugcugaggugggaaagagaagauugccaguagagcagguuucucaggaguucagucuugggcauagcaugguaggggugaauuuggcuggagugaguuggagaguaggagaagagaaauccaaggcaacauuugaccagccugggcaacauagugugacuccgagucugcaaaaauuagacggguguuguggugcgcgucuguggucucagcuaccuggaagguucaggccuuggaaggcucagggagguggaggcugcagugaucugugauugcgccucugcacuccagccugggcgacagagccagacccugucuuaaaacaaaauaaacggccgggcgcgguggcucaagccuguaaucccagcacuuugggaggccgaggcggccggaucacaaggucaggagaucgagaccauccuggcuaacacggugaaaccccgucucuacuacaaauacaaaaaauuagccgggcguggugacgggcgccuguagucccagcuacucgggaggcugaggcaggagaaugucaugaagccgggaggcggagcuugcagugagccgagaucgcgccacugcacuccagccugggcgauagagcaagacuccgucucaaauaaauaaauaaauaaauaaauaaauaauaaaaacaucgguaggcauauuucaaggaauucuauuuaaaaaaaauuuuuuuagagacaaguucgcucucuguggcccaggcuggaguacaguggcaugauccuagcccauggcagcguugaucucuuggccucaagcgacccuccuuuggagucgcugggccuaaaggagugagccaccacgaaauuuuauuauaaauggaggguagagaaauugggcaauaaauggagggggaagugaguuaagaggaauuuuaauuaugugugugugguuuuaaaagaggggggucuugcucuguugcccaggcugcuggggugccaguggcgcaaucaugaaucacuacagccuuggacuccuggccucaagcuauccucccaccucugccucccaaaguacugggauuacuagugugagccacugcacuaagauaggagcaacauguuucagcauguuuguggguugauaggaaagaugagaaugggaaaguugaugucggaaagaagacaauggcuagagcaauguccuagaguagguaagaagggauggauuuggccuuuguuggaaacauuagcgguucuuuuggugacagcuauauaguuaacacaucuaugauacgugaaugggcagauaggauggcaggagauuuugaaaguucucuugauucuuacuguucucuuagugaaagaagcaagguuaucagcuagaagcugggaugggagaggaaagagaagaugggaaguagauaguucuuuagaagagugggcaaggguuggacuagggaaguuuaguggaaauauugcuaggcaacauaaagagccuacuugagauucguggucaugaguugaaggagaccagacagcaagauuguguaugagggcacccacagaguaaauggagaguugaaauuaaugcaguugugauuuuaccacguggauaugaagaagugagggggagaaguacaaaggaguucucuuaaugauugaccauggaauuuaagcuggcuaagaaaggaagugagaggccgggcgcgguggcucacgccuguaaucccagcacuuugggagacugagguggguggauuaccugaggucaggaguuugagaccaaccuggccgauauggcgaaaccccaucucuaauaaaaauacagaaaaauuagccgggaaugguggcaggugccuguaaucccagcuacucaagaggcuguggcaggaguaucccuuggacccaggagguggagguugcagugagccgagaucacgccacuguacuccagccuggacgauauagugagacuucaccucaaaaaaaaaaaaaaagaaaggaagugaggauuuuaagacccugagagacaguuuaaaaagugggaggaucggccgggcgcuguggcugacaccuguaaucccagcacuuugggaggccgaguugggcagaucacaaggucaggaguucgagaccagccuggccaauauggugaaaccuugucucuacuaaaaauacaaaaauuagccgggcauggugucacgugucuauaaucccagcuacucgggaggcugaggcagaaaaauugcuugaaccugggaggcagagguugcagacagcugagaucacuccauugcacuccagccugggcaacaagagcaaaacuuugucuuuaaaaaaaaaaaaaaaaaaagaauacaaaaauuagccgggcgugguggcgcgugccuauaaucccagcuacuugggaggcugaggcaggagaaucaguugaacacgggaggcgagguuugcagugagccgagauugcgccacugcacuccagccugggcgacagagcaggacuccucuuggaaaaaaaaaauuagcugggcaugguggcaggugccuguagucucagcuacuagggaggcugaggcaggaaaaucacuugaacccgggauguggaguuugcagugacccgagaucgugccacuguacuccauccugggcgacaaaaugagacucugccucaaaaaaaaaaaaaaaaaaaagugggaggaucaauguacugccaguccuaaugaaguggaaugauuguccccaucaaaucacuaguaggaguaaguugcagagccuagaaggugaugguuaagagagugggauucuugaaacugcauuuauggagagguugugguuauugguuauaauaaauaaauacaguugaagugagugaguagcugagauuuggggauguaucaguucauucuuacacugcuacaaagacauaccugagaccagguauuuauaaagauaagagguuuaaucagcucacaguucugcugccuguacaggcuucucuuguggaggccuaaggaaacuuacagucaugguggaaggugaaggggaaacaagcacagucuucacauggccagcaggagagagagagaagggggaagugcuacauacuuuaaaacaaccagaucuugugagaacgcuuaucaggaaacagcacuuggggauggugcuaaaucauuagaaaucacccccaugauccagucgccuccuaccaugcccaccuccaacacuggggaucacaauucagcaugagauuuggguaggaacacagagcugcaccacaucagaggauguacaagauugugguggagaggaguuuagagaccugcaaauauaggguaauugaagggaucaucuacauggauauuuaaaucaccaaaaauuaugacaggaguaguguuggagagagaacugcgauguaaacauuaaggaaugaggaagagugacucgguaggcuguaggugacugcaauaggaaacgauaauagacugugagucuggugacaagauuuuccuucuuucuuuuuuuccccccccccgagacagggccucuuuuuguugcccaggugggagugcaguggcgcgaucacggcucacuacaaccuccucccaagcucaagggauucucccacuucagccucucaaguagcuggaacuacaggugcugaccaccaugccuggcuacuuuuugucaggauuuucaaggcugggaauuuugagaggggaauggaggagaauaaucugaaagugcaaguaaggagcagggaagauuucuuuuuucuuuuuuuuuuuuuuuuuugagucggagucuggcucagucgcccaggcuggagugcaguggcgagaucuccgcucacugcaagcuccgccucccguguucacgccauucuccuccuucagccucccgaguagcugggacuacaggcgcccgccaccacgcccagcuaauuguuuuuuuguauuuuuaguagagacgggguuucaccguguuagccaggauggucucaaucuccugacuuugugauccgcccaccccggccucccaaagcgcuugggauuacaggcgugagccaccgcgccagccagagcagggaagauuucuuccccacaucuccaguagguacagugauaugaaguguguggaggagaaaagaggaaacaucuaucauuugagauggcugcgaaaggaaaaggcauccucagggagcuagauuuuacuuagagcaagaaaugaagggaugauucagagguuaaaagaguggauuuuaugaauuacucaagggagcacaguggaaguuucaggaagugguaggagaagguagaagauggcaggguguugggaauaauuugagaaaucugagcuacuggaaaugacugagaaucagauauaaaggcaguccuggugguccguucuggcugccguugcuguguaacgaaucugccaaaacuuaguggcuugaaacaacaaagaacauuuuauuaucucucauuguuucuguggguuaggaauuugugagagccgugcugggcaguuuucgugcggcugucucgugguugcaccuacauaguugcuagagcuacaguagcuggggacugagcagcuagggauuggcaggcuaucucuuuuuuucauguagucucaugaagauuucuuuaugugguuucaaugugugggcugguuuggauuuccuuauagcaugguggccucaguuggauugcuguuuugugauccuuuucaucccuccuuguccuguccccagacaaccacugaucuacuuucugucaccauagauuagccugcauuuuuaagaauuuuuauaaacguggaaugauagaguaccuuuuuugucacguuucuuuuauuuaucauagcuauuuugauuuucauccauuuuauugcugaguaguaucccauugcauguauauacuauacuguauucauucgcuugcuugugaacauuugggcuuuuuccaguuugggacuguuaacaaguagagccacuaugaauauuaguguauaagacuucauauagccaaggcuggcagaucgcuugagcccaggaguuugagaccagccugggaaacauggugaaaccucuauuuuuauuuuaaaaucaaaaauuaaaaauuuucuauaaaaaauuuuaaagaagacuuuguauagacauacgcuuucauuuuucuugagugaauacuuaggucucaggguagauguauuuuaagucuuuaaggagcugucaaacucuuccucaaaguggugguuguaccauguuacuuuuuaauauaacagagauuaauugagcaaagaaaaauucaaaaguuggacagcccccacaacuaaauagguucagaacagcucccccauuuugcauuuugaccagcaauguaugaaaguuccauuugcucagugucccugcaaacaccugguauggucagucuuuuuaauuuuaggcauuauaauagauauaguggcuucuugugauuuuaauuagcauuuccuaaugaccagugcugcuguugaucauuucaugaguguauuugccauccguauaucuuuuuuggugaagugucuauucaaaucauuuggguuuuuuuuuuuuuuguuuuuuuuuuuuggagacagugucucacucugucacccaggcuguugugcaguggugcaaucacacagccuacugcagccuccaccuccugcgcucagucuucuugucucagccuucugaguagcugaaauuacgagcacacgccacaaugccuggcuaauuuuuuaaaauuuuguagaaacaaggucucauuauguugccugggcuugucgugaacuccugggcucaagcaaucuuccugccucagccucccaaagauugggauugcaaguaugagccacugcacccggccaacuuacccaucuuuuaauugaauuuuuuuguuguugagguuugagaguucuucauguuugcuggguacaauaucuuuaucagauagguaacuugcauguauuuucucccgguuuacacuuugguuuuucauuuuguuaacaacgucuuuuuaagaacagaaaaucuuaauuuugcugaaaucuaauuuuucaguuuuuucuuugaugguuuugagagaggagguaaaaaaagacuagguaagccgauaguuagacagaguccucgguagaacuucccuucuaacaaaaagcagcccaagaaaucacuucucuucuaacaaggagcagccuggaagaucgggcuguaaacauguauaaggaagcagcucuggcacagagggggagcuuccuggguaaucagcaagcuucacauacguaagguggguaugugaaguaaacacaguaugugaaguaaacacaguggaccuuaguacauacucagauaaggaagcuggaagcuugcauguugugaguuguugggguugccugcagcugcacggagagaaagggguaccuggggccaggcauguccaccaugguggcuccaccuccccuuauuuagcacaugcacaauaggaaagagauaagcaauguggaguagcucaggccaaggaccugccugcauaauaaaagguuggggugggggaugccagagauucacgcucugugcagauggcaacaccugguccuaacugguuuuuugcucccuauguguagauaagcuacccccuucccauuagcucauuuauaaaaaugcuugcauuucacuguggaaugggaacucuuuucaggaccucucucugcaggagagagcuagucucuuucuuuugccuauuaaacuucugcucuagccucacacccuuggugugucagcguccuugauuuccucagcgugagaccaagaaccucgggugccaccccaggcaacaaggccauuucaguuuguucuuuuguuauaggcaauccaugaucacagauuuuucucucuuuuuuuuuuuuacacaguuuagaguuuuaguuuuacacuuaggucuguaauccauuuuguauuaauucuuauauguggcucaguguagguggaaauuugguuuguuuuugcauaaggauuuccaauaguuuuaccaccauuucuugaaacuacuaugcuuucucuauuaaaccacauuuguaacuuuaguuaaaaucagucacauauaucacagggcuauuucugacucucaauucuguuacauugucuauuaguguauauugaugucaguacuacacuuuuaauuacuauugcuucaggguaugucuuguaaaccaaaaauaaaauuauaggccccccccgccccugcacaaccaacugaauggacccauccucucagccaagggcauuccaaaauuaaccugaaaaacuaguucaagccaugaugggaagggggaguuggacaugucucaucacacccuacuaccuuuuggaauuacugauagaacagacucuuaaagucugaaaagaaacauuuacaaccuacccucucugaagccugcuaccugggagcuucaucugcaugauaaaaccuuggucuccacaaccccuuaugguaacccaaacauuccuuucuguugauaauaacucuuucaacuaguugccaauuagaaaaucuuuaaaucuuccuaugaccuagaaaccucccuacccccacuuugaguuguccugccuuuccugacagaacucauguacaucuuacauauauugauugaugccucaugucucccuaaaauguauaaaacaaagcuguaccccaccaccuuggggacaugucaucaggaccuccuguggcugugucauaggagcgucuuuaacuuuggcaaaauaaacuuucuaaauugauugaaaccugucuuagcuacuucugguuuacagucuuaaaguuagauaauguaaauuguccagcuuugguuuauuuuuguccuuaguaguuccauauaaauuuuagaaucagcuuuucaauuuaauacacuacuuuccucuuagauccacaauuaaauauauuugaugcuaacaauucuguuuuauguuuuucguuuuuuuuuuuugagacaagaguuucgcucuuguugcccaggcuggagugcaguggcgcgaucuuggcucaccacaaccuccaccucccagguucaagcaauucuucugccucagccucccgaguagcugggauuacaggcaugcgccaccacgcccggcuaauuuuguauuuuuaguagagacgggguuucaccauguugaucaggcuggucuugaacuccugaccucaggugauccacccaccucggccucccaaaguguugggauuacaggcgugaaccaccaugccuggccaguucuguuauuuuuaaaacccaaguuucccuggucauaucuugguuggaugaagcguauuuucaauagauuacccuggaaaggcuagugaguacgguauucuucuacauuuuagacuuuucuuagucuugcuacuucaaggacagcuaggcugcauauaaaauucuuggcucauacuuuuuccccauaaauuucuaugagaaagucuaaugauaacugauuuucuuuauuuuguaacuuagucuuuuugcuuagaggcucucugaggaugggaggggguucuuccucccaucccuaggaauuuuucuuuuuuuuaaauuccuaaucacuagaccaccaggaagauuguuuguuuuguuuuguuuuuauucuucagggaccccauuuauacauacguuaaauaaauacuguuugccaauguaucaaccauuuugcuucuuauuuauuuuuguuccuuugguucuuuuucauggcuuugcuuuggugcuccuuagauuuucagucagauguauuuguccuuggguaccuuguaaucaguauuaccuuuucuucugucgcuuuguuuucuguucguuuugaaauuacuuguuuccuggucuggcaauaacaguugagauaugaggaguuugagcugccaucugucuauguaucuugcuuuaagacugcacucuucuauugauaucacuggccuugauuuugugauuucuuuauuucuucaggaccacccuucauuuucuacuguuugcuuccuuuuuuuuugagauggagucucacucugucacucaggcuggagugcagugaucuuggcucauugcaaccucugccucccggguuccagcaauucuccugccucagccucccaaguaucugggacuacaggugugcaccaccaugcccggcuaaguuuuguauuuuuaauagagacgggguuuugccacauuggcaggcuggucucaaacuccugaugucaagugauccacccaccccacccaccucugcaucccaaagugcugggauuacaggaaugagcugccgugcccagccuccccccuaccccccuuuuuuucuuucgagacagagauuauaggugugagccacuggacccagccuguuuuuauuccuuuuaccaaaucuccaaggaauaucuucccuuccaagugcgaauguaaccuuaagucaguuaaccucuuugugauuacuuuucuuaucugcaaagugacuuaaugaucuuaaguacuuuuuuuuuuugagacagggucucacugucacccuggcuggagugcaguggcacgaucucugaucuccacucacugcaaucuccucuucccugguucaagcggcccucccaccuuagccuucuggguagcugggacuacagaugugaaccaccacgcccagcuaauuuuuguacuuuuuguagagaugggguuuugccauguugcccaggcugggauuauuaaguacuuuuuaucauacagcaagauugacauuuuauauuggaauacauuugucucuauauaacggagauuaacaggaaaaugacaagccugggugcgguggcucaugccuguaaucccagcacuuugggaggcugaggugggaggaucacuugaggucaggaguucgagaccaguuuugccaagaugaugaaagcccaugucuacuaaaaauacaaaaauuagcccagcuugauggugggcgccuauaaucccagcuauuugagagacugaggcaggagaaucacuugaaccugggcagcagagguugcagugagccgagaucaugccacugcacuccagccuggguggcauagcgagacucuugucucaagagaaaacaaaacaaaacaaaaaaaaaacaggaaaaugacaaaaaguaauauuacaacucagugaauuuuauaacaaacuuuuuuggaauucauugacuaauacuauaccaaauccaaaauacucucuaguauaccaaauccaacucuacccuauaguauaaauuggauucuauuuggacuugucucacuaaucccucauacaguguguuuuauuuuuuauugaaguaaaaaaauuugucauuuuaaccauuuuuaaguauauaguucaguaauauuaaguauguucauguuguugcgcaauagaucuucggaaguuuuucgucuugcaaccugaaacucuacccauuagcaaauucccauuucuccuuacacuuagcccuugguaaucaucauucuuuuuuuuuuuuuuuugagauggaguuuuacucuuguugcccaggcuggagugcaauggugcaaucucgacucaccacaaccuccgccucccagguucaagcaauucuaccucagccucccgaguagcugggauuacagucaugcaccaccacgcccggcuaauuuuguauuuuuaguagagaagggguuucuccauguugaggcuggucucgaacuccugaccucaggugaucugcccaccucggccucccaaagugcugggauuacaggcgugagccacugcgccuggcccauucuuucuaauucuauaaauuugacuacuuaguuaccuuacauaaauaaauucuuauaguuaguguuauuuuugcuuccaugccuuuuuuguuguuguucaugcucuuacuuggaaugcguucuauuuugucuaccuaugcacauccuguuggguuuuuuuuuuuuuuggggguuuuuuuuguuuuuuuuuguuuuuuuuucccagacaaggucucaauuuguuacccaggcuggagugcagcggcgccaucuccacucacugcauccucaacuuccugggcccaggugauccucucgccucagccccugcagguagcugggacuauaggcaugugccaccaugcccagcuaaauuugguuuuuuuguuuguuuguuuuugagacagagucucacucugucacccaggcuggagugcaguggcacaaucucagcucacugcaaucucugccgcccggguucaagugauucuccugccucagccucccaagcagcugggauuacaggugacugccaccacgccagcuaaguuuuguaguuuuaguagagaugggguuucaccuuguuggccaugcuggucucgaacuccugaccucgugaucugccugcuucugccucccaaagugcuggaauuacaggcaugagccaccacgcccggccagaauuuuuguauuuuuaguagacacaagguucuuacccuguugccuaggcuggucuggaaguccuggacucaagcaauucaccugccuuggccucccaaaaugcugggauuacaagccaccaugcccggccuaaauccuguuguuuuguuuuguuuuauuuuguuuuguuuuguuuuguuuguuuuuugagacagagucucgcuaugucucucaggcuguagugcaguggcgcgaucuuggcucacugccaccucugccucccagguucaagugauucuccugccucagccucccaaguagcugggauuacaggcaugugcuacuauguccggcuaauuuuuguauuuuuaguagagacaggguuucaccauguuggccaggcuggucucgaacuccugaccucgugauccacccaccucggccacccaaagugcugggauuacaggcgugagugguuuuuauuucuuaggccgguuuccuccauaugaucuugcaguagacauuaauuucuuuccuuuuuaauuaaaauacuguuuguauuucacauuuugauguuuguuaagauuuguuuuauauuguuuuuuguuuugucuugugugauagucuuaaaucccuaguuagauaauaacuggagaguaccauguuucuauauaucucucagugacuugcacagugcuagcagauagugcuaaaaaauuauuuauuauuauuauuauuuuguuauuguuguuguuguuguuagacagggucuuccucugucacccaggcuagagggcaaugggaugaucauagcuuacugcagccuccaacaacugggcucauguaauucuccugccucagcuucccaaguagcugggauuacaggcaugagccaccaugucuggacaaaaauauuuccaggugcaguggcucaugccuguaauucccacacuugggaggccgagcgaggcuggaggaucacuugagccuaggaguucaagaccagcuuggcuaagauggcgagaccccgucccuacaaaaaauuuuaaaaacuagccaggcaugguggcaugcaccuauauucccaacuacucagugggcugaggugggagggucauuugaacacaggaauuugaggggagaaaaaaagaagagagaaagagaagugaaggaaggaagaaaggaaggagggagggagagaagaaagaaacgaaagaaaggaaaagaaaaggaaggaaagaaaauugguaccaggaaagcaggaaagggaaauggaaguaaaaaaauaauaauaauaauaaaaugaaaauugguuagucacuauuaacaauuuguauccuuauaaucuggaaacauuauaauuucaaaagaaaaaauauucuuuggaucauagguucugaggucagaacagcauucccguagucuagaugaagucaaguuuuaucugaucuuaauugaaauaaauauagcuggccuugaacaaaucuacucaugguauguggauaggaauuaaauuguaggggcauucacuugauggcauucauucuuagaacauuuaccuaugucuagcuuuuggaguaaagucacauaaccucuaaccagguaaguuuccuguggcuuuauuuaggauuuuaaauacucauuuucaguguaauuuuguuauguguggauuaagaugacucuugguacuaacauacauuuucugauuaaaccuaucugaacaugaguuguuuuuauuucuuacccuuuccagagcgaugauucugacauuugggaugauacagcacugauaaaagcauaugauaaagcuguggcuucauuuaagguaugaaaugcuugcuuagucguuuucuuauuuucucguuauucauuuggaaaggaauugauaacauacgauaaaguguuaaaguacauguuauucaguuuucauuuugaagauuagaugguaguaugaguuaguuaaaucaggugauauccuccuuuagaaguugauagccuauauaugucauccuuuguggaggcaauuuaaauaaaauuuaaaacauuuauuccuggcuggguaugguggcucacuccuguaaucccagcacuuugagaggcugaggcggguggaucaccugaggucaggaguuugagaccagccuggccaacauggugaaaccccgucuuuacuaaaaauacaaaaauuagccaagcaugguggcacgugccuguaaucccagcugcuugggacacugaggcaggagaauugcuugaaccuggggggcagagguugcaaugauugcaccacugcacuccagccugggcgauagagugagacuccaucucagaaaacgaacaaacaauguauuccuuuuaguauuuuuacauuguaucaaacuauggaaguccucuaauugagauuaauaagaaaaagacaaucugaauuauaauuuuaaacauuuaacaagcauguaguaaaauaaugaugaagauaaauagcauuaguacagcaauuaauauuuguagcaugcugacagugcucugugugcguuucauauauuaaauuacucuaaucaucccaaauccuguaaguuggguaucaauucaaguguuccuauuggguaggaauauacaguucuuuuaggaaauguaguaugguucugugucucaaacaggacacuuacacaguuggccaacaucaucaccuucuccauucucugagauguuuagucuuacugagcacuaaauaugggucaucaauaguccagacuaccuugagcaaacaauaguccagacuaccuugagcaaacagagcauauacucauacaguguauaaagagcaccaagcauacagauuucaugucuuucucauaguuacucuuguaacaugagcuaaagaucagaccucuaugucaccuuuguaacugauuucuagauuuuuuuuuuuuuuugagauggggucuugcccugucacccaggcuggaguguaguggcgugaucaugccucauuggagccuucaacucaugagcucaaacaauccuccuaccucagcuuccugaguaguugggaccacaggugugugccaccacacccagcucauuuuuguauucuuuguagagaugcagucucacccuguugcccacgcuggccuggaacuccugagcucaaaagaucccuccgccuugaccuuccaaagugcugggauuacaagcaugaaccacugcacccggccuagauuuuuaaaugugcuuuccaguauacacugaaacuagaagucgacuaaagaauuaccaagagaauucuauaaaauagagauugaaauggggcucgaugugggauggguuggugauauugcagggagaaguaaucugaguaaaggaggaaaagaacugauuugggaaaacgauaguuuuaguagugaguuugaguaugaauuaaguugagauugaauuugaauuaaguugagguugaauaugaauuaaguugagguugaguuugagguaugaauuaagaugugaaauugaucauuggaaauguuagauugagaaaagucacagcuggauuaauagcuucagaaguguguuugcagacaguugcaacuaaaguaauaagaauagauggccuuggccgggcgcgguggcucacgccuguaaucccaguacuuugggaggcugaggcgagcaaaucacgaggucaggaguucaagaccagccuggcccacauggugaaaccccgucuuuauuaaaaauacaaaaauuagcugugcacaguggugcacgccuguaaucccagcuacucgggaggcugagacaggagaaucgcuugaaccugggagguggagguugcagugagcugagaucagugugacugcacuccagcccggugacagagugagacucuguguaaaaaaauaaaauaaauaaaauaauggccguaagcaaguaaagaaggauggccagcucuuauugggaaugccuaaaucuaaggcuugaucagaaguaaugaaaccguuggggcccuacauugcuaugacauccaaagggccaugaauaucaggaagaaagauaauuaacagggucuaauguuacagagagguugagagcaaggagauuugauuaaaagggucuuuagagcugaugucagguguaugaugccuuuaagagcaguuuuuauagugcaggggguggucaaaagagaaaauaggugcuuucugaggugacggagccuugagacuagcuuauaguaguaacuggguuaugucgugacuuuuauucugugcaccacccuguaacauguacauuuuuauuccuauuuucguagcaugcucuaaagaauggugacauuugugaaacuucggguaaaccaaaaaccacaccuaaaagaaaaccugcuaagaagaauaaaagccaaaagaagaauacugcagcuuccuuacaacagguuauuuuaaaauguugagauuuaacuucaaaggaugucucauuaguccuuauuuaauaguguaaaaugucuuuaacuuaagugauuaguacaguguuucuauugacauauacuuauacaacuucaaaaacaacuauuaaauuuucuguuauuuaggaacaugcauauuagucaugaaaguauaaagaauuagaugggaaugauaaaugcuaaaaucaggacauguguuccauuugugaauggaaggcagggagaaggugccguuuggaaggaguacccaagagccguaagcugaauuggcaguguuuuacaucuuaagcugagagauagauuuuuuuuuccccuuuuucuuuaaaaacucuaaaacuguuaauuccaaggaacccagaagucuagguagauuauuucugcuaguuaaaagcaguaguccugaaagcugaauauuuuggugucuuuugagccaacuuuaguuucaucauuaccaagggggaagagagcuaacaguugaugagcacuugcucuaggccaguccagagugcugggcaccauacgcauuuuaucucccucccgcuauucacaacaaauaugggagguaguuuauauuauagccaucuaauaagauggggaaacuaagacucaaagagauucagaaacuuguccaugauuauaaauguaagagaguuggaauucagauuuauguauuuagaccccaagccuuucucauuacaucauuuugccuuccaaaucucuacccucuauccuucaccuccccacugaucaaaacgagaugauaguuugcccucuucaaaagaaaugugugcauguauauaucuuugauuucuuuuguaguggaaaguuggggacaaauguucugccauuuggucagaagacgguugcauuuacccagcuaccauugcuucaauugauuuuaagagagaaaccuguguugugguuuacacuggauauggaaauagagaggagcaaaaucuguccgaucuacuuuccccaaucugugaaguagcuaauaauauagaacaaaaugcucaagagguaaggauacaaaaaaaaaaaaauucaauuucuggaagcagagacuagaugagaaacuguuaaacaguauacacaguugucaguuugauccaccgaggcauuaauuuuuucuuaaucacacccuuauaacaaaaaccugcauauuuuuucuuuuuaaagaaugaaaaugaaagccaaguuucaacagaugaaagugagaacuccaggucuccuggaaauaaaucagauaacaucaagcccaaaucugcuccauggaacucuuuucucccuccaccaccccccaugccagggccaagacugggaccaggaaagguaaaccuucuaugaaaguuuuccagaaaauaguuaaugucgggacauuuaaccucucuguuaacuaauuuguagcucucccaugaaacuuuuguagcuuaaauacacaagaauuuuuugaaaaggaaauaagauaaugaugcaaaauaguuaauuuuuuaaaaaaauguuagacacugcaguggaugcaacaaaauacuuuauaugaaagauuuauccaguuaacuuuuguggaguauuagguauuagacuaauaauuagcacacuuacuuaaguuagaaaguauaauaaugcgccggacgcgguagcucacgccuguaaucccagcacuuugggaggccaaggugggcggaucacaaggucaggagaucgagaccauccuggcuaacacggugaaaccccaucucuacugaaaauacaaaaaaauuugccgggcgugauggcgggcaccuguagucccagcuacucgggaggcugaggcaggaggauggugugaaccccggaggcagagcuugcagugagucaagaucgugccacugcacuccaaccugggcgacagaaugagacuccaucucaaacaaaaaaacaaaacaaaacaaaaaaaaguguaauaauaauuuaucauuagcuggaugauaugcuguuguuucccaugucaccuguauaagauauguaaaauaagaacacauuauuuacaucuaauauagauaaaauccugaggcgcucucagauuguuuuguagaguucaaauguaaauauuguuuucauuuaugguccuuuugguuauaaguaacagaaaucaacucuaaaaagauuuuuauuauagguuagauuaugucauggaaccuuaaggcuugucccuuucuaguucuuuuguguaaagcggugauuucuuccauggagggaaugguauuuaggcaauuuuuuuuuuuuuuucgagauggagucuugcucugucgcucaggcuggagugcaguggcaccauuucagcucacugcaacuuccaccuccuggguucaagugauucuccugcuucagccucccaaguagcugagauuacaggcacccgccaccacacccggcuuauuuuguauuuuuaguagagaugggguuucaccauguuggccaggcuggucuugaacuccugaccucaagugaucuccccaccuuggccuuccaaagugcuaggauuacaggcgccuagccuaggcagucauuuucaaaaaacaagcaugacucaccaaaaguuuuaagauuuucugugauaauguucuuauugaggcuuacauuauauuacaguuucuugaaucuaaaaugauguacccucuuagaauauauacaucaugcuucauuggucucagggggcugauuuuuauaaggagagauuugcuaguuuucacaauauguccucuaaguuggcauguauagcuaaacaggcuuucauaaaaauauacaauuuaguuaaugaaauuugggauauagucuuuuaugauugaaauaauuuugcuaaauagacugucucugauuuauuagguaaucaccacucuuauuuuguuuuacuuccuuaaugucuacauagaaaggaaaugagaaaaauccagagguugucauuugacuuaugagucuguuugacuucaggauuugguacaugaaauuucacuuaaucuuuuugauauguauaaaacaaauauucuggguaauuauuuuuauccuuuugguuuugaguccuuuuuauuccuaucauauugaaauugguaaguuaauuuuccuuugaaauauuccuuauagccaggucuaaaauucaauggcccaccaccgccaccgccaccaccaccaccccacuuacuaucaugcuggcugccuccauuuccuucuggaccaccaguaaguaaaaaagaguauagguuagauuuugcuuucacauacaauuugauaauuagcagaauagaggauuguaaaaugucauuguagaacaucccuugggccagauucuaauggguagaaauuugaacuaaaccucuggguuuuguuuguuuuuaaugccuuucuguuacccagaugcagugcucuuguagucccaagucuaagcucuagguugccuucuuuccuggcagaaguuggugucuaugccauaaggagguaguuccuguuagaagggauuuaauuauaccuuauauaaggaauuaguguuugcccuucuagguauaguuggauguuagcuucugauguaaacuggauuucuuuuucuuucucucucuuuuuuuuuuuuuguuuuggaggcagaguuuugcccuuguaccccaggcuggagugcaguggugugaucucagcucacagcaaccuccgccuccuggguucaagcaauucugccucggccucccaaguagcugggauuacaggcgacugccaccacacccggcuaauuuuuguuuuauuaguagagaugggguuucaccauguuggccagacugaucuugaacuccugaccucaggugauccacccgccuuggccucccaaagcgcugggauuacaggcgugagcugccgcacccagcuguaaacuggauuucuaaugguagauuuuuagguauuaacaauagauaaaaagauacuuuuuggcauacuguguauugggaugggguuagaacagguguucuacccaagacauuuacuuaaaaucgcccucgaaaugcuaugugagcuguguguguguguguguguguguguguauuaaggaaaagcaugaaaguauuuaugcuugauuuuuuuuuuuuacucauagcuucauaguggaacagauacauagucuaaaucaaaauguuuaaacuuuuuaugucacuugcugucuuuucguccucguuaaauuuaauuuuguuggucuuuuguuguuauugguugguuuucuccaaaugcuagcuauguuaagaaauuuaaggccagguacaguggcucaugccuguaaucccggcauuuuagaaggcugaggcaggaggaucacuugagcucaggaguuugagaccagucugggcaacauagcaagaccucgucuuuguuuaggggaaaaaaaagaaauuuaaguaggagauuauauaagcaaaaauacaauuaauuuccagcauucacuauauaauauaaaucuccagacuuuacuuuuuuguuuacuggauauaaacaauaucuuuuucugucuccagauaauucccccaccaccucccauauguccagauucucuugaugaugcugaugcuuugggaaguauguuaauuucaugguacaugaguggcuaucauacuggcuauuauaugguaaguaaucacucagcaucuuuuccugacaauuuuuuuguaguuaugugacuuuguuuuguaaauuuauaaaauacuacuugcuucucucuuuauauuacuaaaaaauaaaaauaaaaaaauacaacugucugaggcuuaaauuacucuugcauugucccuaaguauaauuuuaguuaauuuuaaaaagcuuucaugcuauuguuagauuauuuugauuauacacuuuugaauugaaauuauacuuuuucuaaauaauguuuuaaucucugauuugaaauugauuguagggaauggaaaagaugggauaauuuuucauaaaugaaaaaugaaauucuuuuuuuuuuuuuuuuuuuuuugagacggagucuugcucuguugcccaggcuggagugcaauggcgugaucuuggcucacagcaagcucugccuccuggauucacgccauucuccugccucagccucagagguagcugggacuacaggugccugccaccacgccugucuaauuuuuuguauuuuuuuguaaagacaggguuucacuguguuagccaggauggucucaaucuccugaccccgugauccacccgccucggccuuccaagagaaaugaaauuuuuuuaaugcacaaagaucugggguaauguguaccacauugaaccuuggggaguauggcuucaaacuugucacuuuauacguuagucuccuacggacauguucuauuguauuuuagucagaacauuuaaaauuauuuuauuuuauuuuauuuuuuuuuuuuuuuugagacggagucucgcucugucacccaggcuggaguacaguggcgcagucucggcucacugcaagcuccgccucccggguucacgccauucuccugccucagccucuccgaguagcugggacuacaggcgcccgccaccacgcccggcuaauuuuuuuuuauuuuuaguagagacgggguuucaccguggucucgaucuccugaccucgugauccacccgccucggccucccaaagugcugggauuacaagcgugagccaccgcgcccggccuaaaauuauuuuuaaaaguaagcucuugugcccugcuaaaauuaugaugugauauuguaggcacuuguauuuuuaguaaauuaauauagaagaaacaacugacuuaaagguguauguuuuuaaauguaucaucugugugugcccccauuaauauucuuauuuaaaaguuaaggccagacaugguggcuuacaacuguaaucccaacaguuugugaggccgaggcaggcagaucacuugaggucaggaguuugagaccagccuggccaacaugaugaaaccuugucucuacuaaaaauaccaaaaaaaauuuagccaggcaugguggcacaugccuguaauccgagcuacuugggaggcuguggcaggaaaauugcuuuaaucugggaggcagagguugcagugaguugagauugugccacugcacuccacccuuggugacagagugagauuccaucucaaaaaaagaaaaaggccuggcacgguggcucacaccuauaaucccaguacuuugggagguagaggcagguggaucacuugagguuaggaguucaggaccagccuggccaacauggugacuacuccauuucuacuaaauacacaaaacuuagcccaguggcgggcaguuguaaucccagcuacuugagagguugaggcaggagaaucacuugaaccugggaggcagagguugcagugagccgagaucacaccgcugcacucuagccuggccaacagagugagaauuugcggagggaaaaaaaagucacgcuucaguuguuguaguauaaccuugguauauuguauguaucaugaauuccucauuuuaaugaccaaaaaguaauaaaucaacagcuuguaauuuguuuugagaucaguuaucugacuguaacacuguaggcuuuuguguuuuuuaaauuaugaaauauuugaaaaaaauacauaauguauauauaaaguauugguauaauuuauguucuaaauaacuuucuugagaaauaauucacauggugugcaguuuaccuuugaaaguauacaaguuggcugggcacaauggcucacgccuguaaucccagcacuuugggaggccagggcagguggaucacgaggucaggagaucgagaccauccuggcuaacauggugaaaccccgucucuacuaaaaguacaaaaacaaauuagccgggcauguuggcgggcaccuuuugucccagcugcucgggaggcugaggcaggagaguggcgugaacccaggagguggagcuugcagugagccgagauugugccagugcacuccagccugggcgacagagcgagacucugucucaaaaaauaaaauaaaaaagaaaguauacaagucagugguuuugguuuucaguuaugcaaccaucacuacaauuuaagaacauuuucaucaccccaaaaagaaacccuguuaccuucauuuuccccagcccuaggcagucaguacacuuucugucucuaugaauuugucuauuuuagauauuauauauaaacggaauuauacgauauguggucuuuugugucuggcuucuuucacuuagcaugcuauuuucaagauucauccaugcuguagaaugcaccaguacugcauuccuucuuauugcugaauauucuguuguuugguuauaucacauuuuauccauucaucaguucauggacauuuagguuguuuuuauuuuugggcuauaaugaauaauguugcuaugaacauucguuuguguucuuuuuguuuuuuugguuuuuuggguuuuuuuuguuuuguuuuuguuuuugagacagucuugcucugucuccuaagcuggagugcaguggcaugaucuuggcuuacugcaagcucugccucccggguucacaccauucuccugccucagcccgacaaguagcugggacuacaggcgugugccaccaugcacggcuaauuuuuuguauuuuuaguagagaugggguuucaccguguuagccaggauggucucgaucuccugaccucgugaucugccugccuaggccucccaaagugcugggauuacaggcgugagccacugcaccuggccuuaaguguuuuuaauacgucauugccuuaagcuaacaauucuuaaccuuuguucuacugaagccacgugguugagauaggcucugagucuagcuuuuaaccucuaucuuuuugucuuagaaaucuaagcagaaugcaaaugacuaagaauaauguuguugaaauaacauaaaauagguuauaacuuugauacucauuaguaacaaaucuuucaauacaucuuacggucuguuagguguagauuaguaaugaagugggaagccacugcaagcuaguauacauguagggaaagauagaaagcauugaagccagaagagagacagaggacauuugggcuagaucugacaagaaaaacaaauguuuuaguauuaauuuuugacuuuaaauuuuuuuuuuauuuagugaauacugguguuuaauggucucauuuuaauaaguaugacacagguaguuuaaggucauauauuuuauuugaugaaaauaagguauaggccgggcacgguggcucacaccuguaaucccagcacuuugggaggccgaggcaggcggaucaccugaggucgggaguuagagacuagccucaacauggagaaaccccgucucuacuaaaaaaaauacaaaauuaggcgggcgugguggugcaugccuguaaucccagcuacucaggaggcugaggcaggagaauugcuugaaccugggagguggagguugcggugagccgagaucaccucauugcacuccagccugggcaacaagagcaaaacuccaucucaaaaaaaaaaaaauaagguauaagcgggcucaggaacaucauuggacauacugaaagaagaaaaaucagcugggcgcaguggcucacgccgguaaucccaacacuuugggaggccaaggcaggcgaaucaccugaagucgggaguuccagaucagccugaccaacauggagaaacccugucucuacuaaaaauacaaaacuagccgggcaugguggcgcaugccuguaaucccagcuacuugggaggcugaggcaggagaauugcuugaaccgagaaggcggagguugcggugagccaagauugcaccauugcacuccagccugggcaacaagagcgaaacuccgucucaaaaaaaaaaggaagaaaaauauuuuuuuaaauuaauuaguuuauuuauuuuuuaagauggaguuuugcccugucacccaggcuggggugcaauggugcaaucucggcucacugcaaccuccgccuccuggguucaagugauucuccugccucagcuucccgaguagcugugauuacagccauaugccaccacgcccagccaguuuuguguuuuguuuuguuuuuuguuuuuuuuuuuugagagggugucuugcucugucccccaagcuggagugcagcggcgcgaucuuggcucacugcaagcucugccucccagguucacaccauucucuugccucagccucccgaguagcugggacuacaggugcccgccaccacacccggcuaauuuuuuuguguuuuuaguagagaugggguuucacuguguuagccaggauggucucgaucuccugaccuuuugauccacccgccucagccuccccaagugcugggauuauaggcgugagccacugugcccggccuagucuuguauuuuuaguagagucgggauuucuccauguuggucaggcuguucuccaaauccgaccucaggugauccgcccgccuuggccuccaaaagugcaaggcaaggcauuacaggcaugagccacugugaccggcaauguuuuuaaauuuuuuacauuuaaauuuuauuuuuuagagaccaggucucacucuauugcucaggcuggagugcaagggcacauucacagcucacugcagccuugaccuccagggcucaagcaguccucucaccucaguuucccgaguagcugggacuacagugauaaugccacugcaccuggcuaauuuuuauuuuuauuuauuuauuuuuuuuugagacagagucuugcucugucacccaggcuggagugcagugguguaaaucucagcucacugcagccuccgccuccuggguucaagugauucuccugccucaaccucccaaguagcugggauuagagguccccaccaccaugccuggcuaauuuuuuguacuuucaguagaaacgggguuuugccauguuggccaggcuguucucgaacuccugagcucaggugauccaacugucucggccucccaaagugcugggauuacaggcgugagccacugugccuagccugagccaccacgccggccuaauuuuuaaauuuuuuguagagacagggucucauuauguugcccaggguggugucaagcuccaggucucaagugaucccccuaccuccgccucccaaaguugugggauuguaggcaugagccacugcaagaaaaccuuaacugcagccuaauaauuguuuucuuugggauaacuuuuaaaguacauuaaaagacuaucaacuuaauuucugaucauauuuuguugaauaaaauaaguaaaaugucuugugaaacaaaaugcuuuuuaacauccauauaaagcuaucuauauauagcuaucuauaucuauauagcuauuuuuuuuaacuuccuuuauuuuccuuacaggguuuuagacaaaaucaaaaagaaggaaggugcucacauuccuuaaauuaaggaguaagucugccagcauuaugaaagugaaucuuacuuuuguaaaacuuuaugguuuguggaaaacaaauguuuuugaacauuuaaaaaguucagauguuagaaaguugaaagguuaauguaaaacaaucaauauuaaagaauuuugaugccaaaacuauuagauaaaagguuaaucuacaucccuacuagaauucucauacuuaacugguugguuguguggaagaaacauacuuucacaauaaagagcuuuaggauaugaugccauuuuauaucacuaguaggcagaccagcagacuuuuuuuuauugugauaugggauaaccuaggcauacugcacuguacacucugacauaugaagugcucuagucaaguuuaacugguguccacagaggacaugguuuaacuggaauucgucaagccucugguucuaauuucucauuugcaggaaaugcuggcauagagcagcacuaaaugacaccacuaaagaaacgaucagacagaucuggaaugugaagcguuauagaagauaacuggccucauuucuucaaaauaucaaguguugggaaagaaaaaaggaaguggaauggguaacucuucuugauuaaaaguuauguaauaaccaaaugcaaugugaaauauuuuacuggacucuauuuugaaaaaccaucuguaaaagacugaggugggggugggaggccagcacgguggugaggcaguugagaaaauuugaauguggauuagauuuugaaugauauuggauaauuauugguaauuuuaugagcugugagaaggguguuguaguuuauaaaagacugucuuaauuugcauacuuaagcauuuaggaaugaaguguuagagugucuuaaaauguuucaaaugguuuaacaaaauguaugugaggcguauguggcaaaauguuacagaaucuaacugguggacauggcuguucauuguacuguuuuuuucuaucuucuauauguuuaaaaguauauaauaaaaauauuuaauuuuuuuuuaaauua

SEQ ID NO: 65 - DNA, 18 bp, Artificial Sequence
  Synthetic Polynucleotide
  modified_base (1)..(18): 2′-O-(2-methoxyethyl) modification
  misc_feature (1)..(2): phosphorothioate linkage between the indicated positions
  modified_base (2)..(2): 5-methyl C
  modified_base (4)..(4): 5-methyl C
  modified_base (8)..(8): 5-methyl C
  misc_feature (14)..(15): phosphorothioate linkage between the indicated positions
  modified_base (15)..(15): 5-methyl C
  misc_feature (18)..(18): modified by /iSp18//iSp18//3CholTEG/
tcactttcataatgctgg

SEQ ID NO: 66 - DNA, 18 bp, Artificial Sequence
  Synthetic Polynucleotide
  modified_base (1)..(18): 2′-O-(2-methoxyethyl) modification
  misc_feature (1)..(2): phosphorothioate linkage between the indicated positions
  modified_base (2)..(2): 5-methyl C
  modified_base (4)..(4): 5-methyl C
  modified_base (8)..(8): 5-methyl C
  misc_feature (8)..(9): phosphorothioate linkage between the indicated positions
  misc_feature (14)..(15): phosphorothioate linkage between the indicated positions
  modified_base (15)..(15): 5-methyl C
  misc_feature (18)..(18): modified by /iSp18//iSp18//3CholTEG/
tcactttcataatgctgg

SEQ ID NO: 67 - DNA, 18 bp, Artificial Sequence
  Synthetic Polynucleotide
  modified_base (1)..(18): 2′-O-(2-methoxyethyl) modification
  misc_feature (1)..(2): phosphorothioate linkage between the indicated positions
  modified_base (2)..(2): 5-methyl C
  modified_base (4)..(4): 5-methyl C
  modified_base (8)..(8): 5-methyl C
  misc_feature (8)..(10): phosphorothioate linkage between the indicated positions
  misc_feature (13)..(15): phosphorothioate linkage between the indicated positions
  modified_base (15)..(15): 5-methyl C
  misc_feature (18)..(18): modified by /iSp18//iSp18//3CholTEG/
tcactttcataatgctgg

SEQ ID NO: 68 - DNA, 18 bp, Artificial Sequence
  Synthetic Polynucleotide
  modified_base (1)..(18): 2′-O-(2-methoxyethyl) modification
  modified_base (2)..(2): 5-methyl C
  modified_base (4)..(4): 5-methyl C
  modified_base (8)..(8): 5-methyl C
  misc_feature (8)..(14): phosphorothioate linkage between the indicated positions
  modified_base (15)..(15): 5-methyl C
  misc_feature (18)..(18): modified by /iSp18//iSp18//3CholTEG/
tcactttcataatgctgg

SEQ ID NO: 69 - DNA, 18 bp, Artificial Sequence
  Synthetic Polynucleotide
  misc_feature (1)..(1): modified by /5CholTEG//iSp18//iSp18/
  modified_base (1)..(18): 2′-O-(2-methoxyethyl) modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  misc_feature (8)..(10): phosphorothioate linkage between the indicated positions
  misc_feature (13)..(15): phosphorothioate linkage between the indicated positions
  modified_base (15)..(15): 5-methyl C
tcactttcataatgctgg

SEQ ID NO: 70 - RNA, 18 nt, Artificial Sequence
  Synthetic Polynucleotide
  modified_base (1)..(18): 2-O-methyl modification
  misc_feature (1)..(18): phosphorothioate linkage between the indicated positions
  misc_feature (18)..(18): modified by /iSp18//iSp18//3CholTEG/
ucacuuucauaaugcugg

SEQ ID NO: 71 - RNA, 18 nt, Artificial Sequence
  Synthetic Polynucleotide
  modified_base (1)..(18): 2-O-methyl modification
  misc_feature (18)..(18): modified by /iSp18//iSp18//3CholTEG/
ucacuuucauaaugcugg

SEQ ID NO: 72 - DNA, 18 bp, Artificial Sequence
  Synthetic Polynucleotide
  modified_base (1)..(18): 2-O-methyl modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3CholTEG/
tcactttcataatgctgg

SEQ ID NO: 73 - DNA, 18 bp, Artificial Sequence
  Synthetic Polynucleotide
  modified_base (1)..(18): 2-O-methyl modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
tcactttcataatgctgg

SEQ ID NO: 74 - RNA, 18 nt, Artificial Sequence
  Synthetic Polynucleotide
  modified_base (1)..(18): 2-O-methyl modification
ucacuuucauaaugcugg

SEQ ID NO: 75 - DNA, 18 bp, Artificial Sequence
  Synthetic Polynucleotide
  misc_feature (18)..(18): modified by CholEG
tcactttcataatgctgg

SEQ ID NO: 76 - DNA, 18 bp, Artificial Sequence
  Synthetic Polynucleotide
tcactttcataatgctgg

SEQ ID NO: 77 - DNA, 18 bp, Artificial Sequence
  Synthetic Polynucleotide
  modified_base (1)..(18): 2-O, 4′-C-ethylene-bridged sugar modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3CholTEG/
tcactttcataatgctgg

SEQ ID NO: 78 - DNA, 18 bp, Artificial Sequence
  Synthetic Polynucleotide
  modified_base (1)..(18): 2-O, 4′-C-ethylene-bridged sugar modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
tcactttcataatgctgg

SEQ ID NO: 79 - DNA, 18 bp, Artificial Sequence
  Synthetic Polynucleotide
  modified_base (1)..(18): LNA sugar modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3CholTEG/
tcactttcataatgctgg

SEQ ID NO: 80 - DNA, 18 bp, Artificial Sequence
  Synthetic Polynucleotide
  modified_base (1)..(18): LNA sugar modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
tcactttcataatgctgg

SEQ ID NO: 81 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtactacacttttaattact

SEQ ID NO: 82 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tgtatattgatgtcagtact

SEQ ID NO: 83 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tacattgtctattagtgtat

SEQ ID NO: 84 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tgactctcaattctgttaca

SEQ ID NO: 85 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tcacagggctatttctgact

SEQ ID NO: 86 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aatcagtcacatatatcaca

SEQ ID NO: 87 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tgtaactttagttaaaatca

SEQ ID NO: 88 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ctattaaaccacatttgtaa

SEQ ID NO: 89 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
actactatgctttctctatt

SEQ ID NO: 90 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ccaccatttcttgaaactac

SEQ ID NO: 91 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tttccaatagttttaccacc

SEQ ID NO: 92 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtttttgcataaggatttcc

SEQ ID NO: 93 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtggaaatttggtttgtttt

SEQ ID NO: 94 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tgtggctcagtgtaggtgga

SEQ ID NO: 95 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtattaattcttatatgtgg

SEQ ID NO: 96 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ttagttttacacttaggtct

SEQ ID NO: 97 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
acacagtttagagttttagt

SEQ ID NO: 98 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gatcacagatttttctctct

SEQ ID NO: 99 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ttataggcaatccatgatca

SEQ ID NO: 100 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
catttcagtttgttcttttg

SEQ ID NO: 101 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
cccaggcaacaaggccattt

SEQ ID NO: 102 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gaacctcgggtgccacccca

SEQ ID NO: 103 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
cgtccttgatttcctcagcg

SEQ ID NO: 104 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
acacccttggtgtgtcagcg

SEQ ID NO: 105 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ttctgctctagcctcacacc

SEQ ID NO: 106 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ggagagagctagtctctttc

SEQ ID NO: 107 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aggacctctctctgcaggag

SEQ ID NO: 108 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
atgggaactcttttcaggac

SEQ ID NO: 109 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gcatttcactgtggaatggg

SEQ ID NO: 110 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tttataaaaatgcttgcatt

SEQ ID NO: 111 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
cttcccattagctcatttat

SEQ ID NO: 112 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tagataagctacccccttcc

SEQ ID NO: 113 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tttgctccctatgtgtagat

SEQ ID NO: 114 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ggtcctaactggttttttgc

SEQ ID NO: 115 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
cagatggcaacacctggtcc

SEQ ID NO: 116 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gattcacgctctgtgcagat

SEQ ID NO: 117 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gcctgcataataaaaggttg

SEQ ID NO: 118 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tcaggccaaggacctgcctg

SEQ ID NO: 119 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
taagcaatgtggagtagctc

SEQ ID NO: 120 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
acaataggaaagagataagc

SEQ ID NO: 121 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ttatttagcacatgcacaat

SEQ ID NO: 122 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tggctccacctccccttatt

SEQ ID NO: 123 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gcatgtccaccatggtggct

SEQ ID NO: 124 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
agctgcacggagagaaaggg

SEQ ID NO: 125 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gcatgttgtgagttgttggg

SEQ ID NO: 126 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tcagataaggaagctggaag

SEQ ID NO: 127 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gaccttagtacatactcaga

SEQ ID NO: 128 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gaagtaaacacagtggacct

SEQ ID NO: 129 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtatgtgaagtaaacacagt

SEQ ID NO: 130 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtaaacacagtatgtgaagt

SEQ ID NO: 131 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aggtgggtatgtgaagtaaa

SEQ ID NO: 132 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
atcagcaagcttcacatacg

SEQ ID NO: 133 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ggagcttcctgggtaatcag

SEQ ID NO: 134 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
agcagctctggcacagaggg

SEQ ID NO: 135 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aaacatgtataaggaagcag

SEQ ID NO: 136 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ggaagatcgggctgtaaaca

SEQ ID NO: 137 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
acttctcttctaacaaggag

SEQ ID NO: 138 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
cagagtcctcggtagaactt

SEQ ID NO: 139 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aagccgatagttagacagag

SEQ ID NO: 140 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aaaaaaagactaggtaagcc

SEQ ID NO: 141 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gttttgagagaggaggtaaa

SEQ ID NO: 142 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gttttttctttgatggtttt

SEQ ID NO: 143 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gaaatctaatttttcagttt

SEQ ID NO: 144 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aatcttaattttgctgaaat

SEQ ID NO: 145 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tttttaagaacagaaaatct

SEQ ID NO: 146 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
acactttggtttttcatttt

SEQ ID NO: 147 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
attttctcccggtttacact

SEQ ID NO: 148 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aggtaacttgcatgtatttt

SEQ ID NO: 149 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
aatatctttatcagataggt

SEQ ID NO: 150 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
atgtttgctgggtacaatat

SEQ ID NO: 151 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gtttgagagttcttcatgtt

SEQ ID NO: 152 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
catcttttaattgaattttt

SEQ ID NO: 153 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
cccggccaacttacccatct

SEQ ID NO: 154 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gattgggattgcaagtatga

SEQ ID NO: 155 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
gagcacacgccacaatgcct

SEQ ID NO: 156 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
agtcttcttgtctcagcctt

SEQ ID NO: 157 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
ccacctcctgcgctcagtct

SEQ ID NO: 158 - DNA, 20 bp, Artificial Sequence
  Synthetic Polynucleotide
  misc_feature (1)..(20): phosphorothioate linkage between the indicated positions
  misc_feature (20)..(20): modified by /iSp18//iSp18//3CholTEG/
tcacacagcctactgcagcc

SEQ ID NO: 159 - DNA, 20 bp, Artificial Sequence
  Synthetic Polynucleotide
  misc_feature (1)..(20): phosphorothioate linkage between the indicated positions
  misc_feature (2)..(6): phosphodiester internucleotide linkage between the indicated positions
  misc_feature (16)..(18): phosphodiester internucleotide linkage between the indicated positions
  misc_feature (20)..(20): modified by /iSp18//iSp18//3CholTEG/
cagagtcctcggtagaactt

SEQ ID NO: 160 - DNA, 20 bp, Artificial Sequence
  Synthetic Polynucleotide
  misc_feature (1)..(20): phosphorothioate linkage between the indicated positions
  misc_feature (2)..(6): phosphodiester internucleotide linkage between the indicated positions
  misc_feature (16)..(18): phosphodiester internucleotide linkage between the indicated positions
  misc_feature (20)..(20): modified by /iSp18//iSp18//3CholTEG/
gaaatctaatttttcagttt

SEQ ID NO: 161 - DNA, 1613 bp, Artificial Sequence
  Synthetic Polynucleotide
agtaattaaaagtgtagtactgacatcaatatacactaatagacaatgtaacagaattgagagtcagaaatagccctgtgatatatgtgactgattttaactaaagttacaaatgtggtttaatagagaaagcatagtagtttcaagaaatggtggtaaaactattggaaatccttatgcaaaaacaaaccaaatttccacctacactgagccacatataagaattaatacaaaatggattacagacctaagtgtaaaactaaaactctaaactgtgtaaaaaaaaaaaagagagaaaaatctgtgatcatggattgcctataacaaaagaacaaactgaaatggccttgttgcctggggtggcacccgaggttcttggtctcacgctgaggaaatcaaggacgctgacacaccaagggtgtgaggctagagcagaagtttaataggcaaaagaaagagactagctctctcctgcagagagaggtcctgaaaagagttcccattccacagtgaaatgcaagcatttttataaatgagctaatgggaagggggtagcttatctacacatagggagcaaaaaaccagttaggaccaggtgttgccatctgcacagagcgtgaatctctggcatcccccaccccaaccttttattatgcaggcaggtccttggcctgagctactccacattgcttatctctttcctattgtgcatgtgctaaataaggggaggtggagccaccatggtggacatgcctggccccaggtacccctttctctccgtgcagctgcaggcaaccccaacaactcacaacatgcaagcttccagcttccttatctgagtatgtactaaggtccactgtgtttacttcacatactgtgtttacttcacatacccaccttacgtatgtgaagcttgctgattacccaggaagctccccctctgtgccagagctgcttccttatacatgtttacagcccgatcttccaggctgctccttgttagaagagaagtgatttcttgggctgctttttgttagaagggaagttctaccgaggactctgtctaactatcggcttacctagtctttttttacctcctctctcaaaaccatcaaagaaaaaactgaaaaattagatttcagcaaaattaagattttctgttcttaaaaagacgttgttaacaaaatgaaaaaccaaagtgtaaaccgggagaaaatacatgcaagttacctatctgataaagatattgtacccagcaaacatgaagaactctcaaacctcaacaacaaaaaaattcaattaaaagatgggtaagttggccgggtgcagtggctcatacttgcaatcccaatctttgggaggctgaggcaggaagattgcttgagcccaggagttcacgacaagcccaggcaacataatgagaccttgtttctacaaaattttaaaaaattagccaggcattgtggcgtgtgctcgtaatttcagctactcagaaggctgagacaagaagactgagcgcaggaggtggaggctgcagtaggctgtgtgattgcaccactgcacaacagcctgggtgacagagtgagacactgtctccaaaaaaaaaaaaaaaa

SEQ ID NO: 162 - DNA, 18 bp, Artificial Sequence
  Synthetic Polynucleotide
  misc_feature (1)..(1): modified by 5AmMC6
  modified_base (1)..(18): 2′-O-(2-methoxyethyl) modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
tcactttcataatgctgg

SEQ ID NO: 163 - DNA, 18 bp, Artificial Sequence
  Synthetic Polynucleotide
  misc_feature (1)..(1): modified by 5AmMC6
  modified_base (1)..(18): 2′-O-(2-methoxyethyl) modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3CholTEG/
tcactttcataatgctgg

SEQ ID NO: 164 - DNA, 18 bp, Artificial Sequence
  Synthetic Polynucleotide
  modified_base (1)..(18): 2′-O-(2-methoxyethyl) modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3CholTEG/
tcactttcataatgctgg

SEQ ID NO: 165 - DNA, 15 bp, Artificial Sequence
  Synthetic Polynucleotide
  modified_base (1)..(15): 2′-O-(2-methoxyethyl) modification
  misc_feature (1)..(15): phosphorothioate linkage between the indicated positions
  modified_base (6)..(6): 5-methyl C
  modified_base (8)..(8): 5-methyl C
  misc_feature (15)..(15): modified by /iSp18//iSp18//3CholTEG/
tgtatctcattgtag

SEQ ID NO: 166 - DNA, 18 bp, Artificial Sequence
  Synthetic Polynucleotide
  modified_base (1)..(18): 2′-O-(2-methoxyethyl) modification
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//branch//STA/
tcactttcataatgctgg

SEQ ID NO: 167 - DNA, 15 bp, Artificial Sequence
  Synthetic Polynucleotide
  modified_base (1)..(15): 2′-O-(2-methoxyethyl) modification
  misc_feature (1)..(15): phosphorothioate linkage between the indicated positions
  modified_base (6)..(6): 5-methyl C
  modified_base (8)..(8): 5-methyl C
  misc_feature (15)..(15): modified by /iSp18//iSp18//branch//STA/
tgtatctcattgtag

SEQ ID NO: 168 - DNA, 18 bp, Artificial Sequence
  Synthetic Polynucleotide
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3ThioMC6-D/
tcactttcataatgctgg

SEQ ID NO: 169 - DNA, 15 bp, Artificial Sequence
  Synthetic Polynucleotide
  modified_base (1)..(15): 2′-O-(2-methoxyethyl) modification
  misc_feature (1)..(15): phosphorothioate linkage between the indicated positions
  modified_base (6)..(6): 5-methyl C
  modified_base (8)..(8): 5-methyl C
  misc_feature (15)..(15): modified by /iSp18//iSp18//3ThioMC6-D/
tgtatctcattgtag

SEQ ID NO: 170 - DNA, 18 bp, Artificial Sequence
  Synthetic Polynucleotide
  misc_feature (1)..(18): phosphorothioate linkage between the indicated positions
  modified_base (2)..(2): 5-methyl C
  modified_base (4)..(4): 5-methyl C
  modified_base (8)..(8): 5-methyl C
  modified_base (15)..(15): 5-methyl C
  misc_feature (18)..(18): modified by /iSp18//iSp18//3SerinolDTPA/
tcactttcataatgctgg

SEQ ID NO: 171 - DNA, 15 bp, Artificial Sequence
  Synthetic Polynucleotide
  modified_base (1)..(15): 2′-O-(2-methoxyethyl) modification
  misc_feature (1)..(15): phosphorothioate linkage between the indicated positions
  modified_base (6)..(6): 5-methyl C
  modified_base (8)..(8): 5-methyl C
  misc_feature (15)..(15): modified by /iSp18//iSp18//3SerinolDTPA/
tgtatctcattgtag

SEQ ID NO: 172 - DNA, 18 bp, Artificial Sequence
  Synthetic Polynucleotide
tcactttcataatgctgg

SEQ ID NO: 173 - RNA, 18 nt, Artificial Sequence
  Synthetic Polynucleotide
ucacuuucauaaugcugg